SEQUENCE LISTING (397 sequences)

SEQ ID NO: 1 - protein, 242 aa, synthetic construct
QEECVCENYKLAVNCFVNNNRQCQCTSVGAQNTVICSKLAAKCLVMKAEMNGSKLGRRAKPEGALQNNDGLYDPDCDESGLFKAKQCNGTSMCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLK

SEQ ID NO: 2 - protein, 36 aa, Homo sapiens
QEECVCENYKLAVNCFVNNNRQCQCTSVGAQNTVIC

SEQ ID NO: 3 - protein, 71 aa, unidentified
CLVMKAEMNGSKLGRRAKPEGALQNNDGLYDAADCDESGLFKAKQCNGTSMCWCVNTAGVRRTDKDTEITC

SEQ ID NO: 4 - protein, 130 aa, unidentified
SERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLK

SEQ ID NO: 5 - skipped

SEQ ID NO: 6 - protein, 5 aa, synthetic construct
  VARIANT 3: note = Y, N, F, S, H, D, L, I, or W
NYXIH

SEQ ID NO: 7 - protein, 17 aa, synthetic construct
  VARIANT 2: note = I or F
  VARIANT 3: note = Y or N
  VARIANT 6: note = N or D
  VARIANT 12: note = N or S
  VARIANT 13: note = E or Q
  VARIANT 16: note = K or Q
WXXPGXVYIQYXXKFXG

SEQ ID NO: 8 - protein, 7 aa, synthetic construct
  VARIANT 1: note = D or E
  VARIANT 3: note = P, A, S, Y, F, G, T, or V
  VARIANT 4: note = Y or W
XGXXFAY

SEQ ID NO: 9 - protein, 7 aa, synthetic construct
  VARIANT 4: note = Y or W
DGPXFAY

SEQ ID NO: 10 - protein, 16 aa, synthetic construct
  VARIANT 4: note = R or K
  VARIANT 10: note = N or D
  VARIANT 12: note = F or I
  VARIANT 16: note = Y or S
RSSXSLLHSXGXTYLX

SEQ ID NO: 11 - protein, 9 aa, synthetic construct
  VARIANT 1: note = A, L, or Q
  VARIANT 3: note = S, G, Y, or N
  VARIANT 8: note = N or W
XQXLELPXT

SEQ ID NO: 12 - protein, 9 aa, synthetic construct
  VARIANT 3: note = S, G, Y, or N
AQXLELPNT

SEQ ID NO: 13 - protein, 5 aa, synthetic construct
NYYIH

SEQ ID NO: 14 - protein, 17 aa, synthetic construct
WIYPGNVYIQYNEKFKG

SEQ ID NO: 15 - protein, 7 aa, synthetic construct
DGPWFAY

SEQ ID NO: 16 - protein, 5 aa, synthetic construct
SYYIH

SEQ ID NO: 17 - protein, 5 aa, synthetic construct
NYNIH

SEQ ID NO: 18 - protein, 5 aa, synthetic construct
NYFIH

SEQ ID NO: 19 - protein, 5 aa, synthetic construct
NYSIH

SEQ ID NO: 20 - protein, 5 aa, synthetic construct
NYWIH

SEQ ID NO: 21 - protein, 5 aa, synthetic construct
NYYMH

SEQ ID NO: 22 - protein, 5 aa, synthetic construct
NYHIH

SEQ ID NO: 23 - protein, 5 aa, synthetic construct
NYDIH

SEQ ID NO: 24 - protein, 5 aa, synthetic construct
NYIIH

SEQ ID NO: 25 - protein, 5 aa, synthetic construct
NYLIH

SEQ ID NO: 26 - protein, 17 aa, synthetic construct
WIYPGNVYIQYSQKFQG

SEQ ID NO: 27 - protein, 17 aa, synthetic construct
WIYPGDVYIQYNEKFKG

SEQ ID NO: 28 - protein, 17 aa, synthetic construct
WFYPGNVYIQYNEKFKG

SEQ ID NO: 29 - protein, 17 aa, synthetic construct
WINPGNVYIQYNEKFKG

SEQ ID NO: 30 - protein, 7 aa, synthetic construct
EGPWFAY

SEQ ID NO: 31 - protein, 7 aa, synthetic construct
DGPYFAY

SEQ ID NO: 32 - protein, 7 aa, synthetic construct
DGAWFAY

SEQ ID NO: 33 - protein, 7 aa, synthetic construct
DGYWFAY

SEQ ID NO: 34 - protein, 7 aa, synthetic construct
DGSWFAY

SEQ ID NO: 35 - protein, 7 aa, synthetic construct
DGFWFAY

SEQ ID NO: 36 - protein, 7 aa, synthetic construct
DGGWFAY

SEQ ID NO: 37 - protein, 7 aa, synthetic construct
DGTWFAY

SEQ ID NO: 38 - protein, 7 aa, synthetic construct
DGVWFAY

SEQ ID NO: 39 - protein, 16 aa, synthetic construct
RSSKSLLHSDGFTYLY

SEQ ID NO: 40 - protein, 7 aa, synthetic construct
QTSNLAS

SEQ ID NO: 41 - protein, 9 aa, synthetic construct
AQNLELPNT

SEQ ID NO: 42 - protein, 16 aa, synthetic construct
RSSRSLLHSDGFTYLY

SEQ ID NO: 43 - protein, 16 aa, synthetic construct
RSSRSLLHSNGFTYLY

SEQ ID NO: 44 - protein, 16 aa, synthetic construct
RSSRSLLHSDGITYLY

SEQ ID NO: 45 - protein, 16 aa, synthetic construct
RSSRSLLHSDGFTYLS

SEQ ID NO: 46 - protein, 9 aa, synthetic construct
AQNLELPWT

SEQ ID NO: 47 - protein, 9 aa, synthetic construct
QQNLELPNT

SEQ ID NO: 48 - protein, 9 aa, synthetic construct
LQNLELPNT

SEQ ID NO: 49 - protein, 9 aa, synthetic construct
AQYLELPNT

SEQ ID NO: 50 - protein, 9 aa, synthetic construct
AQGLELPNT

SEQ ID NO: 51 - protein, 9 aa, synthetic construct
AQSLELPNT

SEQ ID NO: 52 - protein, 116 aa, synthetic construct
EVKLEESGPALVKPGASVRISCKASGYTFTNYYIHWVKQRPGQGLDYIGWIYPGNVYIQYNEKFKGKATLTADKSSSTAFMQLSSLTSEDSAVYFCARDGPWFAYWGQGTLVTVSS

SEQ ID NO: 53 - protein, 116 aa, synthetic construct
QVQLVQSGAEVKKPGASVKVSCKASGYTFTNYYIHWVRQAPGQRLEWMGWIYPGNVYIQYSQKFQGRVTITRDTSASTAYMELSSLRSEDTAVYYCARDGPWFAYWGQGTLVTVSS

SEQ ID NO: 54 - protein, 116 aa, synthetic construct
QVQLVQSGAEVKKPGASVKVSCKASGYTFTNYYIHWVRQAPGQRLEYIGWIYPGNVYIQYNEKFKGRATLTADKSASTAYMELSSLRSEDTAVYYCARDGPWFAYWGQGTLVTVSS

SEQ ID NO: 55 - protein, 116 aa, synthetic construct
QVQLVQSGAEVKKPGASVKVSCKASGYTFTNYYIHWVRQAPGQRLEWMGWIYPGNVYIQYNEKFKGRVTITRDTSASTAYMELSSLRSEDTAVYYCARDGPWFAYWGQGTLVTVSS

SEQ ID NO: 56 - protein, 116 aa, synthetic construct
QVQLVQSGAEVKKPGASVKVSCKASGYTFTNYYIHWVRQAPGQRLEYIGWIYPGNVYIQYSQKFQGRVTITADKSASTAYMELSSLRSEDTAVYYCARDGPWFAYWGQGTLVTVSS

SEQ ID NO: 57 - protein, 116 aa, synthetic construct
QVQLVESGAEVKKPGASVKVSCKASGYTFTNYYIHWVRQAPGQRLEYIGWIYPGNVYIQYNEKFKGRATLTADKSASTAYMELSSLRSEDTAVYYCARDGPWFAYWGQGTLVTVSS

SEQ ID NO: 58 - protein, 116 aa, synthetic construct
QVQLVQSGAEVKKPGASVKVSCKASGYTFTSYYIHWVRQAPGQRLEYIGWIYPGNVYIQYNEKFKGRATLTADKSASTAYMELSSLRSEDTAVYYCARDGPWFAYWGQGTLVTVSS

SEQ ID NO: 59 - protein, 116 aa, synthetic construct
QVQLVQSGAEVKKPGASVKVSCKASGYTFTNYNIHWVRQAPGQRLEYIGWIYPGNVYIQYNEKFKGRATLTADKSASTAYMELSSLRSEDTAVYYCARDGPWFAYWGQGTLVTVSS

SEQ ID NO: 60 - protein, 116 aa, synthetic construct
QVQLVQSGAEVKKPGASVKVSCKASGYTFTNYYIHWVRQAPGQRLEYIGWIYPGDVYIQYNEKFKGRATLTADKSASTAYMELSSLRSEDTAVYYCARDGPWFAYWGQGTLVTVSS

SEQ ID NO: 61 - protein, 116 aa, synthetic construct
QVQLVQSGAEVKKPGASVKVSCKASGYTFTNYFIHWVRQAPGQRLEYIGWIYPGNVYIQYNEKFKGRATLTADKSASTAYMELSSLRSEDTAVYYCARDGPWFAYWGQGTLVTVSS

SEQ ID NO: 62 - protein, 116 aa, synthetic construct
QVQLVQSGAEVKKPGASVKVSCKASGYTFTNYSIHWVRQAPGQRLEYIGWIYPGNVYIQYNEKFKGRATLTADKSASTAYMELSSLRSEDTAVYYCARDGPWFAYWGQGTLVTVSS

SEQ ID NO: 63 - protein, 116 aa, synthetic construct
QVQLVQSGAEVKKPGASVKVSCKASGYTFTNYWIHWVRQAPGQRLEYIGWIYPGNVYIQYNEKFKGRATLTADKSASTAYMELSSLRSEDTAVYYCARDGPWFAYWGQGTLVTVSS

SEQ ID NO: 64 - protein, 116 aa, synthetic construct
QVQLVQSGAEVKKPGASVKVSCKASGYTFTNYYIHWVRQAPGQRLEYIGWFYPGNVYIQYNEKFKGRATLTADKSASTAYMELSSLRSEDTAVYYCARDGPWFAYWGQGTLVTVSS

SEQ ID NO: 65 - protein, 116 aa, synthetic construct
QVQLVQSGAEVKKPGASVKVSCKASGYTFTNYYIHWVRQAPGQRLEWIGWIYPGNVYIQYNEKFKGRATLTADKSASTAYMELSSLRSEDTAVYYCARDGPWFAYWGQGTLVTVSS

SEQ ID NO: 66 - protein, 116 aa, synthetic construct
QVQLVQSGAEVKKPGASVKVSCKASGYTFTNYYIHWVRQAPGQRLEYIGWINPGNVYIQYNEKFKGRATLTADKSASTAYMELSSLRSEDTAVYYCARDGPWFAYWGQGTLVTVSS

SEQ ID NO: 67 - protein, 116 aa, synthetic construct
QVQLVQSGAEVKKPGASVKVSCKASGYTFTNYYIHWVRQAPGQRLEYIGWIYPGNVYIQYNEKFKGRATLTADKSASTAYMELSSLRSEDTAVYYCAREGPWFAYWGQGTLVTVSS

SEQ ID NO: 68 - protein, 116 aa, synthetic construct
QVQLVQSGAEVKKPGASVKVSCKASGYTFTNYYIHWVRQAPGQRLEYIGWIYPGNVYIQYNEKFKGRATLTADKSASTAYMELSSLRSEDTAVYYCARDGPYFAYWGQGTLVTVSS

SEQ ID NO: 69 - protein, 116 aa, synthetic construct
QVQLVQSGAEVKKPGASVKVSCKASGYTFTNYYIHWVRQAPGQRLEYIGWIYPGNVYIQYNEKFKGRATLTADKSASTAYMELSSLRSEDTAVYYCARDGAWFAYWGQGTLVTVSS

SEQ ID NO: 70 - protein, 116 aa, synthetic construct
QVQLVQSGAEVKKPGASVKVSCKASGYTFTNYYMHWVRQAPGQRLEYIGWIYPGNVYIQYNEKFKGRATLTADKSASTAYMELSSLRSEDTAVYYCARDGPWFAYWGQGTLVTVSS

SEQ ID NO: 71 - protein, 116 aa, synthetic construct
QVQLVQSGAEVKKPGASVKVSCKASGYTFTNYYIHWVRQAPGQRLEYIGWVYPGNVYIQYNEKFKGRATLTADKSASTAYMELSSLRSEDTAVYYCARDGPWFAYWGQGTLVTVSS

SEQ ID NO: 72 - protein, 116 aa, synthetic construct
QVQLVQSGAEVKKPGASVKVSCKASGYTFTNYYIHWVRQAPGQRLEYIGWIYPGNVYIQYNEKFKGRATITADKSASTAYMELSSLRSEDTAVYYCARDGPWFAYWGQGTLVTVSS

SEQ ID NO: 73 - protein, 116 aa, synthetic construct
QVQLVQSGAEVKKPGASVKVSCKASGYTFTNYYIHWVRQAPGQRLEYIGWIYPGNVYIQYNEKFKGRATLTRDKSASTAYMELSSLRSEDTAVYYCARDGPWFAYWGQGTLVTVSS

SEQ ID NO: 74 - protein, 116 aa, synthetic construct
QVQLVQSGAEVKKPGASVKVSCKASGYTFTNYYIHWVRQAPGQRLEYIGWIYPGNVYIQYNEKFKGRVTLTADKSASTAYMELSSLRSEDTAVYYCARDGPWFAYWGQGTLVTVSS

SEQ ID NO: 75 - protein, 116 aa, synthetic construct
QVQLVQSGAEVKKPGASVKVSCKASGYTFTNYHIHWVRQAPGQRLEYIGWIYPGNVYIQYNEKFKGRATLTADKSASTAYMELSSLRSEDTAVYYCARDGPWFAYWGQGTLVTVSS

SEQ ID NO: 76 - protein, 116 aa, synthetic construct
QVQLVQSGAEVKKPGASVKVSCKASGYTFTNYDIHWVRQAPGQRLEYIGWIYPGNVYIQYNEKFKGRATLTADKSASTAYMELSSLRSEDTAVYYCARDGPWFAYWGQGTLVTVSS

SEQ ID NO: 77 - protein, 116 aa, synthetic construct
QVQLVQSGAEVKKPGASVKVSCKASGYTFTNYYIHWVRQAPGQRLEYIGWIYPGNVYIQYNEKFKGRATLTADKSASTAYMELSSLRSEDTAVYYCARDGYWFAYWGQGTLVTVSS

SEQ ID NO: 78 - protein, 116 aa, synthetic construct
QVQLVQSGAEVKKPGASVKVSCKASGYTFTNYYIHWVRQAPGQRLEYIGWIYPGNVYIQYNEKFKGRATLTADKSASTAYMELSSLRSEDTAVYYCARDGSWFAYWGQGTLVTVSS

SEQ ID NO: 79 - protein, 116 aa, synthetic construct
QVQLVQSGAEVKKPGASVKVSCKASGYTFTNYYIHWVRQAPGQRLEYIGWIYPGNVYIQYNEKFKGRATLTADKSASTAYMELSSLRSEDTAVYYCARDGFWFAYWGQGTLVTVSS

SEQ ID NO: 80 - protein, 116 aa, synthetic construct
QVQLVQSGAEVKKPGASVKVSCKASGYTFTNYYIHWVRQAPGQRLEYIGWIYPGNVYIQYNEKFKGRATLTADKSASTAYMELSSLRSEDTAVYYCARDGGWFAYWGQGTLVTVSS

SEQ ID NO: 81 - protein, 116 aa, synthetic construct
QVQLVQSGAEVKKPGASVKVSCKASGYTFTNYYIHWVRQAPGQRLEYIGWIYPGNVYIQYNEKFKGRATLTADKSASTAYMELSSLRSEDTAVYYCARDGTWFAYWGQGTLVTVSS

SEQ ID NO: 82 - protein, 116 aa, synthetic construct
QVQLVQSGAEVKKPGASVKVSCKASGYTFTNYYIHWVRQAPGQRLEYIGWIYPGNVYIQYNEKFKGRATLTADKSASTAYMELSSLRSEDTAVYYCARDGVWFAYWGQGTLVTVSS

SEQ ID NO: 83 - protein, 116 aa, synthetic construct
QVQLVQSGAEVKKPGASVKVSCKASGYTFTNYIIHWVRQAPGQRLEYIGWIYPGNVYIQYNEKFKGRATLTADKSASTAYMELSSLRSEDTAVYYCARDGPWFAYWGQGTLVTVSS

SEQ ID NO: 84 - protein, 116 aa, synthetic construct
QVQLVQSGAEVKKPGASVKVSCKASGYTFTNYLIHWVRQAPGQRLEYIGWIYPGNVYIQYNEKFKGRATLTADKSASTAYMELSSLRSEDTAVYYCARDGPWFAYWGQGTLVTVSS

SEQ ID NO: 85 - protein, 112 aa, synthetic construct
DIVLTQTPFSNPVTLGTSASISCRSSKSLLHSDGFTYLYWFLQKPGQSPHLLIYQTSNLASGVPDRFSSSGSGTDFTLRISRVEAEDVGVYYCAQNLELPNTFGGGTKLEIK

SEQ ID NO: 86 - protein, 112 aa, synthetic construct
DIVMTQTPLSLSVTPGQPASISCRSSRSLLHSDGFTYLYWYLQKPGQSPQLLIYQTSNLASGVPDRFSGSGSGTDFTLKISRVEAEDVGVYYCAQNLELPNTFGQGTKLEIK

SEQ ID NO: 87 - protein, 112 aa, synthetic construct
DIVLTQTPLSLSVTPGQPASISCRSSKSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 88 - protein, 112 aa, synthetic construct
DIVMTQTPLSLSVTPGQPASISCRSSKSLLHSDGFTYLYWFLQKPGQSPQLLIYQTSNLASGVPDRFSGSGSGTDFTLKISRVEAEDVGVYYCAQNLELPNTFGQGTKLEIK

SEQ ID NO: 89 - protein, 112 aa, synthetic construct
DIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 90 - protein, 112 aa, synthetic construct
DIVLTQTPLSLSVTPGQPASISCRSSRSLLHSNGFTYLYWFLQKPGQSPQLLIYQTSNLASGVPDRFSSSGSGTDFTLKISRVEAEDVGVYYCAQNLELPNTFGQGTKLEIK

SEQ ID NO: 91 - protein, 112 aa, synthetic construct
DIVLTQTPLSLSVTPGQPASISCRSSRSLLHSDGITYLYWFLQKPGQSPQLLIYQTSNLASGVPDRFSSSGSGTDFTLKISRVEAEDVGVYYCAQNLELPNTFGQGTKLEIK

SEQ ID NO: 92 - protein, 112 aa, synthetic construct
DIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPWTFGQGTKLEIK

SEQ ID NO: 93 - protein, 112 aa, synthetic construct
DIVLTQTPLSLSVTPGQPASISCRSSRSLLHSDGFTYLSWFLQKPGQSPQLLIYQTSNLASGVPDRFSSSGSGTDFTLKISRVEAEDVGVYYCAQNLELPNTFGQGTKLEIK

SEQ ID NO: 94 - protein, 112 aa, synthetic construct
DIVLTQTPLSLSVTPGQPASISCRSSRSLLHSDGFTYLYWYLQKPGQSPQLLIYQTSNLASGVPDRFSSSGSGTDFTLKISRVEAEDVGVYYCAQNLELPNTFGQGTKLEIK

SEQ ID NO: 95 - protein, 112 aa, synthetic construct
DIVLTQTPLSLSVTPGQPASISCRSSRSLLHSDGFTYLYWFLQKPGQSPQLLIYQTSNLASGVPDRFSSSGSGTDFTLKISRVEAEDVGVYYCQQNLELPNTFGQGTKLEIK

SEQ ID NO: 96 - protein, 112 aa, synthetic construct
DIVLTQTPLSLSVTPGQPASISCRSSRSLLHSDGFTYLYWFLQKPGQSPQLLIYQTSNLASGVPDRFSSSGSGTDFTLKISRVEAEDVGVYYCLQNLELPNTFGQGTKLEIK

SEQ ID NO: 97 - protein, 112 aa, synthetic construct
DIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQYLELPNTFGQGTKLEIK

SEQ ID NO: 98 - protein, 112 aa, synthetic construct
DIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQGLELPNTFGQGTKLEIK

SEQ ID NO: 99 - protein, 112 aa, synthetic construct
DIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQSLELPNTFGQGTKLEIK

SEQ ID NO: 100 - protein, 452 aa, synthetic construct
EVKLEESGPALVKPGASVRISCKASGYTFTNYYIHWVKQRPGQGLDYIGWIYPGNVYIQYNEKFKGKATLTADKSSSTAFMQLSSLTSEDSAVYFCARDGPWFAYWGQGTLVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 101 - protein, 445 aa, synthetic construct
EVKLEESGPALVKPGASVRISCKASGYTFTNYYIHWVKQRPGQGLDYIGWIYPGNVYIQYNEKFKGKATLTADKSSSTAFMQLSSLTSEDSAVYFCARDGPWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 102 - protein, 445 aa, synthetic construct
QVQLVQSGAEVKKPGASVKVSCKASGYTFTNYYIHWVRQAPGQRLEWMGWIYPGNVYIQYSQKFQGRVTITRDTSASTAYMELSSLRSEDTAVYYCARDGPWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 103 - protein, 445 aa, synthetic construct
QVQLVQSGAEVKKPGASVKVSCKASGYTFTNYYIHWVRQAPGQRLEYIGWIYPGNVYIQYNEKFKGRATLTADKSASTAYMELSSLRSEDTAVYYCARDGPWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 104 - protein, 450 aa, synthetic construct
QVQLVQSGAEVKKPGASVKVSCKASGYTFTNYYIHWVRQAPGQRLEYIGWIYPGNVYIQYNEKFKGRATLTADKSASTAYMELSSLRSEDTAVYYCARDGPWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPGSLSPG

SEQ ID NO: 105 - protein, 445 aa, synthetic construct
QVQLVQSGAEVKKPGASVKVSCKASGYTFTNYYIHWVRQAPGQRLEWMGWIYPGNVYIQYNEKFKGRVTITRDTSASTAYMELSSLRSEDTAVYYCARDGPWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 106 - protein, 445 aa, synthetic construct
QVQLVQSGAEVKKPGASVKVSCKASGYTFTNYYIHWVRQAPGQRLEYIGWIYPGNVYIQYSQKFQGRVTITADKSASTAYMELSSLRSEDTAVYYCARDGPWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 107 - protein, 445 aa, synthetic construct
QVQLVESGAEVKKPGASVKVSCKASGYTFTNYYIHWVRQAPGQRLEYIGWIYPGNVYIQYNEKFKGRATLTADKSASTAYMELSSLRSEDTAVYYCARDGPWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 108 - protein, 445 aa, synthetic construct
QVQLVQSGAEVKKPGASVKVSCKASGYTFTSYYIHWVRQAPGQRLEYIGWIYPGNVYIQYNEKFKGRATLTADKSASTAYMELSSLRSEDTAVYYCARDGPWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 109 - protein, 445 aa, synthetic construct
QVQLVQSGAEVKKPGASVKVSCKASGYTFTNYNIHWVRQAPGQRLEYIGWIYPGNVYIQYNEKFKGRATLTADKSASTAYMELSSLRSEDTAVYYCARDGPWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 110 - protein, 445 aa, synthetic construct
QVQLVQSGAEVKKPGASVKVSCKASGYTFTNYYIHWVRQAPGQRLEYIGWIYPGDVYIQYNEKFKGRATLTADKSASTAYMELSSLRSEDTAVYYCARDGPWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 111 - protein, 445 aa, synthetic construct
QVQLVQSGAEVKKPGASVKVSCKASGYTFTNYFIHWVRQAPGQRLEYIGWIYPGNVYIQYNEKFKGRATLTADKSASTAYMELSSLRSEDTAVYYCARDGPWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 112 - protein, 445 aa, synthetic construct
QVQLVQSGAEVKKPGASVKVSCKASGYTFTNYSIHWVRQAPGQRLEYIGWIYPGNVYIQYNEKFKGRATLTADKSASTAYMELSSLRSEDTAVYYCARDGPWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 113 - protein, 445 aa, synthetic construct
QVQLVQSGAEVKKPGASVKVSCKASGYTFTNYWIHWVRQAPGQRLEYIGWIYPGNVYIQYNEKFKGRATLTADKSASTAYMELSSLRSEDTAVYYCARDGPWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 114 - protein, 445 aa, synthetic construct
QVQLVQSGAEVKKPGASVKVSCKASGYTFTNYYIHWVRQAPGQRLEYIGWFYPGNVYIQYNEKFKGRATLTADKSASTAYMELSSLRSEDTAVYYCARDGPWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 115 - protein, 445 aa, synthetic construct
QVQLVQSGAEVKKPGASVKVSCKASGYTFTNYYIHWVRQAPGQRLEWIGWIYPGNVYIQYNEKFKGRATLTADKSASTAYMELSSLRSEDTAVYYCARDGPWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 116 - protein, 445 aa, synthetic construct
QVQLVQSGAEVKKPGASVKVSCKASGYTFTNYYIHWVRQAPGQRLEYIGWINPGNVYIQYNEKFKGRATLTADKSASTAYMELSSLRSEDTAVYYCARDGPWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 117 - protein, 445 aa, synthetic construct
QVQLVQSGAEVKKPGASVKVSCKASGYTFTNYYIHWVRQAPGQRLEYIGWIYPGNVYIQYNEKFKGRATLTADKSASTAYMELSSLRSEDTAVYYCAREGPWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 118 - protein, 445 aa, synthetic construct
QVQLVQSGAEVKKPGASVKVSCKASGYTFTNYYIHWVRQAPGQRLEYIGWIYPGNVYIQYNEKFKGRATLTADKSASTAYMELSSLRSEDTAVYYCARDGPYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 119 - protein, 445 aa, synthetic construct
QVQLVQSGAEVKKPGASVKVSCKASGYTFTNYYIHWVRQAPGQRLEYIGWIYPGNVYIQYNEKFKGRATLTADKSASTAYMELSSLRSEDTAVYYCARDGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 120 - protein, 445 aa, synthetic construct
QVQLVQSGAEVKKPGASVKVSCKASGYTFTNYYMHWVRQAPGQRLEYIGWIYPGNVYIQYNEKFKGRATLTADKSASTAYMELSSLRSEDTAVYYCARDGPWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 121 - protein, 445 aa, synthetic construct
QVQLVQSGAEVKKPGASVKVSCKASGYTFTNYYIHWVRQAPGQRLEYIGWVYPGNVYIQYNEKFKGRATLTADKSASTAYMELSSLRSEDTAVYYCARDGPWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 122 - protein, 445 aa, synthetic construct
QVQLVQSGAEVKKPGASVKVSCKASGYTFTNYYIHWVRQAPGQRLEYIGWIYPGNVYIQYNEKFKGRATITADKSASTAYMELSSLRSEDTAVYYCARDGPWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 123 - protein, 445 aa, synthetic construct
QVQLVQSGAEVKKPGASVKVSCKASGYTFTNYYIHWVRQAPGQRLEYIGWIYPGNVYIQYNEKFKGRATLTRDKSASTAYMELSSLRSEDTAVYYCARDGPWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 124 - protein, 445 aa, synthetic construct
QVQLVQSGAEVKKPGASVKVSCKASGYTFTNYYIHWVRQAPGQRLEYIGWIYPGNVYIQYNEKFKGRVTLTADKSASTAYMELSSLRSEDTAVYYCARDGPWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 125 - protein, 445 aa, synthetic construct
QVQLVQSGAEVKKPGASVKVSCKASGYTFTNYHIHWVRQAPGQRLEYIGWIYPGNVYIQYNEKFKGRATLTADKSASTAYMELSSLRSEDTAVYYCARDGPWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 126 - protein, 445 aa, synthetic construct
QVQLVQSGAEVKKPGASVKVSCKASGYTFTNYDIHWVRQAPGQRLEYIGWIYPGNVYIQYNEKFKGRATLTADKSASTAYMELSSLRSEDTAVYYCARDGPWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 127 - protein, 445 aa, synthetic construct
QVQLVQSGAEVKKPGASVKVSCKASGYTFTNYYIHWVRQAPGQRLEYIGWIYPGNVYIQYNEKFKGRATLTADKSASTAYMELSSLRSEDTAVYYCARDGYWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 128 - protein, 445 aa, synthetic construct
QVQLVQSGAEVKKPGASVKVSCKASGYTFTNYYIHWVRQAPGQRLEYIGWIYPGNVYIQYNEKFKGRATLTADKSASTAYMELSSLRSEDTAVYYCARDGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 129 - protein, 445 aa, synthetic construct
QVQLVQSGAEVKKPGASVKVSCKASGYTFTNYYIHWVRQAPGQRLEYIGWIYPGNVYIQYNEKFKGRATLTADKSASTAYMELSSLRSEDTAVYYCARDGFWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 130 - protein, 445 aa, synthetic construct
QVQLVQSGAEVKKPGASVKVSCKASGYTFTNYYIHWVRQAPGQRLEYIGWIYPGNVYIQYNEKFKGRATLTADKSASTAYMELSSLRSEDTAVYYCARDGGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 131 - protein, 445 aa, synthetic construct
QVQLVQSGAEVKKPGASVKVSCKASGYTFTNYYIHWVRQAPGQRLEYIGWIYPGNVYIQYNEKFKGRATLTADKSASTAYMELSSLRSEDTAVYYCARDGTWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 132 - protein, 445 aa, synthetic construct
QVQLVQSGAEVKKPGASVKVSCKASGYTFTNYYIHWVRQAPGQRLEYIGWIYPGNVYIQYNEKFKGRATLTADKSASTAYMELSSLRSEDTAVYYCARDGVWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 133 - protein, 445 aa, synthetic construct
QVQLVQSGAEVKKPGASVKVSCKASGYTFTNYIIHWVRQAPGQRLEYIGWIYPGNVYIQYNEKFKGRATLTADKSASTAYMELSSLRSEDTAVYYCARDGPWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 134 - protein, 445 aa, synthetic construct
QVQLVQSGAEVKKPGASVKVSCKASGYTFTNYLIHWVRQAPGQRLEYIGWIYPGNVYIQYNEKFKGRATLTADKSASTAYMELSSLRSEDTAVYYCARDGPWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 135 - protein, 219 aa, synthetic construct
DIVLTQTPFSNPVTLGTSASISCRSSKSLLHSDGFTYLYWFLQKPGQSPHLLIYQTSNLASGVPDRFSSSGSGTDFTLRISRVEAEDVGVYYCAQNLELPNTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 136 - protein, 219 aa, synthetic construct
DIVLTQTPFSNPVTLGTSASISCRSSKSLLHSDGFTYLYWFLQKPGQSPHLLIYQTSNLASGVPDRFSSSGSGTDFTLRISRVEAEDVGVYYCAQNLELPNTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 219 aa, synthetic construct
DIVMTQTPLSLSVTPGQPASISCRSSRSLLHSDGFTYLYWYLQKPGQSPQLLIYQTSNLASGVPDRFSGSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 138 - protein, 219 aa, synthetic construct
DIVLTQTPLSLSVTPGQPASISCRSSKSLLHSDGFTYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 139 - protein, 219 aa, synthetic construct
DIVMTQTPLSLSVTPGQPASISCRSSKSLLHSDGFTYLYWFLQKPGQSPQLLIYQTSNLASGVPDRFSGSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 140 - protein, 219 aa, synthetic construct
DIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 141 - protein, 219 aa, synthetic construct
DIVLTQTPLSLSVTPGQPASISCRSSRSLLHSNGFTYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 142 - protein, 219 aa, synthetic construct
DIVLTQTPLSLSVTPGQPASISCRSSRSLLHSDGITYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 143 - protein, 219 aa, synthetic construct
DIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 144 - protein, 219 aa, synthetic construct
DIVLTQTPLSLSVTPGQPASISCRSSRSLLHSDGFTYLSWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - protein, 219 aa, synthetic construct
DIVLTQTPLSLSVTPGQPASISCRSSRSLLHSDGFTYLYWYLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 146 - protein, 219 aa, synthetic construct
DIVLTQTPLSLSVTPGQPASISCRSSRSLLHSDGFTYLYWFLQKPGQSPQLLIYQTSNLASGVPDRFSSSGSGTDFTLKISRVEAEDVGVYYCQQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 147 - protein, 219 aa, synthetic construct
DIVLTQTPLSLSVTPGQPASISCRSSRSLLHSDGFTYLYWFLQKPGQSPQLLIYQTSNLASGVPDRFSSSGSGTDFTLKISRVEAEDVGVYYCLQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 148 - protein, 219 aa, synthetic construct
DIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQYLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 149 - protein, 219 aa, synthetic construct
DIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQGLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 150 - protein, 219 aa, synthetic construct
DIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQSLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - protein, 15 aa, synthetic construct
PLMTCSDYYTCLNNL

SEQ ID NO: 152 - protein, 15 aa, synthetic construct
LSCTHSRYDMHCPHM

SEQ ID NO: 153 - protein, 15 aa, synthetic construct
HYCHSRTDTITHCNA

SEQ ID NO: 154 - protein, 15 aa, synthetic construct
WCPRLFDRPSMGCPT

SEQ ID NO: 155 - protein, 15 aa, synthetic construct
WWPPCQGGAWCEQRI

SEQ ID NO: 156 - protein, 15 aa, synthetic construct
HSGCPRLFDRCSAPA

SEQ ID NO: 157 - protein, 15 aa, synthetic construct
FICPTLYDRPHCMHT

SEQ ID NO: 158 - protein, 15 aa, synthetic construct
SWCHSATDTILPCSN

SEQ ID NO: 159 - protein, 15 aa, synthetic construct
SPACSDRYYQTCVLN

SEQ ID NO: 160 - protein, 15 aa, synthetic construct
MSCVVDRFDRQCPHL

SEQ ID NO: 161 - protein, 15 aa, synthetic construct
TTRCEHYWFTCPLSP

SEQ ID NO: 162 - protein, 15 aa, synthetic construct
DCTGYSPSVLPACRV

SEQ ID NO: 163 - protein, 15 aa, synthetic construct
FCSGYSPSVLPSCLM

SEQ ID NO: 164 - protein, 15 aa, synthetic construct
SKPCSYMHPYCFYNS

SEQ ID NO: 165 - protein, 15 aa, synthetic construct
LTRCTIAHPYCYYNY

SEQ ID NO: 166 - protein, 15 aa, synthetic construct
PNTCMSERRICSLTY

SEQ ID NO: 167 - protein, 15 aa, synthetic construct
PRPHCAILRQCLAAT

SEQ ID NO: 168 - protein, 18 aa, synthetic construct
AVGLLAPPGGLSGRSDNI

SEQ ID NO: 169 - protein, 13 aa, synthetic construct
ISSGLLSGRSDNI

SEQ ID NO: 170 - protein, 269 aa, synthetic construct
QGQSGQGPLMTCSDYYTCLNNLGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 171 - protein, 269 aa, synthetic construct
QGQSGQGLSCTHSRYDMHCPHMGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 172 - protein, 269 aa, synthetic construct
QGQSGQGHYCHSRTDTITHCNAGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 173 - protein, 269 aa, synthetic construct
QGQSGQGWCPRLFDRPSMGCPTGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 174 - protein, 269 aa, synthetic construct
QGQSGQGWWPPCQGGAWCEQRIGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 175 - protein, 269 aa, synthetic construct
QGQSGQGHSGCPRLFDRCSAPAGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 176 - protein, 269 aa, synthetic construct
QGQSGQGFICPTLYDRPHCMHTGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 177 - protein, 265 aa, synthetic construct
QGQSGQGPLMTCSDYYTCLNNLGGGSSGGSISSGLLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 178 - protein, 265 aa, synthetic construct
QGQSGQGWCPRLFDRPSMGCPTGGGSSGGSISSGLLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 179 - protein, 265 aa, synthetic construct
QGQSGQGWWPPCQGGAWCEQRIGGGSSGGSISSGLLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 180 - protein, 265 aa, synthetic construct
QGQSGQGHSGCPRLFDRCSAPAGGGSSGGSISSGLLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 181 - protein, 265 aa, synthetic construct
QGQSGQGSWCHSATDTILPCSNGGGSSGGSISSGLLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 182 - protein, 265 aa, synthetic construct
QGQSGQGSPACSDRYYQTCVLNGGGSSGGSISSGLLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 183 - protein, 265 aa, synthetic construct
QGQSGQGMSCVVDRFDRQCPHLGGGSSGGSISSGLLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 184 - protein, 265 aa, synthetic construct
QGQSGQGTTRCEHYWFTCPLSPGGGSSGGSISSGLLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 185 - protein, 269 aa, synthetic construct
QGQSGQGSWCHSATDTILPCSNGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 186 - protein, 269 aa, synthetic construct
QGQSGQGSPACSDRYYQTCVLNGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 187 - protein, 269 aa, synthetic construct
QGQSGQGMSCVVDRFDRQCPHLGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 188 - protein, 269 aa, synthetic construct
QGQSGQGTTRCEHYWFTCPLSPGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 189 - protein, 265 aa, synthetic construct
QGQSGQGDCTGYSPSVLPACRVGGGSSGGSISSGLLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 190 - protein, 265 aa, synthetic construct
QGQSGQGFCSGYSPSVLPSCLMGGGSSGGSISSGLLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 191 - protein, 265 aa, synthetic construct
QGQSGQGSKPCSYMHPYCFYNSGGGSSGGSISSGLLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 192 - protein, 265 aa, synthetic construct
QGQSGQGLTRCTIAHPYCYYNYGGGSSGGSISSGLLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 193 - protein, 265 aa, synthetic construct
QGQSGQGPNTCMSERRICSLTYGGGSSGGSISSGLLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 194 - protein, 265 aa, synthetic construct
QGQSGQGPRPHCAILRQCLAATGGGSSGGSISSGLLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 195 - protein, 269 aa, synthetic construct
QGQSGQGDCTGYSPSVLPACRVGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 196 - protein, 269 aa, synthetic construct
QGQSGQGFCSGYSPSVLPSCLMGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 197 - protein, 269 aa, synthetic construct
QGQSGQGSKPCSYMHPYCFYNSGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 198 - protein, 269 aa, synthetic construct
QGQSGQGLTRCTIAHPYCYYNYGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 199 - protein, 269 aa, synthetic construct
QGQSGQGPNTCMSERRICSLTYGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 200 - protein, 269 aa, synthetic construct
QGQSGQGPRPHCAILRQCLAATGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 201 - DNA, 1335 bp, synthetic construct
  source 1..1335: mol_type = other DNA
gaggtgaagctggaggagagcggccccgccctggtgaagcctggagcttccgtgaggatcagctgtaaggcctctggctacacctttacaaactactatatccattgggtgaagcagcggccaggacagggcctggactacatcggctggatctatcccggcaacgtgtacatccagtataatgagaagttcaagggcaaggccaccctgacagctgataagtccagctctaccgcttttatgcagctgtccagcctgacaagcgaggactctgccgtgtacttctgcgctagggatggcccttggtttgcctattggggccagggcaccctggtgacagtgtcttccgcttccaccaagggcccatcagttttccccttggctccaagttctaaatccacaagcggtggaacagctgcactgggatgcctcgttaaagattatttccctgagcctgtgacagtgagctggaatagcggagcattgacttcaggtgtgcacacttttcccgctgtgttgcagtcctccggtctgtactcactgtccagtgtcgtaaccgtcccttctagcagcttgggaacccagacctacatctgtaacgtcaaccataaaccatccaacacaaaggtggataagaaggttgaaccaaagagctgtgataagacacatacatgccctccttgtcctgcaccagagctcctcggaggtccatctgtgttcctgtttccccccaaacccaaggacactcttatgatctctcgtactccagaggtcacctgtgttgttgtcgacgtgagccatgaagatcccgaggttaaattcaactggtacgtggatggagtcgaggttcacaatgccaagaccaagcccagggaggagcaatataattctacatatcgggtagtgagcgttctgaccgtgctccaccaagattggctcaatggaaaagagtacaagtgcaaggtgtccaacaaggctcttcccgctcccattgagaaaactatctccaaagccaaggggcagccacgggaaccccaggtgtatacattgcccccatctagagacgagctgaccaagaaccaggtgagtctcacttgtctggtcaaggggttttacccttctgacattgctgtagagtgggagtctaacggacagccagaaaacaactacaagacaactcccccagtgctggacagcgacgggagcttcttcctctactccaagttgactgtagacaagtctagatggcagcaaggaaacgttttctcctgctcagtaatgcatgaggctctgcacaatcactatacccagaaatcactgtcccttagcccaggg

SEQ ID NO: 202 - DNA, 657 bp, synthetic construct
  source 1..657: mol_type = other DNA
gatattgtgctgacccagactccattctccaatcccgtcactcttggaacatcagcttccatctcctgcaggtctagtaagagtctcctacatagtgatggcttcacttatttgtattggtttctgcagaagccaggccagtctcctcatctcctgatttatcagacgtccaaccttgcctcaggagtcccagacaggttcagtagcagtgggtcaggaactgatttcacactgagaatcagcagagtggaggctgaagatgtgggtgtttattactgtgctcaaaatctagaacttcccaacacgttcggaggggggaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 203 - DNA, 1335 bp, synthetic construct
  source 1..1335: mol_type = other DNA
caggtgcagctggtgcagagcggagctgaggtgaagaagccaggagcttccgtgaaggtgagctgtaaggcctctggctacaccttcacaaactactatatccattgggtgaggcaggctccaggacagcggctggagtacatcggatggatctatcctggcaacgtgtacatccagtataatgagaagtttaagggcagggccaccctgacagctgacaagagcgcctctaccgcttacatggagctgtccagcctgagatctgaggacacagccgtgtactattgcgctcgcgatggcccttggtttgcctattggggccagggcaccctggtgacagtgtcttccgcttccaccaagggcccatcagttttccccttggctccaagttctaaatccacaagcggtggaacagctgcactgggatgcctcgttaaagattatttccctgagcctgtgacagtgagctggaatagcggagcattgacttcaggtgtgcacacttttcccgctgtgttgcagtcctccggtctgtactcactgtccagtgtcgtaaccgtcccttctagcagcttgggaacccagacctacatctgtaacgtcaaccataaaccatccaacacaaaggtggataagaaggttgaaccaaagagctgtgataagacacatacatgccctccttgtcctgcaccagagctcctcggaggtccatctgtgttcctgtttccccccaaacccaaggacactcttatgatctctcgtactccagaggtcacctgtgttgttgtcgacgtgagccatgaagatcccgaggttaaattcaactggtacgtggatggagtcgaggttcacaatgccaagaccaagcccagggaggagcaatataattctacatatcgggtagtgagcgttctgaccgtgctccaccaagattggctcaatggaaaagagtacaagtgcaaggtgtccaacaaggctcttcccgctcccattgagaaaactatctccaaagccaaggggcagccacgggaaccccaggtgtatacattgcccccatctagagacgagctgaccaagaaccaggtgagtctcacttgtctggtcaaggggttttacccttctgacattgctgtagagtgggagtctaacggacagccagaaaacaactacaagacaactcccccagtgctggacagcgacgggagcttcttcctctactccaagttgactgtagacaagtctagatggcagcaaggaaacgttttctcctgctcagtaatgcatgaggctctgcacaatcactatacccagaaatcactgtcccttagcccaggg

SEQ ID NO: 204 - DNA, 657 bp, synthetic construct
  source 1..657: mol_type = other DNA
gacatcgtgctgacccagacaccactgtctctgtccgtgaccccaggacagcctgctagcatctcttgtaggtccagcaggtccctgctgcatagcgatggcttcacctacctgtattggtttctgcagaagccaggccagtctccccagctgctgatctaccagacatctaacctggcttccggcgtgcctgacaggttctcttccagcggcagcggcaccgacttcaccctgaagatctctcgggtggaggctgaggacgtgggcgtgtactattgcgctcagaacctggagctgccaaatacctttggccagggcacaaagctggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 205 - DNA, 1338 bp, synthetic construct
  source 1..1338: mol_type = other DNA
caggtgcagctggtgcagagcggagctgaggtgaagaagccaggagcttccgtgaaggtgagctgtaaggcctctggctacaccttcacaaactaccacatccattgggtgaggcaggctccaggacagcggctggagtacatcggatggatctatcctggcaacgtgtacatccagtataatgagaagtttaagggcagggccaccctgacagctgacaagagcgcctctaccgcttacatggagctgtccagcctgagatctgaggacacagccgtgtactattgcgctcgcgatggcccttggtttgcctattggggccagggcaccctggtgacagtgtcttccgcttccaccaagggcccatcagttttccccttggctccaagttctaaatccacaagcggtggaacagctgcactgggatgcctcgttaaagattatttccctgagcctgtgacagtgagctggaatagcggagcattgacttcaggtgtgcacacttttcccgctgtgttgcagtcctccggtctgtactcactgtccagtgtcgtaaccgtcccttctagcagcttgggaacccagacctacatctgtaacgtcaaccataaaccatccaacacaaaggtggataagaaggttgaaccaaagagctgtgataagacacatacatgccctccttgtcctgcaccagagctcctcggaggtccatctgtgttcctgtttccccccaaacccaaggacactcttatgatctctcgtactccagaggtcacctgtgttgttgtcgacgtgagccatgaagatcccgaggttaaattcaactggtacgtggatggagtcgaggttcacaatgccaagaccaagcccagggaggagcaatataattctacatatcgggtagtgagcgttctgaccgtgctccaccaagattggctcaatggaaaagagtacaagtgcaaggtgtccaacaaggctcttcccgctcccattgagaaaactatctccaaagccaaggggcagccacgggaaccccaggtgtatacattgcccccatctagagacgagctgaccaagaaccaggtgagtctcacttgtctggtcaaggggttttacccttctgacattgctgtagagtgggagtctaacggacagccagaaaacaactacaagacaactcccccagtgctggacagcgacgggagcttcttcctctactccaagttgactgtagacaagtctagatggcagcaaggaaacgttttctcctgctcagtaatgcatgaggctctgcacaatcactatacccagaaatcactgtcccttagcccagggtga

SEQ ID NO: 206 - DNA, 1338 bp, synthetic construct
  source 1..1338: mol_type = other DNA
caggtgcagctggtgcagagcggagctgaggtgaagaagccaggagcttccgtgaaggtgagctgtaaggcctctggctacaccttcacaaactacgacatccattgggtgaggcaggctccaggacagcggctggagtacatcggatggatctatcctggcaacgtgtacatccagtataatgagaagtttaagggcagggccaccctgacagctgacaagagcgcctctaccgcttacatggagctgtccagcctgagatctgaggacacagccgtgtactattgcgctcgcgatggcccttggtttgcctattggggccagggcaccctggtgacagtgtcttccgcttccaccaagggcccatcagttttccccttggctccaagttctaaatccacaagcggtggaacagctgcactgggatgcctcgttaaagattatttccctgagcctgtgacagtgagctggaatagcggagcattgacttcaggtgtgcacacttttcccgctgtgttgcagtcctccggtctgtactcactgtccagtgtcgtaaccgtcccttctagcagcttgggaacccagacctacatctgtaacgtcaaccataaaccatccaacacaaaggtggataagaaggttgaaccaaagagctgtgataagacacatacatgccctccttgtcctgcaccagagctcctcggaggtccatctgtgttcctgtttccccccaaacccaaggacactcttatgatctctcgtactccagaggtcacctgtgttgttgtcgacgtgagccatgaagatcccgaggttaaattcaactggtacgtggatggagtcgaggttcacaatgccaagaccaagcccagggaggagcaatataattctacatatcgggtagtgagcgttctgaccgtgctccaccaagattggctcaatggaaaagagtacaagtgcaaggtgtccaacaaggctcttcccgctcccattgagaaaactatctccaaagccaaggggcagccacgggaaccccaggtgtatacattgcccccatctagagacgagctgaccaagaaccaggtgagtctcacttgtctggtcaaggggttttacccttctgacattgctgtagagtgggagtctaacggacagccagaaaacaactacaagacaactcccccagtgctggacagcgacgggagcttcttcctctactccaagttgactgtagacaagtctagatggcagcaaggaaacgttttctcctgctcagtaatgcatgaggctctgcacaatcactatacccagaaatcactgtcccttagcccagggtga

SEQ ID NO: 207 - DNA, 1338 bp, synthetic construct
  source 1..1338: mol_type = other DNA
caggtgcagctggtgcagagcggagctgaggtgaagaagccaggagcttccgtgaaggtgagctgtaaggcctctggctacaccttcacaaactactatatccattgggtgaggcaggctccaggacagcggctggagtacatcggatggatctatcctggcaacgtgtacatccagtataatgagaagtttaagggcagggccaccctgacagctgacaagagcgcctctaccgcttacatggagctgtccagcctgagatctgaggacacagccgtgtactattgcgctcgcgatggctactggtttgcctattggggccagggcaccctggtgacagtgtcttccgcttccaccaagggcccatcagttttccccttggctccaagttctaaatccacaagcggtggaacagctgcactgggatgcctcgttaaagattatttccctgagcctgtgacagtgagctggaatagcggagcattgacttcaggtgtgcacacttttcccgctgtgttgcagtcctccggtctgtactcactgtccagtgtcgtaaccgtcccttctagcagcttgggaacccagacctacatctgtaacgtcaaccataaaccatccaacacaaaggtggataagaaggttgaaccaaagagctgtgataagacacatacatgccctccttgtcctgcaccagagctcctcggaggtccatctgtgttcctgtttccccccaaacccaaggacactcttatgatctctcgtactccagaggtcacctgtgttgttgtcgacgtgagccatgaagatcccgaggttaaattcaactggtacgtggatggagtcgaggttcacaatgccaagaccaagcccagggaggagcaatataattctacatatcgggtagtgagcgttctgaccgtgctccaccaagattggctcaatggaaaagagtacaagtgcaaggtgtccaacaaggctcttcccgctcccattgagaaaactatctccaaagccaaggggcagccacgggaaccccaggtgtatacattgcccccatctagagacgagctgaccaagaaccaggtgagtctcacttgtctggtcaaggggttttacccttctgacattgctgtagagtgggagtctaacggacagccagaaaacaactacaagacaactcccccagtgctggacagcgacgggagcttcttcctctactccaagttgactgtagacaagtctagatggcagcaaggaaacgttttctcctgctcagtaatgcatgaggctctgcacaatcactatacccagaaatcactgtcccttagcccagggtga

SEQ ID NO: 208 - DNA, 807 bp, synthetic construct
  source 1..807: mol_type = other DNA
cagggacagtctggacagggatggtggccaccttgccagggaggagcttggtgtgagcagaggatcggaggaggctccagcggaggagctgtgggcctgctggctccaccaggaggactgtctggcagatccgacaacatcggcggctccgatatcgtgctgacccagacacccctgagcctgtctgtgacccctggccagccagcctccatcagctgcaggtcttcccggtccctgctgcatagcgacggcttcacctacctgtattggtttctgcagaagcccggccagagccctcagctgctgatctaccagacatctaatctggcttccggcgtgccagacagattcagctcttccggcagcggcaccgacttcaccctgaagatctctcgcgtggaggccgaggatgtgggcgtgtactattgtgctcagaacctggagctgcccaatacctttggccagggcacaaagctggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 209 - DNA, 795 bp, synthetic construct
  source 1..795: mol_type = other DNA
cagggacagagcggacagggatggtggccaccttgccagggaggagcttggtgtgagcagaggatcggaggaggctccagcggaggctctatctcttccggcctgctgagcggcagatctgacaacatcggcggctccgatatcgtgctgacccagacaccactgtccctgagcgtgaccccaggacagccagcctctatctcctgcaggagctctcggtctctgctgcattccgacggcttcacctacctgtattggtttctgcagaagcctggccagtctccacagctgctgatctaccagacaagcaatctggcttctggcgtgcccgacagattctccagctctggctccggcaccgacttcaccctgaagatcagccgcgtggaggctgaggatgtgggcgtgtactattgtgctcagaacctggagctgcctaatacctttggccagggcacaaagctggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 210-306 - skipped (97 entries)

SEQ ID NO: 307 - protein, 216 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 308 - protein, 216 aa, synthetic construct
APELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 309 - protein, 216 aa, synthetic construct
APELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 310 - protein, 4 aa, synthetic construct
GGFG

SEQ ID NO: 311 - protein, 4 aa, synthetic construct
  SITE 1: note = Maleimide modified residue
GGFG

SEQ ID NO: 312 - protein, 13 aa, synthetic construct
ISSGLLSGRSDNH

SEQ ID NO: 313 - protein, 18 aa, synthetic construct
AVGLLAPPGGLSGRSDNH

SEQ ID NO: 314 - protein, 13 aa, synthetic construct
ISSGLLSGRSDIH

SEQ ID NO: 315 - protein, 13 aa, synthetic construct
ISSGLLSGRSDQH

SEQ ID NO: 316 - protein, 13 aa, synthetic construct
ISSGLLSGRSDNP

SEQ ID NO: 317 - protein, 13 aa, synthetic construct
ISSGLLSGRSANP

SEQ ID NO: 318 - protein, 13 aa, synthetic construct
ISSGLLSGRSANI

SEQ ID NO: 319 - protein, 18 aa, synthetic construct
AVGLLAPPGGLSGRSDIH

SEQ ID NO: 320 - protein, 18 aa, synthetic construct
AVGLLAPPGGLSGRSDQH

SEQ ID NO: 321 - protein, 18 aa, synthetic construct
AVGLLAPPGGLSGRSDNP

SEQ ID NO: 322 - protein, 18 aa, synthetic construct
AVGLLAPPGGLSGRSANP

SEQ ID NO: 323 - protein, 18 aa, synthetic construct
AVGLLAPPGGLSGRSANI

SEQ ID NO: 324 - protein, 7 aa, synthetic construct
QGQSGQG

SEQ ID NO: 325 - protein, 6 aa, synthetic construct
QGQSGQ

SEQ ID NO: 326 - protein, 5 aa, synthetic construct
QGQSG

SEQ ID NO: 327 - protein, 4 aa, synthetic construct
QGQS

SEQ ID NO: 328 - protein, 6 aa, synthetic construct
GQSGQG

SEQ ID NO: 329 - protein, 5 aa, synthetic construct
QSGQG

SEQ ID NO: 330 - protein, 4 aa, synthetic construct
SGQG

SEQ ID NO: 331 - protein, 15 aa, synthetic construct
PLMTCSDYYTCKNNL

SEQ ID NO: 332 - protein, 15 aa, synthetic construct
PLMTCSDYYTCHNNL

SEQ ID NO: 333 - protein, 15 aa, synthetic construct
PLMTCSDYYTCRNNL

SEQ ID NO: 334 - protein, 15 aa, synthetic construct
KFSCTTMPHKNCMKP

SEQ ID NO: 335 - protein, 15 aa, synthetic construct
PLMTCTKYNNCINNL

SEQ ID NO: 336 - protein, 15 aa, synthetic construct
PNYMCKLNTCTHHIA

SEQ ID NO: 337 - protein, 15 aa, synthetic construct
AACAGGVLFDRCSVD

SEQ ID NO: 338 - protein, 15 aa, synthetic construct
NMPACQGGSWCRPGY

SEQ ID NO: 339 - protein, 15 aa, synthetic construct
TLCARPPSYQLCSHV

SEQ ID NO: 340 - protein, 15 aa, synthetic construct
NLCPPGETRTQCQIT

SEQ ID NO: 341 - protein, 15 aa, synthetic construct
LYCALYSVQLFCVDL

SEQ ID NO: 342 - protein, 15 aa, synthetic construct
KMAPCDAWYCHQQTA

SEQ ID NO: 343 - protein, 15 aa, synthetic construct
QDECSNKEAENCSDV

SEQ ID NO: 344 - protein, 15 aa, synthetic construct
QTCYGWSWSGSCLWY

SEQ ID NO: 345 - protein, 15 aa, synthetic construct
SNPCISTGHTHCQKI

SEQ ID NO: 346 - protein, 15 aa, synthetic construct
QICFGGSKNRYDCFM

SEQ ID NO: 347 - protein, 15 aa, synthetic construct
PTAPCHFNPPCRNTG

SEQ ID NO: 348 - protein, 15 aa, synthetic construct
VCSGSSPGHPRVCGD

SEQ ID NO: 349 - protein, 15 aa, synthetic construct
LLPCPTSMPSYCFNY

SEQ ID NO: 350 - protein, 15 aa, synthetic construct
RGTPCTINTCTHHFS

SEQ ID NO: 351 - protein, 15 aa, synthetic construct
KNGGCKVPIPCKSWS

SEQ ID NO: 352 - protein, 15 aa, synthetic construct
SRPCSYMHPYCFYNS

SEQ ID NO: 353 - protein, 15 aa, synthetic construct
SHPCSYMHPYCFYNS

SEQ ID NO: 354 - protein, 15 aa, synthetic construct
SLPCSYMHPYCFYNS

SEQ ID NO: 355 - protein, 158 aa, synthetic construct
QGQSGQGPLMTCSDYYTCKNNLGGGSSGGSISSGLLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 356 - protein, 158 aa, synthetic construct
QGQSGQGPLMTCSDYYTCHNNLGGGSSGGSISSGLLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 357 - protein, 158 aa, synthetic construct
QGQSGQGPLMTCSDYYTCLNNLGGGSSGGSISSGLLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 358 - protein, 158 aa, synthetic construct
QGQSGQGPLMTCSDYYTCRNNLGGGSSGGSISSGLLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 359 - protein, 158 aa, synthetic construct
QGQSGQGLSCTHSRYDMHCPHMGGGSSGGSISSGLLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 360 - protein, 158 aa, synthetic construct
QGQSGQGHYCHSRTDTITHCNAGGGSSGGSISSGLLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 361 - protein, 158 aa, synthetic construct
QGQSGQGWCPRLFDRPSMGCPTGGGSSGGSISSGLLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 362 - protein, 158 aa, synthetic construct
QGQSGQGWWPPCQGGAWCEQRIGGGSSGGSISSGLLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 363 - protein, 158 aa, synthetic construct
QGQSGQGKFSCTTMPHKNCMKPGGGSSGGSISSGLLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 364 - protein, 158 aa, synthetic construct
QGQSGQGHSGCPRLFDRCSAPAGGGSSGGSISSGLLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 365 - protein, 158 aa, synthetic construct
QGQSGQGPLMTCTKYNNCINNLGGGSSGGSISSGLLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 366 - protein, 158 aa, synthetic construct
QGQSGQGPNYMCKLNTCTHHIAGGGSSGGSISSGLLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 367 - protein, 158 aa, synthetic construct
QGQSGQGAACAGGVLFDRCSVDGGGSSGGSISSGLLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 368 - protein, 158 aa, synthetic construct
QGQSGQGFICPTLYDRPHCMHTGGGSSGGSISSGLLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 369 - protein, 158 aa, synthetic construct
QGQSGQGNMPACQGGSWCRPGYGGGSSGGSISSGLLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 370 - protein, 162 aa, synthetic construct
QGQSGQGPLMTCSDYYTCKNNLGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 371 - protein, 162 aa, synthetic construct
QGQSGQGPLMTCSDYYTCHNNLGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 372 - protein, 162 aa, synthetic construct
QGQSGQGPLMTCSDYYTCLNNLGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 373 - protein, 162 aa, synthetic construct
QGQSGQGPLMTCSDYYTCRNNLGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 374 - protein, 162 aa, synthetic construct
QGQSGQGLSCTHSRYDMHCPHMGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 375 - protein, 162 aa, synthetic construct
QGQSGQGHYCHSRTDTITHCNAGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 376 - protein, 162 aa, synthetic construct
QGQSGQGWCPRLFDRPSMGCPTGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 377 - protein, 162 aa, synthetic construct
QGQSGQGWWPPCQGGAWCEQRIGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 378 - protein, 162 aa, synthetic construct
QGQSGQGKFSCTTMPHKNCMKPGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 379 - protein, 162 aa, synthetic construct
QGQSGQGHSGCPRLFDRCSAPAGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 380 - protein, 162 aa, synthetic construct
QGQSGQGPLMTCTKYNNCINNLGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 381 - protein, 162 aa, synthetic construct
QGQSGQGPNYMCKLNTCTHHIAGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 382 - protein, 162 aa, synthetic construct
QGQSGQGAACAGGVLFDRCSVDGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 383 - protein, 162 aa, synthetic construct
QGQSGQGFICPTLYDRPHCMHTGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 384 - protein, 162 aa, synthetic construct
QGQSGQGNMPACQGGSWCRPGYGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 385 - protein, 162 aa, synthetic construct
QGQSGQGSWCHSATDTILPCSNGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 386 - protein, 162 aa, synthetic construct
QGQSGQGSPACSDRYYQTCVLNGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 387 - protein, 162 aa, synthetic construct
QGQSGQGMSCVVDRFDRQCPHLGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 388 - protein, 162 aa, synthetic construct
QGQSGQGTTRCEHYWFTCPLSPGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 389 - protein, 162 aa, synthetic construct
QGQSGQGDCTGYSPSVLPACRVGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 390 - protein, 162 aa, synthetic construct
QGQSGQGFCSGYSPSVLPSCLMGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 391 - protein, 162 aa, synthetic construct
QGQSGQGSKPCSYMHPYCFYNSGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 392 - protein, 162 aa, synthetic construct
QGQSGQGLTRCTIAHPYCYYNYGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 393 - protein, 162 aa, synthetic construct
QGQSGQGPNTCMSERRICSLTYGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 394 - protein, 162 aa, synthetic construct
QGQSGQGPRPHCAILRQCLAATGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 395 - protein, 162 aa, synthetic construct
QGQSGQGSRPCSYMHPYCFYNSGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 396 - protein, 162 aa, synthetic construct
QGQSGQGSHPCSYMHPYCFYNSGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 397 - protein, 162 aa, synthetic construct
QGQSGQGSLPCSYMHPYCFYNSGGGSSGGAVGLLAPPGGLSGRSDNIGGSDIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

(19) United States
(12) Patent Application Publication
(10) Pub. No.: US 2026/0355338 A1
(43) Pub. Date: Nov. 12, 2026

(54) EPCAM IMMUNOCONJUGATES AND USES THEREOF

(71) Applicants: CytomX Therapeutics, Inc., South San Francisco, CA (US); ImmunoGen, Inc., Waltham, MA (US)

(72) Inventors: Bob Y. LIU, South San Francisco, CA (US); Vangipuram RANGAN, Pleasant Hill, CA (US); Chaoran JING, South San Francisco, CA (US); Sharmistha SAHA, San Jose, CA (US); Eric Henry WESTIN, Waltham, MA (US)

(21) Appl. No.: 18/880,768

(22) PCT Filed: Jul. 12, 2023
(86) PCT No.: PCT/US2023/070016

§ 371 Date: Jan. 2, 2025

Related U.S. Application Data
(60) Provisional application No. 63/388,486, filed on Jul. 12, 2022.

(51) Int. Cl. A61K 47/68

(52) U.S. Cl. CPC A61K 47/6849; A61K 47/68037; A61K 47/6889

(57) ABSTRACT

The disclosure generally relates to immunoconjugates comprising antibodies and antibody fragments that specifically bind human EpCAM, as well as, methods of making and using human EpCAM antibodies, human EpCAM antibody fragments, human EpCAM activatable antibodies, and human EpCAM immunoconjugates, for the diagnosis and treatment of diseases such as cancer.

### 1. CROSS-REFERENCE TO RELATED APPLICATIONS

This application is a 35 U.S.C. 371 National Phase Entry Application of PCT/US2023/070016, filed Jul. 12, 2023, which claims the priority benefit of U.S. provisional application No. 63/388,486 filed Jul. 12, 2022, pursuant 35 U.S.C. § 119(e), the contents of which are incorporated herein in their entireties by reference thereto.

### 2. SEQUENCE LISTING

The instant application contains a Sequence Listing which has been submitted electronically in XML format and is hereby incorporated by reference in its entirety. Said XML copy, created on Jul. 6, 2023 is named CYTX-097-PCT_SL.xml and is 336,140 bytes in size.

### 3. BACKGROUND

The disclosure generally relates to antibodies and antibody fragments that specifically bind human EpCAM, EpCAM activatable antibodies, and immunoconjugates thereof, as well as, methods of making and using the antibodies, antibody fragments, activatable antibodies, and immunoconjugates, for the diagnosis and treatment of diseases such as cancer.

Epithelial cell adhesion molecule (EpCAM), is a type I trans-membrane glycoprotein comprising an extracellular domain, a transmembrane domain, and a single intracellular domain. EpCAM expression in human is epithelia-specific. The majority of epithelial cells express EpCAM, except squamous epithelium and some specific epithelium cell types, such as epidermal keratinocytes, hepatocytes, gastric parietal cells, and myoepithelial cells (Balzar et al., J. Mol. Med. 77:699-712 (1999); Momburg et al., Cancer Res 47:2883-2891 (1987)).

EpCAM is abundantly and homogeneously expressed on human carcinomas of different origins (Went et al., Br. J. Cancer 94:128-35 (2006); Herlyn et al., Proc Natl. Acad. Sci. USA 76:1438-1442 (1979); Went et al., Hum. Pathol. 35:122-128 (2004)). EpCAM is overexpressed in the vast majority of epithelial cancers, including for example, ovarian cancers, colon cancers, stomach cancers, prostate cancers, and lung cancers. In addition, EpCAM has been shown to be expressed on the majority of primary, metastatic, and disseminated NSCLC (non-small cell lung cancer cells) (Passlick, Int. J. Cancer 87:548-552 (2000)), on gastric and gastro-esophageal junction adenocarcinomas (Martin, J. Clin. Pathol. 52:701-704 (1999)) and in cell lines derived from colorectal, pancreatic carcinomas and breast carcinomas (Szala, Proc. Natl. Acad. Sci. USA 87:3542-3546 (1990), Packeisen, Hybridoma 18:37-40 (1999)). In another study, immunohistochemical analysis of 108 samples of secondary tumors has found that only 4% lacked EpCAM expression. EpCAM is overexpressed also in cancer-initiating or cancer stem cells isolated from colon, breast, pancreas and prostate carcinomas (O'Brien et al., Nature 445:106-110 (2007); Marhaba et al., Curr. Mol. Med. 8:784-804 (2008)).

Normal cells express EpCAM on the basolateral side of the epithelial membrane, whereas cancer cells heavily express EpCAM on the apical surface.

Antibody-based therapeutics have been designed to exploit this characteristic of EpCAM expression, as normal cellular EpCAM is less prominent and less exposed, meaning healthy cells may not be as susceptible to binding by therapeutic anti-EpCAM antibodies.

Camptothecin (CPT) is a pentacyclic alkaloid isolated from the bark and stem of Camptotheca acuminata (Camptotheca, Happy tree), a tree native to China. Camptothecin inhibits topoisomerase I, which leads to cell death. Because of its cytotoxic mechanism and broad-spectrum antitumor activity, there have been substantial efforts towards developing clinical analogues of camptothecin. Poor solubility and inactivity at physiological conditions, however, have limited the clinical development of suitable camptothecin analogues. Camptothecin and most of its derivatives are not soluble in aqueous buffers. Further, camptothecin is in equilibrium in an active lactone form and inactive hydrolyzed carboxylate form, thereby limiting its therapeutic efficacy.

A number of antibodies to EpCAM have been used in the clinic but failed for various reasons. The EpCAM antibodies tested take a number of formats, including naked antibodies, immunotoxins and bi- or tri-specific antibodies (Baeuerle, Br. J. Cancer, 96:417-423 (2007)). For example, adecatumumab (MT201), a naked anti-EpCAM antibody has been tested in clinical studies of treatment in colorectal, prostate and breast cancers. Safety issues facing the current anti-EpCAM antibody-based approaches include systemic intolerability and acute pancreatitis. Thus, although there have been several attempts to develop therapeutic antibodies to EpCAM, there is a significant need for the development of novel therapeutic EpCAM immunoconjugates that overcome the shortcomings and limitations of the previously developed antibodies or antibody derivatives.

### 4. SUMMARY

In one aspect, the present disclosure provides an immunoconjugate comprising: (a) an activatable antibody comprising a full length human IgG1 EpCAM antibody having eight cysteines which, when in a native conformation, form four interchain disulfide bonds, the full length human IgG1 EpCAM antibody comprising a heavy chain comprising the amino acid sequence of SEQ ID NO:103 and a light chain comprising the amino acid sequence of SEQ ID NO:179; and (b) eight linker-payloads depicted by the structure:

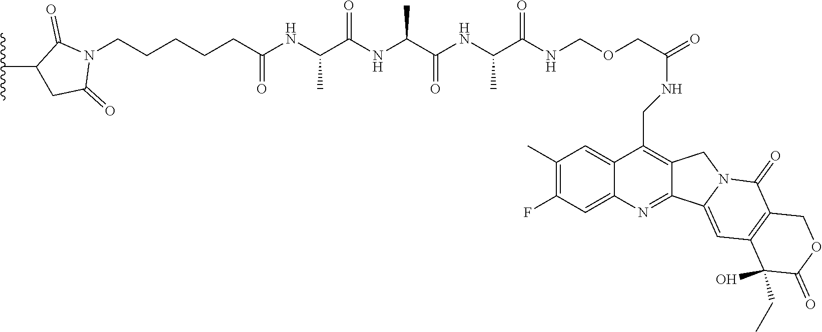 [SMILES: *C1CC(=O)N(CCCCCC(=O)N[C@@H](C)C(=O)N[C@@H](C)C(=O)N[C@@H](C)C(=O)NCOCC(=O)NCc2c3c(nc4cc(F)c(C)cc24)-c2cc4c(c(=O)n2C3)COC(=O)[C@]4(O)CC)C1=O]

or a pharmaceutically acceptable salt thereof, wherein each of the eight linker-payloads is individually covalently bound to the activatable antibody via one of the eight cysteines.

In another aspect, the present disclosure provides an immunoconjugate comprising: (a) an activatable antibody comprising: (i) an EpCAM antibody or EpCAM-binding fragment thereof, comprising: (1) a heavy chain complementarity determining region 1 (VH-CDR1) comprising the amino acid sequence NYYIH (SEQ ID NO:13); (2) a heavy chain complementarity determining region 2 (VH-CDR2) comprising the amino acid sequence WIYPGNVYIQYNEKFKG (SEQ ID NO:14); (3) a heavy chain complementarity determining region 3 (CDR3) (VH-CDR3) comprising the amino acid sequence DGPWFAY (SEQ ID NO:15); (4) a light chain complementarity determining region 1 (VL-CDR1) comprising the amino acid sequence RSSRSLLHSDGFTYLY (SEQ ID NO:42); (5) a light chain complementarity determining region 2 (VL-CDR2) comprising the amino acid sequence QTSNLAS (SEQ ID NO:40); (6) a light chain complementarity determining region 3 (VL-CDR3) comprising the amino acid sequence AQNLELPNT (SEQ ID NO:41); (ii) a cleavable moiety coupled to the EpCAM antibody or EpCAM-binding fragment thereof that comprises the amino acid sequence AVGLLAPPGGLSGRSDNI (SEQ ID NO:168) or the amino acid sequence ISSGLLSGRSDNI (SEQ ID NO:169); and (iii) a masking moiety coupled to the EpCAM antibody or EpCAM-binding fragment thereof that comprises the amino acid sequence WWPPCQGGAWCEQRI (SEQ ID NO:155), wherein the masking moiety inhibits the binding of the antibody or antibody fragment to EpCAM when the activatable antibody is in an uncleaved state, wherein the activatable antibody in an uncleaved state has a structural arrangement from N-terminus to C-terminus of: (masking moiety)-(cleavable moiety)-(antibody or antibody fragment) or (antibody or antibody fragment)-(cleavable moiety)-(masking moiety); and (b) at least one linker-payload depicted by the structure:

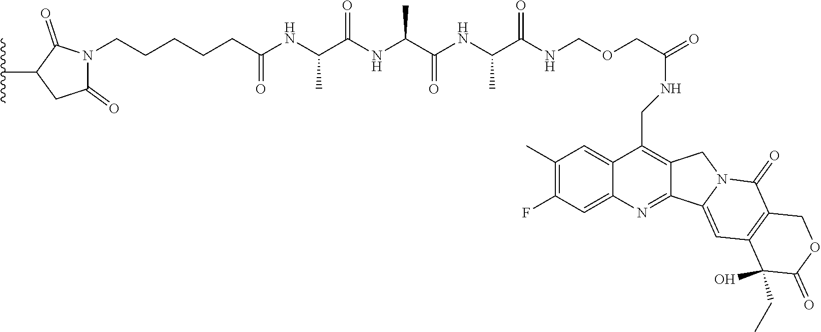 [SMILES: *C1CC(=O)N(CCCCCC(=O)N[C@@H](C)C(=O)N[C@@H](C)C(=O)N[C@@H](C)C(=O)NCOCC(=O)NCc2c3c(nc4cc(F)c(C)cc24)-c2cc4c(c(=O)n2C3)COC(=O)[C@]4(O)CC)C1=O]

or a pharmaceutically acceptable salt thereof.

In some embodiments, the immunoconjugate has a drug-to-antibody ratio (DAR) of 8.

In some embodiments, the activatable antibody is a full length human IgG1 EpCAM antibody having eight cysteines which, when in a native conformation form four interchain disulfide bonds, and wherein each linker-payload is individually covalently bound to the activatable antibody via one of the eight cysteines.

In yet another aspect, the present disclosure provides an immunoconjugate comprising: (a) an activatable antibody comprising: (i) a full length human IgG1 EpCAM antibody having eight cysteines which, when in a native conformation, form four interchain disulfide bonds, the full length human IgG1 EpCAM antibody comprising: (1) a heavy chain complementarity determining region 1 (VH-CDR1) comprising the amino acid sequence NYYIH (SEQ ID NO:13); (2) a heavy chain complementarity determining region 2 (VH-CDR2) comprising the amino acid sequence WIYPGNVYIQYNEKFKG (SEQ ID NO:14); (3) a heavy chain complementarity determining region 3 (CDR3) (VH-CDR3) comprising the amino acid sequence DGPWFAY (SEQ ID NO:15); (4) a light chain complementarity determining region 1 (VL-CDR1) comprising the amino acid sequence RSSRSLLHSDGFTYLY (SEQ ID NO:42); (5) a light chain complementarity determining region 2 (VL-CDR2) comprising the amino acid sequence QTSNLAS (SEQ ID NO:40); (6) a light chain complementarity determining region 3 (VL-CDR3) comprising the amino acid sequence AQNLELPNT (SEQ ID NO:41); (ii) a cleavable moiety coupled to the EpCAM antibody or EpCAM-binding fragment thereof that comprises the amino acid sequence ISSGLLSGRSDNI (SEQ ID NO:169); and (iii) a masking moiety coupled to the EpCAM antibody or EpCAM-binding fragment thereof that comprises the amino acid sequence WWPPCQGGAWCEQRI (SEQ ID NO:155), wherein the masking moiety inhibits the binding of the antibody or antibody fragment to EpCAM when the activatable antibody is in an uncleaved state, wherein the activatable antibody in an uncleaved state has a structural arrangement from N-terminus to C-terminus of: (masking moiety)-(cleavable moiety)-(antibody) or (antibody)-(cleavable moiety)-(masking moiety); and (b) eight linker-payloads depicted by the structure:

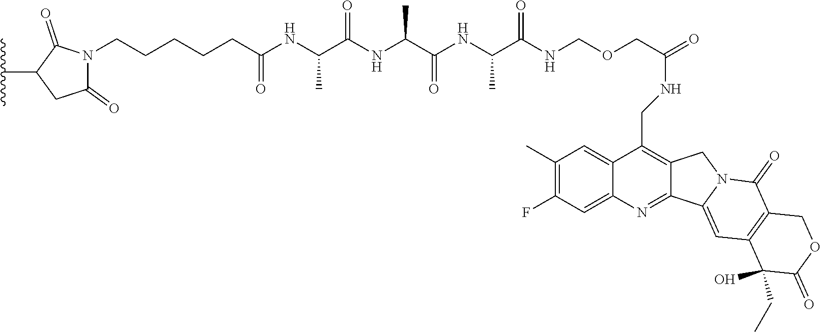 [SMILES: *C1CC(=O)N(CCCCCC(=O)N[C@@H](C)C(=O)N[C@@H](C)C(=O)N[C@@H](C)C(=O)NCOCC(=O)NCc2c3c(nc4cc(F)c(C)cc24)-c2cc4c(c(=O)n2C3)COC(=O)[C@]4(O)CC)C1=O]

or a pharmaceutically acceptable salt thereof, wherein each of the eight linker-payloads is individually covalently bound to the activatable antibody via one of the eight cysteines.

In some embodiments, the full length human IgG1 EpCAM antibody comprises a heavy chain variable domain (VH) comprising the amino acid sequence of SEQ ID NO:54 and a light chain variable domain (VL) comprising the amino acid sequence of SEQ ID NO: 89. In other embodiments, the full length human IgG1 EpCAM antibody comprises a heavy chain comprising the amino acid sequence of SEQ ID NO:103 and a light chain comprising the amino acid sequence of SEQ ID NO:179.

In another aspect, the description provides an immunoconjugate obtainable by the method of Example 7.4.1 provided herein.

In another aspect, the description provides an immunoconjugate obtainable by a method comprising: (a) mixing an activatable EpCAM antibody consisting of a heterotetramer of two heavy chains each consisting of the amino acid sequence of SEQ ID NO:103 and two light chains each consisting of the amino acid sequence of SEQ ID NO:179 with a mixture of EPPS buffer and an aqueous EDTA solution to form an activatable EpCAM reaction mixture; (b) mixing an activatable EpCAM antibody consisting of a heterotetramer of two heavy chains each consisting of the amino acid sequence of SEQ ID NO:103 and two light chains each consisting of the amino acid sequence of SEQ ID NO:179 with a mixture of EPPS buffer and an aqueous EDTA solution to form an activatable EpCAM reaction mixture; (c) mixing the reduced activatable EpCAM reaction mixture with a camptothecin linker-toxin stock solution comprising a linker-toxin represented by the structure

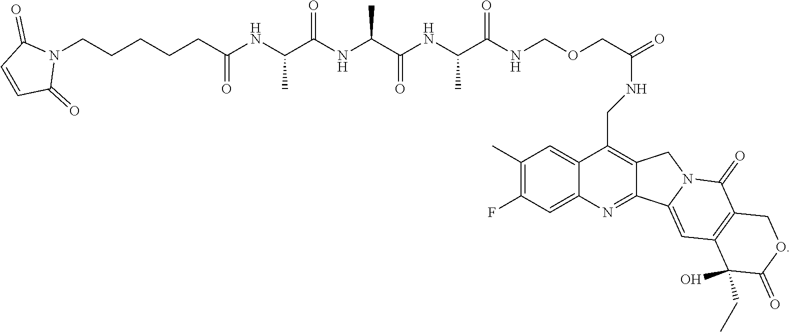 [SMILES: CC[C@@]1(O)C(=O)OCc2c1cc1n(c2=O)Cc2c-1nc1cc(F)c(C)cc1c2CNC(=O)COCNC(=O)[C@H](C)NC(=O)[C@H](C)NC(=O)[C@H](C)NC(=O)CCCCCN1C(=O)C=CC1=O]

Thereby forming the immunoconjugate.

In another aspect, the description provides a nucleic acid comprising one or more coding for the activatable antibody of the immunoconjugates described herein.

Also provided are vectors comprising such nucleic acids. In some embodiments, the vectors are viral vectors.

In another aspect, the description provides a method of producing an immunoconjugate comprising: (a) culturing a host cell comprising a vector of the present disclosure (b) isolating an activatable antibody from the host cell; and (c) conjugating to the activatable antibody at least one linker-payload reactant comprising camptothecin or a derivative thereof.

In another aspect, the description provide a method of producing an immunoconjugate, comprising conjugating at least one linker-payload reactant comprising camptothecin or a derivative thereof to an activatable EpCAM antibody described herein.

In some embodiments, the linker-payload reactant comprises a compound of Formula I:

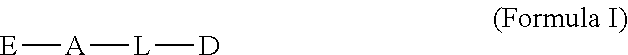

wherein:

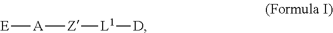

wherein:
D is represented by the following structural formula:

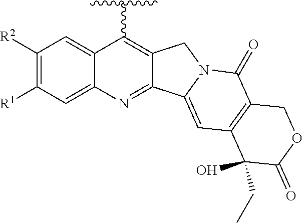

wherein R¹ is F and R² is methyl;
-L¹-Z′—* is —(C₁-C₄ alkylene)—O—CH₂—NR⁸—*, —(C₁-C₄ alkylene)-NR⁸—*, or —(C₁-C₅ alkylene)-NR⁵C(═O)—(C₁-C₅ alkylene)-O—CH₂—NR⁸—*, where * is a site covalently attached to A;
each R⁵ is independently H, methyl, or benzyl;
each R⁸ is independently H, methyl, or benzyl;
A is a peptide comprising 2 to 4 amino acids; and
E is —C(═O)—(C₁-C₁₀ alkylene)-X³ wherein X³ is

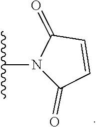 [SMILES: *N1C(=O)C=CC1=O]

In some embodiments, the linker-payload reactant is:

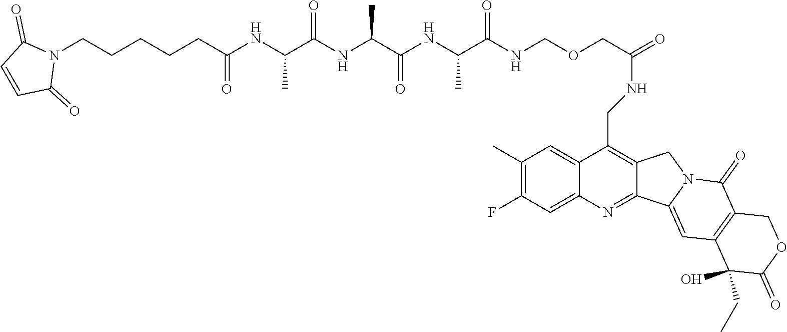 [SMILES: CC[C@@]1(O)C(=O)OCc2c1cc1n(c2=O)Cc2c-1nc1cc(F)c(C)cc1c2CNC(=O)COCNC(=O)[C@H](C)NC(=O)[C@H](C)NC(=O)[C@H](C)NC(=O)CCCCCN1C(=O)C=CC1=O]

or a pharmaceutically acceptable salt thereof.

In some embodiments, the linker-payload reactant is:

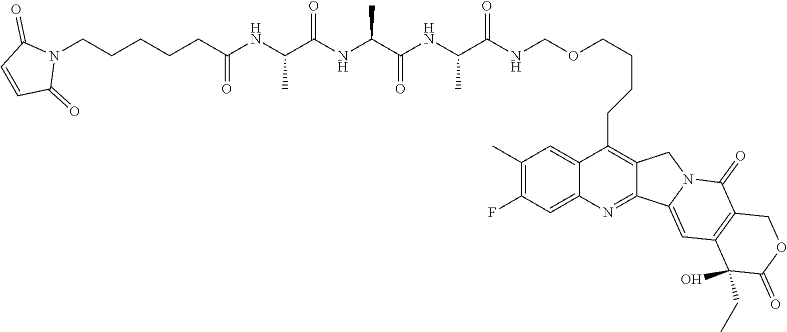 [SMILES: CC[C@@]1(O)C(=O)OCc2c1cc1n(c2=O)Cc2c-1nc1cc(F)c(C)cc1c2CCCCOCNC(=O)[C@H](C)NC(=O)[C@H](C)NC(=O)[C@H](C)NC(=O)CCCCCN1C(=O)C=CC1=O]

or a pharmaceutically acceptable salt thereof.

In certain embodiments, the linker-payload reactant comprises exetecan. In other embodiments, the linker-payload reactant comprises deruxtecan.

Also provided are compositions comprising an immunoconjugate of the present disclosure and a pharmaceutically acceptable carrier.

In some aspects, an immunoconjugate or composition of the present disclosure can be used in the treatment of an EpCAM-expressing cancer. In some embodiments, the EpCAM-expressing cancer is colorectal cancer.

### 5. BRIEF DESCRIPTION OF THE FIGURES

FIG. 1A shows the camptothecin derivative linker-payload of use in accordance with certain embodiments of the disclosure.

FIG. 1B shows the conjugation of a camptothecin-derived (CPT66) linker-payload to an activatable EpCAM antibody in accordance with certain embodiments of the disclosure.

FIG. 2 shows a cartoon depicting an activatable EpCAM antibody immunoconjugate in accordance with certain embodiments of the disclosure. The depicted embodiment comprises an EpCAM antibody, a cleavable substrate linker, and a mask. In an uncleaved (inactive) state, the mask inhibits the binding of the EpCAM antibody to EpCAM. The cleavable substrate linker is cleavable by a protease. Upon cleavage, the mask is released and the antibody is free to bind to EpCAM. The depicted embodiment includes 8 conjugated camptothecin-derived (CPT66) linker-payloads (shown as ovals in cartoon). The conjugation of the linker-payloads to the activatable EpCAM antibody is stochastic, with conjugation occurring at the antibody's inter-chain cysteines.

FIGS. 3A-3D show cytotoxicity assessments for OV90 (FIG. 3A), SNU182 (FIG. 3B), KYSE270 (FIG. 3C), and SNU1 (FIG. 3D) cancer cell lines with various EpCAM linker-payloads. EpCAM-DM21 was conjugated to DM21 maytansinoid linker payload with a DAR of 4, EpCAM-MMAT was a site-specific conjugation of auristatin derivative via a valine-citrulline linker with a DAR of 2, EpCAM-mGGFG-DXd was conjugated to linker-payload used for fam-trastuzumab deruxtecan-nxki with a DAR of 8, EpCAM-mGGFG-CPT66 is CPT66 payload conjugated with GGFG linker (SEQ ID NO: 310) from fam-trastuzumab deruxtecan-nxki with DAR of 8, and EpCAM-Lala-ala-ala-CPT66 was conjugated to CPT66 payload using a tripeptide linker (ala-ala-ala) with DAR of 8. OV90 is an ovarian cancer cell line with ~375,149 EpCAM receptors expressed on the cell surface, SNU182 is a liver cancer cell line with 18,898 EpCAM receptors, KYSE270 is an esophageal cancer cell line with 136,409 EpCAM receptors, and SNU1 is a gastric cancer cell line with 425 EpCAM receptors. The potency of EpCAM-CPT was comparable to that of EpCAM-GGFG-DXd ADC across cell lines with varying EpCAM receptor expression.

FIGS. 4A-4E show cytotoxicity assessment of 43 cancer cell lines treated with various EpCAM linker payloads listed in FIGS. 3A-3D. The cytotoxicity activity was shown as area under the curve (AUC) that integrated the AUC of the cytotoxicity curves shown in FIGS. 3A-D, and the lower the AUC, the higher the potency of the ADC. Average AUC for each cancer indications was plotted, and the number of cell lines tested were indicated in parenthesis. Error bars represented standard error measurements. Data suggest that breast, CRC, ovarian, and head and neck cancer cell lines are relatively sensitive to EpCAM-CPT66 ADC, whereas bladder, uterine, liver, and esophagus cancer cell lines are relatively insensitive to the ADC.

FIGS. 5A-5B shows an in vivo anti-tumor assessment of the EpCAM-CPT66 immunoconjugate in two colorectal cancer cell line-derived xenograft models, HCT116 (FIG. 5A) and HT29 (FIG. 5B). EpCAM activatable antibody was conjugated to CPT66 with a tripeptide (L-ala-L-ala-L-ala) linker and DAR of 8 (EpCAM-CPT66), and it was compared to EpCAM-GGFG-DXd conjugated to the linker-payload similar to that of fam-trastuzumab deruxtecan-nxki with DAR of 8. All immunoconjugate and Isotype control-ADC were given as a single dose with the indicated mg/kg (mpk) via intravenous injection at day 0. Anti-tumor activity of EpCAM-CPT66 was comparable to that of EpCAM-GGFG-DXd.

FIGS. 6A-6F shows an in vivo anti-tumor assessment of EpCAM-CPT66 in colorectal patient-derived xenograft (PDX) models. Each model was dosed with 6 mg/kg (mpk) of EpCAM-CPT66 and was compared with the same dose of EpCAM-GGFG-DXd. The immunoconjugate dosing schedule was once every two weeks for a total of 3 doses (Q2W×3). Overall anti-tumor activity of EpCAM-CPT66 was comparable to that of EpCAM-GGFG-DXd.

FIG. 7 shows an anti-tumor activity assessment of EpCAM-CPT66 in various PDX models. The drug dose was 6 mg/kg and given every 2 weeks for a total of 3 doses (Q2W×3). Percent tumor volume shrinkage relative to start of dosing was calculated by percent tumor volume change=[TV of post treatment−TV of initial)/TV of initial)×100. Smallest (highest negative change) percentage was used to compare drug treatment activities. Breast and colorectal PDX tumors were relatively sensitive to the EpCAM-CPT66 treatment.

FIG. 8 shows a pharmacokinetic graph of EpCAM-CPT66 from the Cyno dose ranging finding experiments. Cyno were dosed with 10, 30, and 60 mg/kg of EpCAM-CPT66 intravenously every 2 weeks and given a total of 3 doses (Q2W×3). Total immunoconjugate (activatable antibody+linker-payload) concentration in monkey plasma was determined by ELISA at various sample collection times. The graphs indicated that drug exposure was consistent through 3 doses of the drug, and no apparent anti-drug antibody that affecting drug clearance was seen.

FIG. 9 shows an in vivo assessment of the cytotoxicity of EpCAM-CPT66 and EpCAM(−)-CPT in the HCT116 colorectal cancer cell line.

FIG. 10 depicts a summary of the cytotoxicity of the unmasked EpCAM(−)-CPT66 on 57 cell lines from 11 cancer indications.

### 6. DETAILED DESCRIPTION

The disclosure provides immunoconjugates having antibodies and antigen-binding antibody fragments that specifically bind human EpCAM, including activatable antibodies (activatable forms of the EpCAM antibodies or EpCAM-binding antibody fragments thereof). In some instances, the immunoconjugates comprise a camptothecin derivative linker-payload with the following structure (where the following structure represents the linker-payload prior to conjugation to the antibody, i.e. the linker-payload reactant):

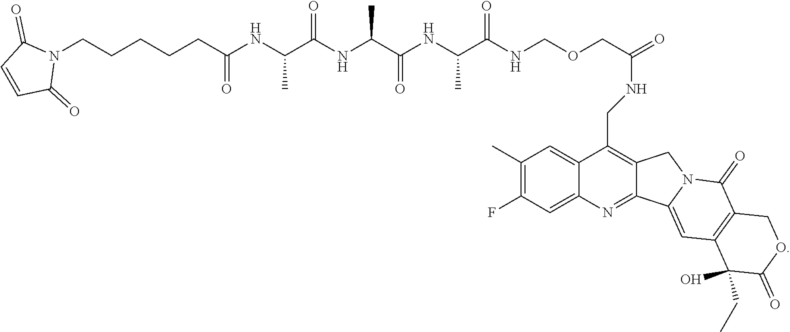 [SMILES: CC[C@@]1(O)C(=O)OCc2c1cc1n(c2=O)Cc2c-1nc1cc(F)c(C)cc1c2CNC(=O)COCNC(=O)[C@H](C)NC(=O)[C@H](C)NC(=O)[C@H](C)NC(=O)CCCCCN1C(=O)C=CC1=O]

Thus, in some instances, the immunoconjugates comprise a camptothecin derivative linker-payload with the following structure (wherein the ** denotes the covalent attachment position to the EpCAM antibody or activatable antibody):

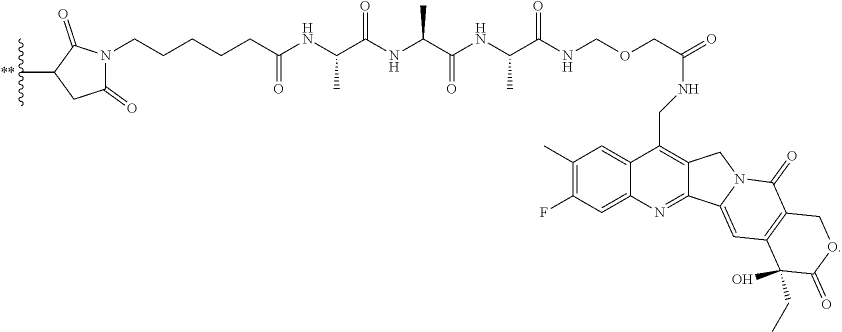 [SMILES: *C1CC(=O)N(CCCCCC(=O)N[C@@H](C)C(=O)N[C@@H](C)C(=O)N[C@@H](C)C(=O)NCOCC(=O)NCc2c3c(nc4cc(F)c(C)cc24)-c2cc4c(c(=O)n2C3)COC(=O)[C@]4(O)CC)C1=O]

EpCAM is known to be associated with cell-cell adhesion in epithelia and to be involved in cell signaling, differentiation, proliferation, and migration. The overexpression of EpCAM has been implicated in the pathogenesis of diseases and disorders, such as cancer. For example, EpCAM is highly expressed in a variety of cancer types such as, for example, breast cancer, lung cancer, liver cancer, stomach cancer, head & neck cancer, prostate cancer, pancreatic cancer, ovarian cancer, and colon cancer, and most cancers (and metastases) of epithelial origin. EpCAM is also highly expressed in tumor initiating/cancer stem cells. The provided EpCAM immunoconjugates have uses that include treating such diseases and cancers.

### 6.1 Definitions

To facilitate an understanding, a number of terms and phrases are defined below.

The terms “epithelial cell adhesion molecule” or “EpCAM”, as used herein, refers to any native human EpCAM unless otherwise indicated. The term also encompasses naturally occurring variants of EpCAM, e.g., splice variants, allelic variants and isoforms. EpCAM polypeptides can be isolated from a variety of sources, such as from human or cynomolgous tissue or other biological samples, or prepared by known recombinant or synthetic methods. EpCAM is also known as CD326, 17-1Aantigen, HEA125, MK-1, EGP-2, EGP314, EGP40, GA733-2, KSA, TACSTD1, TROP1, KS1/4, M4S1, DIAR5, MIC18, HNPCC8, and ESA. Examples of EpCAM sequences include, but are not limited to NCBI reference number NP_002345.2 (amino acid residues 24-314 correspond to mature EpCAM, amino acids 24-265 correspond to the extracellular region of mature EpCAM (SEQ ID NO:1)). The extracellular region of mature EpCAM can further be divided into three domains: D1 (amino acids 1-36 of SEQ ID NO:1 (SEQ ID NO:2)), D2 (amino acids 43-112 of SEQ ID NO:1 (SEQ ID NO:3)), and D3 (amino acids 113-243 of SEQ ID NO:1 (SEQ ID NO:4)).

The terms “antibody” and “antigen-binding antibody fragment” and the like, as used herein, include any protein- or peptide-containing molecule that comprises at least a portion of an immunoglobulin molecule, such as, but not limited to, at least one complementarity determining region (CDR) of a heavy or light chain or an antigen binding portion thereof. Such antibody optionally further affects at least one EpCAM activity, such as, but not limited to, where such antibody modulates, decreases, increases, antagonizes, agonizes, partially agonizes, partially antagonizes, mitigates, alleviates, blocks, inhibits, abrogates and/or interferes with at least one EpCAM activity or binding in vitro, in situ, in vivo and/or ex vivo. In some embodiments, the EpCAM antibody or EpCAM-binding antibody fragment thereof affects at least one EpCAM-mediated activity or function selected from: ligand binding, receptor signaling, membrane association, cell migration, cell proliferation, receptor binding activity, RNA, DNA or protein production and/or synthesis.

Antibodies are heterotetrameric glycoproteins, composed of two identical light chains (LC) and two identical heavy chains (HC). Typically, each light chain is linked to a heavy chain by one covalent disulfide bond, while the number of disulfide linkages varies between the heavy chains of different immunoglobulin isotypes. Each heavy and light chain also has spaced intrachain disulfide bridges. Each heavy chain has at one end a variable region (VH) followed by a number of constant domains. Each light chain has a variable region at one end (VL) and a constant domain at its other end; the constant domain of the light chain is aligned with the first constant domain of the heavy chain and the light chain variable region is aligned with the variable region of the heavy chain. Antibody light chains of any vertebrate species can be assigned to one of two clearly distinct types, namely kappa and lambda, based on the amino acid sequences of their constant domains. Immunoglobulins can be assigned to five major classes, namely IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4.

The term “antibody” also includes fragments, specified portions and variants thereof, including antibody mimetics or comprising portions of antibodies that mimic the structure and/or function of an antibody or specified fragment or portion thereof, including single chain antibodies and antigen (e.g., EpCAM)-binding antibody fragments. Functional fragments include antigen-binding fragments that bind to a mammalian antigen, such as EpCAM, alone or in combination with other antigens.

For example, antibody fragments capable of binding to antigen or portions thereof, including, but not limited to, Fab (e.g., by papain digestion), Fab′ (e.g., by pepsin digestion and partial reduction) and F(ab′)2 (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments, are encompassed by the disclosure (see, e.g., Colligan, Immunology).

Such fragments can be produced by enzymatic cleavage, synthetic or recombinant techniques, as known in the art and/or as disclosed herein. Antibodies can also be produced in a variety of truncated forms using antibody genes in which one or more stop codons have been introduced upstream of the natural stop site. For example, a combination gene encoding a F(ab′)2 heavy chain portion can be designed to include DNA sequences encoding the CH1 domain and/or hinge region of the heavy chain. The various portions of antibodies can be joined together chemically by conventional techniques, or can be prepared as a contiguous protein using genetic engineering techniques.

The term “antibody fragment” refers to a portion of an intact antibody, generally the antigen binding or variable region of an intact antibody. Examples of antibody fragments include, but are not limited to Fab, Fab′, F(ab′)2, single chain (scFv) and Fv fragments, diabodies; linear antibodies; single-chain antibody molecules; single Fab arm “one arm” antibodies and multispecific antibodies formed from antibody fragments (each having a VH and VL), among others.

Antibody fragments include any protein or peptide containing molecule that comprises at least a portion of an immunoglobulin molecule, such as but not limited to, at least one complementarity determining region (CDR) of a heavy or light chain or a ligand binding portion thereof, a heavy chain or light chain variable region, a heavy chain or light chain constant region, a framework region, or any portion thereof, or at least one portion of an antigen or antigen receptor or binding protein, which can be incorporated into an EpCAM antibody provided herein.

Term “full length antibody” refers to an antibody in its substantially intact form, and not antibody fragments as defined above. The term particularly refers to an antibody with heavy chains that contain a hinge region and an Fc region, including a modified Fc region (e.g., a mutated and/or clipped hinge region).

The term “variable” refers to the fact that certain portions of the variable regions of antibodies differ extensively in sequence among antibodies and are used in the binding and specificity of each particular antibody for its particular antigen. However, the variability is not evenly distributed throughout the variable regions of antibodies. The variability is concentrated in three segments called complementarity-determining regions (CDRs) or hypervariable regions both in the light-chain and the heavy-chain variable regions. The more highly conserved portions of variable regions are called the framework (FR). The variable regions of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen-binding site of antibodies. The constant domains are not involved directly in binding an antibody to an antigen, but exhibit various effector functions, such as participation of the antibody in antibody-dependent cellular toxicity. There are at least two techniques for determining CDRs: (1) an approach based on cross-species sequence variability (i.e., Kabat et al., Sequences of Proteins of Immunological Interest, (5th ed., 1991, National Institutes of Health, Bethesda Md.)); and (2) an approach based on crystallographic studies of antigen-antibody complexes (Al-lazikani et al., J. Molec. Biol. 273:927-948 (1997)). In addition, combinations of these two approaches are sometimes used in the art to determine CDRs.

The Kabat numbering system is generally used when referring to a residue in the variable region (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)).

The amino acid position numbering as in Kabat, refers to the numbering system used for heavy chain variable regions or light chain variable regions of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence can contain fewer or additional amino acids corresponding to a shortening of, or insertion into, a FR or CDR of the variable region. For example, a heavy chain variable region can include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues can be determined for a given antibody by alignment at regions of homology of the sequence of the antibody with a “standard” Kabat numbered sequence. Chothia refers instead to the location of the structural loops (Chothia et al., J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

The terms “EpCAM antibody”, “EpCAM antibody”, “antibody that specifically binds to EpCAM”, “EpCAM-binding antibody fragments thereof”, and “antibody fragment that specifically binds EpCAM” refer to an antibody that is capable of binding EpCAM with sufficient affinity such that the antibody is useful as a diagnostic and/or therapeutic agent in targeting EpCAM. The extent of binding of an EpCAM antibody to an unrelated, non-EpCAM protein is less than about 10% of the binding of the antibody to EpCAM as measured, e.g., by a radioimmunoassay (RIA).

The amino acid position numbering as in Kabat, refers to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence can contain fewer or additional amino acids corresponding to a shortening of, or insertion into, a FR or CDR of the variable domain. For example, a heavy chain variable domain can include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues can be determined for a given antibody by alignment at regions of homology of the sequence of the antibody with a “standard” Kabat numbered sequence. Chothia refers instead to the location of the structural loops (Chothia and Lesk, J. Mol. Biol. 196:901-917 (1987)), wherein the loops are also identified as light and heavy chain CDRs (e.g., L1=LC CDR1, etc.). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

TABLE 1
Amino Acid Position Numbering Systems

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |

(Kabat Numbering)

| | | | |
|---|---|---|---|
| H1 | H31-H35 | H26-H35 | H26-H32 |

(Chothia Numbering)

| | | | |
|---|---|---|---|
| H2 | H50-H65 | H50-H58 | H52-H56 |
| H3 | H95-H102 | H95-H102 | H95-H102 |

The term “epitope” refers to a protein determinant capable of specific binding to an antibody. Epitopes usually consist of chemically active surface groupings of molecules such as amino acids or sugar side chains and usually have specific three-dimensional structural characteristics, as well as specific charge characteristics. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation.

“Blocking” antibody is one which inhibits or reduces the biological activity of the antigen it binds such as EpCAM. Particular blocking antibodies substantially or completely inhibit the biological activity of the antigen. Desirably, the biological activity is reduced by 10%, 20%, 30%, 50%, 70%, 80%, 90%, 95%, or even 100%. In one embodiment, the blocking antibody reduces the EpCAM associated tyrosine kinase activity 10%, 20%, 30%, 50%, 70%, 80%, 90%, 95%, or even 100%.

An “isolated” antibody is one separated and/or recovered from its natural environment. Contaminant components of its natural environment are materials which would interfere with diagnostic or therapeutic uses for the antibody, and may include enzymes, hormones, and other proteinaceous or non-proteinaceous solutes. In particular aspects, the antibody will be purified (1) to greater than 95% by weight of antibody as determined by, for example, the Lowry method, and in some instances more than 99% by weight, (2) to a degree sufficient to obtain at least 15 residues of N-terminal or internal amino acid sequence by use of a spinning cup sequenator, or (3) to homogeneity by SDS-PAGE (sodium dodecyl sulfate polyacrylamide gel electrophoresis) under reducing or non-reducing conditions using Coomassie blue or, in some instances, silver stain. Isolated antibody includes the EpCAM antibody in situ within recombinant cells since at least one component of the antibody's natural environment will not be present. Ordinarily, however, isolated antibody will be prepared by at least one purification step.

A “human antibody” refers to an antibody produced by a human or an antibody having an amino acid sequence corresponding to an antibody produced by a human made using any technique known in the art. This definition of a human antibody includes intact or full-length antibodies, activatable antibodies, antigen (e.g., human and/or cynomolgous EpCAM)-binding antibody fragments, and/or antibodies comprising at least one human heavy and/or light chain polypeptide such as, for example, an antibody comprising murine light chain and human heavy chain polypeptides.

The term “chimeric antibodies,” as used herein, refer to antibodies wherein the sequence of the immunoglobulin molecule is derived from two or more species. Typically, the variable region of both light and heavy chains corresponds to the variable region of antibodies derived from one species of mammals (e.g., mouse, rat, rabbit, etc.) with the desired specificity, affinity, and capability while the constant regions are homologous to the sequences in antibodies derived from another (usually human) to avoid eliciting an immune response in that species.

The term “humanized antibody,” as used herein, refers to forms of non-human (e.g., murine) antibodies that are specific immunoglobulin chains, chimeric immunoglobulins, or antigen-binding antibody fragments that contain minimal non-human (e.g., murine) sequences. Typically, humanized antibodies are human immunoglobulins in which residues from the complementarity determining region (CDR) are replaced by residues from the CDR of a non-human species (e.g., mouse, rat, rabbit, hamster) that have the desired specificity, affinity, and capability (Jones et al., Nature 321:522-525 (1986); Riechmann et al., Nature 332:323-327 (1988): Verhoeyen et al., Science 239:1534-1536 (1988)). In some instances, the Fv framework region (FR) residues of a human immunoglobulin are replaced with the corresponding residues in an antibody from a non-human species that has the desired specificity, affinity, and capability. The humanized antibody can be further modified by the substitution of additional residues either in the Fv framework region and/or within the replaced non-human residues to refine and optimize antibody specificity, affinity, and/or capability. In general, the antibody will comprise substantially all of at least one, and typically two or three, variable regions containing all or substantially all of the CDR regions that correspond to the non-human immunoglobulin whereas all or substantially all of the FR regions are those of a human immunoglobulin consensus sequence. The antibody can also comprise at least a portion of an immunoglobulin constant region or domain (Fc), typically that of a human immunoglobulin. Examples of methods used to generate humanized antibodies are described in U.S. Pat. No. 5,225,539.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity (CDC); Fc receptor binding; antibody-dependent cell-mediated cytotoxicity (ADCC); and antibody-dependent cell-mediated phagocytosis (ADCP).

“Human effector cells” are leukocytes which express one or more FcRs and perform effector functions. In certain aspects, the cells express at least FcγRIII and perform ADCC or ADCP effector function(s). Examples of human leukocytes which mediate ADCC or ADCP include peripheral blood mononuclear cells (PBMC), natural killer (NK) cells, monocytes, cytotoxic T cells and neutrophils. The effector cells may be isolated from a native source, e.g., from blood.

The term “Fc region” as used herein includes the polypeptides comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM, Fc may include the J chain. For IgG, Fc comprises immunoglobulin domains Cγ2 and Cγ3 (Cγ2 and Cγ3) and the hinge between Cγ1 (Cγ1) and Cγ2 (Cγ2). Although the boundaries of the Fc region may vary, the human IgG heavy chain Fc region is usually defined to comprise residues C226 or P230 to its carboxyl-terminus, wherein the numbering is according to the EU index as in Kabat et al., (1991, NIH Publication 91-3242, National Technical Information Service, Springfield, Va.). The “EU index as set forth in Kabat” refers to the residue numbering of the human IgG1 EU antibody as described in Kabat et al., supra. Fc may refer to this region in isolation, or this region in the context of an antibody, antibody fragment, or Fc fusion protein. An Fc variant protein may be an antibody, Fc fusion, or any protein or protein domain that comprises an Fc region. Particular proteins comprise variant Fc regions, which are non-naturally occurring variants of an Fc. Polymorphisms have been observed at a number of Fc positions, including, but not limited to, Kabat 270, 272, 312, 315, 356, and 358, and thus slight differences between the presented sequence and sequences in the prior art may exist and would be understood by one skilled in the art based on the present teachings.

The term “conjugate”, “immunoconjugate”, “ADC”, or “AADC” as used herein, refers to a compound or a derivative thereof that is linked to a cell binding agent (i.e., an EpCAM antibody, EpCAM-binding antibody fragments thereof, or EpCAM activatable antibody) and is defined by a generic formula: C-L-A, wherein C=compound, L=linker, and A=EpCAM-binding agent (EpBA) (e.g., an EpCAM antibody, EpCAM-binding antibody fragments thereof, or EpCAM activatable antibody, as disclosed herein). In some embodiments, the generic formula: D-L-A, wherein D=drug, L=linker and A=cell binding agent (e.g., an EpCAM antibody, EpCAM-binding antibody fragments thereof, or EpCAM activatable antibody), may also be used in the same manner. In some instances, the immunoconjugate described herein comprises a camptothecin derivative linker-payload.

A “linker” is any chemical moiety that is capable of linking a compound, usually a drug, such as a maytansinoid, a camptothecin derivative, or an indolinobenzodiazepine compound, to a cell-binding agent such as an anti EpCAM antibody or an EpCAM-binding antibody fragments thereof. Linkers can be susceptible to or be substantially resistant to acid-induced cleavage, light-induced cleavage, peptidase-induced cleavage, esterase-induced cleavage, and disulfide bond cleavage. Suitable linkers are well known in the art and include, for example, disulfide groups, thioether groups, acid labile groups, photolabile groups, peptidase labile groups and esterase labile groups. Linkers also include peptide linkers and charged linkers, and hydrophilic forms thereof, as disclosed herein and know in the art.

“Aberrant cell proliferation”, as used herein, unless otherwise indicated, refers to cell growth that is independent of normal regulatory mechanisms (e.g., loss of contact inhibition). This includes, for example, the abnormal growth of: (1) tumor cells (tumors) that proliferate by expressing a mutated tyrosine kinase or over expression of a receptor tyrosine kinase; (2) benign and malignant cells of other proliferative diseases in which aberrant tyrosine kinase activation occurs; (3) any tumors that proliferate by receptor tyrosine kinases; (4) any tumors that proliferate by aberrant serine/threonine kinase activation; (5) benign and malignant cells of other proliferative diseases in which aberrant serine/threonine kinase activation occurs, and (6) benign and malignant cells of other proliferative diseases.

The terms “cancer” and “cancerous” refer to or describe the physiological condition in mammals that is typically characterized by unregulated cell growth. A “tumor” comprises one or more cancerous cells. The term “cancer” or “cancerous” as defined herein, includes “pre-cancerous” conditions that, if not treated, can evolve into a cancerous condition. In some embodiments, the cancer is an epithelial cancer. In some embodiments, the cancer expresses EpCAM. Examples of cancers include, breast cancer, lung cancer, non-small cell lung cancer, stomach cancer, colorectal cancer, prostate cancer, bladder cancer, ovarian cancer, colon cancer, rectal cancer, a cancer comprising a cancer stem cell, uterine cancer, gastric cancer, head and neck cancer, endometrial cancer, and pancreatic cancer.

The terms “cancer cell,” “tumor cell,” and grammatical equivalents refer to the total population of cells derived from a tumor or a pre-cancerous lesion, including both non-tumorigenic cells, which comprise the bulk of the tumor cell population, and tumorigenic stem cells (cancer stem cells).

As used herein, the term “cytotoxic agent” refers to a substance that inhibits or prevents one or more cellular functions and/or causes cell death.

As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, methods and compositions provided herein are useful in attempts to delay development of a disease or disorder.

Terms such as “treating” or “treatment” or “to treat” or “alleviating” or “to alleviate” refer to both 1) therapeutic measures that cure, slow down, lessen symptoms of, and/or halt progression of a diagnosed pathologic condition or disorder and 2) prophylactic or preventative measures that prevent and/or slow the development of a targeted pathologic condition or disorder. Thus, those in need of treatment include those already with the disorder, those prone to have the disorder, and those in whom the disorder is to be prevented. In certain embodiments, a subject is successfully “treated” for cancer according to the methods provided herein if the patient shows one or more of the following: a reduction in the number of or complete absence of cancer cells; a reduction in the tumor size; inhibition of or an absence of cancer cell infiltration into peripheral organs including, for example, the spread of cancer into soft tissue and bone; inhibition of or an absence of tumor metastasis; inhibition or an absence of tumor growth; relief of one or more symptoms associated with the specific cancer; reduced morbidity and mortality; improvement in quality of life; reduction in tumorigenicity, tumorigenic frequency, or tumorigenic capacity, of a tumor; reduction in the number or frequency of cancer stem cells in a tumor; differentiation of tumorigenic cells to a non-tumorigenic state; or some combination of effects.

An “effective amount” of an antibody as disclosed herein is an amount sufficient to carry out a specifically stated purpose. A “therapeutically effective amount” refers to an amount effective, at dosages and for periods of time necessary, to achieve the desired therapeutic result. A “therapeutically effective amount” of a therapeutic agent (e.g., a conjugate or immunoconjugate) may vary according to factors such as the disease state, age, sex, and weight of the individual, and the ability of the antibody to elicit a desired response in the individual. A therapeutically effective amount is also one in which any toxic or detrimental effects of the therapeutic agent are outweighed by the therapeutically beneficial effects.

A “therapeutic agent” encompasses both a biological agent such as an antibody, a peptide, a protein, an enzyme, a chemotherapeutic agent, or a conjugate or immunoconjugate.

The terms “subject,” “individual,” “animal,” “patient,” and “mammal,” refer to any subject, particularly a mammalian subject, for whom diagnosis, prognosis, or therapy is desired. Mammalian subjects include but are not limited to humans, non-human primates, domestic animals, farm animals, rodents, and the like, which is to be the recipient of a particular treatment.

The terms “polynucleotide” or “nucleic acid”, as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide can comprise modified nucleotides, such as methylated nucleotides and their analogs. If present, modification to the nucleotide structure can be imparted before or after assembly of the polymer. The sequence of nucleotides can be interrupted by non-nucleotide components. A polynucleotide can be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars can be replaced, for example, by phosphonate groups, phosphate groups, protected by standard protecting groups, or activated to prepare additional linkages to additional nucleotides, or can be conjugated to solid supports. The 5′ and 3′ terminal OH can be phosphorylated or substituted with amines or organic capping group moieties of from 1 to 20 carbon atoms. Other hydroxyls can also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′ azido-ribose, carbocyclic sugar analogs, alpha-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages can be replaced by alternative linking groups. These alternative linking groups include, but are not limited to, embodiments, wherein phosphate is replaced by P(O)S (“thioate”), P(S)S (“dithioate”), “(O)NR2 (“amidate”), P(O)R, P(O)OR′, CO or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The term “vector” means a construct, which is capable of delivering, and optionally expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

The terms “polypeptide”, “peptide”, and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The term “identical” or percent “identity”, as known in the art, is a measure of the relationship between two polynucleotides or two polypeptides, as determined by comparing their sequences. Identity or similarity with respect to a sequence is defined herein as the percentage of amino acid residues in the candidate sequence that are identical (i.e., same residue) or similar (i.e., amino acid residue from the same group based on common side-chain properties, see below) to EpCAM antibody residues, after aligning the sequences and introducing gaps, if necessary, to achieve the maximum percent sequence identity. None of N-terminal, C-terminal, or internal extensions, deletions, or insertions into the antibody sequence outside of the variable region shall be construed as affecting sequence identity or similarity. In general, the two sequences to be compared are aligned to give a maximum correlation between the sequences. The alignment of the two sequences is examined and the number of positions giving an exact amino acid or nucleotide correspondence between the two sequences determined, divided by the total length of the alignment and multiplied by 100 to give a % identity figure. This % identity figure may be determined over the whole length of the sequences to be compared, which is particularly suitable for sequences of the same or very similar length and which are highly homologous, or over shorter defined lengths, which is more suitable for sequences of unequal length or which have a lower level of homology. Likewise percent similarity can be determined in an analogous manner based on the presence of both identical and similar residues.

The percent identity can be measured using sequence comparison software or algorithms or by visual inspection. Various algorithms and software known in the art that can be used to obtain alignments of amino acid or nucleotide sequences. One such non-limiting example of a sequence alignment algorithm is the algorithm described in Karlin et al., Proc. Natl. Acad. Sci. 87:2264-2268 (1990), as modified in Karlin et al., Proc. Natl. Acad. Sci. 90:5873-5877 (1993), and incorporated into the NBLAST and XBLAST programs (Altschul et al., Nucleic Acids Res., 25:3389-3402 (1991)). In certain embodiments, Gapped BLAST can be used as described in Altschul et al., Nucleic Acids Res. 25:3389-3402 (1997). BLAST-2, WU-BLAST-2 (Altschul et al., Meth. Enzym. 266:460-480 (1996)), ALIGN, ALIGN-2 (Genentech, South San Francisco, California) or Megalign (DNASTAR®) are additional publicly available software programs that can be used to align sequences. In certain embodiments, the percent identity between two nucleotide sequences is determined using the GAP program in GCG software (e.g., using a NWSgapdna.CMP matrix and a gap weight of 40, 50, 60, 70, or 90 and a length weight of 1, 2, 3, 4, 5, or 6). In certain alternative embodiments, the GAP program in the GCG software package, which incorporates the algorithm of Needleman and Wunsch (J. Mol. Bio 1. (48):444-453 (1970)) can be used to determine the percent identity between two amino acid sequences (e.g., using either a Blossum 62 matrix or a PAM250 matrix, and a gap weight of 16, 14, 12, 10, 8, 6, or 4, and a length weight of 1, 2, 3, 4, 5). Alternatively, in certain embodiments, the percent identity between nucleotide or amino acid sequences is determined using the algorithm of Myers and Miller (CABIOS 4:11-17 (1989)). For example, the percent identity can be determined using the ALIGN program (version 2.0) and using a PAM120 with residue Table, a gap length penalty of 12 and a gap penalty of 4. Appropriate parameters for maximal alignment by particular alignment software can be determined by one skilled in the art. In certain embodiments, the default parameters of the alignment software used. In certain embodiments, the percentage identity “X” of a first amino acid sequence to a second sequence amino acid is calculated as 100× (Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be longer than the percent identity of the second sequence to the first sequence.

As a non-limiting example, whether any particular polynucleotide has a certain percentage sequence identity (e.g., is at least 80% identical, at least 85% identical, at least 90% identical, and in some embodiments, at least 95%, 96%, 97%, 98%, or 99% identical) to a reference sequence can, in certain embodiments, be determined using the Bestfit program (Wisconsin Sequence Analysis Package, Version 8 for Unix, Genetics Computer Group, University Research Park, 575 Science Drive, Madison, WI 53711). Bestfit uses the local homology algorithm of Smith and Waterman, Advances in Applied Mathematics 2: 482-489 (1981), to find the best segment of homology between two sequences. When using Bestfit or any other sequence alignment program to determine whether a particular sequence is, for instance, 95% identical to a reference sequence as provided herein, the parameters are set such that the percentage of identity is calculated over the full length of the reference nucleotide sequence and that gaps in homology of up to 5% of the total number of nucleotides in the reference sequence are allowed.

A “conservative amino acid substitution” is one in which one amino acid residue is replaced with another amino acid residue having a similar side chain. Families of amino acid residues having similar side chains have been defined in the art, including, for example, basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution.

In some embodiments, conservative substitutions in the sequences of the polypeptides and antibodies provided herein do not abrogate the binding of the polypeptide or antibody containing the amino acid sequence, to the antigen(s), to which the polypeptide or antibody binds. Methods of identifying nucleotide and amino acid conservative substitutions which do not eliminate antigen binding are well-known in the art (see, e.g., Brummell et al., Biochem. 32:1180-1187 (1993); Kobayashi et al., Protein Eng. 12(10):879-884 (1999); and Burks et al., Proc. Natl. Acad. Sci. USA 94:412-417 (1997)).

“Alkyl” as used herein refers to a saturated linear or branched-chain monovalent hydrocarbon radical of one to twenty carbon atoms. Examples of alkyl include, but are not limited to, methyl, ethyl, 1-propyl, 2-propyl, 1-butyl, 2-methyl-1-propyl, —CH2CH(CH3)2), 2 butyl, 2-methyl-2-propyl, 1-pentyl, 2-pentyl 3-pentyl, 2-methyl-2-butyl, 3-methyl-2-butyl, 3 methyl-1-butyl, 2-methyl-1-butyl, 1-hexyl), 2-hexyl, 3-hexyl, 2-methyl-2-pentyl, 3-methyl-2 pentyl, 4-methyl-2-pentyl, 3-methyl-3-pentyl, 2-methyl-3-pentyl, 2,3-dimethyl-2-butyl, 3,3 dimethyl-2-butyl, 1-heptyl, 1-octyl, and the like. In some embodiments, the alkyl has one to ten carbon atoms. In some embodiments, the alkyl has one to four carbon atoms.

The number of carbon atoms in a group can be specified herein by the prefix “Cx-xx”, wherein x and xx are integers. For example, “C1-4alkyl” is an alkyl group having from 1 to 4 carbon atoms.

The term “compound” or “cytotoxic compound,” or “cytotoxic agent” are used interchangeably. They are intended to include compounds for which a structure or formula or any derivative thereof has been disclosed herein or a structure or formula or any derivative thereof that has been incorporated by reference. The term also includes, stereoisomers, geometric isomers, tautomers, solvates, metabolites, and salts (e.g., pharmaceutically acceptable salts) of a compound of all the formulae disclosed herein. The term also includes any solvates, hydrates, and polymorphs of any of the foregoing. The specific recitation of “stereoisomers,” “geometric isomers,” “tautomers,” “solvates,” “metabolites,” “salt”, “conjugates,” “conjugates salt,” “solvate,” “hydrate,” or “polymorph” in certain embodiments, provided herein shall not be interpreted as an intended omission of these forms in other disclosed embodiments, where the term “compound” is used without recitation of these other forms. In some instances, the compound comprises a camptothecin derivative linker-payload.

The term “imine reactive reagent” refers to a reagent that is capable of reacting with an imine group. Examples of imine reactive reagent includes, but is not limited to, sulfites (H₂SO₃, H₂SO₂ or a salt of HSO₃⁻, SO₃²⁻ or HSO₂⁻ formed with a cation), metabisulfite (H₂S₂O₅ or a salt of S₂O₅²⁻ formed with a cation), mono, di, tri, and tetra-thiophosphates (PO₃SH₃, PO₂S₂H₃, POS₃H₃, PS₄H₃ or a salt of PO₃S₃⁻, PO₂S₂³⁻, POS₃³⁻ or PS₄³⁻ formed with a cation), thio phosphate esters ((RiO)₂PS(ORi), RiSH, RiSOH, RiSO₂H, RiSO₃H), various amines (hydroxyl amine (e.g., NH₂OH), hydrazine (e.g., NH₂NH₂), NH₂O-Ri, Ri′NH-Ri, NH₂-Ri), NH₂—CO—NH₂, NH₂—C(═S)—NH₂, thiosulfate (H₂S₂O₃ or a salt of S₂O₃²⁻ formed with a cation), dithionite (H₂S₂O₄ or a salt of S₂O₄²⁻ formed with a cation), phosphorodithioate (P(═S)(ORk)(SH)(OH) or a salt thereof formed with a cation), hydroxamic acid (RkC(═O)NHOH or a salt formed with a cation), hydrazide (RkCONHNH₂), formaldehyde sulfoxylate (HOCH₂SO₂H or a salt of HOCH₂SO₂⁻ formed with a cation, such as HOCH₂SO₂—Na⁺), glycated nucleotide (such as GDP-mannose), fludarabine or a mixture thereof, wherein Ri and Ri′ are each independently a linear or branched alkyl having 1 to 10 carbon atoms and are substituted with at least one substituent selected from N(Rj)₂, —CO₂H, SO₃H, and PO3H; Ri and Ri′ can be further optionally substituted with a substituent for an alkyl disclosed herein; Rj is a linear or branched alkyl having 1 to 6 carbon atoms; and Rk is a linear, branched or cyclic alkyl, alkenyl or alkynyl having 1 to 10 carbon atoms, aryl, heterocyclyl or heteroaryl (in some embodiments, Rk is a linear or branched alkyl having 1 to 4 carbon atoms; in some embodiments, Rk is methyl, ethyl or propyl). In some embodiments, the cation is a monovalent cation, such as Na⁺ or K⁺. In some embodiments, the imine reactive reagent is selected from sulfites, hydroxyl amine, urea and hydrazine. In some embodiments, the imine reactive reagent is NaHSO₃ or KHSO₃.

The term “cation” refers to an ion with positive charge. The cation can be monovalent (e.g., Na⁺, K⁺, NH4⁺ etc.), bi-valent (e.g., Ca²⁺, Mg²⁺, etc.) or multi-valent (e.g., Al³⁺ etc.). In some embodiments, the cation is monovalent.

The phrase “pharmaceutically acceptable salt” as used herein, refers to pharmaceutically acceptable organic or inorganic salts of a compound provided herein. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt can involve the inclusion of another molecule such as an acetate ion, a succinate ion or other counter ion. The counter ion can be any organic or inorganic moiety that stabilizes the charge on the parent compound. Furthermore, a pharmaceutically acceptable salt can have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

If the compound provided herein is a base, the desired pharmaceutically acceptable salt can be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the compound provided herein is an acid, the desired pharmaceutically acceptable salt can be prepared by any suitable method, for example, treatment of the free acid with an inorganic or organic base, such as an amine (primary, secondary or tertiary), an alkali metal hydroxide or alkaline earth metal hydroxide, or the like. Illustrative Examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

As used herein, the term “solvate” means a compound that further includes a stoichiometric or non-stoichiometric amount of solvent such as water, isopropanol, acetone, ethanol, methanol, DMSO, ethyl acetate, acetic acid, and ethanolamine dichloromethane, 2 propanol, or the like, bound by non-covalent intermolecular forces. Solvates or hydrates of the compounds are readily prepared by addition of at least one molar equivalent of a hydroxylic solvent such as methanol, ethanol, 1-propanol, 2-propanol or water to the compound to result in solvation or hydration of the imine moiety.

A “metabolite” or “catabolite” is a product produced through metabolism or catabolism in the body of a specified compound, a derivative thereof, or a conjugate thereof, or salt thereof. Metabolites of a compound, a derivative thereof, or a conjugate thereof, can be identified using routine techniques known in the art and their activities determined using tests such as those disclosed herein. Such products can result for example from the oxidation, hydroxylation, reduction, hydrolysis, amidation, deamidation, esterification, deesterification, enzymatic cleavage, and the like, of the administered compound. Accordingly, the disclosure includes metabolites of compounds, a derivative thereof, or a conjugate thereof, of the disclosed EpCAM compositions disclosed herein, including compounds, derivatives thereof, or conjugates thereof, produced by a process comprising contacting a disclosed EpCAM compound, a derivative thereof, or a conjugate thereof, with a mammal for a period of time sufficient to yield a metabolic product thereof.

The phrase “pharmaceutically acceptable” indicates that the substance or composition must be compatible chemically and/or toxicologically, with the other ingredients comprising a formulation, and/or the mammal being treated therewith.

The term “protecting group” or “protecting moiety” refers to a substituent that is commonly employed to block or protect a particular functionality while reacting other functional groups on the compound, a derivative thereof, or a conjugate thereof. For example, an “amine-protecting group” or an “amino-protecting moiety” is a substituent attached to an amino group that blocks or protects the amino functionality in the compound. Such groups are well known in the art (see, for example, P. Wuts and T. Greene, 2007, Protective Groups in Organic Synthesis, Chapter 7, J. Wiley & Sons, NJ) and exemplified by carbamates such as methyl and ethyl carbamate, FMOC, substituted ethyl carbamates, carbamates cleaved by 1,6-β-elimination (also termed “self immolative”), ureas, amides, peptides, alkyl and aryl derivatives. Suitable amino-protecting groups include acetyl, trifluoroacetyl, t-butoxycarbonyl (BOC), benzyloxycarbonyl (CBZ) and 9-fluorenylmethylenoxycarbonyl (Fmoc). For a general description of protecting groups and their use, see P. G. M. Wuts & T. W. Greene, Protective Groups in Organic Synthesis, John Wiley & Sons, New York, 2007.

The term “amino acid” refers to naturally occurring amino acids or non-naturally occurring amino acid. In one embodiment, the amino acid is represented by NH2-C(Raa′Raa)-C(═O)OH, wherein Raa and Raa′ are each independently H, an optionally substituted linear, branched or cyclic alkyl, alkenyl or alkynyl having 1 to 10 carbon atoms, aryl, heteroaryl or heterocyclyl or Raa and the N-terminal nitrogen atom can together form a heteroycyclic ring (e.g., as in proline). The term “amino acid residue” refers to the corresponding residue when one hydrogen atom is removed from the amine and/or carboxy end of the amino acid, such as —NH—C(Raa′Raa)-C(═O)O—.

The term “peptide” refers to short chains of amino acid monomers linked bypeptide (amide) bonds. In some embodiments, the peptides contain 2 to 20 amino acid residues. In other embodiments, the peptides contain 2 to 10 amino acid residues. In yet other embodiments, the peptides contain 2 to 5 amino acid residues. As used herein, when a peptide is a portion of a cytotoxic agent or a linker disclosed herein represented by a specific sequence of amino acids, the peptide can be connected to the rest of the cytotoxic agent or the linker in both directions. For example, a dipeptide X1-X2 includes X1-X2 and X2-X1. Similarly, a tripeptide X1-X2-X₃ includes X1-X2-X₃ and X₃-X2-X1 and a tetrapeptide X1-X2-X₃-X4 includes X1-X2-X₃-X4 and X4-X2-X₃-X1. X1, X2, X₃ and X4 represents an amino acid residue.

The term “reactive ester group” refers to a group an ester group that can readily react with an amine group to form amide bond. Exemplary reactive ester groups include, but are not limited to, N-hydroxysuccinimide esters, N-hydroxyphthalimide esters, N-hydroxy sulfo-succinimide esters, para-nitrophenyl esters, dinitrophenyl esters, pentafluorophenyl esters and their derivatives, wherein the derivatives facilitate amide bond formation. In certain embodiments, the reactive ester group is an N-hydroxysuccinimide ester or an N hydroxy sulfo-succinimide ester.

The term “amine reactive group” refers to a group that can react with an amine group to form a covalent bond. Exemplary amine reactive groups include, but are not limited to, reactive ester groups, acyl halides, sulfonyl halide, imidoester, or a reactive thioester groups. In certain embodiments, the amine reactive group is a reactive ester group. In one embodiment, the amine reactive group is an N-hydroxysuccinimide ester or an N-hydroxy sulfo-succinimide ester.

The term “thiol-reactive group” refers to a group that can react with a thiol (—SH) group to form a covalent bond. Exemplary thiol-reactive groups include, but are not limited to, maleimide, haloacetyl, haloacetamide, vinyl sulfone, vinyl sulfonamide or vinyl pyridine. In one embodiment, the thiol-reactive group is maleimide.

As used in the present disclosure and claims, the singular forms “a,” “an,” and “the” include plural forms unless the context clearly dictates otherwise.

The term “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Wherever embodiments, are disclosed herein with the language “comprising,” otherwise analogous embodiments, described in terms of “consisting of” and/or “consisting essentially of” are also provided.

### 6.2 Immunoconjugates Comprising Epcam Antibodies and Epcam-Binding Antibody Fragments Thereof

Disclosed herein are immunoconjugates comprising EpCAM antibodies and EpCAM-binding antibody fragments thereof. These proteins specifically bind human EpCAM and are provided herein. In some embodiments, the proteins are referred to herein as “EpCAM-binding agents” or EpBAs.”

In additional embodiments, immunoconjugates disclosed herein comprise the EpCAM-binding agent, which is an EpCAM antibody, an EpCAM-binding antibody fragment thereof, or an EpCAM activatable antibody. In some embodiments, the EpBA is a full-length EpCAM antibody (i.e., a full-length antibody that specifically binds EpCAM). In some embodiments, the EpCAM antibody is a monoclonal antibody. In some embodiments, the EpCAM antibody is a recombinant antibody, a human antibody, a humanized antibody, a chimeric antibody, a multi-specific antibody (e.g., a bi-specific antibody), or an EpCAM-binding antibody fragment thereof. In some embodiments, the EpCAM antibody specifically binds human EpCAM. In further embodiments, the EpCAM antibody specifically binds human EpCAM and cyno EpCAM. In some embodiments, the EpCAM antibody or EpCAM-binding antibody fragment thereof is a mouse, other rodent, chimeric, humanized or fully human monoclonal antibody.

In some embodiments, immunoconjugates disclosed herein comprise an EpCAM antibody that is an EpCAM-binding antibody fragment. In some embodiments, immunoconjugates disclosed herein comprise a non-human mammal antibody, a murine antibody, a chimeric antibody, a humanized antibody, or a human antibody. In some embodiments, immunoconjugates disclosed herein comprise a full-length antibody. In some embodiments, immunoconjugates disclosed herein comprise a human IgG1 antibody.

In some embodiments, immunoconjugates disclosed herein comprise a Fab, a Fab′, a F(ab′)2, an scFv, or a disulfide-linked Fv (dsFv). In additional aspects, EpCAM-binding antibody fragment is a single chain Fv (scFv), disulfide linked Fv, IgGΔCH2, minibody, F(ab′)₃, scAb, tetrabody, triabody, diabody, DVD-Ig, Fcab, mAb², (scFv)₂, scFv-Fc or bis-scFv.

In some instances, the immunoconjugates disclosed herein comprise EpCAM antibodies and EpCAM-binding antibody fragments thereof provided herein that optionally bind EpCAM (e.g., human EpCAM and/or murine EpCAM), with a wide range of affinities (Ko). In a particular embodiment, the antibody binds human EpCAM with high affinity. For example, a human or human engineered or humanized or resurfaced mAb can bind human antigen with a K_{D} equal to or less than about 10⁻⁷ M, such as but not limited to, 0.1-9.9 (or any range or value therein between)×10⁻⁷, 10⁻⁸, 10⁻⁹, 10⁻¹⁰, 10⁻¹¹, or 10⁻¹², or any range or value therein, as determined by flow cytometry base assays, enzyme-linked immunoabsorbent assay (ELISA), surface plasmon resonance (SPR) or the KinExA® method using standard operating procedures. In some embodiments, the EpCAM antibodies bind with a Kd of about 10⁻⁹ M or less, more specifically about 10⁻⁹ to 10⁻¹⁰ M.

The affinity or avidity for EpCAM of an antibody or antibody fragment thereof in an immunoconjugate described herein can be determined experimentally using any suitable method known in the art, e.g., flow cytometry, enzyme-linked immunoabsorbent assay (ELISA), or radioimmunoassay (RIA), or kinetics (e.g., BIACORE™ analysis), using standard operating procedures. Direct binding assays as well as competitive binding assay formats can be routinely employed. See, e.g., Berzofsky, et al., “Antibody-Antigen Interactions,” In Fundamental Immunology, Paul, W. E., Ed., Raven Press: New York, N.Y. (1984); Kuby, Janis Immunology, W. H. Freeman and Company: New York, N.Y. (1992); and methods disclosed herein. The measured affinity of a particular antibody-EpCAM interaction can vary if measured under different conditions (e.g., salt concentration, pH, temperature). Thus, measurements of affinity and other EpCAM-binding parameters (e.g., KD or Kd, Kon, Koff) are made with standardized solutions of antibody and EpCAM, and a standardized buffer, as known in the art and such as the buffer disclosed herein.

In one embodiment, binding assays are performed using flow cytometry on cells expressing the EpCAM antigen on the surface. For example, such EpCAM-positive cells are incubated with varying concentrations of EpCAM antibodies using 1×10⁵ cells per sample in 100 μL FACS buffer (RPMI-1640 medium supplemented with 2% normal goat serum). Then, the cells are pelleted, washed, and incubated for 1 h with 100 μL of FITC-conjugated goat anti-mouse IgG-antibody (such as obtainable from Jackson ImmunoResearch) in FACS buffer. The cells are pelleted again, washed with FACS buffer and resuspended in 200 μL of PBS containing 1% formaldehyde. Samples are acquired, for example, using a FACSCalibur™ flow cytometer with the HTS multiwell sampler and analyzed using CellQuest® Pro (all from BD Biosciences, San Diego, US). For each sample the mean fluorescence intensity for FL1 (MFL) is exported and plotted against the antibody concentration in a semi-log plot to generate a binding curve. A sigmoidal dose-response curve is fitted for binding curves and EC₅₀ values are calculated using programs such as GraphPad Prism v4 with default parameters (GraphPad software, San Diego, CA). EC₅₀ values can be used as a measure for the apparent dissociation constant “K_{d}” or “K_{D}” for each antibody.

In certain embodiments, the EpCAM antibodies or fragments thereof in any of the immunoconjugates described herein are modified to alter their binding affinity for EpCAM and/or EpCAM antigenic fragments thereof. Binding properties may be determined by a variety of in vitro assay methods and employing standard operating procedures known in the art, including for example, enzyme-linked immunoabsorbent assay (ELISA), radioimmunoassay (RIA)), or kinetics (e.g., BIACORE™ analysis).

In one embodiment, the EpCAM antibody or EpCAM-binding antibody fragment thereof in any of the immunoconjugates described herein specifically binds human and/or cynomolgus EpCAM, and/or EpCAM antigenic fragments thereof, with a dissociation constant or KD or Kd (koff/kon) of less than 10⁻⁵ M, or of less than 10⁻⁶ M, or of less than 10⁻⁷ M, or of less than 10⁻⁸ M, or of less than 10⁻⁹ M, or of less than 10⁻¹⁰ M, or of less than 10⁻¹¹ M, or of less than 10⁻¹² M, or of less than 10⁻¹³ M. In some embodiments, the EpCAM antibody or EpCAM-binding antibody fragment thereof specifically binds human and/or cynomolgus EpCAM and/or EpCAM antigenic fragments thereof with a KD of 1.0×10⁻⁹ M or less, 2.0×10⁻⁹ M or less, 3.0×10⁻⁹ M or less, 4.0×10⁻⁹ M or less, 5.0×10⁻⁹ M or less, 6.0×10⁻⁹ M or less, 7.0×10⁻⁹ M or less, 8.0×10⁻⁹ M or less, or 9.0×10⁻⁹ M or less. In certain embodiments, the EpCAM antibody or EpCAM-binding antibody fragment thereof binds to both human and cynomolgus EpCAM and/or EpCAM antigenic fragments thereof with a KD of 3.0×10⁻⁹ M or less. In some embodiments, the EpCAM antibody or EpCAM-binding antibody fragment thereof binds to human EpCAM with a KD of about 0.4×10⁻⁹. In some embodiments, the EpCAM antibody or EpCAM-binding antibody fragment thereof binds to human EpCAM with a KD of about 0.8×10⁻⁹. In some embodiments, the EpCAM antibody or EpCAM-binding antibody fragment thereof binds to cynomolgus EpCAM with a KD of about 0.8×10⁻⁹. In some embodiments, the EpCAM antibody or EpCAM-binding antibody fragment thereof binds to cynomolgus EpCAM with a KD of about 2.2×10⁻⁹. In some embodiments, the EpCAM antibody or EpCAM-binding antibody fragment thereof binds to cynomolgus EpCAM with a KD of about 2.8×10⁻⁹.

In some embodiments, the EpCAM antibody or EpCAM-binding antibody fragment thereof in any of the immunoconjugates described herein specifically binds to an epitope within the extracellular region of human EpCAM (SEQ ID NO:1). The extracellular region of human EpCAM may be further divided into three distinct domains: D1 (SEQ ID NO:2), D2 (SEQ ID NO:3), and D3 (SEQ ID NO:4). In certain embodiments, the EpCAM antibody or EpCAM-binding antibody fragment thereof specifically binds to an epitope within the first extracellular domain (D1) of human EpCAM.

In one embodiment, the EpCAM antibody or EpCAM-binding antibody fragment thereof in any of the immunoconjugates described herein comprises a VH-CDR1 comprising X₁YX₃X₄H, wherein X₁ is selected from N and S, X₃ is selected from Y, N, F, S, H, D, L, I, and W₁ and X₄ is selected from I and M; a VH-CDR2 comprising WX₂X₃PGX₆VYIQYX₁₂X₁₃KFX₁₇G, wherein X₂ is selected from I and F, X₃ is selected from Y and N, X₆ is selected from N and D, X₁₂ is selected from N and S, X₁₃ is selected from E and Q, and X₁₇ is selected from K and Q (SEQ ID NO:7); and a VH-CDR3 comprising X₁GX₃X₄FAY, wherein X₁ is selected from D and E, X₃ is selected from P, A, S, Y, F, G, T, and V, and X₄ is selected from Y and W (SEQ ID NO:8).

In additional embodiments, the disclosure provides an EpCAM antibody or EpCAM-binding antibody fragment thereof in any of the immunoconjugates described herein, comprising a light chain CDR1 (VL-CDR1) comprising RSSX₄SLLHSX₁₀GX₁₂TYLX₁₆, wherein X₄ is selected from R and K, X₁₀ is selected from N and D, X₁₂ is selected from F and I, and X₁₆ is selected from Y and S (SEQ ID NO:10); a light chain VL-CDR2 comprising QTSNLAS (SEQ ID NO:40); and a VL-CDR3 comprising X₁QX₃LELPX₈T, wherein X₁ is selected from A, L, and Q, X₃ is selected from S, G, Y, and N, and Xe is selected from N and W (SEQ ID NO:11). In some embodiments, the disclosure provides an EpCAM antibody or EpCAM-binding antibody fragment thereof comprising a heavy chain CDR1 (VH-CDR1) comprising the sequence of SEQ ID NO:13; a heavy chain CDR2 (VH-CDR2) comprising the sequence of SEQ ID NO:14; a heavy chain CDR3 (VH-CDR3) comprising the sequence of SEQ ID NO:15; a light chain CDR1 (VL-CDR1) comprising the sequence of SEQ ID NO:42; a light chain CDR2 (VL-CDR2) comprising the sequence of SEQ ID NO:40; and a light chain CDR3 (VL-CDR3) comprising the sequence of SEQ ID NO:41.

In some embodiments, the VH-CDR1 comprises the sequence NYX₃IH, wherein X₃ is selected from Y, N, F, S, H, D, L, I, and W (SEQ ID NO:6). In some embodiments, the VH-CDR3 comprises the sequence DGPX₄FAY, wherein X₄ is selected from Y and W (SEQ ID NO:9). In some embodiments, the VL-CDR3 comprises the sequence AQX₃LELPNT, wherein X₃ is selected from S, G, Y, and N (SEQ ID NO:12). In some embodiments, the disclosure provides an EpCAM antibody or EpCAM-binding antibody fragment thereof comprising a heavy chain CDR1 (VH-CDR1) comprising the sequence of SEQ ID NO:13; a heavy chain CDR2 (VH-CDR2) comprising the sequence of SEQ ID NO:14; a heavy chain CDR3 (VH-CDR3) comprising the sequence of SEQ ID NO:15; a light chain CDR1 (VL-CDR1) comprising the sequence of SEQ ID NO:42; a light chain CDR2 (VL-CDR2) comprising the sequence of SEQ ID NO:40; and a light chain CDR3 (VL-CDR3) comprising the sequence of SEQ ID NO:41.

In some embodiments, the EpCAM antibody or EpCAM-binding antibody fragment thereof in any of the immunoconjugates described herein comprises a set of complementarity determining regions (CDRs): heavy chain variable region (VH)-CDR1, VH-CDR2, VH-CDR3, light chain variable region (VL) CDR1, VL-CDR2 and VL-CDR3, wherein the heavy chain CDRs are disclosed in Table 2.

TABLE 2
Exemplary heavy chain CDR sequences of EpCAM antibodies.

| | | | |
|---|---|---|---|
| Antibody | VH-CDR1 | VH-CDR2 | VH-CDR3 |

Murine and Chimeric

| | | | |
|---|---|---|---|
| muEpcam23 | NYYIH | WIYPGNVYIQYNEKFKG | DGPWFAY |
| | (SEQ ID NO: 13) | (SEQ ID NO: 14) | (SEQ ID NO: 15) |
| chEpcam23 | NYYIH | WIYPGNVYIQYNEKFKG | DGPWFAY |
| | (SEQ ID NO: 13) | (SEQ ID NO: 14) | (SEQ ID NO: 15) |

Humanized Variants

| | | | |
|---|---|---|---|
| huEpCAM23_VHGv1 | NYYIH | WIYPGNVYIQYSQKFQG | DGPWFAY |
| | (SEQ ID NO: 13) | (SEQ ID NO: 26) | (SEQ ID NO: 15) |
| huEpCAM23_VHGv2 | NYYIH | WIYPGNVYIQYNEKFKG | DGPWFAY |
| | (SEQ ID NO: 13) | (SEQ ID NO: 14) | (SEQ ID NO: 15) |
| huEpCAM23_VHGv3 | NYYIH | WIYPGNVYIQYNEKFKG | DGPWFAY |
| | (SEQ ID NO: 13) | (SEQ ID NO: 14) | (SEQ ID NO: 15) |
| huEpCAM23_VHGv4 | NYYIH | WIYPGNVYIQYSQKFQG | DGPWFAY |
| | (SEQ ID NO: 13) | (SEQ ID NO: 26) | (SEQ ID NO: 15) |

Affinity Variants

| | | | |
|---|---|---|---|
| huEpCAM23HCGv2a | NYYIH | WIYPGNVYIQYNEKFKG | DGPWFAY |
| | (SEQ ID NO: 13) | (SEQ ID NO: 14) | (SEQ ID NO: 15) |
| huEpCAM23HCGv2b | SYYIH | WIYPGNVYIQYNEKFKG | DGPWFAY |
| | (SEQ ID NO: 16) | (SEQ ID NO: 14) | (SEQ ID NO: 15) |
| huEpCAM23HCGv2c | NYNIH | WIYPGNVYIQYNEKFKG | DGPWFAY |
| | (SEQ ID NO: 17) | (SEQ ID NO: 14) | (SEQ ID NO: 15) |
| huEpCAM23HCGv2d | NYYIH | WIYPGDVYIQYNEKFKG | DGPWFAY |
| | (SEQ ID NO: 13) | (SEQ ID NO: 27) | (SEQ ID NO: 15) |
| huEpCAM23HCGv2e | NYFIH | WIYPGNVYIQYNEKFKG | DGPWFAY |
| | (SEQ ID NO: 18) | (SEQ ID NO: 14) | (SEQ ID NO: 15) |
| huEpCAM23HCGv2f | NYSIH | WIYPGNVYIQYNEKFKG | DGPWFAY |
| | (SEQ ID NO: 19) | (SEQ ID NO: 14) | (SEQ ID NO: 15) |
| huEpCAM23HCGv2g | NYWIH | WIYPGNVYIQYNEKFKG | DGPWFAY |
| | (SEQ ID NO: 20) | (SEQ ID NO: 14) | (SEQ ID NO: 15) |
| huEpCAM23HCGv2h | NYYIH | WFYPGNVYIQYNEKFKG | DGPWFAY |
| | (SEQ ID NO: 13) | (SEQ ID NO: 28) | (SEQ ID NO: 15) |
| huEpCAM23HCGv2/20 | NYYIH | WIYPGNVYIQYNEKFKG | DGPWFAY |
| | (SEQ ID NO: 13) | (SEQ ID NO: 14) | (SEQ ID NO: 15) |
| huEpCAM23HCGv2j | NYYIH | WINPGNVYIQYNEKFKG | DGPWFAY |
| | (SEQ ID NO: 13) | (SEQ ID NO: 29) | (SEQ ID NO: 15) |
| huEpCAM23HCGv2k | NYYIH | WIYPGNVYIQYNEKFKG | EGPWFAY |
| | (SEQ ID NO: 13) | (SEQ ID NO: 14) | (SEQ ID NO: 30) |
| huEpCAM23HCGv2l | NYYIH | WIYPGNVYIQYNEKFKG | DGPYFAY |
| | (SEQ ID NO: 13) | (SEQ ID NO: 14) | (SEQ ID NO: 31) |
| huEpCAM23HCGv2m/ | NYYIH | WIYPGNVYIQYNEKFKG | DGAWFAY |
| huEpCam23 HG2-1565-A | (SEQ ID NO: 13) | (SEQ ID NO: 14) | (SEQ ID NO: 32) |
| Heavy Chain | | | |
| huEpCAM23HCGv2n | NYYMH | WIYPGNVYIQYNEKFKG | DGPWFAY |
| | (SEQ ID NO: 21) | (SEQ ID NO: 14) | (SEQ ID NO: 15) |
| huEpCAM23HCGv2p | NYYIH | WIYPGNVYIQYNEKFKG | DGPWFAY |
| | (SEQ ID NO: 13) | (SEQ ID NO: 14) | (SEQ ID NO: 15) |
| huEpCAM23HCGv2q | NYYIH | WIYPGNVYIQYNEKFKG | DGPWFAY |
| | (SEQ ID NO: 13) | (SEQ ID NO: 14) | (SEQ ID NO: 15) |
| huEpCAM23HCGv2r | NYYIH | WIYPGNVYIQYNEKFKG | DGPWFAY |
| | (SEQ ID NO: 13) | (SEQ ID NO: 14) | (SEQ ID NO: 15) |
| huEpCAM23HCGv2s | NYYIH | WIYPGNVYIQYNEKFKG | DGPWFAY |
| | (SEQ ID NO: 13) | (SEQ ID NO: 14) | (SEQ ID NO: 15) |
| huEpCam23HG2-1361-H | NYHIH | WIYPGNVYIQYNEKFKG | DGPWFAY |
| Heavy Chain | (SEQ ID NO: 22) | (SEQ ID NO: 14) | (SEQ ID NO: 15) |
| huEpCam23HG2-1361-D | NYDIH | WIYPGNVYIQYNEKFKG | DGPWFAY |
| Heavy Chain | (SEQ ID NO: 23) | (SEQ ID NO: 14) | (SEQ ID NO: 15) |
| huEpCam23HG2-1565-Y | NYYIH | WIYPGNVYIQYNEKFKG | DGYWFAY |
| Heavy Chain | (SEQ ID NO: 13) | (SEQ ID NO: 14) | (SEQ ID NO: 33) |
| huEpCam23HG2-1565-S | NYYIH | WIYPGNVYIQYNEKFKG | DGSWFAY |
| Heavy Chain | (SEQ ID NO: 13) | (SEQ ID NO: 14) | (SEQ ID NO: 34) |
| huEpCam23HG2-1565-F | NYYIH | WIYPGNVYIQYNEKFKG | DGFWFAY |
| Heavy Chain | (SEQ ID NO: 13) | (SEQ ID NO: 14) | (SEQ ID NO: 35) |
| huEpCam23HG2-1565-G | NYYIH | WIYPGNVYIQYNEKFKG | DGGWFAY |
| Heavy Chain | (SEQ ID NO: 13) | (SEQ ID NO: 14) | (SEQ ID NO: 36) |
| huEpCam23HG2-1565-T | NYYIH | WIYPGNVYIQYNEKFKG | DGTWFAY |
| Heavy Chain | (SEQ ID NO: 13) | (SEQ ID NO: 14) | (SEQ ID NO: 37) |
| huEpCam23HG2-1565-V | NYYIH | WIYPGNVYIQYNEKFKG | DGVWFAY |
| Heavy Chain | (SEQ ID NO: 13) | (SEQ ID NO: 14) | (SEQ ID NO: 38) |
| huEpCam23HG2-1361-I | NYNIH | WIYPGNVYIQYNEKFKG | DGPWFAY |
| Heavy Chain | (SEQ ID NO: 24) | (SEQ ID NO: 14) | (SEQ ID NO: 15) |
| huEpCam23HG2-1361-L | NYLIH | WIYPGNVYIQYNEKFKG | DGPWFAY |
| Heavy Chain | (SEQ ID NO: 25) | (SEQ ID NO: 14) | (SEQ ID NO: 15) |

In some embodiments, the disclosure provides an EpCAM antibody or EpCAM-binding antibody fragment thereof in any of the immunoconjugates described herein comprising the heavy chain CDRs of a single row in Table 2. In some embodiments, the disclosure provides an EpCAM antibody or EpCAM-binding antibody fragment thereof comprising a VH-CDR1 selected from SEQ ID NOs:13, and 16-25; a VH-CDR2 selected from SEQ ID NOs:14, and 26-29; and a VH-CDR3 selected from SEQ ID NOs:15, and 30-38. In some embodiments, the disclosure provides an EpCAM antibody or EpCAM-binding antibody fragment thereof comprising a VH-CDR1 of SEQ ID NO:13; a VH-CDR2 of SEQ ID NO:14; and a VH-CDR3 of SEQ ID NO:15. In some embodiments, the disclosure provides an EpCAM antibody or EpCAM-binding antibody fragment thereof comprising a VH-CDR1 of SEQ ID NO:13; a VH-CDR2 of SEQ ID NO:26; and a VH-CDR3 of SEQ ID NO:15.

In one embodiment, the disclosure provides immunoconjugates having an EpCAM antibody, EpCAM-binding antibody fragment thereof, or EpCAM activatable antibody comprising, a VH-CDR1 comprising NYYIH (SEQ ID NO:13), or a variant thereof comprising 1, 2, 3, or 4, conservative amino acid substitutions; a VH-CDR2 comprising WIYPGNVYIQYNEKFKG (SEQ ID NO:14), or a variant thereof comprising 1, 2, 3, or 4, amino conservative acid substitutions; and a heavy chain CDR3 comprising DGPWFAY (SEQ ID NO:15), or a variant thereof comprising 1, 2, 3, or 4, conservative amino acid substitutions.

In some embodiments, the disclosure provides immunoconjugates having an EpCAM antibody or EpCAM-binding antibody fragment thereof comprising a VH-CDR1 of SEQ ID NO:22; a VH-CDR2 of SEQ ID NO:14; and a VH-CDR3 of SEQ ID NO:15. In some embodiments, the disclosure provides an EpCAM antibody or EpCAM-binding antibody fragment thereof comprising a VH-CDR1 of SEQ ID NO:13; a VH-CDR2 of SEQ ID NO:14; and a VH-CDR3 of SEQ ID NO:33. In some embodiments, the disclosure provides an EpCAM antibody or EpCAM-binding antibody fragment thereof comprising a VH-CDR1 of SEQ ID NO:23; a VH-CDR2 of SEQ ID NO:14; and a VH-CDR3 of SEQ ID NO:15. In some embodiments, the disclosure provides an EpCAM antibody or EpCAM-binding antibody fragment thereof comprising a VH-CDR1 of SEQ ID NO:25; a VH-CDR2 of SEQ ID NO:14; and a VH-CDR3 of SEQ ID NO:15.

In one embodiment, the disclosure provides immunoconjugates having an EpCAM antibody, EpCAM-binding antibody fragment thereof, or EpCAM activatable antibody comprising, a VH-CDR1 comprising NYHIH (SEQ ID NO:22), or a variant thereof comprising 1, 2, 3, or 4, conservative amino acid substitutions; a VH-CDR2 comprising WIYPGNVYIQYNEKFKG (SEQ ID NO:14), or a variant thereof comprising 1, 2, 3, or 4, amino conservative acid substitutions; and a heavy chain CDR3 comprising DGPWFAY (SEQ ID NO:15), or a variant thereof comprising 1, 2, 3, or 4, conservative amino acid substitutions. In one embodiment, the disclosure provides an EpCAM antibody, EpCAM-binding antibody fragment thereof, or EpCAM activatable antibody comprising, a VH-CDR1 comprising NYYIH (SEQ ID NO:13), or a variant thereof comprising 1, 2, 3, or 4, conservative amino acid substitutions: a VH-CDR2 comprising WIYPGNVYIQYNEKFKG (SEQ ID NO:14), or a variant thereof comprising 1, 2, 3, or 4, amino conservative acid substitutions; and a heavy chain CDR3 comprising DGYWFAY (SEQ ID NO:33), or a variant thereof comprising 1, 2, 3, or 4, conservative amino acid substitutions.

In some embodiments, the EpCAM antibody or EpCAM-binding antibody fragment thereof of any of the immunoconjugates described herein comprises a set of complementarity determining regions (CDRs): heavy chain variable region (VH)-CDR1, VH-CDR2, VH-CDR3, light chain variable region (VL) CDR1, VL-CDR2 and VL-CDR3, wherein the light chain CDRs are disclosed in Table 3.

TABLE 3
Exemplary light chain CDR sequences of EpCAM antibodies.

| | | | |
|---|---|---|---|
| Antibody | VL-CDR1 | VL-CDR2 | VL-CDR3 |

Murine and Chimeric

| | | | |
|---|---|---|---|
| muEpcam23 | RSSKSLLHSDGFTYLY | QTSNLAS | AQNLELPNT |
| | (SEQ ID NO: 39) | (SEQ ID NO: 40) | (SEQ ID NO: 41) |
| chEpcam23 | RSSKSLLHSDGFTYLY | QTSNLAS | AQNLELPNT |
| | (SEQ ID NO: 39) | (SEQ ID NO: 40) | (SEQ ID NO: 41) |

Humanized Variants

| | | | |
|---|---|---|---|
| huEpCAM23_VLG v1 | RSSRSLLHSDGFTYLY | QTSNLAS | AQNLELPNT |
| | (SEQ ID NO: 42) | (SEQ ID NO: 40) | (SEQ ID NO: 41) |
| huEpCAM23_VLG v2 | RSSKSLLHSDGFTYLY | QTSNLAS | AQNLELPNT |
| | (SEQ ID NO: 39) | (SEQ ID NO: 40) | (SEQ ID NO: 41) |
| huEpCAM23_VLG v3 | RSSKSLLHSDGFTYLY | QTSNLAS | AQNLELPNT |
| | (SEQ ID NO: 39) | (SEQ ID NO: 40) | (SEQ ID NO: 41) |
| huEpCAM23_VLG v4 | RSSRSLLHSDGFTYLY | QTSNLAS | AQNLELPNT |
| | (SEQ ID NO: 42) | (SEQ ID NO: 40) | (SEQ ID NO: 41) |

Affinity Variants

| | | | |
|---|---|---|---|
| huEpCAM23LCGv4a | RSSRSLLHSNGFTYLY | QTSNLAS | AQNLELPNT |
| | (SEQ ID NO: 43) | (SEQ ID NO: 40) | (SEQ ID NO: 41) |
| huEpCAM23LCGv4b | RSSRSLLHSDGITYLY | QTSNLAS | AQNLELPNT |
| | (SEQ ID NO: 44) | (SEQ ID NO: 40) | (SEQ ID NO: 41) |
| huEpCAM23LCGv4c | RSSRSLLHSDGFTYLY | QTSNLAS | AQNLELPWT |
| | (SEQ ID NO: 42) | (SEQ ID NO: 40) | (SEQ ID NO: 46) |
| huEpCAM23LCGv4e | RSSRSLLHSDGFTYLS | QTSNLAS | AQNLELPNT |
| | (SEQ ID NO: 45) | (SEQ ID NO: 40) | (SEQ ID NO: 41) |
| huEpCAM23LCGv4f | RSSRSLLHSDGFTYLY | QTSNLAS | AQNLELPNT |
| | (SEQ ID NO: 42) | (SEQ ID NO: 40) | (SEQ ID NO: 41) |
| huEpCAM23LCGv4g | RSSRSLLHSDGFTYLY | QTSNLAS | QQNLELPNT |
| | (SEQ ID NO: 42) | (SEQ ID NO: 40) | (SEQ ID NO: 47) |
| huEpCAM23LCGv4h | RSSRSLLHSDGFTYLY | QTSNLAS | LQNLELPNT |
| | (SEQ ID NO: 42) | (SEQ ID NO: 40) | (SEQ ID NO: 48) |
| huEpCAM23LCGv4 \| | RSSRSLLHSDGFTYLY | QTSNLAS | AQYLELPNT |
| | (SEQ ID NO: 42) | (SEQ ID NO: 40) | (SEQ ID NO: 49) |
| huEpCAM23LCGv4j | RSSRSLLHSDGFTYLY | QTSNLAS | AQGLELPNT |
| | (SEQ ID NO: 42) | (SEQ ID NO: 40) | (SEQ ID NO: 50) |
| huEpCAM23LCGv4k | RSSRSLLHSDGFTYLY | QTSNLAS | AQSLELPNT |
| | (SEQ ID NO: 42) | (SEQ ID NO: 40) | (SEQ ID NO: 51) |

In some embodiments, the disclosure provides immunoconjugates having an EpCAM antibody or EpCAM-binding antibody fragment thereof comprising the light chain CDRs of a single row in Table 3. In some embodiments, the disclosure provides immunoconjugates having an EpCAM antibody or EpCAM-binding antibody fragment thereof comprising a VL-CDR1 selected from SEQ ID NOs:39, and 42-45; a VL-CDR2 of SEQ ID NO: 40; and a VL-CDR3 selected from SEQ ID NOs:41, and 46-51. In some embodiments, the disclosure provides immunoconjugates having an EpCAM antibody or EpCAM-binding antibody fragment thereof comprising a VL-CDR1 of SEQ ID NO:42; a VL-CDR2 of SEQ ID NO: 40; and a VL-CDR3 of SEQ ID NO:41. In some embodiments, the disclosure provides immunoconjugates having an EpCAM antibody or EpCAM-binding antibody fragment thereof comprising a VL-CDR1 of SEQ ID NO:39; a VL-CDR2 of SEQ ID NO: 40; and a VL-CDR3 of SEQ ID NO:41.

In one embodiment, the disclosure provides immunoconjugates having an EpCAM antibody, EpCAM-binding antibody fragment thereof, or EpCAM activatable antibody comprising: a VL-CDR1 comprising RSSRSLLHSDGFTYLY (SEQ ID NO:42), or a variant thereof comprising 1, 2, 3, or 4, conservative amino acid substitutions; a VL-CDR2 comprising QTSNLAS (SEQ ID NO:40), or a variant thereof comprising 1, 2, 3, or 4, conservative amino acid substitutions; and a VL-CDR3 comprising AQNLELPNT (SEQ ID NO:41), or a variant thereof comprising 1, 2, 3, or 4, conservative amino acid substitutions. In one embodiment, the disclosure provides immunoconjugates having an EpCAM antibody, EpCAM-binding antibody fragment thereof, or EpCAM activatable antibody comprising: a VL-CDR1 comprising RSSKSLLHSDGFTYLY (SEQ ID NO:39), or a variant thereof comprising 1, 2, 3, or 4, conservative amino acid substitutions; a VL-CDR2 comprising QTSNLAS (SEQ ID NO:40), or a variant thereof comprising 1, 2, 3, or 4, conservative amino acid substitutions; and a VL-CDR3 comprising AQNLELPNT (SEQ ID NO:41), or a variant thereof comprising 1, 2, 3, or 4, conservative amino acid substitutions.

In some embodiments, the disclosure provides immunoconjugates having an EpCAM antibody or EpCAM-binding antibody fragment thereof comprising a VH-CDR1 selected from SEQ ID NOs:13, and 16-25; a VH-CDR2 selected from SEQ ID NOs:14, and 26-29; a VH-CDR3 selected from SEQ ID NOs:15, and 30-38; a VL-CDR1 selected from SEQ ID NOs:39, and 42-45; a VL-CDR2 of SEQ ID NO:40; and a VL-CDR3 selected from SEQ ID NOs:41, and 46-51. In some embodiments, the disclosure provides immunoconjugates having an EpCAM antibody or EpCAM-binding antibody fragment thereof comprising a VH-CDR1, VH-CDR2, VH-CDR3, VL-CDR1, VL-CDR2, and VL-CDR3 having the sequences of SEQ ID NOs: 13-15, 42, 40, and 41, respectively. In some embodiments, the disclosure provides immunoconjugtes having an EpCAM antibody or EpCAM-binding antibody fragment thereof comprising a VH-CDR1, VH-CDR2, VH-CDR3, VL-CDR1, VL-CDR2, and VL-CDR3 having the sequences of SEQ ID NOs: 13-15, and 39-41, respectively. In some embodiments, the disclosure provides immunoconjugates having an EpCAM antibody or EpCAM-binding antibody fragment thereof comprising a VH-CDR1, VH-CDR2, VH-CDR3, VL-CDR1, VL-CDR2, and VL-CDR3 having the sequences of SEQ ID NOs: 13, 26, 15, and 39-41, respectively. In some embodiments, the disclosure provides immunoconjugates having an EpCAM antibody or EpCAM-binding antibody fragment thereof comprising a VH-CDR1, VH-CDR2, VH-CDR3, VL-CDR1, VL-CDR2, and VL-CDR3 having the sequences of SEQ ID NOs: 13, 26, 15, 42, 40, and 41, respectively.

In some embodiments, the disclosure provides immunoconjugates having an EpCAM antibody or EpCAM-binding antibody fragment thereof comprising a VH-CDR1, VH-CDR2, VH-CDR3, VL-CDR1, VL-CDR2, and VL-CDR3 having the sequences of SEQ ID NOs: 22, 14, 15, 42, 40, and 41, respectively. In some embodiments, the disclosure provides immunoconjugates having an EpCAM antibody or EpCAM-binding antibody fragment thereof comprising a VH-CDR1, VH-CDR2, VH-CDR3, VL-CDR1, VL-CDR2, and VL-CDR3 having the sequences of SEQ ID NOs: 13, 14, 33, 42, 40, and 41, respectively. In some embodiments, the disclosure provides immunoconjugates having an EpCAM antibody or EpCAM-binding antibody fragment thereof comprising a VH-CDR1, VH-CDR2, VH-CDR3, VL-CDR1, VL-CDR2, and VL-CDR3 having the sequences of SEQ ID NOs: 23, 14, 15, 42, 40, and 41, respectively. In some embodiments, the disclosure provides immunoconjugates having an EpCAM antibody or EpCAM-binding antibody fragment thereof comprising a VH-CDR1, VH-CDR2, VH-CDR3, VL-CDR1, VL-CDR2, and VL-CDR3 having the sequences of SEQ ID NOs: 25, 14, 15, 42, 40, and 41, respectively.

In some embodiments, immunoconjugates having the EpCAM antibody or EpCAM-binding antibody fragment thereof comprise a heavy chain variable region (VH) sequence disclosed in Table 4. In some embodiments, the EpCAM antibody or EpCAM-binding antibody fragment thereof comprises a VH sequence that is at least 90%, 91%, 92%, 93%, 94%, 95%, 96%, 97%, 98%, or 99%, identical to a VH sequence disclosed in Table 4. In some embodiments, the percent identity in each instance is 95%. In some embodiments, the percent identity in each instance is 98%.

TABLE 4
Exemplary Heavy Chain Variable Sequences of EpCAM
Antibodies.

| | |
|---|---|
| Antibody name | Sequence |

Murine and Chimeric

| | |
|---|---|
| muEpCAM23 | EVKLEESGPALVKPGASVRISCKASGYTFTNYYIHWVKQRPGQGLDYI |
| | GWIYPGNVYIQYNEKFKGKATLTADKSSSTAFMQLSSLTSEDSAVYFC |
| | ARDGPWFAYWGQGTLVTVSS (SEQ ID NO: 52) |
| chEpCAm23 | EVKLEESGPALVKPGASVRISCKASGYTFTNYYIHWVKQRPGQGLDYI |
| | GWIYPGNVYIQYNEKFKGKATLTADKSSSTAFMQLSSLTSEDSAVYFC |
| | ARDGPWFAYWGQGTLVTVSS (SEQ ID NO: 52) |

Humanized Variants

| | |
|---|---|
| huEpCAM23_VHGv1 | QVQLVQSGAEVKKPGASVKVSCKASGYTFTNYYIHWVRQAPGQRLE |
| | WMGWIYPGNVYIQYSQKFQGRVTITRDTSASTAYMELSSLRSEDTAV |
| | YYCARDGPWFAYWGQGTLVTVSS (SEQ ID NO: 53) |
| huEpCAM23_VHGv2 | QVQLVQSGAEVKKPGASVKVSCKASGYTFTNYYIHWVRQAPGQRLEY |
| | IGWIYPGNVYIQYNEKFKGRATLTADKSASTAYMELSSLRSEDTAVYY |
| | CARDGPWFAYWGQGTLVTVSS (SEQ ID NO: 54) |
| huEpCAM23_VHGv3 | QVQLVQSGAEVKKPGASVKVSCKASGYTFTNYYIHWVRQAPGQRLE |
| | WMGWIYPGNVYIQYNEKFKGRVTITRDTSASTAYMELSSLRSEDTAVY |
| | YCARDGPWFAYWGQGTLVTVSS (SEQ ID NO: 55) |
| huEpCAM23_VHGv4 | QVQLVQSGAEVKKPGASVKVSCKASGYTFTNYYIHWVRQAPGQRLEY |
| | IGWIYPGNVYIQYSQKFQGRVTITADKSASTAYMELSSLRSEDTAVYYC |
| | ARDGPWFAYWGQGTLVTVSS (SEQ ID NO: 56) |

Affinity Variants

| | |
|---|---|
| huEpCAM23HCGv2a | QVQLVESGAEVKKPGASVKVSCKASGYTFTNYYIHWVRQAPGQRLEY |
| | IGWIYPGNVYIQYNEKFKGRATLTADKSASTAYMELSSLRSEDTAVYY |
| | CARDGPWFAYWGQGTLVTVSS (SEQ ID NO: 57) |
| huEpCAM23HCGv2b | QVQLVQSGAEVKKPGASVKVSCKASGYTFTSYYIHWVRQAPGQRLEY |
| | IGWIYPGNVYIQYNEKFKGRATLTADKSASTAYMELSSLRSEDTAVYY |
| | CARDGPWFAYWGQGTLVTVSS (SEQ ID NO: 58) |
| huEpCAM23HCGv2c | QVQLVQSGAEVKKPGASVKVSCKASGYTFTNYNIHWVRQAPGQRLE |
| | YIGWIYPGNVYIQYNEKFKGRATLTADKSASTAYMELSSLRSEDTAVYY |
| | CARDGPWFAYWGQGTLVTVSS (SEQ ID NO: 59) |
| huEpCAM23HCGv2d | QVQLVQSGAEVKKPGASVKVSCKASGYTFTNYYIHWVRQAPGQRLEY |
| | IGWIYPGDVYIQYNEKFKGRATLTADKSASTAYMELSSLRSEDTAVYY |
| | CARDGPWFAYWGQGTLVTVSS (SEQ ID NO: 60) |
| huEpCAM23HCGv2e | QVQLVQSGAEVKKPGASVKVSCKASGYTFTNYFIHWVRQAPGQRLEY |
| | IGWIYPGNVYIQYNEKFKGRATLTADKSASTAYMELSSLRSEDTAVYY |
| | CARDGPWFAYWGQGTLVTVSS (SEQ ID NO: 61) |
| huEpCAM23HCGv2f | QVQLVQSGAEVKKPGASVKVSCKASGYTFTNYSIHWVRQAPGQRLEY |
| | IGWIYPGNVYIQYNEKFKGRATLTADKSASTAYMELSSLRSEDTAVYY |
| | CARDGPWFAYWGQGTLVTVSS (SEQ ID NO: 62) |
| huEpCAM23HCGv2g | QVQLVQSGAEVKKPGASVKVSCKASGYTFTNYWIHWVRQAPGQRLEYI |
| | GWIYPGNVYIQYNEKFKGRATLTADKSASTAYMELSSLRSEDTAVYYCA |
| | RDGPWFAYWGQGTLVTVSS (SEQ ID NO: 63) |
| huEpCAM23HCGv2h | QVQLVQSGAEVKKPGASVKVSCKASGYTFTNYYIHWVRQAPGQRLEY |
| | IGWFYPGNVYIQYNEKFKGRATLTADKSASTAYMELSSLRSEDTAVYY |
| | CARDGPWFAYWGQGTLVTVSS (SEQ ID NO: 64) |
| huEpCAM23HCGv2i | QVQLVQSGAEVKKPGASVKVSCKASGYTFTNYYIHWVRQAPGQRLE |
| | WIGWIYPGNVYIQYNEKFKGRATLTADKSASTAYMELSSLRSEDTAVY |
| | YCARDGPWFAYWGQGTLVTVSS (SEQ ID NO: 65) |
| huEpCAM23HCGv2j | QVQLVQSGAEVKKPGASVKVSCKASGYTFTNYYIHWVRQAPGQRLEY |
| | IGWINPGNVYIQYNEKFKGRATLTADKSASTAYMELSSLRSEDTAVYY |
| | CARDGPWFAYWGQGTLVTVSS (SEQ ID NO: 66) |
| huEpCAM23HCGv2k | QVQLVQSGAEVKKPGASVKVSCKASGYTFTNYYIHWVRQAPGQRLEY |
| | IGWIYPGNVYIQYNEKFKGRATLTADKSASTAYMELSSLRSEDTAVYY |
| | CAREGPWFAYWGQGTLVTVSS (SEQ ID NO: 67) |
| huEpCAM23HCGv21 | QVQLVQSGAEVKKPGASVKVSCKASGYTFTNYYIHWVRQAPGQRLEY |
| | IGWIYPGNVYIQYNEKFKGRATLTADKSASTAYMELSSLRSEDTAVYY |
| | CARDGPYFAYWGQGTLVTVSS(SEQ ID NO: 68) |
| huEpCAM23HCGv2m/ | QVQLVQSGAEVKKPGASVKVSCKASGYTFTNYYIHWVRQAPGQRLEY |
| huEpCam 23HG2-1565-A | IGWIYPGNVYIQYNEKFKGRATLTADKSASTAYMELSSLRSEDTAVYY |
| Heavy Chain | CARDGAWFAYWGQGTLVTVSS (SEQ ID NO: 69) |
| huEpCAM23HCGv2n | QVQLVQSGAEVKKPGASVKVSCKASGYTFTNYYMHWVRQAPGQRLEY |
| | IGWIYPGNVYIQYNEKFKGRATLTADKSASTAYMELSSLRSEDTAVYYC |
| | ARDGPWFAYWGQGTLVTVSS (SEQ ID NO: 70) |
| huEpCAM23HCGv2p | QVQLVQSGAEVKKPGASVKVSCKASGYTFTNYYIHWVRQAPGQRLEY |
| | IGWVYPGNVYIQYNEKFKGRATLTADKSASTAYMELSSLRSEDTAVYY |
| | CARDGPWFAYWGQGTLVTVSS (SEQ ID NO: 71) |
| huEpCAM23HCGv2q | QVQLVQSGAEVKKPGASVKVSCKASGYTFTNYYIHWVRQAPGQRLEY |
| | IGWIYPGNVYIQYNEKFKGRATITADKSASTAYMELSSLRSEDTAVYYC |
| | ARDGPWFAYWGQGTLVTVSS (SEQ ID NO: 72) |
| huEpCAM23HCGv2r | QVQLVQSGAEVKKPGASVKVSCKASGYTFTNYYIHWVRQAPGQRLEY |
| | IGWIYPGNVYIQYNEKFKGRATLTRDKSASTAYMELSSLRSEDTAVYY |
| | CARDGPWFAYWGQGTLVTVSS (SEQ ID NO: 73) |
| huEpCAM23HCGv2s | QVQLVQSGAEVKKPGASVKVSCKASGYTFTNYYIHWVRQAPGQRLEY |
| | IGWIYPGNVYIQYNEKFKGRVTLTADKSASTAYMELSSLRSEDTAVYY |
| | CARDGPWFAYWGQGTLVTVSS (SEQ ID NO: 74) |
| huEpCam23HG2-1361-H | QVQLVQSGAEVKKPGASVKVSCKASGYTFTNYHIHWVRQAPGQRLE |
| Heavy Chain | YIGWIYPGNVYIQYNEKFKGRATLTADKSASTAYMELSSLRSEDTAVYY |
| | CARDGPWFAYWGQGTLVTVSS (SEQ ID NO: 75) |
| huEpCam23HG2-1361-D | QVQLVQSGAEVKKPGASVKVSCKASGYTFTNYDIHWVRQAPGQRLE |
| Heavy Chain | YIGWIYPGNVYIQYNEKFKGRATLTADKSASTAYMELSSLRSEDTAVYY |
| | CARDGPWFAYWGQGTLVTVSS (SEQ ID NO: 76) |
| huEpCam23HG2-1565-Y | QVQLVQSGAEVKKPGASVKVSCKASGYTFTNYYIHWVRQAPGQRLEY |
| Heavy Chain | IGWIYPGNVYIQYNEKFKGRATLTADKSASTAYMELSSLRSEDTAVYY |
| | CARDGYWFAYWGQGTLVTVSS (SEQ ID NO: 77) |
| huEpCam23HG2-1565-S | QVQLVQSGAEVKKPGASVKVSCKASGYTFTNYYIHWVRQAPGQRLEY |
| Heavy Chain | IGWIYPGNVYIQYNEKFKGRATLTADKSASTAYMELSSLRSEDTAVYY |
| | CARDGSWFAYWGQGTLVTVSS (SEQ ID NO: 78) |
| huEpCam23HG2-1565-F | QVQLVQSGAEVKKPGASVKVSCKASGYTFTNYYIHWVRQAPGQRLEY |
| Heavy Chain | IGWIYPGNVYIQYNEKFKGRATLTADKSASTAYMELSSLRSEDTAVYY |
| | CARDGFWFAYWGQGTLVTVSS (SEQ ID NO: 79 |
| huEpCam23HG2-1565-G | QVQLVQSGAEVKKPGASVKVSCKASGYTFTNYYIHWVRQAPGQRLEY |
| Heavy Chain | IGWIYPGNVYIQYNEKFKGRATLTADKSASTAYMELSSLRSEDTAVYY |
| | CARDGGWFAYWGQGTLVTVSS (SEQ ID NO: 80) |
| huEpCam23HG2-1565-T | QVQLVQSGAEVKKPGASVKVSCKASGYTFTNYYIHWVRQAPGQRLEY |
| Heavy Chain | IGWIYPGNVYIQYNEKFKGRATLTADKSASTAYMELSSLRSEDTAVYY |
| | CARDGTWFAYWGQGTLVTVSS (SEQ ID NO: 81) |
| huEpCam23HG2-1565-V | QVQLVQSGAEVKKPGASVKVSCKASGYTFTNYYIHWVRQAPGQRLEY |
| Heavy Chain | IGWIYPGNVYIQYNEKFKGRATLTADKSASTAYMELSSLRSEDTAVYY |
| | CARDGWWFAYWGQGTLVTVSS (SEQ ID NO: 82) |
| huEpCam23HG2-1361-1 | QVQLVQSGAEVKKPGASVKVSCKASGYTFTNYIIHWVRQAPGQRLEYI |
| Heavy Chain | GWIYPGNVYIQYNEKFKGRATLTADKSASTAYMELSSLRSEDTAVYYC |
| | ARDGPWFAYWGQGTLVTVSS (SEQ ID NO: 83) |
| huEpCam23HG2-1361-L | QVQLVQSGAEVKKPGASVKVSCKASGYTFTNYLIHWVRQAPGQRLEY |
| Heavy Chain | IGWIYPGNVYIQYNEKFKGRATLTADKSASTAYMELSSLRSEDTAVYY |
| | CARDGPWFAYWGQGTLVTVSS (SEQ ID NO: 84) |

In some embodiments, an immunoconjugate as described herein comprises an EpCAM antibody or EpCAM-binding antibody fragment thereof comprising a VH sequence having a total of one, two, three, four, five, six, seven, eight, nine, ten, fewer than fifteen, or zero, amino acid substitutions, deletions, and/or insertions from a reference VH sequence selected from SEQ ID NOs: 53-84. In some embodiments, the insertions, substitutions, deletions, and/or insertions are in framework regions(s) of the reference sequence. In some embodiments, the substitutions are conservative. In other embodiments, the substitutions are non-conservative.

In some embodiments, an immunoconjugate as described herein comprises an EpCAM antibody or EpCAM-binding antibody fragment thereof comprising a VH having a sequence selected from SEQ ID NOs:53-84. In some embodiments, the EpCAM antibody or EpCAM-binding antibody fragment thereof comprises a VH having a sequence selected from SEQ ID NOs:53-56. In some embodiments, the EpCAM antibody or EpCAM-binding antibody fragment thereof comprises a VH having the sequence of SEQ ID NO:54. In some embodiments, the EpCAM antibody or EpCAM-binding antibody fragment thereof comprises a VH sequence that is at least 90%, 91%, 92%, 93%, 94%, 95%, 96%, 97%, 98%, or 99%, identical to a sequence selected from SEQ ID NOs: 53-56. In some embodiments, the EpCAM antibody or EpCAM-binding antibody fragment thereof comprises a VH sequence that is at least 90%, 91%, 92%, 93%, 94%, 95%, 96%, 97%, 98%, and 99%, identical to the sequence of SEQ ID NO: 54. In some embodiments, the percent identity in each instance is 95%. In some embodiments, the percent identity in each instance is 98%.

In some embodiments, the immunoconjugates disclosed herein comprise EpCAM antibody or EpCAM-binding antibody fragment thereof comprising a VH having a sequence selected from SEQ ID NOs: 75-77, and 84. In some embodiments, the EpCAM antibody or EpCAM-binding antibody fragment thereof comprises a VH having the sequence of SEQ ID NO:75. In some embodiments, the EpCAM antibody or EpCAM-binding antibody fragment thereof comprises a VH having the sequence of SEQ ID NO:77. In some embodiments, the EpCAM antibody or EpCAM-binding antibody fragment thereof comprises a VH sequence that is at least 90%, 91%, 92%, 93%, 94%, 95%, 96%, 97%, 98%, or 99%, identical to a sequence selected from SEQ ID NOs:75-77, and 84. In some embodiments, the EpCAM antibody or EpCAM-binding antibody fragment thereof comprises a VH sequence that is at least 90%, 91%, 92%, 93%, 94%, 95%, 96%, 97%, 98%, and 99%, identical to the sequence of SEQ ID NO: 75. In some embodiments, the EpCAM antibody or EpCAM-binding antibody fragment thereof comprises a VH sequence that is at least 90%, 91%, 92%, 93%, 94%, 95%, 96%, 97%, 98%, and 99%, identical to the sequence of SEQ ID NO: 77. In some embodiments, the percent identity in each instance is 95%. In some embodiments, the percent identity in each instance is 98%.

In some embodiments, the immunoconjugates disclosed herein comprise EpCAM antibody or EpCAM-binding antibody fragment thereof comprising a light chain variable region (VL) sequence disclosed in Table 5. In some embodiments, the EpCAM antibody or EpCAM-binding antibody fragment thereof comprises a VL sequence that is at least 90%, 91%, 92%, 93%, 94%, 95%, 96%, 97%, 98%, or 99%, identical to a VL sequence disclosed in Table 5. In some embodiments, the percent identity in each instance is 95%. In some embodiments, the percent identity in each instance is 98%.

TABLE 5
Exemplary Light Chain Variable Sequences of EpCAM Antibodies.

| | |
|---|---|
| Antibody name | Sequence |

Murine and Chimeric

| | |
|---|---|
| muEpcam23 | DIVLTQTPFSNPVTLGTSASISCRSSKSLLHSDGFTYLYWFLQKPGQSPH |
| | LLIYQTSNLASGVPDRFSSSGSGTDFTLRISRVEAEDVGVYYCAQNLELP |
| | NTFGGGTKLEIK (SEQ ID NO: 85) |
| chEpcam23 | DIVLTQTPFSNPVTLGTSASISQRSSKSLLHSDGFTYLYWFLQKPGQSPH |
| | LLIYQTSNLASGVPDRFSSSGSGTDFTLRISRVEAEDVGVYYCAQNLELP |
| | NTFGGGTKLEIK (SEQ ID NO: 85) |

Humanized Variants

| | |
|---|---|
| huEpCAM23_VLGv1 | DIVMTQTPLSLSVTPGQPASISCRSSRSLLHSDGFTYLYWYLQKPGQSP |
| | QLLIYQTSNLASGVPDRFSGSGSGTDFTLKISRVEAEDVGVYYCAQNLEL |
| | PNTFGQGTKLEIK (SEQ ID NO: 86) |
| huEpCAM23_VLGv2 | DIVLTQTPLSLSVTPGQPASISCRSSKSLLHSDGFTYLYWFLQKPGQSPQ |
| | LLIYQTSNLASGVPDRFSSSGSGTDFTLKISRVEAEDVGVYYCAQNLELP |
| | NTFGQGTKLEIK (SEQ ID NO: 87) |
| huEpCAM23_VLGv3 | DIVMTQTPLSLSVTPGQPASISCRSSKSLLHSDGFTYLYWFLQKPGQSP |
| | QLLIYQTSNLASGVPDRFSGSGSGTDFTLKISRVEAEDVGVYYCAQNLEL |
| | PNTFGQGTKLEIK (SEQ ID NO: 88) |
| huEpCAM23_VLGv4 | DIVLTQTPLSLSVTPGQPASISCRSSRSLLHSDGFTYLYWFLQKPGQSPQ |
| | LLIYQTSNLASGVPDRFSSSGSGTDFTLKISRVEAEDVGVYYCAQNLELP |
| | NTFGQGTKLEIK (SEQ ID NO: 89) |

Affinity Variants

| | |
|---|---|
| huEpCAM23LCGv4a | DIVLTQTPLSLSVTPGQPASISCRSSRSLLHSNGFTYLYWFLQKPGQSPQ |
| | LLIYQTSNLASGVPDRFSSSGSGTDFTLKISRVEAEDVGVYYCAQNLELP |
| | NTFGQGTKLEIK (SEQ ID NO: 90) |
| huEpCAM23LCGv4b | DIVLTQTPLSLSVTPGQPASISCRSSRSLLHSDGITYLYWFLQKPGQSPQ |
| | LLIYQTSNLASGVPDRFSSSGSGTDFTLKISRVEAEDVGVYYCAQNLELP |
| | NTFGQGTKLEIK (SEQ ID NO: 91) |
| huEpCAM23LCGv4c | DIVLTQTPLSLSVTPGQPASISCRSSRSLLHSDGFTYLYWFLQKPGQSPQ |
| | LLIYQTSNLASGVPDRFSSSGSGTDFTLKISRVEAEDVGVYYCAQNLELP |
| | WTFGQGTKLEIK (SEQ ID NO: 92) |
| huEpCAM23LCGv4e | DIVLTQTPLSLSVTPGQPASISCRSSRSLLHSDGFTYLSWFLQKPGQSPQ |
| | LLIYQTSNLASGVPDRFSSSGSGTDFTLKISRVEAEDVGVYYCAQNLELP |
| | NTFGQGTKLEIK (SEQ ID NO: 93) |
| huEpCAM23LCGv4f | DIVLTQTPLSLSVTPGQPASISCRSSRSLLHSDGFTYLYWYLQKPGQSPQ |
| | LLIYQTSNLASGVPDRESSSGSGTDFTLKISRVEAEDVGVYYCAQNLELP |
| | NTFGQGTKLEIK (SEQ ID NO: 94) |
| huEpCAM23LCGv4g | DIVLTQTPLSLSVTPGQPASISCRSSRSLLHSDGFTYLYWFLQKPGQSPQ |
| | LLIYQTSNLASGVPDRFSSSGSGTDFTLKISRVEAEDVGVYYCQQNLELP |
| | NTFGQGTKLEIK (SEQ ID NO: 95) |
| huEpCAM23LCGv4h | DIVLTQTPLSLSVTPGQPASISCRSSRSLLHSDGFTYLYWFLQKPGQSPQ |
| | LLIYQTSNLASGVPDRESSSGSGTDFTLKISRVEAEDVGVYYCLQNLELP |
| | NTFGQGTKLEIK (SEQ ID NO: 96) |
| huEpCAM23LCGv41 | DIVLTQTPLSLSVTPGQPASISCRSSRSLLHSDGFTYLYWFLQKPGQSPQ |
| | LLIYQTSNLASGVPDRESSSGSGTDFTLKISRVEAEDVGVYYCAQYLELP |
| | NTFGQGTKLEIK (SEQ ID NO: 97) |
| huEpCAM23LCGv4] | DIVLTQTPLSLSVTPGQPASISCRSSRSLLHSDGFTYLYWFLQKPGQSPQ |
| | LLIYQTSNLASGVPDRFSSSGSGTDFTLKISRVEAEDVGVYYCAQGLELP |
| | NTFGQGTKLEIK (SEQ ID NO: 98) |
| huEpCAM23LCGv4k | DIVLTQTPLSLSVTPGQPASISCRSSRSLLHSDGFTYLYWFLQKPGQSPQ |
| | LLIYQTSNLASGVPDRESSSGSGTDFTLKISRVEAEDVGVYYCAQSLELP |
| | NTFGQGTKLEIK (SEQ ID NO: 99) |

In some embodiments, the immunoconjugates disclosed herein comprise EpCAM antibody or EpCAM-binding antibody fragment thereof comprising a VL sequence having a total of one, two, three, four, five, six, seven, eight, nine, ten, fewer than fifteen, or zero, amino acid substitutions, deletions, and/or insertions from a reference VH sequence selected from SEQ ID NOs: 86-99. In some embodiments, the insertions, substitutions, deletions, and/or insertions are in framework regions(s) of the reference sequence. In some embodiments, the substitutions are conservative. In other embodiments, the substitutions are non-conservative.

In some embodiments, the immunoconjugates disclosed herein comprise an EpCAM antibody or EpCAM-binding antibody fragment thereof comprising a VL having a sequence selected from SEQ ID NOs:86-99. In some embodiments, the EpCAM antibody or EpCAM-binding antibody fragment thereof comprises a VL having the sequence of SEQ ID NO:89. In some embodiments, the EpCAM antibody or EpCAM-binding antibody fragment thereof comprises a VL having the sequence of SEQ ID NO:87. In some embodiments, the EpCAM antibody or EpCAM-binding antibody fragment thereof comprises a VL sequence that is at least 90%, 91%, 92%, 93%, 94%, 95%, 96%, 97%, 98%, or 99%, identical to a sequence selected from SEQ ID NOs: 86-89. In some embodiments, the EpCAM antibody or EpCAM-binding antibody fragment thereof comprises a VL sequence that is at least 90%, 91%, 92%, 93%, 94%, 95%, 96%, 97%, 98%, and 99%, identical to the sequence of SEQ ID NO: 89. In some embodiments, the EpCAM antibody or EpCAM-binding antibody fragment thereof comprises a VL sequence that is at least 90%, 91%, 92%, 93%, 94%, 95%, 96%, 97%, 98%, and 99%, identical to the sequence of SEQ ID NO: 87. In some embodiments, the percent identity in each instance is 95%. In some embodiments, the percent identity in each instance is 98%.

In some embodiments, the immunoconjugates disclosed herein comprise EpCAM antibody or EpCAM-binding antibody fragment thereof comprising a VH comprising a sequence selected from SEQ ID NOs:53-84 and a VL comprising a sequence selected from SEQ ID NOs: 86-89. In some embodiments, the EpCAM antibody or EpCAM-binding antibody fragment thereof comprises a VH comprising the sequence of SEQ ID NO:54 and a VL comprising the sequence of SEQ ID NO: 89. In some embodiments, the EpCAM antibody or EpCAM-binding antibody fragment thereof comprises a VH comprising the sequence of SEQ ID NO:54 and a VL comprising the sequence of SEQ ID NO: 87. In some embodiments, the EpCAM antibody or EpCAM-binding antibody fragment thereof comprises a VH comprising the sequence of SEQ ID NO:55, and a VL comprising the sequence of SEQ ID NO: 87. In some embodiments, the EpCAM antibody or EpCAM-binding antibody fragment thereof comprises a VH comprising the sequence of SEQ ID NO:56, and a VL comprising the sequence of SEQ ID NO: 88. In some embodiments, the EpCAM antibody or EpCAM-binding antibody fragment thereof comprises a VH comprising the sequence of SEQ ID NO:55, and a VL comprising the sequence of SEQ ID NO: 89. In some embodiments, the EpCAM antibody or EpCAM-binding antibody fragment thereof comprises a VH comprising the sequence of SEQ ID NO:56, and a VL comprising the sequence of SEQ ID NO: 89.

In some embodiments, the immunoconjugates disclosed herein comprise EpCAM antibody or EpCAM-binding antibody fragment thereof comprising a VH comprising the sequence of SEQ ID NO:75, and a VL comprising the sequence of SEQ ID NO: 89. In some embodiments, the EpCAM antibody or EpCAM-binding antibody fragment thereof comprises a VH comprising the sequence of SEQ ID NO:77, and a VL comprising the sequence of SEQ ID NO: 89. In some embodiments, the EpCAM antibody or EpCAM-binding antibody fragment thereof comprises a VH comprising the sequence of SEQ ID NO:76, and a VL comprising the sequence of SEQ ID NO: 89. In some embodiments, the EpCAM antibody or EpCAM-binding antibody fragment thereof comprises a VH comprising the sequence of SEQ ID NO:84, and a VL comprising the sequence of SEQ ID NO: 89.

In some embodiments, the immunoconjugates disclosed herein comprise EpCAM antibody or EpCAM-binding antibody fragment thereof that competes for binding to human EpCAM with an antibody comprising a VH and a VL sequence disclosed in Tables 4 and 5, respectively.

In one embodiment, the disclosure provides immunoconjugates comprising an EpCAM antibody or EpCAM-binding antibody fragment thereof that competes for binding to human EpCAM with an antibody comprising a heavy chain variable region (VH) and a light chain variable region (VL) selected from:
(a) a heavy chain variable region (VH) of SEQ ID NO:54 and a light chain variable region (VL) of SEQ ID NO:89;
(b) a VH of SEQ ID NO:54 and a VL of SEQ ID NO: 87;
(c) a VH of SEQ ID NO:75 and a VL of SEQ ID NO: 89; and
(d) a VH of SEQ ID NO:77 and a VL of SEQ ID NO: 89.

An EpCAM antibody or EpCAM-binding antibody fragment thereof (or an immunoconjugate having the EpCAM antibody or EpCAM-binding antibody fragment thereof) is said to “compete” with a reference molecule for binding to EpCAM if it binds to human EpCAM to the extent that it blocks, to some degree, binding of the reference molecule to human EpCAM. The ability of proteins to compete for binding to EpCAM and thus to interfere with, block or “cross-block” one anothers' binding to EpCAM can be determined by any standard competitive binding assay known in the art including, for example, a competition ELISA assay, surface plasmon resonance (SPR; BIACORE®, Biosensor, Piscataway, N.J.) or according to methods described by Scatchard et al. (Ann. N. Y. Acad. Sci. 51:660-672 (1949)). An antibody may be said to competitively inhibit binding of the reference EpCAM antibody to human EpCAM, for example, by at least 90%, at least 80%, at least 70%, at least 60%, or at least 50%.

In some embodiments, immunoconjugates having the EpCAM antibody or EpCAM-binding antibody fragment thereof further includes a heavy chain constant region or fragment thereof. In some aspects, the antibody or antibody fragment comprises a heavy chain immunoglobulin constant region selected from the group consisting of: (a) a human IgA constant region, or fragment thereof; (b) a human IgD constant region, or fragment thereof; (c) a human IgE constant domain, or fragment thereof: (d) a human IgG1 constant region, or fragment thereof; (e) a human IgG2 constant region, or fragment thereof; (f) a human IgG3 constant region, or fragment thereof; (g) a human IgG4 constant region, or fragment thereof; and (h) a human IgM constant region, or fragment thereof. In certain embodiments, the EpCAM antibody or EpCAM-binding antibody fragment thereof comprises a heavy chain constant region or fragment thereof, e.g., a human IgG constant region or fragment thereof. In further embodiments, the EpCAM antibody or EpCAM-binding antibody fragment thereof comprises a heavy chain immunoglobulin constant domain that has, or has been mutated to have altered effector function and/or half-life.

In some embodiments, immunoconjugates having the EpCAM antibody or EpCAM-binding antibody fragment thereof comprises a heavy chain sequence disclosed in Table 6.

TABLE 6
Exemplary heavy chain full-length sequences of EpCAM
antibodies.

| | |
|---|---|
| Antibody name | Sequence |

Murine and Chimeric

| | |
|---|---|
| muEpCAM-23HC | EVKLEESGPALVKPGASVRISCKASGYTFTNYYIHWVKQRPGQGLDYIGWI |
| | YPGNVYIQYNEKFKGKATLTADKSSSTAFMQLSSLTSEDSAVYFCARDGP |
| | WFAYWGQGTLVTVSSAKTTPPSVYPLAPGCGDTTGSSVTLGCLVKGYFP |
| | ESVTVTWNSGSLSSSVHTFPALLQSGLYTMSSSVTVPSSTWPSQTVTCSV |
| | AHPASSTTVDKKLEPSGPISTINPCPPCKECHKCPAPNLEGGPSVFIFPPNI |
| | KDVLMISLTPKVTCVVVDVSEDDPDVRISWFVNNVEVHTAQTQTHREDYN |
| | STIRVVSALPIQHQDWMSGKEFKCKVNNKDLPSPIERTISKIKGLVRAPQVY |
| | ILPPPAEQLSRKDVSLTCLVVGFNPGDISVEWTSNGHTEENYKDTAPVLDS |
| | DGSYFIYSKLDIKTSKWEKTDSFSCNVRHEGLKNYYLKKTISRSPGK |
| | (SEQ ID NO: 100) |
| chEpcam23HC | EVKLEESGPALVKPGASVRISCKASGYTFTNYYIHWVKQRPGQGLDYIGWI |
| | YPGNVYIQYNEKFKGKATLTADKSSSTAFMQLSSLTSEDSAVYFCARDGP |
| | WFAYWGQGTLVTVSSASTKGPSVFPLAPSSKSTSGGTAALGQLVKDYFPE |
| | PVTVSWNSGALTSGVHTFPAVLQSSGLYSLSSVVTVPSSSLGTQTYICNVN |
| | HKPSNTKVDKKVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPS |
| | RDELTKNQVSLTCLVKGFYPSDIAVEWESNGQPENNYKTTPPVLDSDGSF |
| | FLYSKLTVDKSRWQQGNVFSCSVMHEALHNHYTQKSLSLSPG |
| | (SEQ ID NO: 101) |

Humanized variants

| | |
|---|---|
| huEpCAM23 VHGv1 | QVQLVQSGAEVKKPGASVKVSCKASGYTFTNYYIHWVRQAPGQRLEWM |
| | GWIYPGNVYIQYSQKFQGRVTITRDTSASTAYMELSSLRSEDTAVYYCARD |
| | GPWFAYWGQGTLVTVSSASTKGPSVFPLAPSSKSTSGGTAALGQLVKDY |
| | FPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| | (SEQ ID NO: 102) |
| huEpCAM23 VHGv2 | QVQLVQSGAEVKKPGASVKVSCKASGYTFTNYYIHWVRQAPGQRLEYIG |
| (lysine-linked) | WIYPGNVYIQYNEKFKGRATLTADKSASTAYMELSSLRSEDTAVYYCARD |
| | GPWFAYWGQGTLVTVSSASTKGPSVFPLAPSSKSTSGGTAALGCLVKDY |
| | FPEPVTVSWNSGALTSGVHTFPAVLQSSGLYSLSSVVTVPSSSLGTQTYIC |
| | NVNHKPSNTKVDKKVEPKSC*DKTHTC*PPC*PAPELLGGPSVFLFPPKPK |
| | DTLMISRTPEVTCVVVDVSHEDPEVKFNWYVDGVEVHNAKTKPREEQYNS |
| | TYRVVSVLTVLHQDWLNGKEYKCKVSNKALPAPIEKTISKAKGQPREPQVY |
| | TLPPSRDELTKNQVSLTCLVKGFYPSDIAVEWESNGQPENNYKTTPPVLD |
| | SDGSFFLYSKLTVDKSRWQQGNVFSCSVMHEALHNHYTQKSLSLSPG |
| | (SEQ ID NO: 103) |
| | * denotes cysteines which, when in a native |
| | conformation (e.g., not reduced), participate |
| | in forming interchain disulfide bonds. |
| huEpCAM23 VHGv2- | QVQLVQSGAEVKKPGASVKVSCKASGYTFTNYYIHWVRQAPGQRLEYIG |
| C442 (site-specific) | WIYPGNVYIQYNEKFKGRATLTADKSASTAYMELSSLRSEDTAVYYCARD |
| | GPWFAYWGQGTLVTVSSASTKGPSVFPLAPSSKSTSGGTAALGQLVKDY |
| | FPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYTL |
| | PPSRDELTKNQVSLTQLVKGFYPSDIAVEWESNGQPENNYKTTPPVLDSD |
| | GSFFLYSKLTVDKSRWQQGNVFSCSVMHEALHNHYTQKSLCLSPG |
| | SLSPG (SEQ ID NO: 104) |
| huEpCAM23 VHGv3 | QVQLVQSGAEVKKPGASVKVSCKASGYTFTNYYIHWVRQAPGQRLEWM |
| | GWIYPGNVYIQYNEKFKGRVTITRDTSASTAYMELSSLRSEDTAVYYCARD |
| | GPWFAYWGQGTLVTVSSASTKGPSVFPLAPSSKSTSGGTAALGCLVKDY |
| | FPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| | (SEQ ID NO: 105) |
| huEpCAM23 VHGv4 | QVQLVQSGAEVKKPGASVKVSCKASGYTFTNYYIHWVRQAPGQRLEYIG |
| | WIYPGNVYIQYSQKFQGRVTITADKSASTAYMELSSLRSEDTAVYYCARDG |
| | PWFAYWGQGTLVTVSSASTKGPSVFPLAPSSKSTSGGTAALGCLVKDYFP |
| | EPVTVSWNSGALTSGVHTFPAVLQSSGLYSLSSVVTVPSSSLGTQTYICNV |
| | NHKPSNTKVDKKVEPKSCDKTHTCPPCPAPELLGGPSVFLFPPKPKDTLMI |
| | SRTPEVTCVWVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SRDELTKNQVSLTCLVKGFYPSDIAVEWESNGQPENNYKTTPPVLDSDGS |
| | FFLYSKLTVDKSRWQQGNVFSCSVMHEALHNHYTQKSLSLSPG |
| | (SEQ ID NO: 106) |

Affinity variants

| | |
|---|---|
| huEpCAM23HCGv2a | QVQLVESGAEVKKPGASVKVSCKASGYTFTNYYIHWVRQAPGQRLEYIG |
| | WIYPGNVYIQYNEKFKGRATLTADKSASTAYMELSSLRSEDTAVYYCARD |
| | GPWFAYWGQGTLVTVSSASTKGPSVFPLAPSSKSTSGGTAALGCLVKDY |
| | FPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| | (SEQ ID NO: 107) |
| huEpCAM23HCGv2b | QVQLVQSGAEVKKPGASVKVSCKASGYTFTSYYIHWVRQAPGQRLEYIG |
| | WIYPGNVYIQYNEKFKGRATLTADKSASTAYMELSSLRSEDTAVYYCARD |
| | GPWFAYWGQGTLVTVSSASTKGPSVFPLAPSSKSTSGGTAALGCLVKDY |
| | FPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| | (SEQ ID NO: 108) |
| huEpCAM23HCGv2c | QVQLVQSGAEVKKPGASVKVSCKASGYTFTNYNIHWVRQAPGQRLEYIG |
| | WIYPGNVYIQYNEKFKGRATLTADKSASTAYMELSSLRSEDTAVYYCARD |
| | GPWFAYWGQGTLVTVSSASTKGPSVFPLAPSSKSTSGGTAALGQLVKDY |
| | FPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDT |
| | LMISRTPEVTCVWDVSHEDPEVKFNWYVDGVEVHNAKTKPREEQYNSTY |
| | RVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| | (SEQ ID NO: 109) |
| huEpCAM23HCGv2d | QVQLVQSGAEVKKPGASVKVSCKASGYTFTNYYIHWVRQAPGQRLEYIG |
| | WIYPGDVYIQYNEKFKGRATLTADKSASTAYMELSSLRSEDTAVYYCARD |
| | GPWFAYWGQGTLVTVSSASTKGPSVFPLAPSSKSTSGGTAALGQLVKDY |
| | FPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| | (SEQ ID NO: 110) |
| huEpCAM23HCGv2e | QVQLVQSGAEVKKPGASVKVSCKASGYTFTNYFIHWVRQAPGQRLEYIG |
| | WIYPGNVYIQYNEKFKGRATLTADKSASTAYMELSSLRSEDTAVYYCARD |
| | GPWFAYWGQGTLVTVSSASTKGPSVFPLAPSSKSTSGGTAALGCLVKDY |
| | FPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDT |
| | LMISRTPEVTQVVVDVSHEDPEVKFNWYVDGVEVHNAKTKPREEQYNSTY |
| | RVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| | (SEQ ID NO: 111) |
| huEpCAM23HCGv2f | QVQLVQSGAEVKKPGASVKVSCKASGYTFTNYSIHWVRQAPGQRLEYIG |
| | WIYPGNVYIQYNEKFKGRATLTADKSASTAYMELSSLRSEDTAVYYCARD |
| | GPWFAYWGQGTLVTVSSASTKGPSVFPLAPSSKSTSGGTAALGQLVKDY |
| | FPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| | (SEQ ID NO: 112) |
| huEpCAM23HCGv2g | QVQLVQSGAEVKKPGASVKVSCKASGYTFTNYWIHWVRQAPGQRLEYIG |
| | WIYPGNVYIQYNEKFKGRATLTADKSASTAYMELSSLRSEDTAVYYCARD |
| | GPWFAYWGQGTLVTVSSASTKGPSVFPLAPSSKSTSGGTAALGQLVKDY |
| | FPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDT |
| | LMISRTPEVTCVWVDVSHEDPEVKFNWYVDGVEVHNAKTKPREEQYNSTY |
| | RVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| | (SEQ ID NO: 113) |
| huEpCAM23HCGv2h | QVQLVQSGAEVKKPGASVKVSCKASGYTFTNYYIHWVRQAPGQRLEYIG |
| | WFYPGNVYIQYNEKFKGRATLTADKSASTAYMELSSLRSEDTAVYYCARD |
| | GPWFAYWGQGTLVTVSSASTKGPSVFPLAPSSKSTSGGTAALGCLVKDY |
| | FPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| | (SEQ ID NO: 114) |
| huEpCAM23HCGv2i/2o | QVQLVQSGAEVKKPGASVKVSCKASGYTFTNYYIHWVRQAPGQRLEWIG |
| | WIYPGNVYIQYNEKFKGRATLTADKSASTAYMELSSLRSEDTAVYYCARD |
| | GPWFAYWGQGTLVTVSSASTKGPSVFPLAPSSKSTSGGTAALGCLVKDY |
| | FPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDT |
| | LMISRTPEVTCVWVDVSHEDPEVKFNWYVDGVEVHNAKTKPREEQYNSTY |
| | RVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| | (SEQ ID NO: 115) |
| huEpCAM23HCGv2j | QVQLVQSGAEVKKPGASVKVSCKASGYTFTNYYIHWVRQAPGQRLEYIG |
| | WINPGNVYIQYNEKFKGRATLTADKSASTAYMELSSLRSEDTAVYYCARD |
| | GPWFAYWGQGTLVTVSSASTKGPSVFPLAPSSKSTSGGTAALGQLVKDY |
| | FPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| | (SEQ ID NO: 116) |
| huEpCAM23HCGv2k | QVQLVQSGAEVKKPGASVKVSCKASGYTFTNYYIHWVRQAPGQRLEYIG |
| | WIYPGNVYIQYNEKFKGRATLTADKSASTAYMELSSLRSEDTAVYYCARE |
| | GPWFAYWGQGTLVTVSSASTKGPSVFPLAPSSKSTSGGTAALGQLVKDY |
| | FPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDT |
| | LMISRTPEVTQVVVDVSHEDPEVKFNWYVDGVEVHNAKTKPREEQYNSTY |
| | RVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| | (SEQ ID NO: 117) |
| huEpCAM23HCGv2L | QVQLVQSGAEVKKPGASVKVSCKASGYTFTNYYIHWVRQAPGQRLEYIG |
| | WIYPGNVYIQYNEKFKGRATLTADKSASTAYMELSSLRSEDTAVYYCARD |
| | GPYFAYWGQGTLVTVSSASTKGPSVFPLAPSSKSTSGGTAALGQLVKDYF |
| | PEPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| | (SEQ ID NO: 118) |
| huEpCAM23HCGv2m/ | QVQLVQSGAEVKKPGASVKVSCKASGYTFTNYYIHWVRQAPGQRLEYIG |
| huEpCam23HG2- | WIYPGNVYIQYNEKFKGRATLTADKSASTAYMELSSLRSEDTAVYYCARD |
| 1565-A Heavy Chain | GAWFAYWGQGTLVTVSSASTKGPSVFPLAPSSKSTSGGTAALGQLVKDY |
| | FPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| | (SEQ ID NO: 119) |
| huEpCAM23HCGv2n | QVQLVQSGAEVKKPGASVKVSCKASGYTFTNYYMHWVRQAPGQRLEYIG |
| | WIYPGNVYIQYNEKFKGRATLTADKSASTAYMELSSLRSEDTAVYYCARD |
| | GPWFAYWGQGTLVTVSSASTKGPSVFPLAPSSKSTSGGTAALGQLVKDY |
| | FPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDT |
| | LMISRTPEVTQVVVDVSHEDPEVKFNWYVDGVEVHNAKTKPREEQYNSTY |
| | RVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| | (SEQ ID NO: 120) |
| huEpCAM23HCGv2p | QVQLVQSGAEVKKPGASVKVSCKASGYTFTNYYIHWVRQAPGQRLEYIG |
| | WVYPGNVYIQYNEKFKGRATLTADKSASTAYMELSSLRSEDTAVYYCARD |
| | GPWFAYWGQGTLVTVSSASTKGPSVFPLAPSSKSTSGGTAALGCLVKDY |
| | FPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| | (SEQ ID NO: 121) |
| huEpCAM23HCGv2q | QVQLVQSGAEVKKPGASVKVSCKASGYTFTNYYIHWVRQAPGQRLEYIG |
| | WIYPGNVYIQYNEKFKGRATITADKSASTAYMELSSLRSEDTAVYYCARDG |
| | PWFAYWGQGTLVTVSSASTKGPSVFPLAPSSKSTSGGTAALGCLVKDYFP |
| | EPVTVSWNSGALTSGVHTFPAVLQSSGLYSLSSVVTVPSSSLGTQTYICNV |
| | NHKPSNTKVDKKVEPKSCDKTHTCPPCPAPELLGGPSVFLFPPKPKDTLMI |
| | SRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SRDELTKNQVSLTQLVKGFYPSDIAVEWESNGQPENNYKTTPPVLDSDGS |
| | FFLYSKLTVDKSRWQQGNVFSCSVMHEALHNHYTQKSLSLSPG |
| | (SEQ ID NO: 122) |
| huEpCAM23HCGv2r | QVQLVQSGAEVKKPGASVKVSCKASGYTFTNYYIHWVRQAPGQRLEYIG |
| | WIYPGNVYIQYNEKFKGRATLTRDKSASTAYMELSSLRSEDTAVYYCARD |
| | GPWFAYWGQGTLVTVSSASTKGPSVFPLAPSSKSTSGGTAALGCLVKDY |
| | FPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDT |
| | LMISRTPEVTCVWVDVSHEDPEVKFNWYVDGVEVHNAKTKPREEQYNSTY |
| | RVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| | (SEQ ID NO: 123) |
| huEpCAM23HCGv2s | QVQLVQSGAEVKKPGASVKVSCKASGYTFTNYYIHWVRQAPGQRLEYIG |
| | WIYPGNVYIQYNEKFKGRVTLTADKSASTAYMELSSLRSEDTAVYYCARD |
| | GPWFAYWGQGTLVTVSSASTKGPSVFPLAPSSKSTSGGTAALGQLVKDY |
| | FPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYTL |
| | PPSRDELTKNQVSLTQLVKGFYPSDIAVEWESNGQPENNYKTTPPVLDSD |
| | GSFFLYSKLTVDKSRWQQGNVFSCSVMHEALHNHYTQKSLSLSPG |
| | (SEQ ID NO: 124) |
| huEpCam23HG2- | QVQLVQSGAEVKKPGASVKVSCKASGYTFTNYHIHWVRQAPGQRLEYIG |
| 1361-H Heavy Chain | WIYPGNVYIQYNEKFKGRATLTADKSASTAYMELSSLRSEDTAVYYCARD |
| | GPWFAYWGQGTLVTVSSASTKGPSVFPLAPSSKSTSGGTAALGCLVKDY |
| | FPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDT |
| | LMISRTPEVTCVWVDVSHEDPEVKFNWYVDGVEVHNAKTKPREEQYNSTY |
| | RVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| | (SEQ ID NQ.125) |
| huEpCam23HG2- | QVQLVQSGAEVKKPGASVKVSCKASGYTFTNYDIHWVRQAPGQRLEYIG |
| 1361-D Heavy Chain | WIYPGNVYIQYNEKFKGRATLTADKSASTAYMELSSLRSEDTAVYYCARD |
| | GPWFAYWGQGTLVTVSSASTKGPSVFPLAPSSKSTSGGTAALGQLVKDY |
| | FPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| | (SEQ ID NO: 126) |
| huEpCam23HG2- | QVQLVQSGAEVKKPGASVKVSCKASGYTFTNYYIHWVRQAPGQRLEYIG |
| 1565-Y Heavy Chain | WIYPGNVYIQYNEKFKGRATLTADKSASTAYMELSSLRSEDTAVYYCARD |
| | GYWFAYWGQGTLVTVSSASTKGPSVFPLAPSSKSTSGGTAALGCLVKDY |
| | FPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDT |
| | LMISRTPEVTQVVDVSHEDPEVKFNWYVDGVEVHNAKTKPREEQYNSTY |
| | RVVSVLTVLHQDWLNGKEYKCKVSNKALPAPIEKTISKAKGQPREPQVYTL |
| | PPSRDELTKNQVSLTQLVKGFYPSDIAVEWESNGQPENNYKTTPPVLDSD |
| | GSFFLYSKLTVDKSRWQQGNVFSCSVMHEALHNHYTQKSLSLSPG |
| | (SEQ ID NO: 127) |
| huEpCam23HG2- | QVQLVQSGAEVKKPGASVKVSCKASGYTFTNYYIHWVRQAPGQRLEYIG |
| 1565-S Heavy Chain | WIYPGNVYIQYNEKFKGRATLTADKSASTAYMELSSLRSEDTAVYYCARD |
| | GSWFAYWGQGTLVTVSSASTKGPSVFPLAPSSKSTSGGTAALGQLVKDY |
| | FPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYTL |
| | PPSRDELTKNQVSLTQLVKGFYPSDIAVEWESNGQPENNYKTTPPVLDSD |
| | GSFFLYSKLTVDKSRWQQGNVFSCSVMHEALHNHYTQKSLSLSPG |
| | (SEQ ID NO: 128) |
| huEpCam23HG2- | QVQLVQSGAEVKKPGASVKVSCKASGYTFTNYYIHWVRQAPGQRLEYIG |
| 1565-F Heavy Chain | WIYPGNVYIQYNEKFKGRATLTADKSASTAYMELSSLRSEDTAVYYCARD |
| | GFWFAYWGQGTLVTVSSASTKGPSVFPLAPSSKSTSGGTAALGCLVKDY |
| | FPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| | (SEQ ID NO: 129) |
| huEpCam23HG2- | QVQLVQSGAEVKKPGASVKVSCKASGYTFTNYYIHWVRQAPGQRLEYIG |
| 1565-G Heavy Chain | WIYPGNVYIQYNEKFKGRATLTADKSASTAYMELSSLRSEDTAVYYCARD |
| | GGWFAYWGQGTLVTVSSASTKGPSVFPLAPSSKSTSGGTAALGCLVKDY |
| | FPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDT |
| | LMISRTPEVTCVWVDVSHEDPEVKFNWYVDGVEVHNAKTKPREEQYNSTY |
| | RVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| | (SEQ ID NO: 130) |
| huEpCam23HG2- | QVQLVQSGAEVKKPGASVKVSCKASGYTFTNYYIHWVRQAPGQRLEYIG |
| 1565-T Heavy Chain | WIYPGNVYIQYNEKFKGRATLTADKSASTAYMELSSLRSEDTAVYYCARD |
| | GTWFAYWGQGTLVTVSSASTKGPSVFPLAPSSKSTSGGTAALGCLVKDY |
| | FPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYTL |
| | PPSRDELTKNQVSLTQLVKGFYPSDIAVEWESNGQPENNYKTTPPVLDSD |
| | GSFFLYSKLTVDKSRWQQGNVFSCSVMHEALHNHYTQKSLSLSPG |
| | (SEQ ID NO: 131) |
| huEpCam23HG2- | QVQLVQSGAEVKKPGASVKVSCKASGYTFTNYYIHWVRQAPGQRLEYIG |
| 1565-V Heavy Chain | WIYPGNVYIQYNEKFKGRATLTADKSASTAYMELSSLRSEDTAVYYCARD |
| | GVWFAYWGQGTLVTVSSASTKGPSVFPLAPSSKSTSGGTAALGQLVKDY |
| | FPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| | (SEQ ID NO: 132) |
| huEpCam23HG2- | QVQLVQSGAEVKKPGASVKVSCKASGYTFTNYIIHWVRQAPGQRLEYIGW |
| 1361-I Heavy Chain | IYPGNVYIQYNEKFKGRATLTADKSASTAYMELSSLRSEDTAVYYCARDGP |
| | WFAYWGQGTLVTVSSASTKGPSVFPLAPSSKSTSGGTAALGCLVKDYFPE |
| | PVTVSWNSGALTSGVHTFPAVLQSSGLYSLSSVVTVPSSSLGTQTYICNVN |
| | HKPSNTKVDKKVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPS |
| | RDELTKNQVSLTCLVKGFYPSDIAVEWESNGQPENNYKTTPPVLDSDGSF |
| | FLYSKLTVDKSRWQQGNVFSCSVMHEALHNHYTQKSLSLSPG |
| | (SEQ ID NO: 133) |
| huEpCam23HG2- | QVQLVQSGAEVKKPGASVKVSCKASGYTFTNYLIHWVRQAPGQRLEYIG |
| 1361-L Heavy Chain | WIYPGNVYIQYNEKFKGRATLTADKSASTAYMELSSLRSEDTAVYYCARD |
| | GPWFAYWGQGTLVTVSSASTKGPSVFPLAPSSKSTSGGTAALGCLVKDY |
| | FPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDT |
| | LMISRTPEVTCVWVDVSHEDPEVKFNWYVDGVEVHNAKTKPREEQYNSTY |
| | RVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| | (SEQ ID NO: 134) |

In some embodiments, immunoconjugates having the EpCAM antibody or EpCAM-binding antibody fragment thereof comprise a heavy chain (HC) sequence selected from SEQ ID NOs:102-134. In some embodiments, the EpCAM antibody or EpCAM-binding antibody fragment thereof of immunoconjugates described herein comprises a heavy chain (HC) sequence selected from SEQ ID NOs:102-106. In a specific embodiment, the EpCAM antibody or EpCAM-binding antibody fragment thereof of immunoconjugates described herein comprises a HC sequence of SEQ ID NO:103. In some embodiments, the EpCAM antibody or EpCAM-binding antibody fragment thereof comprises a HC sequence that is at least 90%, 91%, 92%, 93%, 94%, 95%, 96%, 97%, 98%, or 99%, identical to a sequence selected from SEQ ID NOs:102-134. In some embodiments, the EpCAM antibody or EpCAM-binding antibody fragment thereof comprises a HC sequence that is at least 90%, 91%, 92%, 93%, 94%, 95%, 96%, 97%, 98%, or 99%, identical to a sequence selected from SEQ ID NOs:102-106. In some embodiments, the EpCAM antibody or EpCAM-binding antibody fragment thereof comprises a HC sequence that is at least 90%, 91%, 92%, 93%, 94%, 95%, 96%, 97%, 98%, and 99%, identical to the sequence of SEQ ID NO: 103. In some embodiments, the percent identity in each instance is 95%. In some embodiments, the percent identity in each instance is 98%.

In alternative embodiments, the EpCAM antibody or EpCAM-binding antibody fragment thereof of immunoconjugates described herein comprises a HC sequence selected from SEQ ID NOs:125-127 and 134. In a specific embodiment, the EpCAM antibody or EpCAM-binding antibody fragment thereof of immunoconjugates described herein comprises a HC sequence of SEQ ID NO:125. In a specific embodiment, the EpCAM antibody or EpCAM-binding antibody fragment thereof of immunoconjugates described herein comprises a HC sequence of SEQ ID NO:127.

In some embodiments, the EpCAM antibody or EpCAM-binding antibody fragment thereof comprises a HC sequence that is at least 90%, 91%, 92%, 93%, 94%, 95%, 96%, 97%, 98%, or 99%, identical to a sequence selected from SEQ ID NOs:125-127 and 134. In some embodiments, the EpCAM antibody or EpCAM-binding antibody fragment thereof comprises a HC sequence that is at least 90%, 91%, 92%, 93%, 94%, 95%, 96%, 97%, 98%, and 99%, identical to the sequence of SEQ ID NO: 125. In some embodiments, the percent identity in each instance is 95%. In some embodiments, the percent identity in each instance is 98%.

In additional aspects, the EpCAM antibody or EpCAM-binding antibody fragment thereof of immunoconjugates described herein comprises a light chain immunoglobulin constant region. In a further aspect, the antibody comprises a human Ig kappa constant region or a human Ig lambda constant region.

In some embodiments, the EpCAM antibody or EpCAM-binding antibody fragment thereof of immunoconjugates described herein comprises a light chain sequence disclosed in Table 7.

TABLE 7
Exemplary light chain full-length sequences of EpCAM
antibodies.

| | |
|---|---|
| Antibody name | Sequence |

Murine and Chimeric

| | |
|---|---|
| muEpcam23LC | DIVLTQTPFSNPVTLGTSASISCRSSKSLLHSDGFTYLYWFLQKPGQSPH |
| | LLIYQTSNLASGVPDRFSSSGSGTDFTLRISRVEAEDVGVYYCAQNLELP |
| | NTFGGGTKLEIKRADAAPTVSIFPPSSEQLTSGGASVVCFLNNFYPKDIN |
| | VKWKIDGSERQNGVLNSWTDQDSKDSTYSMSSTLTLTKDEYERHNSYT |
| | CEATHKTSTSPIVKSFNRNEC (SEQ ID NO: 135) |
| chEpCam23LC | DIVLTQTPFSNPVTLGTSASISCRSSKSLLHSDGFTYLYWFLQKPGQSPHL |
| | LIYQTSNLASGVPDRFSSSGSGTDFTLRISRVEAEDVGVYYCAQNLELPNT |
| | FGGGTKLEIKRTVAAPSVFIFPPSDEQLKSGTASVVCLLNNFYPREAKVQ |
| | WKVDNALQSGNSQESVTEQDSKDSTYSLSSTLTLSKADYEKHKVYACEV |
| | THQGLSSPVTKSFNRGEC (SEQ ID NO: 136) |

Humanized variants

| | |
|---|---|
| huEpCAM23_VLGv1 | DIVMTQTPLSLSVTPGQPASISCRSSRSLLHSDGFTYLYWYLQKPGQSPQ |
| | LLIYQTSNLASGVPDRFSGSGSGTDFTLKISRVEAEDVGVYYCAQNLELPN |
| | TFGQGTKLEIKRTVAAPSVFIFPPSDEQLKSGTASVVCLLNNFYPREAKVQ |
| | WKVDNALQSGNSQESVTEQDSKDSTYSLSSTLTLSKADYEKHKVYACEV |
| | THQGLSSPVTKSFNRGEC (SEQ ID NO: 137) |
| huEpCAM23_VLGv2 | DIVLTQTPLSLSVTPGQPASISCRSSKSLLHSDGFTYLYWFLQKPGQSPQL |
| | LIYQTSNLASGVPDRFSSSGSGTDFTLKISRVEAEDVGVYYCAQNLELPNT |
| | FGQGTKLEIKRTVAAPSVFIFPPSDEQLKSGTASVVCLLNNFYPREAKVQ |
| | WKVDNALQSGNSQESVTEQDSKDSTYSLSSTLTLSKADYEKHKVYACEV |
| | THQGLSSPVTKSFNRGEC (SEQ ID NO: 138) |
| huEpCAM23_VLGv3 | DIVMTQTPLSLSVTPGQPASISCRSSKSLLHSDGFTYLYWFLQKPGQSPQ |
| | LLIYQTSNLASGVPDRFSGSGSGTDFTLKISRVEAEDVGVYYCAQNLELPN |
| | TFGQGTKLEIKRTVAAPSVFIFPPSDEQLKSGTASVVCLLNNFYPREAKVQ |
| | WKVDNALQSGNSQESVTEQDSKDSTYSLSSTLTLSKADYEKHKVYACEV |
| | THQGLSSPVTKSFNRGEC (SEQ ID NO: 139) |
| huEpCAM23_VLGv4 | DIVLTQTPLSLSVTPGQPASISCRSSRSLLHSDGFTYLYWFLQKPGQSPQ |
| | LLIYQTSNLASGVPDRFSSSGSGTDFTLKISRVEAEDVGVYYCAQNLELP |
| | NTFGQGTKLEIKRTVAAPSVFIFPPSDEQLKSGTASVVQLLNNFYPREAK |
| | VQWKVDNALQSGNSQESVTEQDSKDSTYSLSSTLTLSKADYEKHKVYA |
| | CEVTHQGLSSPVTKSFNRGEC* (SEQ ID NO: 140) |
| | * denotes cysteine which, when in a native |
| | conformation (e.g., not reduced), participate in |
| | forming interchain disulfide bond. |

Affinity variants

| | |
|---|---|
| huEpCAM23LCGv4a | DIVLTQTPLSLSVTPGQPASISCRSSRSLLHSNGFTYLYWFLQKPGQSPQ |
| | LLIYQTSNLASGVPDRFSSSGSGTDFTLKISRVEAEDVGVYYCAQNLELP |
| | NTFGQGTKLEIKRTVAAPSVFIFPPSDEQLKSGTASVVCLLNNFYPREAK |
| | VQWKVDNALQSGNSQESVTEQDSKDSTYSLSSTLTLSKADYEKHKVYA |
| | CEVTHQGLSSPVTKSFNRGEC (SEQ ID NO: 141) |
| huEpCAM23LCGv4b | DIVLTQTPLSLSVTPGQPASISCRSSRSLLHSDGITYLYWFLQKPGQSPQ |
| | LLIYQTSNLASGVPDRFSSSGSGTDFTLKISRVEAEDVGVYYCAQNLELP |
| | NTFGQGTKLEIKRTVAAPSVFIFPPSDEQLKSGTASVVCLLNNFYPREAK |
| | VQWKVDNALQSGNSQESVTEQDSKDSTYSLSSTLTLSKADYEKHKVYA |
| | CEVTHQGLSSPVTKSFNRGEC (SEQ ID NO: 142) |
| huEpCAM23LCGv4 | DIVLTQTPLSLSVTPGQPASISCRSSRSLLHSDGFTYLYWFLQKPGQSPQ |
| | LLIYQTSNLASGVPDRFSSSGSGTDFTLKISRVEAEDVGVYYCAQNLELP |
| | WTFGQGTKLEIKRTVAAPSVFIFPPSDEQLKSGTASVVCLLNNFYPREAK |
| | VQWKVDNALQSGNSQESVTEQDSKDSTYSLSSTLTLSKADYEKHKVYA |
| | CEVTHQGLSSPVTKSFNRGEC (SEQ ID NO: 143) |
| huEpCAMGvLC4e | DIVLTQTPLSLSVTPGQPASISCRSSRSLLHSDGFTYLSWFLQKPGQSPQ |
| | LLIYQTSNLASGVPDRESSSGSGTDFTLKISRVEAEDVGVYYCAQNLELP |
| | NTFGQGTKLEIKRTVAAPSVFIFPPSDEQLKSGTASVVCLLNNFYPREAK |
| | VQWKVDNALQSGNSQESVTEQDSKDSTYSLSSTLTLSKADYEKHKVYA |
| | CEVTHQGLSSPVTKSFNRGEC (SEQ ID NO: 144) |
| huEpCAMGvLC4f | DIVLTQTPLSLSVTPGQPASISCRSSRSLLHSDGFTYLYWYLQKPGQSPQ |
| | LLIYQTSNLASGVPDRFSSSGSGTDFTLKISRVEAEDVGVYYCAQNLELP |
| | NTFGQGTKLEIKRTVAAPSVFIFPPSDEQLKSGTASVVQLLNNFYPREAK |
| | VQWKVDNALQSGNSQESVTEQDSKDSTYSLSSTLTLSKADYEKHKVYA |
| | CEVTHQGLSSPVTKSFNRGEC (SEQ ID NO: 145) |
| huEpCAMGvLC4g | DIVLTQTPLSLSVTPGQPASISCRSSRSLLHSDGFTYLYWFLQKPGQSPQ |
| | LLIYQTSNLASGVPDRESSSGSGTDFTLKISRVEAEDVGVYYCQQNLELP |
| | NTFGQGTKLEIKRTVAAPSVFIFPPSDEQLKSGTASVVCLLNNFYPREAK |
| | VQWKVDNALQSGNSQESVTEQDSKDSTYSLSSTLTLSKADYEKHKVYA |
| | CEVTHQGLSSPVTKSFNRGEC (SEQ ID NO: 146) |
| huEpCAMGvLC4h | DIVLTQTPLSLSVTPGQPASISCRSSRSLLHSDGFTYLYWFLQKPGQSPQ |
| | LLIYQTSNLASGVPDRFSSSGSGTDFTLKISRVEAEDVGVYYCLQNLELP |
| | NTFGQGTKLEIKRTVAAPSVFIFPPSDEQLKSGTASVVQLLNNFYPREAK |
| | VQWKVDNALQSGNSQESVTEQDSKDSTYSLSSTLTLSKADYEKHKVYA |
| | CEVTHQGLSSPVTKSFNRGEC (SEQ ID NO: 147) |
| huEpCAMGvLC4i | DIVLTQTPLSLSVTPGQPASISCRSSRSLLHSDGFTYLYWFLQKPGQSPQ |
| | LLIYQTSNLASGVPDRFSSSGSGTDFTLKISRVEAEDVGVYYCAQYLELP |
| | NTFGQGTKLEIKRTVAAPSVFIFPPSDEQLKSGTASVVQLLNNFYPREAK |
| | VQWKVDNALQSGNSQESVTEQDSKDSTYSLSSTLTLSKADYEKHKVYA |
| | CEVTHQGLSSPVTKSFNRGEC (SEQ ID NO: 148) |
| huEpCAMGvLC4j | DIVLTQTPLSLSVTPGQPASISCRSSRSLLHSDGFTYLYWFLQKPGQSPQ |
| | LLIYQTSNLASGVPDRESSSGSGTDFTLKISRVEAEDVGVYYCAQGLELP |
| | NTFGQGTKLEIKRTVAAPSVFIFPPSDEQLKSGTASVVCLLNNFYPREAK |
| | VQWKVDNALQSGNSQESVTEQDSKDSTYSLSSTLTLSKADYEKHKVYA |
| | CEVTHQGLSSPVTKSFNRGEC (SEQ ID NO: 149) |
| huEpCAMGvLC4k | DIVLTQTPLSLSVTPGQPASISCRSSRSLLHSDGFTYLYWFLQKPGQSPQ |
| | LLIYQTSNLASGVPDRFSSSGSGTDFTLKISRVEAEDVGVYYCAQSLELP |
| | NTFGQGTKLEIKRTVAAPSVFIFPPSDEQLKSGTASVVQLLNNFYPREAK |
| | VQWKVDNALQSGNSQESVTEQDSKDSTYSLSSTLTLSKADYEKHKVYA |
| | CEVTHQGLSSPVTKSFNRGEC (SEQ ID NO: 150) |

In some embodiments, the EpCAM antibody or EpCAM-binding antibody fragment thereof of immunoconjugates described herein comprises a light chain (LC) sequence selected from SEQ ID NOs:137-150. In a specific embodiment, the EpCAM antibody or EpCAM-binding antibody fragment thereof of immunoconjugates described herein comprises a LC sequence of SEQ ID NO:140. In another embodiment, the EpCAM antibody or EpCAM-binding antibody fragment thereof of immunoconjugates described herein comprises a LC sequence of SEQ ID NO:138. In some embodiments, the EpCAM antibody or EpCAM-binding antibody fragment thereof comprises a LC sequence that is at least 90%, 91%, 92%, 93%, 94%, 95%, 96%, 97%, 98%, or 99% identical to a sequence selected from SEQ ID NOs:137-150. In some embodiments, the EpCAM antibody or EpCAM-binding antibody fragment thereof comprises a LC sequence that is at least 90%, 91%, 92%, 93%, 94%, 95%, 96%, 97%, 98%, and 99%, identical to the sequence of SEQ ID NO: 140. In some embodiments, the EpCAM antibody or EpCAM-binding antibody fragment thereof comprises a LC sequence that is at least 90%, 91%, 92%, 93%, 94%, 95%, 96%, 97%, 98%, and 99%, identical to the sequence of SEQ ID NO:130. In some embodiments, the percent identity in each instance is 95%. In some embodiments, the percent identity in each instance is 98%.

In additional embodiments, the EpCAM antibody or EpCAM-binding antibody fragment thereof of immunoconjugates described herein comprises a HC having a sequence selected from SEQ ID NOs:102-134 and an LC having a sequence selected from SEQ ID NOs:137-150. In additional embodiments, the EpCAM antibody or EpCAM-binding antibody fragment thereof of immunoconjugates described herein comprises a HC having the sequence of SEQ ID NO:103 and an LC having the sequence of SEQ ID NO:140. In additional embodiments, the EpCAM antibody or EpCAM-binding antibody fragment thereof of immunoconjugates described herein comprises a HC having the sequence of SEQ ID NO:103 and an LC having the sequence of SEQ ID NO:138. In additional embodiments, the EpCAM antibody or EpCAM-binding antibody fragment thereof of immunoconjugates described herein comprises a HC having the sequence of SEQ ID NO:105 and an LC having the sequence of SEQ ID NO:138. In additional embodiments, the EpCAM antibody or EpCAM-binding antibody fragment thereof of immunoconjugates described herein comprises a HC having the sequence of SEQ ID NO:106 and an LC having the sequence of SEQ ID NO:139. In additional embodiments, the EpCAM antibody or EpCAM-binding antibody fragment thereof of immunoconjugates described herein comprises a HC having the sequence of SEQ ID NO:105 and an LC having the sequence of SEQ ID NO:140. In additional embodiments, the EpCAM antibody or EpCAM-binding antibody fragment thereof of immunoconjugates described herein comprises a HC having the sequence of SEQ ID NO:106 and an LC having the sequence of SEQ ID NO:140. In some embodiments, the EpCAM antibody or EpCAM-binding antibody fragment thereof of immunoconjugates described herein comprises a HC having the sequence of SEQ ID NO:125 and an LC having the sequence of SEQ ID NO:140. In additional embodiments, the EpCAM antibody or EpCAM-binding antibody fragment thereof of immunoconjugates described herein comprises a HC having the sequence of SEQ ID NO:127 and an LC having the sequence of SEQ ID NO:140. In additional embodiments, the EpCAM antibody or EpCAM-binding antibody fragment thereof of immunoconjugates described herein comprises a HC having the sequence of SEQ ID NO:126 and an LC having the sequence of SEQ ID NO:140. In additional embodiments, the EpCAM antibody or EpCAM-binding antibody fragment thereof of immunoconjugates described herein comprises a HC having the sequence of SEQ ID NO:134 and an LC having the sequence of SEQ ID NO:140.

In some embodiments, the EpCAM antibody of immunoconjugates described herein comprises an altered (e.g., mutated or engineered) Fc region. For example, in some aspects, the Fc region has been altered to reduce or enhance the effector functions of the antibody, alter serum half-life or other functional properties of the antibody. Reduction or elimination of effector function is desirable in certain cases, for example in the case of antibodies whose mechanism of action involves blocking or antagonism, but not killing of the cells bearing a target antigen. Increased effector function is generally desirable when directed to undesirable cells, such as tumor and foreign cells, where the FcγRs are expressed at low levels, for example, tumor-specific B cells with low levels of FcγRIIB (e.g., non-Hodgkin's lymphoma, CLL, and Burkitt's lymphoma). Immunoconjugates of the invention possessing such conferred or altered effector function activity are useful for the treatment and/or prevention of a disease, disorder or infection in which an enhanced efficacy of effector function activity is desired. In some aspects, the Fc region is an isotype selected from IgM, IgA, IgG, IgE, or other isotype.

Although the Fc Region of the EpCAM antibodies and EpCAM-binding antibody fragment thereof may possess the ability to bind to one or more Fc receptors (e.g., FcγR(s)), in certain embodiments the antibody or antibody fragment comprises a variant Fc region having an altered binding to FcγRIA (CD64), FcγRIIA (CD32A), FcγRIIB (CD32B), FcγRIIIA (CD16a) or FcγRIIIB (CD16b) (relative to the binding exhibited by a wild-type Fc Region), e.g., will have enhanced binding to an activating receptor and/or will have substantially reduced or no ability to bind to inhibitory receptor(s). Thus, the Fc region of the EpCAM antibody or EpCAM-binding antibody fragment thereof may include some or all of the CH2 domain and/or some or all of the CH3 domain of a complete Fc region, or may comprise a variant CH2 and/or a variant CH3 sequence (that may include, for example, one or more insertions and/or one or more deletions with respect to the CH2 or CH3 domains of a complete Fc Region). Such Fc regions may comprise non-Fc polypeptide portions, or may comprise portions of non-naturally complete Fc regions, or may comprise non-naturally occurring orientations of CH2 and/or CH3 domains (such as, for example, two CH2 domains or two CH3 domains, or in the N-terminal to C-terminal direction, a CH3 domain linked to a CH2 domain, etc.).

Fc Region modifications identified as altering effector function are known in the art, including modifications that increase binding to activating receptors (e.g., FcγRIIA (CD16A) and reduce binding to inhibitory receptors (e.g., FCγRIIB (CD32B) (see, e.g., Stavenhagen, et al., Cancer Res. 57(18):8882-8890 (2007)). Table 8 lists exemplary single, double, triple, quadruple and quintuple substitutions (numbering is that of the EU index as in Kabat, and substitutions are relative to the amino acid sequence of SEQ ID NO:304) of exemplary modification that increase binding to activating receptors and/or reduce binding to inhibitory receptors.

TABLE 8
Variations of Particular Activating Fc Regions
Single-Site Variations

| | | | |
|---|---|---|---|
| F243L | R292G | D270E | R292P |
| Y300L | P396L | | |

Double-Site Variations

| | | | |
|---|---|---|---|
| F243L and R292P | F243L and Y300L | F243L and P396L | R292P and Y300L |
| D270E and P396L | R292P and V305I | P396L and Q419H | P247L and N421K |
| R292P and P396L | Y300L and P396L | R255L and P396L | R292P and P305I |
| K392T and P396L | | | |

Triple-Site Variations

| | |
|---|---|
| F243L, P247L and N421K | P247L, D270E and N421K |
| F243L, R292P and Y300L | R255L, D270E and P396L |
| F243L, R292P and V305I | D270E, G316D and R416G |
| F243L, R292P and P396L | D270E, K392T and P396L |
| F243L, Y300L and P396L | D270E, P396L and Q419H |
| V284M, R292L and K370N | R292P, Y300L and P396L |

Quadruple-Site Variations

| | |
|---|---|
| L234F, F243L, R292P and Y300L | F243L, P247L, D270E and N421K |
| L234F, F243L, R292P and Y300L | F243L, R255L, D270E and P396L |
| L235I, F243L, R292P and Y300L | F243L, D270E, G316D and R416G |
| L235Q, F243L, R292P and Y300L | F243L, D270E, K392T and P396L |
| P247L, D270E, Y300L and N421K | F243L, R292P, Y300L, and P396L |
| R255L, D270E, R292G and P396L | F243L, R292P, V305I and P396L |
| R255L, D270E, Y300L and P396L | F243L, D270E, P396L and Q419H |
| D270E, G316D, P396L and R416G | |

Quintuple-Site Variations

| | |
|---|---|
| L235V, F243L, R292P, Y300L and P396L | F243L, R292P, V305I, Y300L and P396L |
| L235P, F243L, R292P, Y300L and P396L | |

Exemplary variants of human IgG1 Fc Regions with reduced binding to CD32B and/or increased binding to CD16A contain F243L, R292P, Y300L, V3051 or P396L substitutions, wherein the numbering is that of the EU index as in Kabat. These amino acid substitutions may be present in a human IgG1 Fc Region in any combination. In one embodiment, the variant human IgG1 Fc Region contains a F243L, R292P and Y300L substitution. In another embodiment, the variant human IgG1 Fc Region contains a F243L, R292P, Y300L, V3051 and P396L substitution.

In some embodiments, the EpCAM antibody or EpCAM-binding antibody fragment thereof of immunoconjugates described herein comprises an immunoglobulin heavy chain constant region containing a modification that decreases effector function (see, e.g., Idusogie et al., J. Immunol. 166:2571-2575 (2001); Sazinsky et al., PNAS USA 105:20167-20172 (2008): Davis et al., J. Rheumatol. 34:2204-2210 (2007); Bolt et al., Eur. J. Immunol. 23:403-411 (1993); Alegre et al., Transplantation 57:1537-1543 (1994); Xu et al., Cell Immunol. 200:16-26 (2000); Cole et al., Transplantation 68:563-571 (1999); Hutchins et al., PNAS USA 92:11980-11984 (1995); Reddy et al., J. Immunol. 164:1925-1933 (2000); WO97/11971, and WO07/106585; U.S. Appl. Publ. 2007/0148167A1; McEarchem et al., Blood 109:1185-1192 (2007); Strohl, Curr. Op. Biotechnol. 20:685-691 (2009); and Kumagai et al., J. Clin. Pharmacol. 47:1489-1497 (2007), the contents of each of which is herein incorporated by reference in its entirety).

In some embodiments, the Fc region of the EpCAM antibody or EpCAM-binding antibody fragment thereof of immunoconjugates described herein to exhibit decreased (or substantially no) binding to an effector receptor selected from the group consisting of: FcγRIA (CD64), FcγRIIA (CD32A)(allotypes R131 and H131), FcγRIIB (CD32B), FcγRIIIA (CD16a) (allotype V158 and F158) and FcγRIIIB (CD16b)(allotype FcγIIIb-NA1 and FcγIIIb-NA2); relative to the binding exhibited by the wild-type IgG Fc Region (SEQ ID NO:304). In some embodiments, the EpCAM antibody or EpCAM-binding antibody fragment thereof Fc region variant effector receptor binding affinity has been reduced to 1/10 or less, 1/50 or less, or 1/100 or less as, compared to the binding affinity of the corresponding antibody or antibody binding fragment comprising the wildtype Fc region of the corresponding immunoglobulin.

In a specific embodiment, the EpCAM antibody or EpCAM-binding antibody fragment thereof of immunoconjugates described herein comprises an IgG Fc region that exhibits reduced effector function (e.g., reduced ADCC) and comprise a modification at one or more amino acid positions selected from the group consisting of 233, 234, 235, 236, 237, 238, 239, 265, 266, 267, 269, 270, 271, 295, 296, 297, 298, 300, 324, 325, 327, 328, 329, 331, and 332, wherein the amino acid position numbering is according to the EU index as set forth in Kabat. In one embodiment, the CH2-CH3 domain of the EpCAM antibody include any 1, 2, 3, or 4 of the substitutions: L234A, L235A, D265A, N297Q, N297A, and N297G, wherein the numbering is that of the EU index as in Kabat. In another embodiment, the CH2-CH3 domains contain an N297Q substitution, an N297A substitution, or L234A and L235A substitutions, as these mutations abolish FcR binding. Alternatively, the EpCAM antibody or EpCAM-binding antibody fragment thereof of immunoconjugates described herein comprises a CH2-CH3 domain of a naturally occurring Fc region that inherently exhibits decreased (or substantially no) binding to FcγRIIIA (CD16a) and/or reduced effector function (relative to the binding and effector function exhibited by the wild-type IgG1 Fc region (SEQ ID NO:304). In a specific embodiment, the Fc constant region of the EpCAM antibody comprises an IgG2 Fc region (SEQ ID NO:305) or an IgG4 Fc region (SEQ ID: NO:306). Since the N297A, N297G, N297Q, L234A, L235A and D265A substitutions abolish effector function, in circumstances in which effector function is desired, these substitutions would in some instances not be employed.

A particular IgG1 sequence for the CH2 and CH3 domains of the Fc region-containing EpCAM antibody or EpCAM-binding antibody fragment thereof of immunoconjugates described herein that has reduced or abolished effector function comprises the substitutions L234A/L235A(shown underlined) (SEQ ID NO:307):

APEAAGGPSV FLFPPKPKDT LMISRTPEVT CVVVDVSHED
PEVKENWYVD GVEVHNAKTK PREEQYNSTY RVVSVLTVLH
QDWINGKEYK CKVSNKALPA PIEKTISKAK GQPREPQVYT
LPPSRDELTK NQVSLTCLVK GFYPSDIAVE WESNGQPENN
YKTTPPVLDS DGSFFLYSKL TVDKSRWQQG NVFSCSVMHE
ALHNHYTQKS LSLSPG

A particular IgG1 sequence for the CH2 and CH3 Domains of the Fc region-containing EpCAM antibody or EpCAM-binding antibody fragment thereof of immunoconjugates described herein that has reduced or abolished effector function comprises the substitution N297A (shown underlined) (SEQ ID NO:308):

APELLGGPSV FLEPPKPKDT LMISRTPEVT CVVVDVSHED
PEVKENWYVD GVEVHNAKTK PREEQYASTY RVVSVLTVLH
QDWLNGKEYK CKVSNKALPA PIEKTISKAK GQPREPQVYT
LPPSRDELTK NQVSLTCLVK GFYPSDIAVE WESNGQPENN
YKITPPVLDS DGSFFLYSKL TVDKSRWQQG NVESCSVMHE
ALHNHYTQKS LSLSPG

A particular IgG1 sequence for the CH2 and CH₃ Domains of the Fc region-containing EpCAM antibody or EpCAM-binding antibody fragment thereof of immunoconjugates described herein that has reduced or abolished effector function comprises the substitution N297Q (shown underlined) (SEQ ID NO:309):

APELLGGPSV FLFPPKPKDT LMISRTPEVT CVVVDVSHED
PEVKENWYVD GVEVHNAKTK PREEQYQSTY RVVSVLTVLH
QDWLNGKEYK CKVSNKALPA PIEKTISKAK GQPREPQVYT
LPPSRDELTK NQVSLTCLVK GFYPSDIAVE WESNGQPENN
YKTTPPVLDS DGSFFLYSKL TVDKSRWQQG NVESCSVMHE
ALHNHYTQKS LSLSPG

In some embodiments, the EpCAM antibody or EpCAM-binding antibody fragment thereof of immunoconjugates described herein comprises one or more modifications corresponding to: IgG1-C220S, C226S, C229S, P238S: IgG1-C226S, C229S; IgG1-C226S, C229S, E233P, L234V, L235A; IgG1-L234A, L235A; IgG1-L234F, L235E, P331S; IgG1-L234F, L235E, P331S; IgG1-H268Q, A330S, P331S; IgG1-G236R, L328R; IgG1-L235G, G236R, IgG1-N297A; IgG1-N325A, L328R; IgG1-N325L, L328R; IgG1-K326W, E333S; IgG2-V234A, G237A; IgG2-E333S; IgG2 H268Q, V309L, A330S, A331S; IgG4-S228P, L236E; IgG4-F234A, L235A; IgG4-F234A, G237A, E318A; IgG4-L235A, G237A, E318A; IgG4-L236E; IgG2-EU sequence 118-260; and IgG4-EU sequence 261-447; wherein the position numbering is according to the EU index as in Kabat.

In some embodiments, the EpCAM antibody or EpCAM-binding antibody fragment thereof of immunoconjugates described herein comprises a heavy chain immunoglobulin constant domain that has reduced CDC activity. In particular aspects, EpCAM antibody or EpCAM-binding antibody fragment thereof of immunoconjugates described herein comprises an IgG1 heavy chain constant region containing a mutation that decreases CDC activity (see, e.g., WO 1997/11971 and WO 2007/106585; U.S. Appl. Publ. 2007/0148167A1; McEarchem et al., Blood 109:1185-1192 (2007); Hayden-Ledbetter et al., Clin. Cancer 15:2739-2746 (2009); Lazar et al., PNAS USA 103:4005-4010 (2006); Bruckheimer et al., Neoplasia 11:509-517 (2009); Strohl, Curr. Op. Biotechnol. 20:685-691 (2009); and Sazinsky et al., PNAS USA 105:20167-20172 (2008); each of which is herein incorporated by reference in its entirety). Examples of heavy chain constant domain sequence modifications that decrease CDC include one or more modifications corresponding to: IgG1-C226S, C229S, E233P, L234V, L235A; IgG1-C226S, P230S; IgG1-L234F, L235E, P331S; IgG1-S239D, A330L, I332E; IgG2 EU sequence 118-260; IgG4-EU sequence 261-447; and IgG2-H268Q, V309L, A330S, A331S, according to the EU index

In some embodiments, the provided EpCAM antibody or EpCAM-binding antibody fragment thereof of immunoconjugates described herein comprises a heavy chain immunoglobulin constant domain that contains one or more half-life extending amino acid modifications (e.g., substitutions). Numerous mutations capable of increasing the half-life of an Fc region-containing molecule are known in the art and are encompassed as components of the EpCAM antibodies and EpCAM-binding antibody fragment thereof provided herein. See, e.g., U.S. Pat. Nos. 6,277,375; 7,083,784; 7,217,797, and 8,088,376; U.S. Publ. Nos. 2002/0147311; and 2007/0148164; and PCT Publication Nos. WO 1998/23289; WO 2009/058492; and WO 2010/033279, the contents of each of which is herein incorporated by reference in its entirety.

The serum half-life of proteins comprising Fc regions may be increased by increasing the binding affinity of the Fc Region for FcRn. The term “half-life” as used herein means a pharmacokinetic property of a molecule that is a measure of the mean survival time of the molecules following their administration. Half-life can be expressed as the time required to eliminate fifty percent (50%) of a known quantity of the molecule from a subject's (e.g., a human patient or other mammal) body or a specific compartment thereof, for example, as measured in serum, i.e., circulating half-life, or in other tissues. In general, an increase in half-life results in an increase in mean residence time (MRT) in circulation for the administered molecule.

In some embodiments, the EpCAM antibody or EpCAM-binding antibody fragment thereof of immunoconjugates described herein comprises a half-life extending amino acid substitution at one or more positions selected from the group consisting of: 238, 250, 252, 254, 256, 257, 256, 265, 272, 286, 288, 303, 305, 307, 308, 309, 311, 312, 317, 340, 356, 360, 362, 376, 378, 380, 382, 413, 424, 428, 433, 434, 435, and 436, wherein the amino acid position numbering is according to the EU index. In some embodiments, the EpCAM antibody or EpCAM-binding antibody fragment thereof contains one or more amino acid substitutions of amino acid residues at positions 251-257, 285-290, 308-314, 385-389, and 428-436, wherein the amino acid position numbering is according to the EU index. In some embodiments, the EpCAM antibody or EpCAM-binding antibody fragment thereof contains one or more of a substitution of the amino acid at Kabat position 252 with Tyr, Phe, Trp, or Thr; a substitution of the amino acid at Kabat position 254 with Thr; a substitution of the amino acid at Kabat position 256 with Ser, Arg, Gin, Glu, Asp, or Thr, a substitution of the amino acid at Kabat position 257 with Leu; a substitution of the amino acid at Kabat position 309 with Pro; a substitution of the amino acid at Kabat position 311 with Ser; a substitution of the amino acid at Kabat position 428 with Thr, Leu, Phe, or Ser; a substitution of the amino acid at Kabat position 433 with Arg, Ser, Iso, Pro, or Gin; or a substitution of the amino acid at Kabat position 434 with Trp, Met, Ser, His, Phe, or Tyr. More specifically, the EpCAM antibody or EpCAM-binding antibody fragment thereof domain can contain amino acid substitutions relative to a wild-type human IgG constant domain including a substitution of the amino acid at Kabat position 252 with Tyr, a substitution of the amino acid at Kabat position 254 with Thr, and a substitution of the amino acid at Kabat position 256 with Glu.

In some embodiments, the EpCAM antibody or EpCAM-binding antibody fragment thereof of immunoconjugates described herein comprises a least one substitution selected from: T250Q, M252Y, S254T, T256E, K288D, T307Q, V308P, A378V, M428L, N434A, N434S, N434H, N434Y, H435K, and Y4361, wherein the numbering is that of the EU index as in Kabat. In further embodiments, the EpCAM antibody or EpCAM-binding antibody fragment thereof comprises substitutions selected from: (a) M252Y, S254T and T256E; (b) M252Y and S254T; (c) M252Y and T256E; (d) T250Q and M428L; (e) T307Q and N434A; (f) A378V and N434A; (g) N434A and Y4361; (h) V308P and N434A; and (i) K288D and H435K.

In a particular embodiment, the EpCAM antibody or EpCAM-binding antibody fragment thereof of immunoconjugates described herein contains a variant IgG Fc Region comprising any 1, 2, or 3 of the substitutions: M252Y, S254T and T256E. The disclosure further provides EpCAM antibody or EpCAM-binding antibody fragment thereof possessing variant Fc regions comprising: (a) one or more mutations which alter effector function and/or FcγR; and (b) one or more mutations which extend serum half-life.

### 6.3 Immunoconjugates Comprising Epcam Activatable Antibodies

In additional embodiments, the disclosure provides immunoconjugates comprising EpCAM activatable antibodies (e.g., activatable EpCAM antibodies and activatable EpCAM-binding antibody fragments thereof). In some embodiments, the EpCAM activatable antibody comprises an EpCAM antibody or EpCAM-binding antibody fragment thereof that specifically binds EpCAM (e.g., human EpCAM) coupled to a masking moiety (MM), such that coupling of the MM reduces the ability of the EpCAM antibody or EpCAM-binding antibody fragment thereof to bind EpCAM. In some embodiments, the MM is coupled via a sequence that includes a substrate for a protease, for example, a protease that is active in diseased tissue and/or a protease that is co-localized with EpCAM at a treatment site in a subject. In some embodiments, the immunoconjugates comprising an EpCAM activatable antibody includes an EpCAM antibody described in Section 6.2 coupled to a MM via a protease substrate.

The EpCAM activatable antibodies are in some instances stable in circulation, activated at intended sites of therapy and/or diagnosis but not in normal, e.g., healthy tissue or other tissue not targeted for treatment and/or diagnosis, and, when activated, exhibit binding to EpCAM that is at least comparable to the corresponding, unmodified antibody. Immunoconjugates comprising the EpCAM activatable antibody are also provided, as are nucleic acids or sets of nucleic acids encoding the EpCAM activatable antibodies, and vectors and host cells comprising the nucleic acids. Pharmaceutical compositions comprising the activatable antibodies, immunoconjugates, nucleic acids, vectors, and host cells, are also provided.

In some embodiments, immunoconjugates having the EpCAM activatable antibody or antibody fragment comprise:
(a) a cleavable moiety coupled to the antibody or antibody fragment, wherein the cleavable moiety is a polypeptide that functions as a substrate for a protease; and
(b) a masking moiety coupled to the antibody or antibody fragment, wherein the masking moiety inhibits the binding of the antibody or antibody fragment to EpCAM when the activatable antibody is in an uncleaved state,
wherein the activatable antibody in the uncleaved state has the structural arrangement from N-terminus to C-terminus as follows: (masking moiety)-(cleavable moiety)-(antibody or antibody fragment) or (antibody or antibody fragment)-(cleavable moiety)-(masking moiety).

In some instances, an immunoconjugate disclosed herein comprises an EpCAM activatable antibody comprising:
(a) an EpCAM antibody or EpCAM-binding antibody fragment thereof comprising a VH-CDR1, VH-CDR2, VH-CDR3, VL-CDR1, VL-CDR2, and VL-CDR3 having the sequences of a member selected from the group:
   (i) SEQ ID NOs: 13-15, 42, 40, and 41, respectively;
   (ii) SEQ ID NOs: 13-15, and 39-41, respectively;
   (iii) SEQ ID NOs: 13, 26, 15, and 39-41, respectively; and
   (iv) SEQ ID NOs: 13, 24, 15, 42, 40, and 41, respectively;
(b) a masking moiety coupled to the EpCAM antibody or EpCAM-binding antibody fragment thereof, wherein the masking moiety inhibits the binding of the antibody or antibody fragment to EpCAM when the activatable antibody is in an uncleaved state; and
(c) a cleavable moiety coupled to the EpCAM antibody or EpCAM-binding antibody fragment thereof, wherein the cleavable moiety is a polypeptide that functions as a substrate for a protease;
wherein the activatable antibody in the uncleaved state has the structural arrangement from N-terminus to C-terminus as follows: (masking moiety)-(cleavable moiety)-(antibody or antibody fragment) or (antibody or antibody fragment)-(cleavable moiety)-(masking moiety).

The EpCAM activatable antibodies of immunoconjugates described herein in an activated state bind human EpCAM and include (i) an EpCAM antibody or a EpCAM-binding antibody fragment thereof (Ab) of immunoconjugates described herein that specifically binds to human EpCAM (as disclosed herein in, e.g., Section 6.2); (ii) a masking moiety (MM) that, when the EpCAM activatable antibody, is in an uncleaved state, inhibits the binding of the EpCAM activatable antibody to EpCAM; and (c) a cleavable moiety (CM) coupled to the EpCAM antibody or EpCAM-binding antibody fragment thereof, wherein the CM is a polypeptide that functions as a substrate for a protease. In some embodiments, the EpCAM activatable antibody, in the uncleaved state has the structural arrangement from N-terminus to C-terminus as follows: MM-CM-Ab or Ab-CM-MM. In some embodiments, the EpCAM activatable antibody, comprises a linking peptide between the MM and the CM. In some embodiments, the EpCAM activatable antibody, comprises a linking peptide between the CM and the Ab.

In some embodiments, the EpCAM activatable antibody of immunoconjugates described herein, in an uncleaved state specifically binds to mammalian EpCAM with a dissociation constant less than or equal to 1 nM, less than or equal to 5 nM, less than or equal to 10 nM, less than or equal to 15 nM, less than or equal to 20 nM, less than or equal to 25 nM, less than or equal to 50 nM, less than or equal to 100 nM, less than or equal to 150 nM, less than or equal to 250 nM, less than or equal to 500 nM, less than or equal to 750 nM, less than or equal to 1000 nM, and 122. /or less than or equal to 2000 nM.

In some embodiments, the EpCAM activatable antibody of immunoconjugates described herein, in an uncleaved state specifically binds to mammalian EpCAM (e.g., human EpCAM or cynomolgous EpCAM) with a dissociation constant greater than or equal to 1 nM, greater than or equal to 5 nM, greater than or equal to 10 nM, greater than or equal to 15 nM, greater than or equal to 20 nM, greater than or equal to 25 nM, greater than or equal to 50 nM, greater than or equal to 100 nM, greater than or equal to 150 nM, greater than or equal to 250 nM, greater than or equal to 500 nM, greater than or equal to 750 nM, greater than or equal to 1000 nM, and 122. /or greater than or equal to 2000 nM.

In some embodiments, the EpCAM activatable antibody of immunoconjugates described herein, in an uncleaved state specifically binds to the mammalian EpCAM (e.g., human EpCAM or cynomolgous EpCAM) with a dissociation constant in the range of 1 nM to 2000 nM, 1 nM to 1000 nM, 1 nM to 750 nM, 1 nM to 500 nM, 1 nM to 250 nM, 1 nM to 150 nM, 1 nM to 100 nM, 1 nM to 50 nM, 1 nM to 25 nM, 1 nM to 15 nM, 1 nM to 10 nM, 1 nM to 5 nM, 5 nM to 2000 nM, 5 nM to 1000 nM, 5 nM to 750 nM, 5 nM to 500 nM, 5 nM to 250 nM, 5 nM to 150 nM, 5 nM to 100 nM, 5 nM to 50 nM, 5 nM to 25 nM, 5 nM to 15 nM, 5 nM to 10 nM, 10 nM to 2000 nM, 10 nM to 1000 nM, 10 nM to 750 nM, 10 nM to 500 nM, 10 nM to 250 nM, 10 nM to 150 nM, 10 nM to 100 nM, 10 nM to 50 nM, 10 nM to 25 nM, 10 nM to 15 nM, 15 nM to 2000 nM, 15 nM to 1000 nM, 15 nM to 750 nM, 15 nM to 500 nM, 15 nM to 250 nM, 15 nM to 150 nM, 15 nM to 100 nM, 15 nM to 50 nM, 15 nM to 25 nM, 25 nM to 2000 nM, 25 nM to 1000 nM 25 nM to 750 nM, 25 nM to 500 nM, 25 nM to 250 nM 25 nM to 150 nM, 25 nM to 100 nM, 25 nM to 50 nM, 50 nM to 2000 nM, 50 nM to 1000 nM, 50 nM to 750 nM, 50 nM to 500 nM, 50 nM to 250 nM, 50 nM to 150 nM, 50 nM to 100 nM, 100 nM to 2000 nM, 100 nM to 1000 nM, 100 nM to 750 nM, 100 nM to 500 nM, 100 nM to 250 nM, 100 nM to 150 nM, 150 nM to 2000 nM, 150 nM to 1000 nM, 150 nM to 750 nM 150 nM to 500 nM, 150 nM to 250 nM, 250 nM to 2000 nM 250 nM to 1000 nM, 250 nM to 750 nM, 250 nM to 500 nM, 500 nM to 2000 nM, 500 nM to 1000 nM, 500 nM to 750 nM, 500 nM to 500 nM, 500 nM to 250 nM, 500 nM to 150 nM, 500 nM to 100 nM, 500 nM to 50 nM, 750 nM to 2000 nM, 750 nM to 1000 nM, or 1000 nM to 2000 nM.

In some embodiments, the EpCAM activatable antibody, in an activated state specifically binds to mammalian EpCAM (e.g., human EpCAM or cynomolgous EpCAM) with a dissociation constant that is less than or equal to 0.01 nM, 0.05 nM, 0.1 nM, 0.5 nM, 1 nM, 5 nM, or 10 nM. In some embodiments, the EpCAM activatable antibody, in an activated state specifically binds to mammalian EpCAM with a dissociation constant is greater than or equal to 0.01 nM, 0.05 nM, 0.1 nM, 0.5 nM, 1 nM, 5 nM, or 10 nM.

In some embodiments, the EpCAM activatable antibody, in an activated state specifically binds to the mammalian EpCAM (e.g., human EpCAM or cynomolgous EpCAM) with a dissociation constant in the range of 0.01 nM to 100 nM, 0.01 nM to 10 nM, 0.01 nM to 5 nM, 0.01 nM to 1 nM, 0.01 to 0.5 nM, 0.01 nm to 0.1 nM, 0.01 nm to 0.05 nM, 0.05 nM to 100 nM, 0.05 nM to 10 nM, 0.05 nM to 5 nM, 0.05 nM to 1 nM, 0.05 to 0.5 nM, 0.05 nm to 0.1 nM, 0.1 nM to 100 nM, 0.1 nM to 10 nM, 0.1 nM to 5 nM, 0.1 nM to 1 nM, 0.1 to 0.5 nM, 0.5 nM to 100 nM, 0.5 nM to 10 nM, 0.5 nM to 5 nM, 0.5 nM to 1 nM, 1 nM to 100 nM, 1 nM to 10 nM, 1 nM to 5 nM, 5 nM to 100 nM, 5 nM to 10 nM, or 10 nM to 100 Nm.

In some embodiments, the EpCAM activatable antibody of immunoconjugates described herein specifically binds to human EpCAM with a dissociation constant of less than 1 nM. In some embodiments, the EpCAM activatable antibody specifically binds to cynomolgus EpCAM with a dissociation constant of less than 1 nM. In some embodiments, the EpCAM activatable antibody specifically binds to human EpCAM and cynomolgus EpCAM with a dissociation constant of less than 1 nM.

In some embodiments, the serum half-life of the EpCAM activatable antibody of immunoconjugates described herein is longer than that of the corresponding antibody; e.g., the pK of the EpCAM activatable antibody is longer than that of the corresponding antibody. In some embodiments, the serum half-life of the EpCAM activatable antibody is similar to that of the corresponding antibody.

In some embodiments, the EpCAM activatable antibody of immunoconjugates described herein comprises an EpCAM antibody or EpCAM-binding antibody fragment thereof comprising a VH-CDR1, a VH-CDR2, and a VH-CDR3 having the sequences set forth in one row of Table 2. In some embodiments, the EpCAM activatable antibody comprises an EpCAM antibody or EpCAM-binding antibody fragment thereof comprising a VL-CDR1, a VL-CDR2, and a VL-CDR3 having the sequences set forth in one row of Table 3.

In some embodiments, the EpCAM activatable antibody of immunoconjugates described herein comprises an EpCAM antibody or EpCAM-binding antibody fragment thereof comprising a VH-CDR1 selected from SEQ ID NOs:13, and 16-25; a VH-CDR2 selected from SEQ ID NOs:14, and 26-29; a VH-CDR3 selected from SEQ ID NOs:15, and 30-38; a VL-CDR1 selected from SEQ ID NOs:39, and 42-45; a VL-CDR2 of SEQ ID NO:40; and a VL-CDR3 selected from SEQ ID NOs:41, and 46-51.

In some embodiments, the EpCAM activatable antibody of immunoconjugates described herein comprises an EpCAM antibody or EpCAM-binding antibody fragment thereof comprising a VH-CDR1, VH-CDR2, VH-CDR3, VL-CDR1, VL-CDR2, and VL-CDR3 having the sequences selected from the group consisting of: (i) SEQ ID NOs: 13-15, 42, 40, and 41, respectively; (ii) SEQ ID NOs: 13-15, and 39-41, respectively; (iii) SEQ ID NOs: 13, 26, 15, and 39-41, respectively; and (iv) SEQ ID NOs: 13, 26, 15, 42, 40, and 41, respectively. In some embodiments, the EpCAM activatable antibody comprises an EpCAM antibody or EpCAM-binding antibody fragment thereof comprising a VH-CDR1, VH-CDR2, VH-CDR3, VL-CDR1, VL-CDR2, and VL-CDR3 having the sequences of SEQ ID NOs: 13-15, 42, 40, and 41, respectively.

In some embodiments, the EpCAM activatable antibody of immunoconjugates described herein comprises an EpCAM antibody or EpCAM-binding antibody fragment thereof comprising a VH-CDR1, VH-CDR2, VH-CDR3, VL-CDR1, VL-CDR2, and VL-CDR3 having the sequences selected from the group consisting of: (i) SEQ ID NOs: 22, 14, 15, 42, 40, and 41, respectively; (ii) SEQ ID NOs: 13, 14, 33, 42, 40, and 41, respectively; (iii) SEQ ID NOs: 23, 14, 15, 42, 40, and 41, respectively, and; (iv) SEQ ID NOs: 25, 14, 15, 42, 40, and 41, respectively. In some embodiments, the EpCAM activatable antibody comprises an EpCAM antibody or EpCAM-binding antibody fragment thereof comprising a VH-CDR1, VH-CDR2, VH-CDR3, VL-CDR1, VL-CDR2, and VL-CDR3 having the sequences of SEQ ID NOs: 22, 14, 15, 42, 40, and 41, respectively. In some embodiments, the EpCAM activatable antibody comprises an EpCAM antibody or EpCAM-binding antibody fragment thereof comprising a VH-CDR1, VH-CDR2, VH-CDR3, VL-CDR1, VL-CDR2, and VL-CDR3 having the sequences of SEQ ID NOs: 13, 14, 33, 42, 40, and 41, respectively.

In some embodiments, the EpCAM activatable antibody of immunoconjugates described herein comprises a VH disclosed in Table 4. In some embodiments, the EpCAM activatable antibody of immunoconjugates described herein comprises a VL disclosed in Table 5. In some embodiments, the EpCAM activatable antibody of immunoconjugates described herein comprises a VH having the sequence of SEQ ID NO: 54 and a VL having the sequence of SEQ ID NO: 89. In some embodiments, the EpCAM activatable antibody of immunoconjugates described herein comprises an EpCAM antibody or EpCAM-binding antibody fragment thereof of immunoconjugates described herein comprising a VH having the sequence of SEQ ID NO: 75 and a VL having the sequence of SEQ ID NO: 89. In some embodiments, the EpCAM activatable antibody of immunoconjugates described herein comprises an EpCAM antibody or EpCAM-binding antibody fragment thereof comprising a VH having the sequence of SEQ ID NO: 77 and a VL having the sequence of SEQ ID NO: 89.

In some embodiments, the EpCAM activatable antibody of immunoconjugates described herein comprises a VH sequence that is at least 90%, 91%, 92%, 93%, 94%, 95%, 96%, 97%, 98% or 99% identical to a sequence selected from SEQ ID NO: 54, 75, and 77. In some embodiments, the EpCAM activatable antibody comprises a VL sequence that is at least 90%, 91%, 92%, 93%, 94%, 95%, 96%, 97%, 98% or 99% identical to a sequence comprising SEQ ID NO:89. In some embodiments, the percent identity in each instance is 95%. In some embodiments, the percent identity in each instance is 98%.

In some embodiments, the EpCAM activatable antibody of immunoconjugates described herein comprises a HC disclosed in Table 6. In some embodiments, the EpCAM activatable antibody of immunoconjugates described herein comprises a LC disclosed in Table 7. In some embodiments, the EpCAM activatable antibody of immunoconjugates described herein comprises a HC having the sequence of SEQ ID NO: 103 and a LC having the sequence of SEQ ID NO: 140. In some embodiments, the EpCAM activatable antibody of immunoconjugates described herein comprises a HC having the sequence of SEQ ID NO: 125 and a LC having the sequence of SEQ ID NO: 140. In some embodiments, the EpCAM activatable antibody of immunoconjugates described herein comprises a HC having the sequence of SEQ ID NO: 127 and a light chain having the sequence of SEQ ID NO: 140.

In some embodiments, the EpCAM activatable antibody of immunoconjugates described herein comprises an EpCAM antibody or EpCAM-binding antibody fragment thereof that specifically binds to an epitope within the extracellular region of human EpCAM (SEQ ID NO:1). In certain embodiments, the EpCAM activatable antibody of immunoconjugates described herein comprises an EpCAM antibody or EpCAM-binding antibody fragment thereof that specifically binds to an epitope within the first extracellular domain (D1) of human EpCAM (SEQ ID NO:2).

In some embodiments, the EpCAM activatable antibody of immunoconjugates described herein comprises a VH-CDR1 comprising X₁YX₃X₄H, wherein X₁ is selected from N and S, X₃ is selected from Y, N, F, S, H, D, L, I, and W, and X₄ is selected from I and M; a VH-CDR2 comprising WX₂X₃PGX₆VYIQYX₁₂X₁₃KFX₁₇G, wherein X₂ is selected from I and F, X₃ is selected from Y and N, X₆ is selected from N and D, X₁₂ is selected from N and S, X₁₃ is selected from E and Q, and X₁₇ is selected from K and Q (SEQ ID NO:7); and a VH-CDR3 comprising X₁GX₃X₄FAY, wherein X₁ is selected from D and E, X₃ is selected from P, A, S, Y, F, G, T, and V, and X₄ is selected from Y and W (SEQ ID NO:8). In some embodiments, the EpCAM activatable antibody comprises a VL-CDR1 comprising RSSX₄SLLHSX₁₀G X₁₂TYLX₁₆, wherein X₄ is selected from R and K, X₁₀ is selected from N and D, X₁₂ is selected from F and I, and X₁₆ is selected from Y and S (SEQ ID NO:10); a light chain VL-CDR2 comprising QTSNLAS (SEQ ID NO:40); and a VL-CDR3 comprising X₁QX₃LELPX₈T, wherein X₁ is selected from A, L, and Q, X₃ is selected from S, G, Y, and N, and X₈ is selected from N and W (SEQ ID NO:11). In some embodiments, the EpCAM activatable antibody comprises a VH-CDR1 comprising the sequence of SEQ ID NO:13; a VH-CDR2 comprising the sequence of SEQ ID NO:14; a VH-CDR3 comprising the sequence of SEQ ID NO:15; a VL-CDR1 comprising the sequence of SEQ ID NO:42; a VL-CDR2 comprising the sequence of SEQ ID NO:40; and a VL-CDR3 comprising the sequence of SEQ ID NO:41.

In some embodiments, the VH-CDR1 of the EpCAM activatable antibody of immunoconjugates described herein comprises the sequence NYX₃IH, wherein X₃ is selected from Y, N, F, S, H, D, L, I, and W (SEQ ID NO:6). In some embodiments, the VH-CDR3 of the EpCAM activatable antibody comprises the sequence DGPX₄FAY, wherein X₄ is selected from Y and W (SEQ ID NO:9). In some embodiments, the VL-CDR3 of the EpCAM activatable antibody comprises the sequence AQX₃LELPNT, wherein X₃ is selected from S, G, Y, and N (SEQ ID NO:12). In some embodiments, the EpCAM activatable antibody comprises a VH-CDR1 comprising the sequence of SEQ ID NO:13; a VH-CDR2 comprising the sequence of SEQ ID NO:14; a VH-CDR3 comprising the sequence of SEQ ID NO:15; a VL-CDR1 comprising the sequence of SEQ ID NO:42; a VL-CDR2 comprising the sequence of SEQ ID NO:40; and a VL-CDR3 comprising the sequence of SEQ ID NO:41.

Suitable components of the disclosed EpCAM activatable antibody of immunoconjugates described herein also include an EpCAM antibody or EpCAM-binding antibody fragment thereof, that cross-competes for binding to human EpCAM and/or cynomolgus EpCAM with an EpCAM antibody comprising a VH having the sequence of SEQ ID NO: 54 and a VL having the sequence of SEQ ID NO: 89. Additional suitable EpCAM activatable antibodies cross-compete for binding to human EpCAM and/or cynomolgus EpCAM with an EpCAM antibody comprising a VH having the sequence of SEQ ID NO: 75 and a VL having the sequence of SEQ ID NO: 89. Additional suitable EpCAM activatable antibodies cross-compete for binding to human EpCAM and/or cynomolgus EpCAM with an EpCAM antibody comprising a VH having the sequence of SEQ ID NO: 77 and a VL having the sequence of SEQ ID NO: 89.

The EpCAM activatable antibodies of immunoconjugates described herein provided herein include a masking moiety (MM). In some embodiments, the masking moiety (or “mask”) is an amino acid sequence that is coupled or otherwise attached to the EpCAM antibody and is positioned within the EpCAM activatable antibody construct such that the masking moiety reduces the ability of the EpCAM antibody to specifically bind EpCAM. Suitable masking moieties are identified using any of a variety of known techniques. For example, peptide masking moieties are identified using the methods described in WO 2009/025846, the contents of which is herein incorporated by reference in its entirety.

In some embodiments, the MM of the activatable antibody has a dissociation constant for binding to the Ab which is greater than the dissociation constant of the Ab to EpCAM. In some embodiments, the MM has a dissociation constant for binding to the Ab which is no more than the dissociation constant of the Ab to EpCAM.

In some embodiments, the MM has a dissociation constant for binding to the Ab which is less than the dissociation constant of the Ab to EpCAM.

In some embodiments, the dissociation constant (K_{d}) of the MM towards the Ab is no more than 2, 3, 4, 5, 10, 25, 50, 100, 250, 500, 1,000, 2,500, 5,000, 10,000, 50,000, 100,000, 500,000, 1,000,000, 5,000,000, 10,000,000, 50,000,000 times or greater, or between 1-5, 5-10, 10-100, 10-1,000, 10-10,000, 10-100,000, 10-1,000,000, 10-10,000,000, 100-1,000, 100-10,000, 100-100,000, 100-1,000,000, 100-10,000,000, 1,000-10,000, 1,000-100,000, 1,000-1,000,000, 1000-10,000.000, 10,000-100,000, 10,000-1,000,000, 10,000-10,000,000, 100,000-1,000,000, or 100,000-10,000,000 times or greater than the dissociation constant of the Ab towards the target.

In some embodiments, the MM does not interfere or compete with the Ab for binding to EpCAM when the EpCAM activatable antibody of immunoconjugates described herein is in a cleaved state. In some embodiments, the MM is a polypeptide of about 2 to 40 amino acids in length. In some embodiments, the MM is a polypeptide of up to about 40 amino acids in length.

In some embodiments, the MM polypeptide sequence is different from that of EpCAM. In some embodiments, the MM polypeptide sequence is no more than 50% identical to any natural binding partner of the Ab. In some embodiments, the MM polypeptide sequence is different from that of EpCAM and is no more than 40%, 30%, 25%, 20%, 15%, or 10% identical to any natural binding partner of the Ab.

In some embodiments, the coupling of the MM to the Ab reduces the ability of the Ab to bind EpCAM such that the dissociation constant (IQ) of the Ab when coupled to the MM towards EpCAM is at least 2, 5, 10, 20, 40, 100, 1,000, 10,000 greater than the Kd of the Ab when not coupled to the MM towards EpCAM.

In some embodiments, in the presence of EpCAM, the MM reduces the ability of the Ab to bind EpCAM by at least 90% when the CM is uncleaved, as compared to when the CM is cleaved when assayed in vitro using a target displacement assay such as, for example, the assay described in WO 2010/081173, the contents of which are hereby incorporated by reference in its entirety.

When the Ab is modified with a MM and is in the presence of human EpCAM, specific binding of the Ab to human EpCAM is reduced or inhibited, as compared to the specific binding of the Ab not modified with an MM or the specific binding of the parental Ab to human EpCAM.

The Kd of the Ab modified with a MM towards human EpCAM is at least 5, 10, 25, 50, 100, 250, 500, 1,000, 2,500, 5,000, 10,000, 50,000, 100,000, 500,000, 1,000,000, 5,000,000, 10,000,000, 50,000,000 or greater, or between 5-10, 10-100, 10-1,000, 10-10,000, 10-100,000, 10-1,000,000, 10-10,000,000, 100-1,000, 100-10,000, 100-100,000, 100-1,000,000, 100-10,000,000, 1,000-10,000, 1,000-100,000, 1,000-1,000,000, 1000-10,000,000, 10,000-100,000, 10,000-1,000,000, 10,000-10,000,000, 100,000-1,000,000, or 100,000-10,000,000 times greater than the Kd of the Ab not modified with an MM or of the parental Ab towards human EpCAM. Conversely, the binding affinity of the Ab modified with a MM towards human EpCAM is at least 2, 3, 4, 5, 10, 25, 50, 100, 250, 500, 1,000, 2,500, 5,000, 10,000, 50,000, 100,000, 500,000, 1,000,000, 5,000,000, 10,000,000, 50,000,000 or greater, or between 5-10, 10-100, 10-1,000, 10-10,000, 10-100,000, 10-1,000,000, 10-10,000,000, 100-1,000, 100-10,000, 100-100,000, 100-1,000,000, 100-10,000,000, 1,000-10,000, 1,000-100,000, 1,000-1,000,000, 1000-10,000,000, 10,000-100,000, 10,000-1,000,000, 10,000-10,000,000, 100,000-1,000,000, or 100,000-10,000,000 times lower than the binding affinity of the Ab not modified with an MM or of the parental Ab towards human EpCAM.

In some embodiments, the dissociation constant (Kd) of the MM towards the Ab is approximately equal to the Kd of the Ab towards human EpCAM. In some embodiments, the dissociation constant (Kd) of the MM towards the Ab is no more than the dissociation constant of the Ab towards human EpCAM. In some embodiments, the dissociation constant (Kd) of the MM towards the Ab is less than the dissociation constant of the Ab towards human EpCAM. In some embodiments, the dissociation constant (Kd) of the MM towards the Ab is greater than the dissociation constant of the Ab towards human EpCAM.

In some embodiments, the MM has a Kd for binding to the Ab that is no more than the Kd for binding of the Ab to human EpCAM.

In some embodiments, the MM has a Kd for binding to the Ab that is no less than the Kd for binding of the Ab to human EpCAM. In some embodiments, the MM has a Kd for binding to the Ab that is approximately equal to the Kd for binding of the Ab to human EpCAM. In some embodiments, the MM has a Kd for binding to the Ab that is less than the Kd for binding of the Ab to human EpCAM. In some embodiments, the MM has a Kd for binding to the Ab that is greater than the Kd for binding of the Ab to human EpCAM. In some embodiments, the MM has a Kd for binding to the Ab that is no more than 2, 3, 4, 5, 10, 25, 50, 100, 250, 500, or 1,000 fold greater than the Kd for binding of the Ab to human EpCAM. In some embodiments, the MM has a Kd for binding to the Ab that is between 1-5, 2-5, 2-10, 5-10, 5-20, 5-50, 5-100, 10-100, 10-1,000, 20-100, 20-1000, or 100-1,000 fold greater than the Kd for binding of the Ab to human EpCAM.

In some embodiments, the MM has an affinity for binding to the Ab that is less than the affinity of binding of the Ab to human EpCAM. In some embodiments, the MM has an affinity for binding to the Ab that is no more than the affinity of binding of the Ab to human EpCAM. In some embodiments, the MM has an affinity for binding to the Ab that is approximately equal of the affinity of binding of the Ab to human EpCAM. In some embodiments, the MM has an affinity for binding to the Ab that is no less than the affinity of binding of the Ab to human EpCAM. In some embodiments, the MM has an affinity for binding to the Ab that is greater than the affinity of binding of the Ab to human EpCAM.

In some embodiments, the MM has an affinity for binding to the Ab that is 2, 3, 4, 5, 10, 25, 50, 100, 250, 500, or 1,000 less than the affinity of binding of the Ab to human EpCAM. I In some embodiments, the MM has an affinity for binding to the Ab that is between 1-5, 2-5, 2-10, 5-10, 5-20, 5-50, 5-100, 10-100, 10-1,000, 20-100, 20-1000, or 100-1,000 fold less than the affinity of binding of the Ab to human EpCAM. In some embodiments, the MM has an affinity for binding to the Ab that is 2 to 20 fold less than the affinity of binding of the Ab to human EpCAM. In some embodiments, a MM not covalently linked to the Ab and at equimolar concentration to the EpCAM activatable antibody does not inhibit the binding of the Ab to human EpCAM.

When the Ab is modified with a MM and is in the presence of human EpCAM specific binding of the Ab to human EpCAM is reduced or inhibited, as compared to the specific binding of the Ab not modified with an MM or the specific binding of the parental Ab to human EpCAM. When compared to the binding of the Ab not modified with an MM or the binding of the parental Ab to human EpCAM, the Ab's ability to bind human EpCAM when modified with an MM can be reduced by at least 50%, 60%, 70%, 80%, 90%, 92%, 93%, 94%, 95%, 96%, 97%, 98%, 99% and even 100% for at least 2, 4, 6, 8, 12, 28, 24, 30, 36, 48, 60, 72, 84, or 96 hours, or 5, 10, 15, 30, 45, 60, 90, 120, 150, or 180 days, or 1, 2, 3, 4, 5, 6, 7, 8, 9, 10, 11, or 12 months or more when measured in vivo or in an in vitro assay.

The MM inhibits the binding of the Ab to human EpCAM. The MM binds the antigen binding domain of the Ab and inhibits binding of the Ab to human EpCAM. The MM can sterically inhibit the binding of the Ab to human EpCAM. The MM can allosterically inhibit the binding of the Ab to its target. In these embodiments, when the Ab is modified or coupled to a MM and in the presence of target there is no binding or substantially no binding of the Ab to human EpCAM, or no more than 0.001%, 0.01%, 0.1%, 1%, 2%, 3%, 4%, 5%, 6%, 7%, 8%, 9%, 10%, 15%, 20%, 25%, 30%, 35%, 40%, or 50% binding of the Ab to human EpCAM, as compared to the binding of the Ab not modified with an MM, the parental Ab, or the Ab not coupled to an MM to human EpCAM, for at least 2, 4, 6, 8, 12, 28, 24, 30, 36, 48, 60, 72, 84, or 96 hours, or 5, 10, 15, 30, 45, 60, 90, 120, 150, or 180 days, or 1, 2, 3, 4, 5, 6, 7, 8, 9, 10, 11, or 12 months or longer when measured in vivo or in an in vitro assay.

When an Ab is coupled to or modified by a MM, the MM ‘masks’ or reduces or otherwise inhibits the specific binding of the Ab to human EpCAM. When an Ab is coupled to or modified by a MM, such coupling or modification can effect a structural change that reduces or inhibits the ability of the Ab to specifically bind its target.

An Ab coupled to or modified with an MM can be represented by the following formulae (in order from an amino (N) terminal region to carboxyl (C) terminal region:

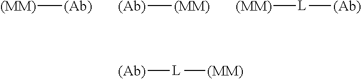

where MM is a masking moiety, the Ab is an EpCAM antibody, EpCAM-binding antigen fragment, and the L is a linker. In many embodiments, it may be desirable to insert one or more linkers, e.g., flexible linkers, into the composition so as to provide for flexibility.

In certain embodiments, the MM is not a natural binding partner of the Ab. In some embodiments, the MM contains no or substantially no homology to any natural binding partner of the Ab. In some embodiments, the MM is no more than 5%, 10%, 15%, 20%, 25%, 30%, 35%, 40%, 45%, 50%, 55%, 60%, 65%, 70%, 75%, or 80% similar to any natural binding partner of the Ab. In some embodiments, the MM is no more than 5%, 10%, 15%, 20%, 25%, 30%, 35%, 40%, 45%, 50%, 55%, 60%, 65%, 70%, 75%, or 80% identical to any natural binding partner of the Ab. In some embodiments, the MM is no more than 25% identical to any natural binding partner of the Ab. In some embodiments, the MM is no more than 50% identical to any natural binding partner of the Ab. In some embodiments, the MM is no more than 20% identical to any natural binding partner of the Ab. In some embodiments, the MM is no more than 10% identical to any natural binding partner of the Ab.

In some embodiments, the MM comprises a sequence disclosed in Table 9. In some embodiments, the MM comprises a sequence selected from SEQ ID NOs:151-157. In some embodiments, the MM comprises a sequence selected from SEQ ID NOs: 158-161. In some embodiments, the MM comprises a sequence selected from SEQ ID NOs:162-167. In some embodiments, the MM comprises the sequence of SEQ ID NO: 155.

TABLE 9
Exemplary Masking Moiety Sequences,

| | |
|---|---|
| Mask name | Sequence |

Masks for EpCAM23Gv4.2 humanized antibody

| | |
|---|---|
| Ep01-2 | PLMTCSDYYTQLNNL (SEQ ID NO: 151) |
| Ep02 | LSCTHSRYDMHCPHM (SEQ ID NO: 152) |
| Ep03 | HYCHSRTDTITHQNA (SEQ ID NO: 153) |
| Ep04 | WCPRLFDRPSMGCPT (SEQ ID NO: 154) |
| Ep05 | WWPPCQGGAWCEQRI (SEQ ID NO: 155) |
| Ep07 | HSGCPRLFDRCSAPA (SEQ ID NO: 156) |
| Ep11 | FICPTLYDRPHCMHT (SEQ ID NO: 157) |

Masks for 1565-Y affinity variant

| | |
|---|---|
| Ep101 | SWCHSATDTILPQSN (SEQ ID NO: 158) |
| Ep102 | SPACSDRYYQTCVLN (SEQ ID NO: 159) |
| Ep103 | MSCVVDRFDRQCPHL (SEQ ID NO: 160) |
| Ep104 | TTRCEHYWFTCPLSP (SEQ ID NO: 161) |

Masks for 1361-H affinity variant

| | |
|---|---|
| Ep105 | DCTGYSPSVLPACRV (SEQ ID NO: 162) |
| Ep106 | FCSGYSPSVLPSCLM (SEQ ID NO: 163) |
| Ep107 | SKPQSYMHPYCFYNS (SEQ ID NO: 164) |
| Ep108 | LTRCTIAHPYCYYNY (SEQ ID NO: 165) |
| Ep109 | PNTCMSERRICSLTY (SEQ ID NO: 166) |
| Ep110 | PRPHCAILRQQLAAT (SEQ ID NO: 167) |

The EpCAM activatable antibodies of immunoconjugates described herein provided herein include a cleavable moiety. In some embodiments, the cleavable moiety (or“substrate”) includes an amino acid sequence that is a substrate fora protease, usually an extracellular protease. Suitable substrates are identified using any of a variety of known techniques. For example, peptide substrates are identified using the methods described in U.S. Pat. Nos. 7,666,817 and 8,563,269; and WO 2014/026136, the contents of each of which is herein incorporated by reference in its entirety. (See also, Boulware et al., Biotechnol Bioeng. 106(3):339-346 (2010)).

In some embodiments, the EpCAM activatable antibodies of immunoconjugates described herein include an Ab that is modified by an MM and also includes one or more cleavable moieties (CM). Such EpCAM activatable antibodies exhibit activatable/switchable binding, to human EpCAM. The EpCAM activatable antibodies generally include an antibody or antigen-binding antibody fragment (Ab), modified by or coupled to a masking moiety (MM) and a modifiable or cleavable moiety (CM). In some embodiments, the CM contains an amino acid sequence that serves as a substrate for at least one protease. In some embodiments, the MM and the cleavable moiety are coupled to the EpCAM activatable antibody directly. In other embodiments, the MM and cleavable moiety are coupled to the EpCAM activatable antibody indirectly (e.g., via one or more linkers).

The elements of the EpCAM activatable antibodies of immunoconjugates described herein are arranged so that the MM and CM are positioned such that in a cleaved (or relatively active) state and in the presence of human EpCAM, the EpCAM activatable antibody binds human EpCAM, but when the EpCAM activatable antibody is in an uncleaved (or relatively inactive) state in the presence of human EpCAM, specific binding of the EpCAM activatable antibody to human EpCAM is reduced or inhibited. The specific binding of the EpCAM activatable antibody to human EpCAM can be reduced due to the inhibition or masking of the EpCAM activatable antibody's ability to specifically bind human EpCAM by the MM.

The Kd of the EpCAM activatable antibody of immunoconjugates described herein modified with a MM and a CM towards human EpCAM is at least 5, 10, 25, 50, 100, 250, 500, 1,000, 2,500, 5,000, 10,000, 50,000, 100,000, 500,000, 1,000,000, 5,000,000, 10,000,000, 50,000,000 or greater, or between 5-10, 10-100, 10-1,000, 10-10,000, 10-100,000, 10-1,000,000, 10-10,000,000, 100-1,000, 100-10,000, 100-100,000, 100-1,000,000, 100-10,000,000, 1,000-10,000, 1,000-100,000, 1,000-1,000,000, 1000-10,000,000, 10,000-100,000, 10,000-1,000,000, 10,000-10,000,000, 100,000-1,000,000, or 100,000-10,000,000 times greater than the Kd of the EpCAM activatable antibody not modified with an MM and a CM or of the parental Ab towards human EpCAM. Conversely, the binding affinity of the Ab modified with a MM and a CM towards human EpCAM is at least 5, 10, 25, 50, 100, 250, 500, 1,000, 2,500, 5,000, 10,000, 50,000, 100,000, 500,000, 1,000,000, 5,000,000, 10,000,000, 50,000,000 or greater, or between 5-10, 10-100, 10-1,000, 10-10,000, 10-100,000, 10-1,000,000, 10-10,000,000, 100-1,000, 100-10,000, 100-100,000, 100-1,000,000, 100-10,000,000, 1,000-10,000, 1,000-100,000, 1,000-1,000,000, 1000-10,000,000, 10,000-100,000, 10,000-1,000,000, 10,000-10,000,000, 100,000-1,000,000, or 100,000-10,000,000 times lower than the binding affinity of the EpCAM activatable antibody not modified with an MM and a CM or of the parental Ab towards human EpCAM.

When the EpCAM activatable antibody of immunoconjugates described herein is modified with a MM and a CM and is in the presence of human EpCAM but not in the presence of a modifying agent (for example at least one protease), specific binding of the EpCAM activatable antibody to human EpCAM is reduced or inhibited, as compared to the specific binding of the EpCAM activatable antibody not modified with an MM and a CM or of the parental Ab to human EpCAM. When compared to the binding of the parental Ab or the binding of an EpCAM activatable antibody not modified with an MM and a CM to human EpCAM, the EpCAM activatable antibody's ability to bind human EpCAM when modified with an MM and a CM can be reduced by at least 50%, 60%, 70%, 80%, 90%, 92%, 93%, 94%, 95%, 96%, 97%, 98%, 99% and even 100% for at least 2, 4, 6, 8, 12, 28, 24, 30, 36, 48, 60, 72, 84, or 96 hours or 5, 10, 15, 30, 45, 60, 90, 120, 150, or 180 days, or 1, 2, 3, 4, 5, 6, 7, 8, 9, 10, 11, or 12 months or longer when measured in vivo or in an in vitro assay.

As used herein, the term “cleaved state” refers to the condition of the EpCAM activatable antibodies of immunoconjugates described herein following modification of the CM by at least one protease. The term uncleaved or intact state, as used herein, refers to the condition of the EpCAM activatable antibodies in the absence of cleavage of the CM by a protease. As discussed above, the term “activatable antibody” or “activatable antibody” is used herein to refer to an EpCAM activatable antibody, in both its uncleaved (native or intact) state, as well as in its cleaved state. It will be apparent to ordinarily skilled artisan that in some embodiments, a cleaved EpCAM activatable antibody, may lack an MM due to cleavage of the CM by protease, resulting in release of at least the MM (e.g., where the MM is not joined to the EpCAM activatable antibody), by a covalent bond (e.g., a disulfide bond between cysteine residues).

By activatable or switchable it is meant that the EpCAM activatable antibody of immunoconjugates described herein, exhibits a first level of binding to a target when the EpCAM activatable antibody, is in a inhibited, masked, intact or uncleaved state (i.e., a first conformation), and a second level of binding to human EpCAM in the uninhibited, unmasked and/or cleaved state (i.e., a second conformation), where the second level of target binding is greater than the first level of binding. In general, the access of human EpCAM to the Ab of the EpCAM activatable antibody, is greater in the presence of a cleaving agent capable of cleaving the CM, i.e., a protease, than in the absence of such a cleaving agent. Thus, when the EpCAM activatable antibody is in the uncleaved state, the Ab is inhibited from binding human EpCAM and can be masked from human EpCAM-binding (i.e., the first conformation is such that the Ab cannot bind human EpCAM), and in the cleaved state the Ab is not inhibited or is unmasked to target binding.

The CM and Ab of the EpCAM activatable antibodies of immunoconjugates described herein are selected so that the Ab represents a binding moiety for a given target, and the CM represents a substrate for a protease. In some embodiments, the protease is co-localized with human EpCAM at a treatment site or diagnostic site in a subject. As used herein, co-localized refers to being at the same site or relatively close nearby. In some embodiments, a protease cleaves a CM yielding an activated antibody that binds to a target located nearby the cleavage site. The EpCAM activatable antibodies disclosed herein find particular use where, for example, a protease capable of cleaving a site in the CM, i.e., a protease, is present at relatively higher levels in target-containing tissue of a treatment site or diagnostic site than in tissue of non-treatment sites (for example in healthy tissue). In some embodiments, a CM of the disclosure is also cleaved by one or more other proteases. In some embodiments, it is the one or more other proteases that is co-localized with human EpCAM and that is responsible for cleavage of the CM in vivo.

In some embodiments, EpCAM activatable antibodies of immunoconjugates described herein provide for reduced toxicity and/or adverse side effects that could otherwise result from binding of the EpCAM activatable antibodies at non-treatment sites if the EpCAM activatable antibodies were not masked or otherwise inhibited from binding to human EpCAM.

In general, an EpCAM activatable antibody of immunoconjugates described herein, can be designed by selecting an Ab of interest and constructing the remainder of the EpCAM activatable antibody, so that, when conformationally constrained, the MM provides for masking of the EpCAM activatable antibodies or reduction of binding of the EpCAM activatable antibodies to human EpCAM. Structural design criteria can be to be taken into account to provide for this functional feature.

EpCAM activatable antibodies of immunoconjugates described herein exhibiting a switchable phenotype of a desired dynamic range for target binding in an inhibited versus an uninhibited conformation are provided. Dynamic range generally refers to a ratio of (a) a maximum detected level of a parameter under a first set of conditions to (b) a minimum detected value of that parameter under a second set of conditions. For example, in the context of an EpCAM activatable antibody, the dynamic range refers to the ratio of (a) a maximum detected level of target protein binding to an EpCAM activatable antibody, in the presence of at least one protease capable of cleaving the CM of the EpCAM activatable antibodies to (b) a minimum detected level of target protein binding to an EpCAM activatable antibody, in the absence of the protease. The dynamic range of an EpCAM activatable antibody, can be calculated as the ratio of the dissociation constant of an EpCAM activatable antibody, cleaving agent (e.g., enzyme) treatment to the dissociation constant of the EpCAM activatable antibodies cleaving agent treatment. The greater the dynamic range of an EpCAM activatable antibody, the better the switchable phenotype of the EpCAM activatable antibody.

EpCAM activatable antibodies of immunoconjugates described herein having relatively higher dynamic range values (e.g., greater than 1) exhibit more desirable switching phenotypes such that target protein binding by the EpCAM activatable antibodies occurs to a greater extent (e.g., predominantly occurs) in the presence of a cleaving agent (e.g., enzyme) capable of cleaving the CM of the EpCAM activatable antibodies than in the absence of a cleaving agent.

EpCAM activatable antibodies of immunoconjugates described herein can be provided in a variety of structural configurations. Exemplary formulae for EpCAM activatable antibodies are provided below. It is specifically contemplated that the N- to C-terminal order of the Ab, MM and CM may be reversed within an EpCAM activatable antibody. It is also specifically contemplated that the CM and MM may overlap in amino acid sequence, e.g., such that the CM is contained within the MM.

For example, EpCAM activatable antibodies of immunoconjugates described herein can be represented by the following formula (in order from an amino (N) terminal region to carboxyl (C) terminal region:

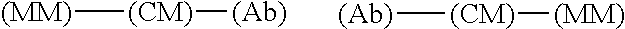

where MM is a masking moiety, CM is a cleavable moiety, and Ab is an EpCAM antibody or an EpCAM-binding antibody fragment thereof. It should be noted that although MM and CM are indicated as distinct components in the formulae above, in all exemplary embodiments, (including formulae) disclosed herein it is contemplated that the amino acid sequences of the MM and the CM could overlap, e.g., such that the CM is completely or partially contained within the MM. In addition, the formulae above provide for additional amino acid sequences that may be positioned N-terminal or C-terminal to the EpCAM activatable antibodies elements.

In some embodiments, the EpCAM activatable antibody of immunoconjugates described herein comprises a CM that is cleavable by a protease. In some embodiments, the protease that cleaves the CM is active, e.g., up-regulated or otherwise unregulated, in diseased tissue, and the protease cleaves the CM in the EpCAM activatable antibody, when the EpCAM activatable antibody, is exposed to the protease.

In some embodiments, the protease is co-localized with EpCAM in a tissue, and the protease cleaves the CM in the EpCAM activatable antibody, when the EpCAM activatable antibody, is exposed to the protease.

In some embodiments, the CM is positioned in the EpCAM activatable antibody of immunoconjugates described herein, such that when the EpCAM activatable antibody, is in the uncleaved state, binding of the EpCAM activatable antibody, to EpCAM is reduced to occur with a dissociation constant that is at least 2, 5, 10, 20, 40, 50, 100, or 200, greater than the dissociation constant of an unmodified Ab binding to EpCAM, whereas in the cleaved state (i.e., when the EpCAM activatable antibody, is in the cleaved state), the Ab binds EpCAM.

In some embodiments, the CM is a polypeptide of up to 15 amino acids in length.

In some embodiments, the CM is a polypeptide that includes a first cleavable moiety (CM1) that is a substrate for at least one matrix metalloprotease (MMP) and a second cleavable moiety (CM2) that is a substrate for at least one serine protease (SP). In some embodiments, each of the CM1 substrate sequence and the CM2 substrate sequence of the CM1-CM2 substrate is independently a polypeptide of up to 15 amino acids in length.

In some embodiments, the CM is a substrate for at least one protease that is or is believed to be up-regulated or otherwise unregulated in cancer. In some embodiments, the CM is a substrate for at least one protease that is or is believed to be up-regulated in inflammation. In some embodiments, the CM is a substrate for at least one protease that is or is believed to be up-regulated or otherwise unregulated in autoimmunity.

In some embodiments, the CM is a substrate for at least one protease selected from a matrix metalloprotease (MMP), thrombin, a neutrophil elastase, a cysteine protease, legumain, and a serine protease, such as matriptase (MT-SPI), and urokinase (uPA). Without being bound by theory, it is believed that these proteases are up-regulated or otherwise unregulated in at least one of cancer, inflammation, and/or autoimmunity.

Exemplary substrates include but are not limited to substrates cleavable by one or more of the following enzymes or proteases: an ADAMS/ADAMTS, (e.g., ADAM8, ADAM9, ADAM 10, ADAM 12, ADAM 15, ADAM 17/TACE, ADAMDEC1, ADAMTS1, ADAMTS4, ADAMTS5); an aspartate protease (e.g., BACE, Renin); an aspartic cathepsin (e.g., Cathepsin D and Cathepsin E); a caspase (e.g., Caspase 1-10, and Caspase 14); a cysteine cathepsin (e.g., Cathepsin B, Cathepsin C, Cathepsin K, Cathepsin L, Cathepsin S, Cathepsin V/L2, and Cathepsin X/Z/P); a cysteine proteinase (e.g., Cruzipain, Legumain, and Otubain-2); a KLK (e.g., KLK4-8, KLK10, KLK 11, KLK 13, and KLK14); a metalloproteinase (e.g., Meprin, Neprilysin, PSMA, and BMP1); an MMP (e.g., MMP1-3, MMP 7-17, MMP 19, MMP 20, MMP 23, MMP 24, MMP 26, and MMP 27); a serine protease (e.g., activated protein C, Cathepsin A, Cathepsin C, Chymase, and a coagulation factor protease such as FVIIa, FIXa, FXa, FXIa, and FXIIa), an Elastase (e.g., human Neutrophil Elastase); Granzyme B: Guanidinobenzoatase: HtrAI; Lactoferrin: Marapsin; NS3/4A; PACE4; Plasmin; PSA, tPA; Thrombin; Tryptase; uPA; a Type II Transmembrane Serine Protease (TTSP) (e.g., DESC1, DPP-4, FAP, Hepsin, Matriptase-2, MT-SPI/Matriptase, and TMPRSS2-4).

In some embodiments, the CM is selected for use with a specific protease, for example a protease that is known to be co-localized with the target of the EpCAM activatable antibody.

In some embodiments, the CM is a substrate for at least one MMP. Examples of MMPs include MMP1-3, MMP 7-17, MMP19, MMP20, MMP23, MMP24, MMP26, and MMP27. In some embodiments, the CM is a substrate for a protease selected from MMP 9, MMP 14, MMP1, MMP3, MMP13, MMP 17, MMP11, and MMP19. In some embodiments, the CM is a substrate for MMP9. In some embodiments, the CM is a substrate for MMP14.

Suitable CM that can routinely be incorporated into the provided activatable antibodies are known in the art. See, for example, WO 2016/179285, e.g., pages 40-47, the contents of which is herein incorporated by reference in its entirety.

In some embodiments, the CM is a substrate for a neutrophil elastase. In some embodiments, the CM is a substrate for a serine protease. In some embodiments, the CM is a substrate for legumain. In some embodiments, the CM is a substrate for matriptase. In some embodiments, the CM is a substrate for a cysteine protease. In some embodiments, the CM is a substrate for a cysteine protease, such as a cathepsin. In other embodiments, the CM is a substrate for a uPA.

In particular embodiments, the CM is a substrate for uPA. In some embodiments, the CM comprises a sequence disclosed in Table 10.

TABLE 10
Exemplary CM Sequences.

| Substrate | |
|---|---|
| name | Sequence |
| 3014 | AVGLLAPPGGLSGRSDNI (SEQ ID NO: 168) |
| 2014 | ISSGLLSGRSDNI (SEQ ID NO: 169) |

In some embodiments, the CM comprises the sequence AVGLLAPPGGLSGRSDNI (SEQ ID NO:168). In some embodiments, the CM comprises the sequence ISSGLLSGRSDNI (SEQ ID NO:169).

In some embodiments, the CM is a substrate for at least two proteases. In some embodiments, each protease is selected from an ADAMS/ADAMTS, (e.g., ADAM8, ADAM9, ADAM 10, ADAM 12, ADAM 15, ADAM 17/TACE, ADAMDEC1, ADAMTS1, ADAMTS4, ADAMTS5); an aspartate protease (e.g., BACE, Renin); an aspartic cathepsin (e.g., Cathepsin D and Cathepsin E); a caspase (e.g., Caspase 1-10, and Caspase 14); a cysteine cathepsin (e.g., Cathepsin B, Cathepsin C, Cathepsin K, Cathepsin L, Cathepsin S, Cathepsin V/L2, and Cathepsin X/Z/P); a cysteine proteinase (e.g., Cruzipain, Legumain, and Otubain-2); a KLK (e.g., KLK4-8, KLK10, KLK 11, KLK 13, and KLK14): a metalloproteinase (e.g., Meprin, Neprilysin, PSMA, and BMP1); an MMP (e.g., MMP1-3, MMP 7-17, MMP 19, MMP 20, MMP 23, MMP 24, MMP 26, and MMP 27); a serine protease (e.g., activated protein C, Cathepsin A, Cathepsin C, Chymase, and a coagulation factor protease such as FVIIa, FIXa, FXa, FXIa, and FXIIa), an Elastase (e.g., human Neutrophil Elastase); Granzyme B: Guanidinobenzoatase: HtrAI; Lactoferrin: Marapsin; NS3/4A; PACE4; Plasmin; PSA, tPA; Thrombin; Tryptase; uPA; a Type II Transmembrane Serine Protease (TTSP) (e.g., DESC1, DPP-4, FAP, Hepsin, Matriptase-2, MT-SPI/Matriptase, and TMPRSS2-4. In some embodiments, the CM is a substrate for at least two proteases, wherein one of the proteases is selected from: a MMP, thrombin, a neutrophil elastase, a cysteine protease, uPA, legumain and matriptase and the other protease is selected from those listed above. In some embodiments, the CM is a substrate for at least two proteases selected from the group: a MMP, thrombin, a neutrophil elastase, a cysteine protease, uPA, legumain and matriptase.

In some embodiments, the EpCAM activatable antibody of immunoconjugates described herein includes at least a first CM and a second CM. In some embodiments, the first CM and the second CM are each polypeptides of no more than 15 amino acids long. In some embodiments, the first CM and the second CM in the EpCAM activatable antibody, in the uncleaved state have the structural arrangement from N-terminus to C-terminus as follows: MM-CM1-CM2-Ab or Ab-CM2-CM1-MM. In some embodiments, at least one of the first CM and the second CM is a polypeptide that functions as a substrate for a protease selected from a MMP, thrombin, a neutrophil elastase, a cysteine protease, uPA, legumain, and matriptase. In some embodiments, the first CM is cleaved by a first cleaving agent selected from MMP, thrombin, a neutrophil elastase, a cysteine protease, uPA, legumain, and matriptase in a target tissue and the second CM is cleaved by a second cleaving agent in a target tissue. In some embodiments, the other protease is selected from the list presented in the preceding paragraph. In some embodiments, the first cleaving agent and the second cleaving agent are the same protease selected from a MMP, thrombin, a neutrophil elastase, a cysteine protease, uPA, legumain, and matriptase, and the first CM and the second CM are different substrates for the enzyme. In some embodiments, the first cleaving agent and the second cleaving agent are the same protease selected from the list in the preceding paragraph. In some embodiments, the first cleaving agent and the second cleaving agent are different proteases. In some embodiments, the first cleaving agent and the second cleaving agent are co-localized in the target tissue. In some embodiments, the first CM and the second CM are cleaved by at least one cleaving agent in the target tissue.

In some embodiments, the EpCAM activatable antibody of immunoconjugates described herein, also includes a signal peptide. In some embodiments, the signal peptide is conjugated to the EpCAM activatable antibody, via a spacer. In some embodiments, the spacer is conjugated to the EpCAM activatable antibody, in the absence of a signal peptide. In some embodiments, the spacer is joined directly to the MM of the EpCAM activatable antibody. In some embodiments, the spacer is joined directly to the MM of the EpCAM activatable antibody, in the structural arrangement from N-terminus to C-terminus of spacer-MM-CM-Ab.

Suitable spacers and spacer technology is known in the art and can routinely be used to incorporate spacers in some embodiments of the provided activatable antibodies. See, for example, WO 2016/179285 (e.g., at pages 52-53), the contents of which is herein incorporated by reference in its entirety.

In many embodiments, it may be desirable to insert one or more linkers, e.g., flexible linkers, into the EpCAM activatable antibody, construct so as to provide for flexibility at one or more of the MM-CM junction, the CM-Ab junction, or both. For example, the Ab, MM, and/or CM may not contain a sufficient number of residues (e.g., Gly, Ser, Asp, Asn, especially Gly and Ser, particularly Gly) to provide the desired flexibility. As such, the switchable phenotype of such EpCAM activatable antibody, constructs may benefit from introduction of one or more amino acids to provide for a flexible linker. In addition, as described below, where the EpCAM activatable antibody, is provided as a conformationally constrained construct, a flexible linker can be operably inserted to facilitate formation and maintenance of a cyclic structure in the uncleaved EpCAM activatable antibody.

For example, in certain embodiments, an EpCAM activatable antibody of immunoconjugates described herein, comprises one of the following formulae (where the formula below represent an amino acid sequence in either N- to C-terminal direction or C- to N-terminal direction):

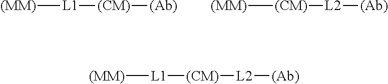

wherein MM, CM, and Ab are as defined above; wherein LI and L2 are each independently and optionally present or absent, are the same or different flexible linkers that include at least 1 flexible amino acid (e.g., Gly). In addition, the formulae above provide for additional amino acid sequences that may be positioned N-terminal or C-terminal to the EpCAM activatable antibodies elements. Examples include, but are not limited to, targeting moieties (e.g., a ligand for a receptor of a cell present in a target tissue) and serum half-life extending moieties (e.g., polypeptides that bind serum proteins, such as immunoglobulin (e.g., IgG) or serum albumin (e.g., human serum albumin (HSA)).

In some embodiments, the EpCAM activatable antibody of immunoconjugates described herein is exposed to and cleaved by a protease such that, in the activated or cleaved state, the activated antibody includes a light chain sequence that includes at least a portion of LP2 and/or CM sequence after the protease has cleaved the CM.

The CM is specifically cleaved by at least one protease at a rate of about 0.001-1500×10⁴ M⁻¹S⁻¹ or at least 0.001, 0.005, 0.01, 0.05, 0.1, 0.5, 1, 2.5, 5, 7.5, 10, 15, 20, 25, 50, 75, 100, 125, 150, 200, 250, 500, 750, 1000, 1250, or 1500×10⁴ M⁻¹S⁻¹. In some embodiments, the CM is specifically cleaved at a rate of about 100,000 MS¹. In some embodiments, the CM is specifically cleaved at a rate from about l×l0² to about 1×10⁶ M⁻¹S⁻¹ (i.e., from about 1×10² to about 1×l0⁶ M⁻¹ S⁻¹).

For specific cleavage by an enzyme, contact between the enzyme and CM is made. When the EpCAM activatable antibody, comprising an Ab (e.g., an EpCAM antibody or EpCAM-binding antibody fragment thereof) coupled to a MM and a CM is in the presence of EpCAM and sufficient enzyme activity, the CM can be cleaved. Sufficient enzyme activity can refer to the ability of the enzyme to make contact with the CM and effect cleavage. It can readily be envisioned that an enzyme may be in the vicinity of the CM but unable to cleave because of other cellular factors or protein modification of the enzyme.

Linkers suitable for use in EpCAM activatable antibody of immunoconjugates described herein, compositions disclosed herein are generally ones that provide flexibility of the modified Ab (e.g., an EpCAM antibody or EpCAM-binding antibody fragment thereof) or the EpCAM activatable antibody, to facilitate the inhibition of the binding of the EpCAM activatable antibody to human EpCAM. Such linkers are generally referred to as flexible linkers. Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, 7, 8, 9, 10, 11, 12, 13, 14, 15, 16, 17, 18, 19, or 20 amino acids in length.

Exemplary flexible linkers for the activatable antibodies, antibodies, and antibody fragments provided herein include, glycine polymers (G)n, glycine-serine polymers (including, for example, (GS)n. Suitable linkers and linker technology are known in the art and can routinely be used to incorporate spacers in some embodiments of the provided activatable antibodies. See, for example, WO 2016/179285 (e.g., at pages 26, 113-116), the contents of which is herein incorporated by reference in its entirety. The ordinarily skilled artisan will recognize that design of an EpCAM activatable antibodies can include linkers that are all or partially flexible, such that the linker can include a flexible linker as well as one or more portions that confer less flexible structure to provide for a desired EpCAM activatable antibodies structure.

In some embodiments, the EpCAM activatable antibody of immunoconjugates described herein comprises a first linking peptide (LP1) and a second linking peptide (LP2), and wherein the EpCAM activatable antibody, in the uncleaved state has the structural arrangement from N-terminus to C-terminus as follows: MM-LP1-CM-LP2-Ab or Ab-LP2-CM-LP1-MM. In some embodiments, the two linking peptides need not be identical to each other.

In some embodiments, the EpCAM activatable antibody of immunoconjugates described herein, comprises a first linking peptide (LP1) and a second linking peptide (LP2), and wherein the EpCAM activatable antibody, in the uncleaved state has the structural arrangement from N-terminus to C-terminus as follows: MM-LP1-CM-LP2-Ab or Ab-LP2-CM-LP1-MM. In some embodiments, the two linking peptides need not be identical to each other.

In some embodiments, at least one of LP1 or LP2 of the EpCAM activatable antibody of immunoconjugates described herein comprises a flexible linker. Suitable linkers and linker technology are known in the art and can routinely be used to incorporate spacers in some embodiments of the provided activatable antibodies. See, for example, WO 2016/179285 (e.g., at pages 26, 113-116), the contents of which is herein incorporated by reference in its entirety.

In some embodiments, the EpCAM activatable antibody of immunoconjugates described herein comprises a light chain having a sequence disclosed in Table 11. In some embodiments, the activatable antibody comprises a light chain having the sequence of SEQ ID NO:174. In some embodiments, the activatable antibody comprises a light chain having the sequence of SEQ ID NO:179.

TABLE 11
Exemplary light chain activatable antibody sequences.

| | |
|---|---|
| Activatable | |
| antibody name | Sequence |

For use with heavy chain of HuEpCAM23Gv4.2, 1361-H, or 1565-Y

| | |
|---|---|
| Ep01-2 3014 Lv | QGQSGQGPLMTCSDYYTCLNNLGGGSSGGAVGLLAPPGGLSGRSDNIGGSDIV |
| | LTQTPLSLSVTPGQPASISCRSSRSLLHSDGFTYLYWFLQKPGQSPQLLIYQTSN |
| | LASGVPDRFSSSGSGTDFTLKISRVEAEDVGVYYCAQNLELPNTFGQGTKLEIKR |
| | TVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| | (SEQ ID NO: 170) |
| Ep02 3014 Lv | QGQSGQGLSCTHSRYDMHCPHMGGGSSGGAVGLLÅPPGGLSGRSDNIGGSDI |
| | VLTQTPLSLSVTPGQPASISCRSSRSLLHSDGFTYLYWFLQKPGQSPQLLIYQTS |
| | NLASGVPDRESSSGSGTDFTLKISRVEAEDVGVYYCAQNLELPNTFGQGTKLEIK |
| | RTVAAPSVFIFPPSDEQLKSGTASVVCLLNNFYPREAKVQWKVDNALQSGNSQE |
| | SVTEQDSKDSTYSLSSTLTLSKADYEKHKVYACEVTHQGLSSPVTKSFNRGEC |
| | (SEQ ID NO: 171) |
| Ep03 3014 LV | QGQSGQGHYCHSRTDTITHCNAGGGSSGGAVGLLAPPGGLSGRSDNIGGSDIV |
| | LTQTPLSLSVTPGQPASISCRSSRSLLHSDGFTYLYWFLQKPGQSPQLLIYQTSN |
| | LASGVPDRFSSSGSGTDFTLKISRVEAEDVGVYYCAQNLELPNTFGQGTKLEIKR |
| | TVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| | (SEQ ID NO: 172) |
| Ep04 3014 LV | QGQSGQGWCPRLFDRPSMGCPTGGGSSGGAVGLLAPPGGLSGRSDNIGGSDI |
| | VLTQTPLSLSVTPGQPASISCRSSRSLLHSDGFTYLYWFLQKPGQSPQLLIYQTS |
| | NLÅSGVPDRFSSSGSGTDFTLKISRVEAEDVGVYYCAQNLELPNTFGQGTKLEIK |
| | RTVAAPSVFIFPPSDEQLKSGTASVVCLLNNFYPREAKVQWKVDNALQSGNSQE |
| | SVTEQDSKDSTYSLSSTLTLSKADYEKHKVYACEVTHQGLSSPVTKSFNRGEC |
| | (SEQ ID NO: 173) |
| Ep05 3014 Lv | QGQSGQGWWPPCQGGAWCEQRIGGGSSGGAVGLLAPPGGLSGRSDNIGGSD |
| | IVLTQTPLSLSVTPGQPASISCRSSRSLLHSDGFTYLYWFLQKPGQSPQLLIYQTS |
| | NLASGVPDRFSSSGSGTDFTLKISRVEAEDVGVYYCAQNLELPNTFGQGTKLEIK |
| | RTVAAPSVFIFPPSDEQLKSGTASVVCLLNNFYPREAKVQWKVDNALQSGNSQE |
| | SVTEQDSKDSTYSLSSTLTLSKADYEKHKVYACEVTHQGLSSPVTKSFNRGEC |
| | (SEQ ID NO: 174) |
| Ep07 3014 Lv | QGQSGQGHSGCPRLFDRCSAPAGGGGSSGGAVGLLAPPGGLSGRSDNIGGSDIV |
| | LTQTPLSLSVTPGQPASISCRSSRSLLHSDGFTYLYWFLQKPGQSPQLLIYQTSN |
| | LASGVPDRESSSGSGTDFTLKISRVEAEDVGVYYCAQNLELPNTFGQGTKLEIKR |
| | TVAAPSVFIFPPSDEQLKSGTASVVQLLNNFYPREAKVQWKVDNALQSGNSQES |
| | VTEQDSKDSTYSLSSTLTLSKADYEKHKVYACEVTHQGLSSPVTKSFNRGEC |
| | (SEQ ID NO: 175) |
| Ep11 3014 Lv | QGQSGQGFICPTLYDRPHCMHTGGGSSGGÅVGLLAPPGGLSGRSDNIGGSDIV |
| | LTQTPLSLSVTPGQPASISCRSSRSLLHSDGFTYLYWFLQKPGQSPQLLIYQTSN |
| | LASGVPDRESSSGSGTDFTLKISRVEAEDVGVYYCAQNLELPNTFGQGTKLEIKR |
| | TVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| | (SEQ ID NO: 176) |
| Ep01-2 2014 Lv | QGQSGQGPLMTGSDYYTCLNNLGGGSSGGSISSGLLSGRSDNIGGSDIVLTQTP |
| | LSLSVTPGQPASISCRSSRSLLHSDGFTYLYWFLQKPGQSPQLLIYQTSNLASGV |
| | PDRFSSSGSGTDFTLKISRVEAEDVGVYYCAQNLELPNTFGQGTKLEIKRTVAAP |
| | SVFIFPPSDEQLKSGTASVVCLLNNFYPREAKVQWKVDNALQSGNSQESVTEQD |
| | SKDSTYSLSSTLTLSKADYEKHKVYACEVTHQGLSSPVTKSFNRGEC |
| | (SEQ ID NO: 177) |
| Ep04 2014 Lv | QGQSGQGWCPRLFDRPSMGCPTGGGSSGGSISSGLLSGRSDNIGGSDIVLTQT |
| | PLSLSVTPGQPASISCRSSRSLLHSDGFTYLYWFLQKPGQSPQLLIYQTSNLASG |
| | VPDRESSSGSGTDFTLKISRVEAEDVGVYYCAQNLELPNTFGQGTKLEIKRTVAA |
| | PSVFIFPPSDEQLKSGTASVVCLLNNFYPREAKVQWKVDNALQSGNSQESVTEQ |
| | DSKDSTYSLSSTLTLSKADYEKHKVYACEVTHQGLSSPVTKSFNRGEC |
| | (SEQ ID NO: 178) |
| Ep05 2014 Lv | QGQSGQGWWPPCQGGAWCEQRIGGGSSGGSISSGLLSGRSDNIGGSDIVLTQ |
| | TPLSLSVTPGQPASISCRSSRSLLHSDGFTYLYWFLQKPGQSPQLLIYQTSNLAS |
| | GVPDRFSSSGSGTDFTLKISRVEAEDVGVYYCAQNLELPNTFGQGTKLEIKRTVA |
| | APSVFIFPPSDEQLKSGTASVVCLLNNFYPREAKVQWKVDNALQSGNSQESVTE |
| | QDSKDSTYSLSSTLTLSKADYEKHKVYACEVTHQGLSSPVTKSFNRGEC* |
| | (SEQ ID NO: 179) |
| | * denotes cysteine which, when in a native conformation (e.g., not reduced), |
| | participate in forming interchain disulfide bond. |
| Ep07 2014 Lv | QGQSGQGHSGCPRLFDRCSAPAGGGSSGGSISSGLLSGRSDNIGGSDIVLTQT |
| | PLSLSVTPGQPASISCRSSRSLLHSDGFTYLYWFLQKPGQSPQLLIYQTSNLASG |
| | VPDRFSSSGSGTDFTLKISRVEAEDVGVYYCAQNLELPNTFGQGTKLEIKRTVAA |
| | PSVFIFPPSDEQLKSGTASVVCLLNNFYPREAKVQWKVDNALQSGNSQESVTEQ |
| | DSKDSTYSLSSTLTLSKADYEKHKVYACEVTHQGLSSPVTKSFNRGEC |
| | (SEQ ID NO: 180) |

For use with heavy chain of 1565-Y

| | |
|---|---|
| Ep101 2014 Lv | QGQSGQGSWCHSATDTILPCSNGGGSSGGSISSGLLSGRSDNIGGSDIVLTQTP |
| | LSLSVTPGQPASISCRSSRSLLHSDGFTYLYWFLQKPGQSPQLLIYQTSNLASGV |
| | PDRFSSSGSGTDFTLKISRVEAEDVGVYYCAQNLELPNTFGQGTKLEIKRTVAAP |
| | SVFIFPPSDEQLKSGTASVVCLLNNFYPREAKVQWKVDNALQSGNSQESVTEQD |
| | SKDSTYSLSSTLTLSKADYEKHKVYACEVTHQGLSSPVTKSFNRGEC |
| | (SEQ ID NO: 181) |
| Ep102 2014 Lv | QGQSGQGSPACSDRYYQTCVLNGGGSSGGSISSGLLSGRSDNIGGSDIVLTQTP |
| | LSLSVTPGQPASISCRSSRSLLHSDGFTYLYWFLQKPGQSPQLLIYQTSNLASGV |
| | PDRFSSSGSGTDFTLKISRVEAEDVGVYYCAQNLELPNTFGQGTKLEIKRTVAAP |
| | SVFIFPPSDEQLKSGTASVVCLLNNFYPREAKVQWKVDNALQSGNSQESVTEQD |
| | SKDSTYSLSSTLTLSKADYEKHKVYACEVTHQGLSSPVTKSFNRGEC |
| | (SEQ ID NO: 182) |
| Ep103 2014 Lv | QGQSGQGMSCVVDRFDRQCPHLGGGSSGGSISSGLLSGRSDNIGGSDIVLTQT |
| | PLSLSVTPGQPASISCRSSRSLLHSDGFTYLYWFLQKPGQSPQLLIYQTSNLASG |
| | VPDRFSSSGSGTDFTLKISRVEAEDVGVYYCAQNLELPNTFGQGTKLEIKRTVAA |
| | PSVFIFPPSDEQLKSGTASVVCLLNNFYPREAKVQWKVDNALQSGNSQESVTEQ |
| | DSKDSTYSLSSTLTLSKADYEKHKVYACEVTHQGLSSPVTKSFNRGEC |
| | (SEQ ID NO: 183) |
| Ep104 2014 Lv | QGQSGQGTTRCEHYWFTCPLSPGGGSSGGSISSGLLSGRSDNIGGSDIVLTQTP |
| | LSLSVTPGQPASISCRSSRSLLHSDGFTYLYWFLQKPGQSPQLLIYQTSNLASGV |
| | PDRFSSSGSGTDFTLKISRVEAEDVGVYYCAQNLELPNTFGQGTKLEIKRTVAAP |
| | SVFIFPPSDEQLKSGTASVVCLLNNFYPREAKVQWKVDNALQSGNSQESVTEQD |
| | SKDSTYSLSSTLTLSKADYEKHKVYACEVTHQGLSSPVTKSFNRGEC |
| | (SEQ ID NO: 184) |
| Ep101 3014 Lv | QGQSGQGSWCHSATDTILPCSNGGGSSGGAVGLLAPPGGLSGRSDNIGGSDIV |
| | LTQTPLSLSVTPGQPASISCRSSRSLLHSDGFTYLYWFLQKPGQSPQLLIYQTSN |
| | LASGVPDRESSSGSGTDFTLKISRVEAEDVGVYYCAQNLELPNTFGQGTKLEIKR |
| | TVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| | (SEQ ID NO: 185) |
| Ep102 3014 Lv | QGQSGQGSPACSDRYYQTCVLNGGGSSGGAVGLLAPPGGLSGRSDNIGGSDIV |
| | LTQTPLSLSVTPGQPASISCRSSRSLLHSDGFTYLYWFLQKPGQSPQLLIYQTSN |
| | LASGVPDRESSSGSGTDFTLKISRVEAEDVGVYYCAQNLELPNTFGQGTKLEIKR |
| | TVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| | (SEQ ID NO: 186) |
| Ep103 3014 Lv | QGQSGQGMSCVVDRFDRQCPHLGGGSSGGAVGLLAPPGGLSGRSDNIGGSDI |
| | VLTQTPLSLSVTPGQPASISCRSSRSLLHSDGFTYLYWFLQKPGQSPQLLIYQTS |
| | NLASGVPDRFSSSGSGTDFTLKISRVEAEDVGVYYCAQNLELPNTFGQGTKLEIK |
| | RTVAAPSVFIFPPSDEQLKSGTASVVCLLNNFYPREAKVQWKVDNALQSGNSQE |
| | SVTEQDSKDSTYSLSSTLTLSKADYEKHKVYACEVTHQGLSSPVTKSFNRGEC |
| | (SEQ ID NO: 187) |
| fEp104 3014 Lv | QGQSGQGTTRCEHYWFTCPLSPGGGSSGGAVGLLAPPGGLSGRSDNIGGSDIV |
| | LTQTPLSLSVTPGQPASISCRSSRSLLHSDGFTYLYWFLQKPGQSPQLLIYQTSN |
| | LASGVPDRFSSSGSGTDFTLKISRVEAEDVGVYYCAQNLELPNTFGQGTKLEIKR |
| | TVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| | (SEQ ID NO: 188) |

For use with heavy chain of 1361-H

| | |
|---|---|
| Ep105 2014 Lv | QGQSGQGDCTGYSPSVLPACRVGGGSSGGSISSGLLSGRSDNIGGSDIVLTQTP |
| | LSLSVTPGQPASISCRSSRSLLHSDGFTYLYWFLQKPGQSPQLLIYQTSNLASGV |
| | PDRFSSSGSGTDFTLKISRVEAEDVGVYYCAQNLELPNTFGQGTKLEIKRTVAAP |
| | SVFIFPPSDEQLKSGTASVVCLLNNFYPREAKVQWKVDNALQSGNSQESVTEQD |
| | SKDSTYSLSSTLTLSKADYEKHKVYACEVTHQGLSSPVTKSFNRGEC (SEQ ID |
| | NO: 189) |
| Ep106 2014 Lv | QGQSGQGFCSGYSPSVLPSCLMGGGSSGGSISSGLLSGRSDNIGGSDIVLTQTP |
| | LSLSVTPGQPASISCRSSRSLLHSDGFTYLYWFLQKPGQSPQLLIYQTSNLASGV |
| | PDRFSSSGSGTDFTLKISRVEAEDVGVYYCAQNLELPNTFGQGTKLEIKRTVAAP |
| | SVFIFPPSDEQLKSGTASVVCLLNNFYPREAKVQWKVDNALQSGNSQESVTEQD |
| | SKDSTYSLSSTLTLSKADYEKHKVYACEVTHQGLSSPVTKSFNRGEC (SEQ ID |
| | NO: 190) |
| Ep107 2014 Lv | QGQSGQGSKPCSYMHPYCFYNSGGGSSGGSISSGLLSGRSDNIGGSDIVLTQT |
| | PLSLSVTPGQPASISCRSSRSLLHSDGFTYLYWFLQKPGQSPQLLIYQTSNLASG |
| | VPDRFSSSGSGTDFTLKISRVEAEDVGVYYCAQNLELPNTFGQGTKLEIKRTVAA |
| | PSVFIFPPSDEQLKSGTASVVCLLNNFYPREAKVQWKVDNALQSGNSQESVTEQ |
| | DSKDSTYSLSSTLTLSKADYEKHKVYACEVTHQGLSSPVTKSFNRGEC (SEQ |
| | ID NO: 191) |
| Ep108 2014 Lv | QGQSGQGLTRCTIAHPYCYYNYGGGSSGGSISSGLLSGRSDNIGGSDIVLTQTPL |
| | SLSVTPGQPASISCRSSRSLLHSDGFTYLYWFLQKPGQSPQLLIYQTSNLASGVP |
| | DRFSSSGSGTDFTLKISRVEAEDVGVYYCAQNLELPNTFGQGTKLEIKRTVAAPS |
| | VFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGEC (SEQ ID |
| | NO: 192) |
| Ep109 2014 Lv | QGQSGQGPNTCMSERRICSLTYGGGSSGGSISSGLLSGRSDNIGGSDIVLTQTP |
| | LSLSVTPGQPASISCRSSRSLLHSDGFTYLYWFLQKPGQSPQLLIYQTSNLASGV |
| | PDRFSSSGSGTDFTLKISRVEAEDVGVYYCAQNLELPNTFGQGTKLEIKRTVAAP |
| | SVFIFPPSDEQLKSGTASVVCLLNNFYPREAKVQWKVDNALQSGNSQESVTEQD |
| | SKDSTYSLSSTLTLSKADYEKHKVYACEVTHQGLSSPVTKSFNRGEC (SEQ ID |
| | NO: 193) |
| Ep110 2014 Lv | QGQSGQGPRPHCAILRQCLAATGGGSSGGSISSGLLSGRSDNIGGSDIVLTQTP |
| | LSLSVTPGQPASISCRSSRSLLHSDGFTYLYWFLQKPGQSPQLLIYQTSNLASGV |
| | PDRFSSSGSGTDFTLKISRVEAEDVGVYYCAQNLELPNTFGQGTKLEIKRTVAAP |
| | SVFIFPPSDEQLKSGTASVVCLLNNFYPREAKVQWKVDNALQSGNSQESVTEQD |
| | SKDSTYSLSSTLTLSKADYEKHKVYACEVTHQGLSSPVTKSFNRGEC (SEQ ID |
| | NO: 194) |
| Ep105 3014 Lv | QGQSGQGDCTGYSPSVLPACRVGGGSSGGAVGLLAPPGGLSGRSDNIGGSDIV |
| | LTQTPLSLSVTPGQPASISCRSSRSLLHSDGFTYLYWFLQKPGQSPQLLIYQTSN |
| | LASGVPDRFSSSGSGTDFTLKISRVEAEDVGVYYCAQNLELPNTFGQGTKLEIKR |
| | TVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| | (SEQ ID NO: 195) |
| Ep106 3014 Lv | QGQSGQGFCSGYSPSVLPSCLMGGGSSGGAVGLLAPPGGLSGRSDNIGGSDIV |
| | LTQTPLSLSVTPGQPASISCRSSRSLLHSDGFTYLYWFLQKPGQSPQLLIYQTSN |
| | LASGVPDRFSSSGSGTDFTLKISRVEAEDVGVYYCAQNLELPNTFGQGTKLEIKR |
| | TVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| | (SEQ ID NO: 196) |
| Ep107 3014 Lv | QGQSGQGSKPCSYMHPYCFYNSGGGSSGGAVGLLAPPGGLSGRSDNIGGSDI |
| | VLTQTPLSLSVTPGQPASISCRSSRSLLHSDGFTYLYWFLQKPGQSPQLLIYQTS |
| | NLASGVPDRFSSSGSGTDFTLKISRVEAEDVGVYYCAQNLELPNTFGQGTKLEIK |
| | RTVAAPSVFIFPPSDEQLKSGTASVVCLLNNFYPREAKVQWKVDNALQSGNSQE |
| | SVTEQDSKDSTYSLSSTLTLSKADYEKHKVYACEVTHQGLSSPVTKSFNRGEC |
| | (SEQ ID NO: 197) |
| Ep108 3014 LV | QGQSGQGLTRCTIAHPYCYYNYGGGSSGGAVGLLAPPGGLSGRSDNIGGSDIVL |
| | TQTPLSLSVTPGQPASISCRSSRSLLHSDGFTYLYWFLQKPGQSPQLLIYQTSNL |
| | ASGVPDRFSSSGSGTDFTLKISRVEAEDVGVYYCAQNLELPNTFGQGTKLEIKRT |
| | VAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| | (SEQ ID NO: 198) |
| Ep109 2014 Lv | QGQSGQGPNTCMSERRICSLTYGGGSSGGAVGLLAPPGGLSGRSDNIGGSDIV |
| | LTQTPLSLSVTPGQPASISCRSSRSLLHSDGFTYLYWFLQKPGQSPQLLIYQTSN |
| | LASGVPDRESSSGSGTDFTLKISRVEAEDVGVYYCAQNLELPNTFGQGTKLEIKR |
| | TVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| | (SEQ ID NO: 199) |
| Ep110 3014 Lv | QGQSGQGPRPHCAILRQCLAATGGGSSGGAVGLLAPPGGLSGRSDNIGGSDIVL |
| | TQTPLSLSVTPGQPASISCRSSRSLLHSDGFTYLYWFLQKPGQSPQLLIYQTSNL |
| | ASGVPDRESSSGSGTDFTLKISRVEAEDVGVYYCAQNLELPNTFGQGTKLEIKRT |
| | VAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| | (SEQ ID NO: 200) |

In some embodiments, the EpCAM activatable antibody of immunoconjugates described herein comprises a light chain having a sequence selected from SEQ ID NOs: 170-180 and a heavy chain having a sequence selected from SEQ ID NOs: 103, 125, and 127. In some embodiments, the EpCAM activatable antibody comprises a light chain having the sequence selected from SEQ ID NOs: 170-180 and a heavy chain having the sequence of SEQ ID NO:103. In some embodiments, the EpCAM activatable antibody comprises a light chain having the sequence of SEQ ID NO:174 and a heavy chain having the sequence of SEQ ID NO:103. In some embodiments, the EpCAM activatable antibody comprises a light chain having the sequence of SEQ ID NO:179 and a heavy chain having the sequence of SEQ ID NO:103.

In some embodiments, the EpCAM activatable antibody comprises a light chain having an amino acid sequence that is at least 90%, 91%, 92%, 93%, 94%, 95%, 96%, 97%, 98%, or 99% identical to a sequence selected from SEQ ID NOs:170-180 and a heavy chain having an amino acid sequence that is at least 90%, 91%, 92%, 93%, 94%, 95%, 96%, 97%, 98%, or 99%, identical to a sequence selected from SEQ ID NOs:103, 125, and 127. In some embodiments, the EpCAM activatable antibody comprises a light chain having an amino acid sequence that is at least 90%, 91%, 92%, 93%, 94%, 95%, 96%, 97%, 98%, or 99% identical to a sequence selected from SEQ ID NOs:170-180 and a heavy chain having an amino acid sequence that is at least 90%, 91%, 92%, 93%, 94%, 95%, 96%, 97%, 98%, or 99%, identical to a from SEQ ID NO:103. In some embodiments, the EpCAM activatable antibody comprises a light chain having an amino acid sequence that is at least 90%, 91%, 92%, 93%, 94%, 95%, 96%, 97%, 98%, or 99% identical SEQ ID NO:174 and a heavy chain having an amino acid sequence that is at least 90%, 91%, 92%, 93%, 94%, 95%, 96%, 97%, 98%, or 99%, identical to SEQ ID NO:103.

In some embodiments, the EpCAM activatable antibody comprises a light chain having an amino acid sequence that is at least 90%, 91%, 92%, 93%, 94%, 95%, 96%, 97%, 98%, or 99% identical SEQ ID NO:179 and a heavy chain having an amino acid sequence that is at least 90%, 91%, 92%, 93%, 94%, 95%, 96%, 97%, 98%, or 99%, identical to SEQ ID NO:103. In some embodiments, the percent identity in each instance is 95%. In some embodiments, the percent identity in each instance is 98%.

In some embodiments, the EpCAM activatable antibody of immunoconjugates described herein comprises a light chain having a sequence selected from SEQ ID NOs: 181-188 and a heavy chain having the sequence of SEQ ID NO:127. In some embodiments, the EpCAM activatable antibody comprises a light chain having a sequence selected from SEQ ID NOs: 189-200 and a heavy chain having the sequence of SEQ ID NO:125.

In certain embodiments, the huEpCAM23 antibody is encoded by the plasmids deposited with the American Type Culture Collection (ATCC), located at University Boulevard, Manassas, Va. 20110 on Oct. 4, 2018 under the terms of the Budapest Treaty and having ATCC deposit nos. PTA-125343 and PTA-125344 or PTA-125345. Examples of EpCAM antibody, EpCAM-binding antibody fragments thereof, and EpCAM activatable antibody, immunoconjugates are provided herein.

### 6.4 Polynucleotides, Vectors, Host Cells, and Recombinant Methods

The disclosure further provides polynucleotides comprising a nucleotide sequence encoding the EpCAM antibodies, EpCAM-binding antibody fragments thereof, and EpCAM activatable antibodies disclosed herein.

The polynucleotides may be obtained, and the nucleotide sequence of the polynucleotides determined, using methods known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)) which, briefly, involves the synthesis of overlapping oligonucleotides containing portions of the sequence encoding the antibody, annealing and ligation of those oligonucleotides, and then amplification of the ligated oligonucleotides by PCR.

In some embodiments, a polynucleotide of the disclosure comprises a sequence set forth in Table 12.

TABLE 12
Exemplary Nucleic Acid Sequences.

| | |
|---|---|
| Antibody | |
| name | Sequence |

Chimeric Antibody 23

| | |
|---|---|
| Ch | gaggtgaagctggaggagagcggccccgccctggtgaagcctggagcttccgtgaggatcagctgtaaggcctctggc |
| Epcam23HC | tacacctttacaaactactatatccattgggtgaagcagcggccaggacagggcctggactacatcggctggatctatccc |
| | ggcaacgtgtacatccagtataatgagaagttcaagggcaaggccaccctgacagctgataagtccagctctaccgctttt |
| | atgcagctgtccagcctgacaagcgaggactctgccgtgtacttctgcgctagggatggcccttggtttgcctattggggcc |
| | agggcaccctggtgacagtgtcttccgcttccaccaagggcccatcagttttccccttggctccaagttctaaatccacaag |
| | cggtggaacagctgcactgggatgcctcgttaaagattatttccctgagcctgtgacagtgagctggaatagcggagcatt |
| | gacttcaggtgtgcacacttttcccgtgtgttgcagtcctccggtctgtactcactgtccagtgtcgtaaccgtcccttctagca |
| | gcttgggaacccagacctacatctgtaacgtcaaccataaaccatccaacacaaaggtggataagaaggttgaaccaa |
| | agagctgtgataagacacatacatgccctccttgtcctgcaccagagctcctcggaggtccatctgtgttcctgtttcccccc |
| | aaacccaaggacactcttatgatctctcgtactccagaggtcacctgtgttgttgtcgacgtgagccatgaagatcccgagg |
| | ttaaattcaactggtacgtggatggagtcgaggttcacaatgccaagaccaagcccagggaggagcaatataattctaca |
| | tatcgggtagtgagcgttctgaccgtgctccaccaagattggctcaatggaaaagagtacaagtgcaaggtgtccaacaa |
| | ggctcttcccgctcccattgagaaaactatctccaaagccaaggggcagccacgggaaccccaggtgtatacattgccc |
| | ccatctagagacgagctgaccaagaaccaggtgagtctcacttgtctggtcaaggggttttacccttctgacattgctgtag |
| | agtgggagtctaacggacagccagaaaacaactacaagacaactcccccagtgctggacagcgacgggagcttcttc |
| | ctctactccaagttgactgtagacaagtctagatggcagcaaggaaacgttttctcctgctcagtaatgcatgaggctctgc |
| | acaatcactatacccagaaatcactgtcccttagcccaggg (SEQ ID NO: 201) |
| Ch | gatattgtgctgacccagactccattctccaatccgtcactcttggaacatcagcttccatctcctgcaggtctagtaagagt |
| EpCam23LC | ctcctacatagtgatggcttcacttatttgtattggtttctgcagaagccaggccagtctcctcatctcctgatttatcagacg |
| | tccaaccttgcctcaggagtcccagacaggttcagtagcagtgggtcaggaactgatttcacactgagaatcagcagagtgg |
| | aggctgaagatgtgggtgtttattactgtgctcaaaatctagaacttcccaacacgttcggaggggggaccaagctggaaa |
| | tcaaacgtacggtggctgcaccatctgtcttcatcttcccgccatctgatgagcagttgaaatctggaactgcctctgttgtgtg |
| | cctgctgaataacttctatcccagagaggccaaagtacagtggaaggtggataacgccctccaatcgggtaactcccag |
| | gagagtgtcacagagcaggacagcaaggacagcacctacagcctcagcagcaccctgacgctgagcaaagcagac |
| | tacgagaaacacaaagtctacgcctgcgaagtcacccatcagggcctgagctcgcccgtcacaaagagcttcaacagg |
| | ggagagtgt |
| | (SEQ ID NO: 202) |

Humanized variant 23Gv4.2

| | |
|---|---|
| Hu | caggtgcagctggtgcagagcggagctgaggtgaagaagccaggagcttccgtgaaggtgagctgtaaggcctctggc |
| EpCAM23_ | tacaccttcacaaactactatatccattgggtgaggcaggctccaggacagcggctggagtacatcggatggatctatcct |
| VHGv2 | ggcaacgtgtacatccagtataatgagaagtttaagggcagggccaccctgacagctgacaagagcgcctctaccgctt |
| | acatggagctgtccagcctgagatctgaggacacagccgtgtactattgcgctcgcgatggcccttggtttgcctattgggg |
| | ccagggcaccctggtgacagtgtcttccgcttccaccaagggcccatcagttttccccttggctccaagttctaaatccaca |
| | agcggtggaacagctgcactgggatgcctcgttaaagattatttccctgagcctgtgacagtgagctggaatagcggagc |
| | attgacttcaggtgtgcacacttttcccgctgtgttgcagtcctccggtctgtactcactgtccagtgtcgtaaccgtcccttc |
| | tagcagcttgggaacccagacctacatctgtaacgtcaaccataaaccatccaacacaaaggtggataagaaggttgaacc |
| | aaagagctgtgataagacacatacatgccctccttgtcctgcaccagagctcctcggaggtccatctgtgttcctgtttcccc |
| | ccaaacccaaggacactcttatgatctctcgtactccagaggtcacctgtgttgttgtcgacgtgagccatgaagatcccga |
| | ggttaaattcaactggtacgtggatggagtcgaggttcacaatgccaagaccaagcccagggaggagcaatataattct |
| | acatatcgggtagtgagcgttctgaccgtgctccaccaagattggctcaatggaaaagagtacaagtgcaaggtgtccaa |
| | caaggctcttcccgctcccattgagaaaactatctccaaagccaaggggcagccacgggaaccccaggtgtatacattg |
| | cccccatctagagacgagctgaccaagaaccaggtgagtctcacttgtctggtcaaggggttttacccttctgacattgctgt |
| | agagtgggagtctaacggacagccagaaaacaactacaagacaactcccccagtgctggacagcgacgggagcttct |
| | tcctctactccaagttgactgtagacaagtctagatggcagcaaggaaacgttttctcctgctcagtaatgcatgaggctctg |
| | cacaatcactatacccagaaatcactgtcccttagcccaggg (SEQ ID NO: 203) |
| Hu | gacatcgtgctgacccagacaccactgtctctgtccgtgaccccaggacagcctgctagcatctcttgtaggtccagcagg |
| EpCAM23_ | tccctgctgcatagcgatggcttcacctacctgtattggtttctgcagaagccaggccagtctccccagctgctgatctacca |
| VLGv4 | gacatctaacctggcttccggcgtgcctgacaggttctcttccagcggcagcggcaccgacttcaccctgaagatctctcg |
| | ggtggaggctgaggacgtgggcgtgtactattgcgctcagaacctggagctgccaaataccittggccagggcacaaag |
| | ctggagatcaagcgtacggtggctgcaccatctgtcttcatcttcccgccatctgatgagcagttgaaatctggaactgcctc |
| | tgttgtgtgcctgctgaataacttctatcccagagaggccaaagtacagtggaaggtggataacgccctccaatcgggtaa |
| | ctcccaggagagtgtcacagagcaggacagcaaggacagcacctacagcctcagcagcaccctgacgctgagcaa |
| | agcagactacgagaaacacaaagtctacgcctgcgaagtcacccatcagggcctgagctgcccgtcacaaagagct |
| | tcaacaggggagagtgt (SEQ ID NO: 204) |

Affinity variants (Heavy Chain)

| | |
|---|---|
| Hu | caggtgcagctggtgcagagcggagctgaggtgaagaagccaggagcttccgtgaaggtgagctgtaaggcctctggc |
| EpCam23HG2- | tacaccttcacaaactaccacatccattgggtgaggcaggctccaggacagcggctggagtacatcggatggatctatcc |
| 1361-H | tggcaacgtgtacatccagtataatgagaagtttaagggcagggccaccctgacagctgacaagagcgcctctaccgctt |
| Heavy | acatggagctgtccagcctgagatctgaggacacagccgtgtactattgcgctcgcgatggcccttggtttgcctattgggg |
| Chain | ccagggcaccctggtgacagtgtcttccgcttccaccaagggcccatcagtittccccttggctccaagttctaaatccaca |
| | agcggtggaacagctgcactgggatgcctcgttaaagattatttccctgagcctgtgacagtgagctggaatagcggagc |
| | attgacttcaggtgtgcacacttttcccgctgtgttgcagtcctccggtctgtactcactgtccagtgtcgtaaccgtcccttc |
| | tagcagcttgggaacccagacctacatctgtaacgtcaaccataaaccatccaacacaaaggtggataagaaggttgaacc |
| | aaagagctgtgataagacacatacatgccctccttgtcctgcaccagagctcctcggaggtccatctgtgttcctgtttcccc |
| | ccaaacccaaggacactcttatgatctctcgtactccagaggtcacctgtgttgttgtcgacgtgagccatgaagatcccga |
| | ggttaaattcaactggtacgtggatggagtcgaggttcacaatgccaagaccaagcccagggaggagcaatataattct |
| | acatatcgggtagtgagcgttctgaccgtgctccaccaagattggctcaatggaaaagagtacaagtgcaaggtgtccaa |
| | caaggctcttcccgctcccattgagaaaactatctccaaagccaaggggcagccacgggaaccccaggtgtatacattg |
| | cccccatctagagacgagctgaccaagaaccaggtgagtctcacttgtctggtcaaggggttttacccttctgacattgctgt |
| | agagtgggagtctaacggacagccagaaaacaactacaagacaactcccccagtgctggacagcgacgggagcttct |
| | tcctctactccaagttgactgtagacaagtctagatggcagcaaggaaacgtittctcctgctcagtaatgcatgaggctctg |
| | cacaatcactatacccagaaatcactgtcccttagcccagggtga (SEQ ID NO: 205) |
| Hu | caggtgcagctggtgcagagcggagctgaggtgaagaagccaggagcttccgtgaaggtgagctgtaaggcctctggc |
| EpCam23HG2- | tacaccttcacaaactacgacatccattgggtgaggcaggctccaggacagcggctggagtacatcggatggatctatcc |
| 1361-D | tggcaacgtgtacatccagtataatgagaagtttaagggcagggccaccctgacagctgacaagagcgcctctaccgctt |
| Heavy | acatggagctgtccagcctgagatctgaggacacagccgtgtactattgcgctcgcgatggcccttggtttgcctattgggg |
| Chain | ccagggcaccctggtgacagtgtcttccgcttccaccaagggcccatcagttttccccttggctccaagttctaaatccaca |
| | agcggtggaacagctgcactgggatgcctcgttaaagattatttccctgagcctgtgacagtgagctggaatagcggagc |
| | attgacttcaggtgtgcacacttttcccgctgtgttgcagtcctccggtctgtactcactgtccagtgtcgtaaccgtcccttc |
| | tagcagcttgggaacccagacctacatctgtaacgtcaaccataaaccatccaacacaaaggtggataagaaggttgaacc |
| | aaagagctgtgataagacacatacatgccctccttgtcctgcaccagagctcctcggaggtccatctgtgttcctgtttcccc |
| | ccaaacccaaggacactcttatgatctctcgtactccagaggtcacctgtgttgttgtcgacgtgagccatgaagatcccga |
| | ggttaaattcaactggtacgtggatggagtcgaggttcacaatgccaagaccaagcccagggaggagcaatataattct |
| | acatatcgggtagtgagcgttctgaccgtgctccaccaagattggctcaatggaaaagagtacaagtgcaaggtgtccaa |
| | caaggctcttcccgctcccattgagaaaactatctccaaagccaaggggcagccacgggaaccccaggtgtatacattg |
| | cccccatctagagacgagctgaccaagaaccaggtgagtctcacttgtctggtcaaggggttttacccttctgacattgctgt |
| | agagtgggagtctaacggacagccagaaaacaactacaagacaactcccccagtgctggacagcgacgggagcttct |
| | tcctctactccaagttgactgtagacaagtctagatggcagcaaggaaacgttttctcctgctcagtaatgcatgaggctctg |
| | cacaatcactatacccagaaatcactgtcccttagcccagggtga (SEQ ID NO: 206) |
| Hu | caggtgcagctggtgcagagcggagctgaggtgaagaagccaggagcttccgtgaaggtgagctgtaaggcctctggc |
| EpCam23HG2- | tacaccttcacaaactactatatccattgggtgaggcaggctccaggacagcggctggagtacatcggatggatctatcct |
| 1565-Y | ggcaacgtgtacatccagtataatgagaagtttaagggcagggccaccctgacagctgacaagagcgcctctaccgctt |
| Heavy | acatggagctgtccagcctgagatctgaggacacagccgtgtactattgcgctcgcgatggctactggtttgcctattgggg |
| Chain | ccagggcaccctggtgacagtgtcttccgcttccaccaagggcccatcagttttccccttggctccaagttctaaatccaca |
| | agcggtggaacagctgcactgggatgcctcgttaaagattatttccctgagcctgtgacagtgagctggaatagcggagc |
| | attgacttcaggtgtgcacacttttcccgctgtgttgcagtcctccggtctgtactcactgtccagtgtcgtaaccgtcccttc |
| | tagcagcttgggaacccagacctacatctgtaacgtcaaccataaaccatccaacacaaaggtggataagaaggttgaacc |
| | aaagagctgtgataagacacatacatgccctccttgtcctgcaccagagctcctcggaggtccatctgtgttcctgtttcccc |
| | ccaaacccaaggacactcttatgatctctcgtactccagaggtcacctgtgttgttgtcgacgtgagccatgaagatcccga |
| | ggttaaattcaactggtacgtggatggagtcgaggttcacaatgccaagaccaagcccagggaggagcaatataattct |
| | acatatcgggtagtgagcgttctgaccgtgctccaccaagattggctcaatggaaaagagtacaagtgcaaggtgtccaa |
| | caaggctcttcccgctcccattgagaaaactatctccaaagccaaggggcagccacgggaaccccaggtgtatacattg |
| | cccccatctagagacgagctgaccaagaaccaggtgagtctcacttgtctggtcaaggggttttacccttctgacattgtgt |
| | agagtgggagtctaacggacagccagaaaacaactacaagacaactcccccagtgctggacagcgacgggagcttct |
| | tcctctactccaagttgactgtagacaagtctagatggcagcaaggaaacgttttctcctgctcagtaatgcatgaggctctg |
| | cacaatcactatacccagaaatcactgtcccttagcccagggtga (SEQ ID NO: 207) |

Activatable antibody Light Chains (huEpCAMGv4.2 Antibody)

| | |
|---|---|
| Ep05 | cagggacagtctggacagggatggtggccaccttgccagggaggagcttggtgtgagcagaggatcggaggaggctc |
| 3014 Lv | cagcggaggagctgtgggcctgctggctccaccaggaggactgtctggcagatccgacaacatcggcggctccgatat |
| | cgtgctgacccagacacccctgagcctgtctgtgacccctggccagccagcctccatcagctgcaggtcttcccggtccct |
| | gctgcatagcgacggcttcacctacctgtattggtttctgcagaagcccggccagagccctcagctgctgatctaccagac |
| | atctaatctggcttccggcgtgccagacagattcagctcttccggcagcggcaccgacttcaccctgaagatctctcgcgtg |
| | gaggccgaggatgtgggcgtgtactattgtgctcagaacctggagctgcccaatacctitggccagggcacaaagctgg |
| | agatcaagcgtacggtggctgcaccatctgtcttcatcttcccgccatctgatgagcagttgaaatctggaactgcctctgttg |
| | tgtgcctgctgaataacttctatcccagagaggccaaagtacagtggaaggtggataacgccctccaatcgggtaactcc |
| | caggagagtgtcacagagcaggacagcaaggacagcacctacagcctcagcagcaccctgacgctgagcaaagca |
| | gactacgagaaacacaaagtctacgcctgcgaagtcacccatcagggcctgagctcgcccgtcacaaagagcttcaac |
| | aggggagagtgt (SEQ ID NO: 208) |
| Ep05 | cagggacagagcggacagggatggtggccaccttgccagggaggagcttggtgtgagcagaggatcggaggaggct |
| 2014 Lv | ccagcggaggctctatctcttccggcctgctgagcggcagatctgacaacatcggcggctccgatatcgtgctgacccag |
| | acaccactgtccctgagcgtgaccccaggacagccagcctctatctcctgcaggagctctcggtctctgctgcattccgac |
| | ggcttcacctacctgtattggtttctgcagaagcctggccagtctccacagctgctgatctaccagacaagcaatctggcttct |
| | ggcgtgcccgacagattctccagctctggctccggcaccgacttcaccctgaagatcagccgcgtggaggctgaggatgt |
| | gggcgtgtactattgtgctcagaacctggagctgcctaatacctttggccagggcacaaagctggagatcaagcgtacgg |
| | tggctgcaccatctgtcttcatcttcccgccatctgatgagcagttgaaatctggaactgcctctgttgtgtgcctgctgaataa |
| | cttctatcccagagaggccaaagtacagtggaaggtggataacgccctccaatcgggtaactcccaggagagtgtcaca |
| | gagcaggacagcaaggacagcacctacagcctcagcagcaccctgacgctgagcaaagcagactacgagaaaca |
| | caaagtctacgcctgcgaagtcacccatcagggcctgagctcgcccgtcacaaagagcttcaacaggggagagtgt |
| | (SEQ ID NO: 209) |

In some embodiments, an EpBA of the disclosure comprises a heavy chain nucleic acid sequence of SEQ ID NO:201 and a light chain nucleic acid sequence of SEQ ID NO:202. In some embodiments, an EpBA of the disclosure comprises a heavy chain nucleic acid sequence of SEQ ID NO:203 and a light chain nucleic acid sequence of SEQ ID NO:204. In some embodiments, an EpBA of the disclosure comprises a heavy chain nucleic acid sequence of SEQ ID NO:205 and a light chain nucleic acid sequence of SEQ ID NO:204. In some embodiments, an EpBA of the disclosure comprises a heavy chain nucleic acid sequence of SEQ ID NO:206 and a light chain nucleic acid sequence of SEQ ID NO:204. In some embodiments, an EpBA of the disclosure comprises a heavy chain nucleic acid sequence of SEQ ID NO:207 and a light chain nucleic acid sequence of SEQ ID NO:204.

In some embodiments, an EpBA of the disclosure comprises a heavy chain nucleic acid sequence of SEQ ID NO:203 and a light chain nucleic acid sequence of SEQ ID NO:208. In some embodiments, an EpBA of the disclosure comprises a heavy chain nucleic acid sequence of SEQ ID NO:203 and a light chain nucleic acid sequence of SEQ ID NO:209.

In some embodiments, an EpBA of the disclosure comprises (i) a heavy chain variable region comprising the same amino acid sequence as the amino acid sequence of the heavy chain variable region encoded by the plasmid deposited with the American Type Culture Collection (ATCC®) as PTA-125343 and (ii) a light chain variable region comprising the same amino acid sequence as the amino acid sequence of the light chain variable region encoded by the plasmid deposited with the ATCC® as PTA-125342. Methods of making and using EpCAM antibodies and EpCAM-binding antibody fragments thereof comprising the EpBA are also encompassed by the disclosure.

In some embodiments, the disclosure provides an EpCAM antibody comprising (i) a heavy chain comprising the same amino acid sequence as the amino acid sequence of the heavy chain encoded by the plasmid deposited with the ATCC® as PTA-125343 and (ii) a light chain comprising the same amino acid sequence as the amino acid sequence of the light chain variable region encoded by the plasmid deposited with the ATCC® as PTA-125342. Methods of making and using the EpCAM antibody are also encompassed by the disclosure.

In some embodiments, the disclosure provides an EpCAM activatable antibody or EpCAM-binding activatable antibody fragment comprising (i) a heavy chain variable region comprising the same amino acid sequence as the amino acid sequence of the heavy chain variable region encoded by the plasmid deposited with the ATCC® as PTA-125343 and (ii) a light chain variable region comprising the same amino acid sequence as the amino acid sequence of the light chain variable region encoded by the plasmid deposited with the ATCC® as PTA-125344. Methods of making and using the EpCAM activatable antibody or EpCAM-binding activatable antibody fragment are also encompassed by the disclosure.

In other embodiments, the disclosure provides an EpCAM activatable antibody or EpCAM-binding activatable antibody fragment comprising (i) a heavy chain variable region comprising the same amino acid sequence as the amino acid sequence of the heavy chain variable region encoded by the plasmid deposited with the ATCC® as PTA-125343 and (ii) a light chain variable region comprising the same amino acid sequence as the amino acid sequence of the light chain variable region encoded by the plasmid deposited with the ATCC® as PTA-125345. Methods of making and using the EpCAM activatable antibody or EpCAM-binding activatable antibody fragment are also encompassed by the disclosure.

Methods for the construction of recombinant vectors containing antibody coding sequences and appropriate transcriptional and translational control signals are well known in the art. Once an antibody has been recombinantly expressed, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. In this regard, U.S. Pat. No. 7,538,195 has been referred to in the present disclosure, the contents of which is hereby incorporated by reference in its entirety.

The disclosure also provides methods of producing an EpCAM antibody, EpCAM-binding antibody fragments thereof, or EpCAM activatable antibody disclosed herein by culturing a cell under conditions that lead to expression of the antibody and/or EpCAM activatable antibody, wherein the cell comprises nucleic acid molecules encoding the antibody, antibody fragment or activatable antibody. In some embodiments, the cell is a Chinese hamster ovary (CHO) cell.

The disclosure also provides a method of manufacturing EpCAM activatable antibodies that in an activated state binds EpCAM by (a) culturing a cell comprising a nucleic acid construct that encodes the EpCAM activatable antibody, under conditions that lead to expression of the EpCAM activatable antibody, wherein the EpCAM activatable antibody, comprises a masking moiety (MM), a cleavable moiety (CM), and an Ab (e.g., and EpCAM antibody or EpCAM-binding antibody fragments thereof), (i) wherein the CM is a polypeptide that functions as a substrate for a protease; and (ii) wherein the CM is positioned in the EpCAM activatable antibody, such that, when the EpCAM activatable antibody, is in an uncleaved state, the MM interferes with specific binding of the Ab to EpCAM and in a cleaved state the MM does not interfere or compete with specific binding of the Ab to EpCAM; and (b) recovering the EpCAM activatable antibody. Suitable Ab, MM, and/or CM include any of the Ab, MM, and/or CM disclosed herein. In some embodiments, the cell is a Chinese hamster ovary (CHO) cell.

### 6.5 Immunoconjugates

The EpCAM immunoconjugates as disclosed herein comprise camptothecin and/or derivatives thereof. Camptothecin and derivatives thereof have been disclosed in US Appl. Publ. No. 2021/0077482 A1 and U.S. Pat. No. 11,229,639 B2, each of which is incorporated by reference herein in its entirety.

In some instances, the EpCAM immunoconjugates are generated by conjugating at least one linker-payload reactant comprising a compound of Formula I or a pharmaceutically acceptable salt thereof with an EpCAM antibody as described in Section 6.2 or an EpCAM activatable antibody as described in Section 6.3:

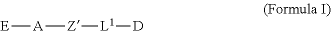

D is represented by the following structural formula:

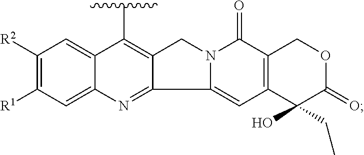

R¹ is F;
R² is methyl;
-L¹-Z′—* is —(C₁-C₄ alkylene)-O—CH₂—NR⁸—*, —(C₁-C₄ alkylene)-NR⁸, or —(C₁-C₅ alkylene)-NR⁵C(═O)—(C₁-C₅ alkylene)-O—CH₂—NR⁸*, where * is a site covalently attached to A;
each R⁵ is independently H, methyl, or benzyl;
each R⁸ is independently H, methyl, or benzyl;
A is a peptide comprising 2 to 4 amino acids; and
E is C(═O)—(C₁-C₁₀ alkylene)-X³ wherein X³ is

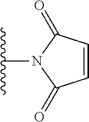 [SMILES: *N1C(=O)C=CC1=O]

In some instances, the linker-payload reactant comprises:

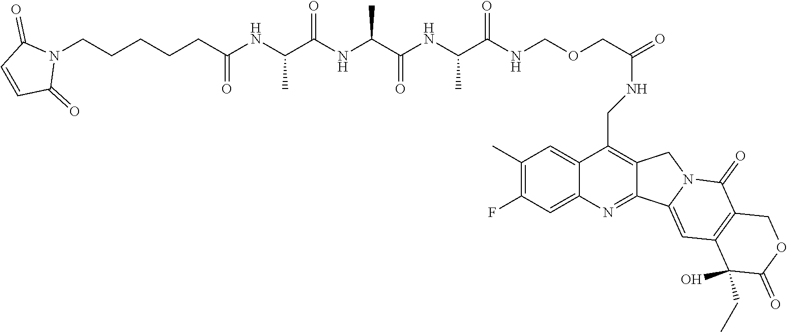 [SMILES: CC[C@@]1(O)C(=O)OCc2c1cc1n(c2=O)Cc2c-1nc1cc(F)c(C)cc1c2CNC(=O)COCNC(=O)[C@H](C)NC(=O)[C@H](C)NC(=O)[C@H](C)NC(=O)CCCCCN1C(=O)C=CC1=O]

or a pharmaceutically acceptable salt thereof.

In some instances, the linker-payload reactant comprises:

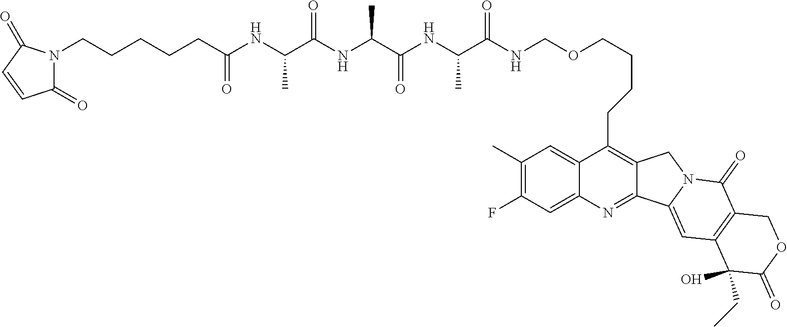 [SMILES: CC[C@@]1(O)C(=O)OCc2c1cc1n(c2=O)Cc2c-1nc1cc(F)c(C)cc1c2CCCCOCNC(=O)[C@H](C)NC(=O)[C@H](C)NC(=O)[C@H](C)NC(=O)CCCCCN1C(=O)C=CC1=O]

or a pharmaceutically acceptable saft thereof.

Where the linker-payload reactant comprises a compound of Formula I or a pharmaceutically acceptable salt thereof, the maleimide group X³ reacts with a thiol (—SH) group in the EpCAM antibody or activatable antibody to form a covalent bond in the immunoconjugate, as shown below:

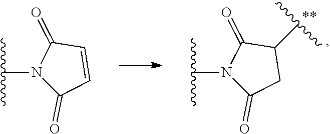 [SMILES: *N1C(=O)C=CC1=O ; *C1CC(=O)N(*)C1=O]

where ** indicates the attachment to the EpCAM antibody or activatable antibody via the sulfur of the thiol group.

In some embodiments, the thiol (—SH) group of the EpCAM antibody or activatable antibody is a cysteine thiol group. Those of skill in the art will recognize that once the maleimide of group X³ in Formula I of the linker-payload reactant above is reacted with a thiol of the EpCAM antibody or activatable antibody, the maleimide group is reduced to succinimide. The linker-payload, when conjugated to the EpCAM antibody or activatable antibody, thus comprises succinimide.

In some embodiments, the EpCAM antibody or activatable antibody is an IgG1 antibody having eight cysteines which, when in a native conformation (e.g., not reduced), are involved in forming four interchain disulfide bonds. See, e.g., SEQ ID NOs:103, 140, and 179 in Tables 6, 7, and 11, respectively for exemplary identification of such cysteine residues. In an IgG1 antibody, each light chain is linked to a heavy chain by one covalent disulfide bond, while the two heavy chains are linked together via two disulfide bonds. As provided herein, these disulfide bonds may be reduced, resulting in up to eight free cysteines and cysteine thiol groups. One linker-payload reactant can react with one cysteine's thiol group, with up to eight linker-payload reactants reacting with each IgG1 EpCAM antibody or activatable antibody. In some embodiments, each EpCAM antibody or activatable antibody reacts with up to eight linker-payload reactants.

In some instances, the EpCAM immunoconjugates comprise at least one linker-payload comprising a compound of Formula I or a pharmaceutically acceptable salt thereof (where the compound of Formula I or a pharmaceutically acceptable salt thereof represents the linker-payload reactant, i.e. the linker-payload prior to conjugation to the EpCAM antibody or activatable antibody):

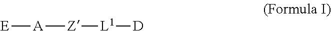

wherein:
D is represented by the following structural formula:

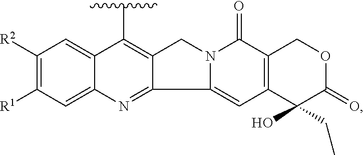

R¹ is F;
R² is methyl;
L¹-Z′—* is —(C₁-C₄ alkylene)-O—CH₂—NR⁸—*, —(C₁-C₄ alkylene)-NR⁸, or —(C₁-C₅ alkylene)-NR⁵C(═O)—(C₁-C₅ alkylene)-O—CH₂—NR⁸*, where * is a site covalently attached to A;
each R⁵ is independently H, methyl, or benzyl;
each R⁸ is independently H, methyl, or benzyl;
A is a peptide comprising 2 to 4 amino acids; and
E is C(═O)—(C₁-C₁₀ alkylene)-X³ wherein X³ is

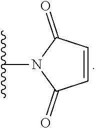 [SMILES: *N1C(=O)C=CC1=O]

Thus, in some instances, the EpCAM immunoconjugates comprise at least one linker-payload comprising the structure of Formula (Ia) or a pharmaceutically acceptable salt thereof:

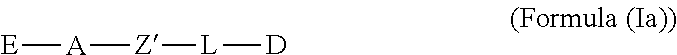

wherein:
D is represented by the following structural formula:

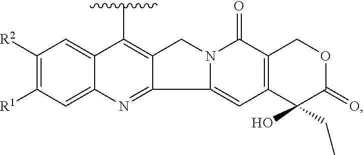

R¹ is F;
R² is methyl;
L¹-Z′—* is —(C₁-C₄ alkylene)-O—CH₂—NR⁸—*, —(C₁-C₄ alkylene)-NR⁸, or —(C₁-C₅ alkylene)-NR⁵C(═O)—(C₁-C₅ alkylene)-O—CH₂—NR⁸*, where * is a site covalently attached to A;
each R⁵ is independently H, methyl, or benzyl;
each R⁸ is independently H, methyl, or benzyl;
A is a peptide comprising 2 to 4 amino acids; and
E is C(═O)—(C₁-C₁ alkylene)-X³ wherein X³ is

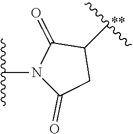 [SMILES: *C1CC(=O)N(*)C1=O]

wherein ** is a site covalently attached to the EpCAM antibody or activatable antibody.

In some instances, A in Formula (I) or A in Formula (Ia) is substituted with one or more polyol. In some instances, the polyol is —(C1-C6 alkylene)-X⁵—Y³, wherein X⁵ is —NR¹²C(═O)— or —C(═O)NR¹²—; Y³ is C6 alkyl substituted with 5 OH groups; and R¹² is —H, C1-C6 alkyl, C1-C6 fluoroalkyl, C3-C6 cycloalkyl, aryl, heteroaryl, or benzyl.

In some instances, the polyol of the immunoconjugate comprises

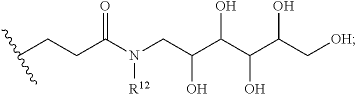

wherein R12 is H or methyl.

In some instances, the immunoconjugate comprises the following linker-payload conjugate (i.e. linker-payload prior to conjugation with the EpCAM antibody or activatable antibody):

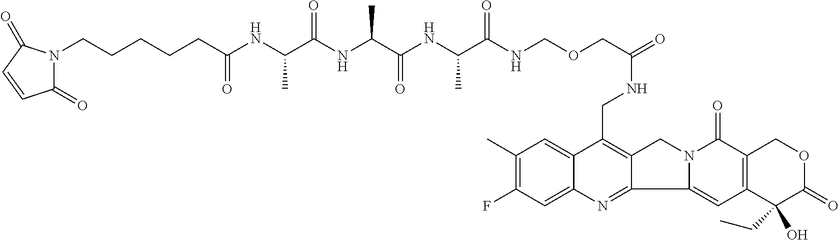 [SMILES: CC[C@@]1(O)C(=O)OCc2c1cc1n(c2=O)Cc2c-1nc1cc(F)c(C)cc1c2CNC(=O)COCNC(=O)[C@H](C)NC(=O)[C@H](C)NC(=O)[C@H](C)NC(=O)CCCCCN1C(=O)C=CC1=O]

or a pharmaceutically acceptable salt thereof.

Thus, in some instances, the immunoconjugate comprises the following linker-payload:

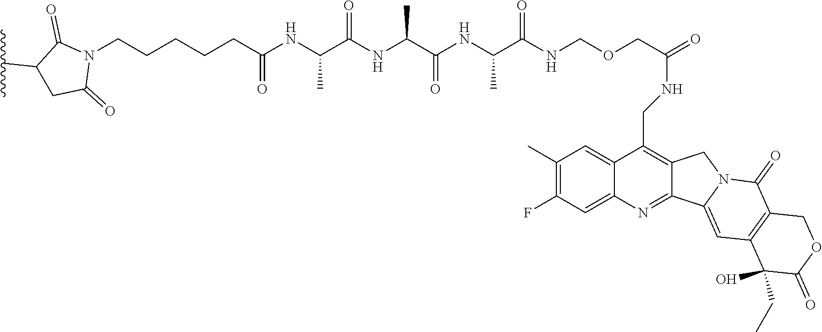 [SMILES: *C1CC(=O)N(CCCCCC(=O)N[C@@H](C)C(=O)N[C@@H](C)C(=O)N[C@@H](C)C(=O)NCOCC(=O)NCc2c3c(nc4cc(F)c(C)cc24)-c2cc4c(c(=O)n2C3)COC(=O)[C@]4(O)CC)C1=O]

or a pharmaceutically acceptable salt thereof.

In some instances, the immunoconjugate comprises the following linker-payload conjugate (i.e., linker-payload prior to conjugation with the EpCAM antibody or activatable antibody):

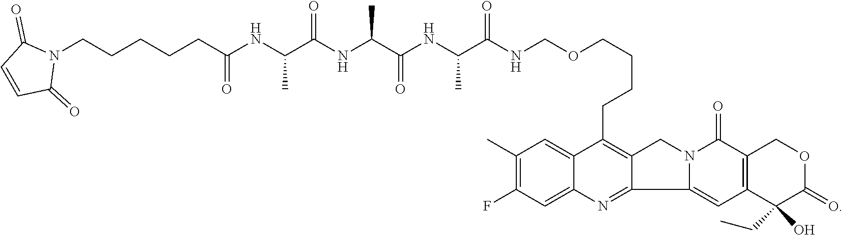 [SMILES: CC[C@@]1(O)C(=O)OCc2c1cc1n(c2=O)Cc2c-1nc1cc(F)c(C)cc1c2CCCCOCNC(=O)[C@H](C)NC(=O)[C@H](C)NC(=O)[C@H](C)NC(=O)CCCCCN1C(=O)C=CC1=O]

or a pharmaceutically acceptable salt thereof.

Thus, in some instances, the immunoconjugate comprises the following linker-payload:

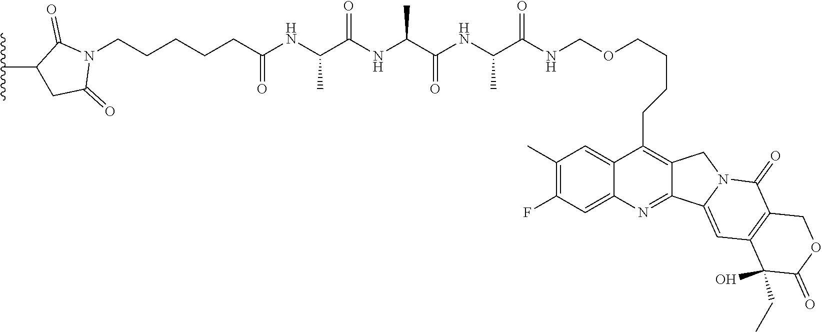 [SMILES: *C1CC(=O)N(CCCCCC(=O)N[C@@H](C)C(=O)N[C@@H](C)C(=O)N[C@@H](C)C(=O)NCOCCCCc2c3c(nc4cc(F)c(C)cc24)-c2cc4c(c(=O)n2C3)COC(=O)[C@]4(O)CC)C1=O]

or a pharmaceutically acceptable salt thereof.

In the various embodiments disclosing -L¹-Z′—* herein, * is the site covalently attached to A. In some embodiments, A is a peptide comprising 2 to 8 amino acids. In some embodiments, A is a peptide comprising 2 to 4 amino acids. In some embodiments, at least one amino acid in said peptide is a L amino acid. In some embodiments, each amino acid in said peptide is a L amino acid. In some embodiments, at least one amino acid in said peptide is a D amino acid. In some embodiments, A is -(AA¹)-(AA²)ₐ₁-*, where * is the site covalently attached to E; AA¹ and AA² are each independently an amino acid residue; and a1 is an integer from 1-9. In some embodiments, -AA¹-(AA²)ₐ₁-* is -Gly-Gly-Gly-*, -Ala-Val-*, -Val-Ala-*, -Val-Cit-*, -Val-Lys-* , -Lys-Val-*, -Phe-Lys-*, -Lys-Phe-*, -Lys-Lys-*, -Ala-Lys-*, -Lys-Ala-*, -Phe-Cit-*, -Cit-Phe-*, -Leu-Cit-*, -Cit-Leu-* -Ile-Cit-*, -Phe-Ala-*, -Ala-Phe-*, -Phe-N⁹-tosyl-Arg-* , —N⁹-tosyl-Arg-Phe-*, -Phe-N⁹-nitro-Arg-*, —N⁹-nitro-Arg-Phe*, -Phe-Phe-Lys-*, -Lys-Phe-Phe-*, -Gly-Phe-Lys-*, Lys-Phe-Gly-*, -Leu-Ala-Leu-*, -Ile-Ala-Leu-*, -Leu-Ala-Ile-*, -Val-Ala-Val-*, -Ala-Leu-Ala-Leu-*, -Leu-Ala-Leu-Ala-*, -\hat{ }-Ala-Leu-Ala-Leu-*, -Gly-Phe-Leu-Gly-*, -Gly-Leu-Phe-Gly-*, -Val-Arg-*, -Arg-Val-*, -Arg-Arg-*, -Ala-Ala-*, -Ala-Met-*, -Met-Ala-*, -Thr-Thr-*, -Thr-Met-*, -Met-Thr-*, -Leu-Ala-*, -Ala-Leu-*, -Cit-Val-*, -Gln-Val-*, -Val-Gln-*, -Ser-Val-*, -Val-Ser-*, -Ser-Ala-*, -Ser-Gly-*, -Ala-Ser-*, -Gly-Ser-*, -Leu-Gln-*, -Gln-Leu-*, -Phe-Arg-*, -Arg-Phe-*, -Tyr-Arg-*, -Arg-Tyr-*, -Phe-Gln-*, -Gln-Phe-*, -Val-Thr-*, -Thr-Val-*, -Met-Tyr-*, and -Tyr-Met-*. In some embodiments, -AA¹-(AA²)a1-* is -Val-D-Lys-*, -Val-D-Arg-*, -L-Val-Cit-*, -L-Val-Lys-*, -L-Val-Arg-*, -L-Val-D-Cit-*, -L-Phe-Phe-Lys-*, -L-Val-D-Lys-*, -L-Val-D-Arg-*, -L-Arg-D-Arg-*, -L-Ala-Ala-*, -L-Ala-D-Ala-*, -Ala-D-Ala-*, -Val-D-Cit-*, -L-Ala-L-Ala-*, -L-Ala-L-Val-*, -L-Gln-L-Val-*, -L-Gln-L-Leu-*, or -L-Ser-L-Val-*. In some embodiments, -AA¹-(AA²)a1-* is: -Ala-Ala-*, -Ala-Val-*, -Val-Ala-*, -Gln-Leu-*, -Leu-Gln-*, -Ala-Ala-Ala-*, -Ala-Ala-Ala-Ala-*, -Gly-Ala-Gly-Gly-*, -Gly-Gly-Ala-Gly-*, -Gly-Val-Gly-Gly-*, -Gly-Gly-Val-Gly-*, -Gly-Phe-Gly-Gly-*, or -Gly-Gly-Phe-Gly-*. In some embodiments, -AA¹-(AA²)ₐ₁-* is: -L-Ala-L-Ala-*, -L-Ala-D-Ala-*, -L-Ala-L-Val-*, -L-Ala-D-Val-*, -L-Val-L-Ala-*, -L-Val-D-Ala-*, -L-Gln-L-Leu-*, -L-Gln-D-Leu-*, -L-Leu-L-Gln-*, -L-Leu-D-Gln-*, -L-Ala-L-Ala-L-Ala-*, -L-Ala-D-Ala-L-Ala-*, -L-Ala-L-Ala-D-Ala-*, -L-Ala-L-Ala-L-Ala-L-Ala-*, -L-Ala-D-Ala-L-Ala-L-Ala-*, -L-Ala-L-Ala-D-Ala-L-Ala-*, -L-Ala-L-Ala-L-Ala-D-Ala-*, -Gly-L-Ala-Gly-Gly-*, -Gly-Gly-L-Ala-Gly-*, -Gly-D-Ala-Gly-Gly-*, Gly-Gly-D-Ala-Gly-*, -Gly-L-Val-Gly-Gly-*, Gly-Gly-L-Val-Gly-*, -Gly-D-Val-Gly-Gly-*, Gly-Gly-D-Val-Gly-*, -Gly-L-Phe-Gly-Gly-*, or Gly-Gly-L-Phe-Gly-*. In some embodiments, -AA¹-(AA²)ₐ₁-* is: -L-Ala-L-Ala-*, -L-Ala-D-Ala-LAla-*, -L-Ala-L-Ala-L-Ala-*, or -L-Ala-L-Ala-L-Ala-L-Ala-*.

In some embodiments, -AA¹-(AA²)ₐ₁-* is -L-Ala-L-Ala-L-Ala-*.

In the various embodiments disclosing -AA¹-(AA²)ₐ₁-* herein,* is the site covalently attached to E.

In some embodiments, the EpCAM antibody or activatable antibody is an IgG1 antibody having eight cysteines which, when in a native conformation (e.g., not reduced), are involved in forming four disulfide bonds. See, e.g., SEQ ID NOs:103, 140, and 179 in Tables 6, 7, and 11, respectively for exemplary identification of such cysteine residues. As provided herein, these disulfide bonds may be reduced, resulting in eight free cysteines and cysteine thiol groups. One linker-payload reactant can react with one cysteine's thiol group, with up to eight linker-payload reactants reacting with each EpCAM antibody or activatable antibody. In some embodiments, each EpCAM antibody or activatable antibody reacts with up to eight linker-payload reactants resulting in a linker-payload to antibody ratio (i.e., a drug-to-antibody ratio (DAR)) ranging from 2 to 8. In certain embodiments, the DAR represents the average number of cytotoxic linker-payload agents per antibody or activatable antibody molecule. In some embodiments the DAR is 2. In some embodiments the DAR is 3. In some embodiments the DAR is 4. In some embodiments the DAR is 5. In some embodiments the DAR is 6. In some embodiments the DAR is 7. In some embodiments the DAR is 8.

In some instances, the linker-payload to antibody ratio (i.e., a drug-to-antibody ratio (DAR)) ranges from 2 to 12. In some instances, the DAR is at least 2, at least 3, at least 4, 4, at least 5, 5, at least 6, 6, at least 7, 7, at least 8, 8, at least 9, 9, at least 10, 10, at least 11, 11, at least 12, or 12. In certain embodiments, the DAR represents the average number of cytotoxic linker-payload agents per antibody or activatable antibody molecule. In some embodiments, the DAR is 8.

In some embodiments, the disclosure provides an EpCAM immunoconjugate comprising at least one linker-payload comprising a compound of the following formula, or a pharmaceutically acceptable salt thereof:

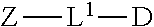

wherein D is represented by the following structural formula:

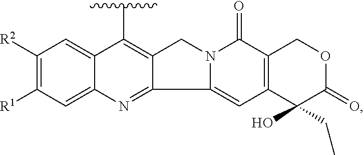

R¹ is —F, —CH₃, or —CF₃;
R² is —H, —F, —OR³, —SR³, —S(O)R⁴, —S(O)₂R⁴, C₁-C₆ alkyl, or C₁-C₆fluoroalkyl; or R¹ and R² taken together with the carbon atoms to which they are attached form a methylenedioxy or a difluoromethylenedioxy ring;
R³ is H or C₁-C₆ alkyl;
R⁴ is C₁-C₆alkyl:
L¹ is absent, —(C₁-C₆ alkylene)-, —(C₁-C₆ alkylene)-X¹—(C₁-C₆ alkylene)-, —X^{1′}—(C₁-C₆ alkylene)-*, or —(C₁-C₆ alkylene)-X¹-L²-*; where * is the site covalently attached to Z:
X¹ is —O—, —S—, —S(O)—, —S(O)₂—, —C(═O)—, —NR⁵—, —NR⁵C(═O)—, or —C(═O)NR⁵—;
X^{1′} is —O—, —S—, —S(O)—, or —S(O)₂—;
L² is phenylene;
each R⁵ is independently —H, C₁-C₆ alkyl, C₁-C₆ fluoroalkyl, C₃-C₆ cycloalkyl, aryl, heteroaryl, or benzyl;
Z is —H or —X²;
X² is —OR⁶, —SR⁶, —S(O)R⁶, —S(O)₂R⁶, —SSR⁶, or —N(R⁶)₂;
each R⁶ is independently —H, C₁-C₆ alkyl, C₁-C₆ fluoroalkyl, C₃-C₆ cycloalkyl, aryl, heteroaryl, or benzyl;
L¹ and L² are each independently optionally substituted with 1-4 substituents selected from halogen, —CN, —SR⁷, —N(R⁷)₂, C₁-C₆ alkyl, C₁-C₆ fluoroalkyl, C₁-C₆ heteroalkyl, C₃-C₆ cycloalkyl, C₂-C₁₀ heterocycloalkyl, aryl, or heteroaryl; and each R⁷ is independently H, C₁-C₆ alkyl, C₁-C₆ fluoroalkyl, C₃-C₆ cycloalkyl, aryl, heteroaryl, or benzyl;
with the proviso that if R¹ is F, then L¹ is —(C₁-C₆ alkylene)-, —(C₁-C₆ alkylene)-X¹—(C₁-C₆ alkylene)-, —X^{1′}—(C₁-C₆ alkylene)-*, or —(C₁-C₆ alkylene)-X¹-L²-*; where * is the site covalently attached to Z; and Z is —X²;
with the proviso that if R¹ is F and R² is —OMe, then cannot be NH₂; and
with the proviso that if R¹ is F and R² is -Me, then -L₁-Z cannot be —CH₂OH.

Additional camptothecin derivatives are disclosed in US Appl. Publ. No. 2021/0077482 A1 and U.S. Pat. No. 11,229,639 B2, each of which is incorporated by reference herein in its entirety.

In some embodiments, the linker-payload of the immunoconjugate comprises exatecan or a derivative thereof. In some embodiments, the linker-payload of the immunoconjugate comprises reduced deruxtecan, in which the maleimide group of deruxtecan is reduced to succinimide during the conjugation reaction thereby linking deruxtecan to the EpCAM antibody or activatable antibody via a thiol group. Deruxtecan comprises an exatecan derivative and a maleimide-GGFG linker (SEQ ID NO:311). Exatecan, its derivatives, and deruxtecan are described in, e.g., WO2015/115091, the contents of which are herein incorporated by reference in its entirety.

### 6.6 Methods of Making Immunoconjugates

The immunoconjugates comprising an EpCAM-binding agent (“EpBA”, e.g., an EpCAM antibody, EpCAM-binding antibody fragment thereof, or EpCAM activatable antibody) covalently linked to a cytotoxic agent through inter-chain cysteines residues located on the EpBA as described as an embodiment above or any specific embodiments described herein can be prepared according to any methods known in the art, see, e.g., WO 2020/219287, WO 2014/197612, WO 2012/128868, and WO 2012/112687, the entire contents of each of which is herein incorporated by reference in its entirety.

Methods of making EpCAM-specific conjugates are disclosed in WO 2020/086665, the entire contents of each of which is herein incorporated by reference in its entirety.

In some embodiments, the immunoconjugates of the instant disclosure are prepared by a method comprising the steps of reacting the EpBA (e.g., an EpCAM antibody, EpCAM-binding antibody fragment thereof, or EpCAM activatable antibody, disclosed herein) with a cytotoxic agent of the disclosure having a thiol reactive group. In certain embodiments, the thiol reactive group is maleimide. In particular embodiments, the thiol reactive group of the cytotoxic agent (e.g., a linker-payload of the disclosure) reacts with a thiol group of a cysteine residue of the EpBA.

In certain embodiments, the thiol reactive group (e.g., maleimide) of the cytotoxic agent is coupled to the thiol group of the EpBA (e.g., a cysteine thiol) via a thiol-Michael addition.

In some embodiments, conjugation of a linker-payload reactant to an EpCAM activatable antibody comprises partial reduction of the activatable antibody. This exposes the reactive native cysteines that typically form disulfide bridges between the heavy chains of the activatable antibody, as well as between light and heavy chains, but without disturbing the one or more disulfide bonds occurring within the mask moiety (MM) of the activatable antibody. The MM thus retains the ability to effectively mask the EpCAM antibody or EpCAM binding fragment thereof. When reduction and subsequent conjugation is not controlled properly, the activatable antibody is completely reduced and the masking efficiency of the activatable antibody is/will be compromised.

In some embodiments, the activatable EpCAM antibody is an IgG1 antibody. In certain embodiments, the activatable EpCAM antibody is a humanized IgG1 antibody. Thus, in certain embodiments, the activatable EpCAM antibody has four disulfide bonds formed by eight cysteine residues. When the activatable antibody is partially reduced, these four disulfide bonds are reduced and the eight cysteines and their respective thiol groups become available to react with the thiol reacting group (e.g., maleimide) of the linker-payload.

Examples of reducing agents suitable for use in the partial reduction of the EpCAM activatable antibody include, but are not limited to, BMS (bis(2-mercaptoethyl)sulfone), cysteamine, cysteine, DMH (dimethyl-bis-mercaptoacetyl hydrazine), DTBA (dithiobutylamine), DTT (dithiothreitol), GILT (gamma interferon inducible lysosomal thiol reductase; for enzymatic reduction), glutathione, -mercaptoethanol, MEA (2-mercaptoethylamine), pyridine -2-thione, sodium borohydride, sodium phosphorothioate, TCEP ((tris(2-carboxyethyl)phosphine)), and thiopropyl-agarose. In some embodiments, the reducing agent is DTT, 3-mercaptoethanol or TCEP.

In some embodiments, the reducing agent is TCEP. TCEP is often used as a reducing agent to cleave disulfide bonds within and between proteins. TCEP is very selective and does not react with other function groups found within proteins, nor does it react with buried disulfides. Relative to DTT and (3-mercaptoethanol, which are two other common reducing agents, TCEP has the advantages of being a more powerful reducing agent, more hydrophilic, more resistant to oxidation in air, and odorless. Unlike DTT, TCEP is active in both alkaline and acidic conditions. TCEP is particularly useful when reacting cysteine residues with maleimides. TCEP can keep the cysteines from forming disulfide bonds, and unlike DTT and β-mercaptoethanol, TCEP will not react as readily with the maleimide.

In some embodiments, the ratio of reducing agent (e.g., TCEP) to EpCAM activatable antibody will be in a range from about 20:1 to 1:1, from about 10:1 to 1:1, from about 9:1 to 1:1, from about 8:1 to 1:1, from about 7:1 to 1:1, from about 6:1 to 1:1, from about 5:1 to 1:1, from about 4:1 to 1:1, from about 3:1 to 1:1, from about 2:1 to 1:1, from about 20:1 to 1:1.5, from about 10:1 to 1:1.5, from about 9:1 to 1:1.5, from about 8:1 to 1:1.5, from about 7:1 to 1:1.5, from about 6:1 to 1:1.5, from about 5:1 to 1:1.5, from about 4:1 to 1:1.5, from about 3:1 to 1:1.5, from about 2:1 to 1:1.5, from about 1.5:1 to 1:1.5, or from about 1:1 to 1:1.5. In some embodiments, the ratio is in a range of from about 5:1 to 1:1. In some embodiments, the ratio is in a range of from about 5:1 to 1.5:1. In some embodiments, the ratio is in a range of from about 4:1 to 1:1. In some embodiments, the ratio is in a range from about 4:1 to 1.5:1. In some embodiments, the ratio is in a range from about 8:1 to about 1:1. In some embodiments, the ratio is in a range of from about 2.5:1 to 1:1.

The maleimide functional group in the linker-payload reactant reacts with a thiol (—SH) group on the activatable antibody to create a covalent thioester linkage.

In some embodiments, native cysteines on the activatable antibody comprise the thiol (—SH) group. In some embodiments, the EpCAM activatable antibody is partially reduced with 4.5 molar equivalents of TCEP in solution at room temperature for about 3 hours. The partially reduced activatable antibody is reacted with 10 molar equivalents of a linker-payload reactant, with DMAc as a co-solvent of up to about 8.3% (v/v) in aqueous buffered solution at room temperature for about 1 hour. Thereafter the reaction is quenched with 20 molar equivalents of N-acetyl-L-cysteine (NAC) at room temperature for about 1 hour. After quenching, the resulting immunoconjugate may be purified to remove small molecule impurities, and buffer exchanged by UF/DF into a basal formulation buffer. In some embodiments, the basal formulation buffer comprises one or more pharmaceutically acceptable excipients. The basal formulation buffer comprising the immunoconjugate may then be sterile filtered into contains.

Exemplary methods for partially reducing an activatable antibody and conjugating a linker-payload thereto are provided herein, for example, at Examples 7.4.1-7.4.5.

In some embodiments, the conjugation methods provided herein using the EpCAM antibodies or activatable antibodies and the linker-payload reactants results in a generally homogeneous immunoconjugate having 8 linker-payloads conjugated to each antibody or activatable antibody. Few immunoconjugates prepared using the methods and materials provided herein will have a number of linker-payloads conjugated to each antibody or activatable antibody that is not 8. This is in contrast to huEpCAM23Gv4.2-GMBS-DM21L (see, e.g., Example 7.4.5), which had a DAR of between 3.5 and 5. The consistently higher DAR of 8 of the immunoconjugates described herein provides for a strong bystander effect.

A representative method for preparing immunoconjugates of the disclosure is provided in Example 7.4.1.

In certain embodiments, the immunoconjugates are prepared by a method comprising the steps of reacting the EpBA (e.g., an EpCAM antibody, EpCAM-binding antibody fragment thereof, or EpCAM activatable antibody, disclosed herein) with the cytotoxic agent having an amine reactive group.

In some embodiments, the reaction is carried out in the presence of an imine reactive reagent, such as NaHSO₃.

In certain embodiments, the immunoconjugates are prepared by a method comprising the steps of:
(a) reacting the cytotoxic agent with a linker compound having an amine reactive group and a thiol reactive group to form a cytotoxic agent-linker compound (i.e., a linker-payload reactant) having the amine reactive group bound thereto; and
(b) reacting the EpBA with the cytotoxic agent-linker compound.

In one embodiment, the reaction in step (a) is carried out in the presence of an imine reactive reagent (e.g., NaHSO₃). In one embodiment, the cytotoxic agent-linker compound is reacted with the EpBA without purification. Alternatively, the cytotoxic agent-linker compound (i.e., the linker-payload reactant) is first purified before reacting with the EpBA.

In certain embodiments, the immunoconjugates of an embodiment is prepared by a method comprising the steps of:
(a) reacting the EpBA with a linker compound having an amine reactive group and a thiol reactive group to form a modified EpBA having a thiol reactive group bound thereto; and
(b) reacting the modified EpBA with the cytotoxic agent.

In one embodiment, the reaction in step (b) is carried out in the presence of an imine reactive reagent (e.g., NaHSO₃).

In certain embodiments, the immunoconjugates are prepared by a method comprising the steps of reacting the EpBA, a cytotoxic compound and a linker compound having an amine reactive group and a thiol reactive group. In one embodiment, the reaction is carried out in the presence of an imine reactive agent (e.g., NaHSO₃).

In a 1st aspect, for the method described above, the reaction of step (a) is carried out at a pH of 1.9 to 5.0. More specifically, the pH is 2.5 to 4.9, 1.9 to 4.8, 2.0 to 4.8, 2.5 to 4.5, 2.9 to 4.5, 2.9 to 4.0, 2.9 to 3.7, 3.1 to 3.5, or 3.2 to 3.4. In another specific embodiment, the reaction of step (a) is carried out at a pH of 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.5, 4.6, 4.7, 4.8, 4.9 or 5.0. In yet another specific embodiment, the reaction of step (a) is carried out at a pH of 3.3. As used herein, a specific pH value means the specific value ±0.05.

In some embodiments, the reaction of step (a) is carried out in the presence of a buffer solution. Any suitable buffer solution known in the art can be used in the provided methods. Suitable buffer solutions include, for example, but are not limited to, a citrate buffer, an acetate buffer, a succinate buffer, a phosphate buffer, a glycine-containing buffer (e.g., glycine-HCl buffer), a phthalate buffer (e.g., a buffer solution comprising sodium or potassium hydrogen phthalate), and a combination thereof. In some embodiments, the buffer solution is a succinate buffer. In some embodiments, the buffer solution is a phosphate buffer. In some embodiments, the buffer is a citrate-phosphate buffer. In some embodiments, the buffer is a citrate-phosphate buffer comprising citric acid and Na2HPO4. In other embodiments, the buffer is a citrate-phosphate buffer comprising citric acid and K2HPO4. In some embodiments, the concentration of the buffer solution described above can be in the range of 10 to 250 mM, 10 to 200 mM, 10 to 150 mM, 10 to 100 mM, 25 to 100 mM 25 to 75 mM, 10 to 50 mM, or 20 to 50 mM.

In a 2nd aspect, the reaction step (a) is carried out in the absence of a buffer solution (e.g., the buffers described in the 1st aspect). In some embodiments, the present method comprises the steps of: (a) reacting the imine-moiety in the imine-containing cytotoxic agent having a thiol-reactive group described above (i.e., formula (C1a′), (C1a′1), (C1b′), (C1b′1), (C2a″), (C2a″1), (C2b″) or (C2b″1), wherein the double line between N and C represents a double bond, X is absent and Y is —H) with sulfur dioxide, a bisulfite salt or a metabisulfite salt in an aqueous solution to form a modified cytotoxic agent comprising a modified imine moiety represented by the following formula:

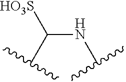 [SMILES: *NC(*)S(=O)(=O)O]

or a pharmaceutically acceptable salt thereof, wherein the aqueous solution does not comprise a buffer; and (b) reacting the modified cytotoxic agent with the EpCAM-binding agent (e.g., an EpCAM antibody, EpCAM-binding antibody fragment thereof, or an EpCAM activatable antibody, disclosed herein) disclosed herein to form the immunoconjugate. In some embodiments, the reaction of step (a) is carried out in a mixture of an organic solvent and water. More specifically, the reaction of step (a) is carried out in a mixture of dimethyacetamide (DMA) and water. In some embodiments, the mixture of DMA and water comprises less than 60% of DMA by volume. Even more specifically, the volume ratio of DMA and water is 1:1.

In a 3rd aspect, for the methods described above or in the 1st or 2nd aspect, 0.5 to 5.0 equivalents of the bisulfite salt or 0.25 or 2.5 equivalents of the metabisulfite salt is used for every 1 equivalent of the imine-containing cytotoxic agent in the reaction of step (a). In some embodiments, 0.5 to 4.5, 0.5 to 4.0, 0.5 to 3.5.0.5 to 4.0, 0.5 to 3.5, 0.5 to 3.0, 0.5 to 2.5, 0.8 to 2.0, 0.9 to 1.8, 1.0 to 1.7, 1.1 to 1.6, or 1.2 to 1.5 equivalents of the bisulfite salt or 0.25 to 2.25, 0.25 to 2.0.0.25 to 1.75, 0.25 to 2.0, 0.25 to 1.75, 0.25 to 1.5, 0.25 to 1.25, 0.4 to 1.0, 0.45 to 0.9, 0.5 to 0.85, 0.55 to 0.8, or 0.6 to 0.75 equivalents of the metabisulfite salt is used for every 1 equivalent of the imine-containing cytotoxic agent. In other embodiments. 0.5, 0.6, 0.7, 0.8, 0.9, 1.0, 1.1, 1.2, 1.3 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 4.0, 4.5 or 5.0 equivalents of the bisulfite salt or 0.25, 0.3, 0.35, 0.4, 0.45, 0.5, 0.55, 0.6, 0.65 0.7, 0.75, 0.8, 0.85, 0.9, 0.95, 1.0, 1.05, 1.1, 1.15, 1.2, 1.25, 1.3, 1.35, 1.4, 1.45, 1.5, 1.55, 1.6, 1.65, 1.7, 1.75, 2.0, 2.25 or 2.5 equivalents of the metabisulfite salt is used for every 1 equivalent of the imine-containing cytotoxic agent. In yet other embodiments, 1.4 equivalents of the bisulfite salt or 0.7 equivalent of the metabisulfite salt is used for every 1 equivalent of the imine-containing cytotoxic agent. In other embodiments, 1.2 equivalents of the bisulfite salt or 0.6 equivalent of the metabisulfite salt is used for every 1 equivalent of the imine-containing cytotoxic agent. As used herein, a specific equivalent means the specific value ±0.05.

In a 4th aspect, for methods described above, the reaction of step (a) is carried out at a pH of 2.9 to 3.7 and 1.0 to 1.8 equivalents of the bisulfite salt or 0.5 to 0.9 equivalents of the metabisulfite salt is reacted with 1 equivalent of the imine-containing cytotoxic agent. In some embodiments, the reaction of step (a) is carried out at a pH of 3.1 to 3.5 and 1.1 to 1.6 equivalents of the bisulfite salt or 0.55 to 0.8 equivalents of the metabisulfite salt is reacted with 1 equivalent of the imine-containing cytotoxic agent. In other embodiments, the reaction of step (a) is carried out at a pH of 3.2 to 3.4 and 1.3 to 1.5 equivalents of the bisulfite salt or 0.65 to 0.75 equivalents of the metabisulfite is reacted with 1 equivalent of the imine-containing cytotoxic agent. In other embodiments, the reaction of step (a) is carried out at a pH of 3.3 and 1.4 equivalents of the bisulfite salt or 0.7 equivalent of the metabisulfite salt is reacted with 1 equivalent of the imine-containing cytotoxic agent. In yet other embodiments, the reaction of step (a) is carried out at a pH of 3.3 and 1.4 equivalents of sodium bisulfite is reacted with 1 equivalent of the imine-containing cytotoxic agent.

In a 5th aspect, for the methods described above or in the 1st, 2nd, 3rd or 4th aspect, the reaction of step (a) is carried out in a mixture of an organic solvent and water. Any suitable organic solvent can be used. Exemplary organic solvents include, but are not limited to, alcohols (e.g., methanol, ethanol, propanol, etc.), dimethylformamide (DMF), dimethylsulfoxide (DMSO), acetonitrile, acetone, methylene chloride, etc. In some embodiments, the organic solvent is miscible with water. In other embodiments, the organic solvent is not miscible with water, i.e., the reaction of step (a) is carried out in a biphasic solution. In some embodiments, the organic solvent is dimethylacetamide (DMA). The organic solvent (e.g., DMA) can be present in the amount of 1%-99%, 1-95%, 10-80%, 20-70%, 30-70%, 1-60%, 5-60%, 10-60%, 20-60%, 30-60%, 40-60%, 45-55%, 10-50%, or 20-40%, by volume of the total volume of water and the organic solvent. In some embodiments, the reaction of step (a) is carried out in a mixture of DMA and water, wherein the volume ratio of DMA and water is 1:1.

In a 6th aspect, for the methods described above or in the 1st, 2nd, 3rd, 4th or 5th aspect, the reaction of step (a) can be carried out at any suitable temperature. In some embodiments, the reaction is carried out at a temperature from 0 °C to 50 °C, from 10 °C to 50 °C, from 10 °C to 40 °C, or from 10 °C to 30 °C. In other embodiments, the reaction is carried out at a temperature from 15 °C to 30 °C, from 20 °C to 30 °C, from 15 °C to 25 °C, from 16 °C to 24 °C, from 17 °C to 23 °C, from 18 °C to 22 °C or from 19 °C to 21 °C. In yet other embodiments, the reaction can be carried out at 15 °C, 16 °C, 17 °C, 18 °C, 19 °C, 20 °C, 21 °C, 22 °C, 23 °C, 24 °C or 25 °C. In some embodiments, the reaction can be carried out from 0 °C to 15 °C, from 0 °C to 10 °C, from 1 °C to 10 °C, 5 °C to 15 °C, or from 5 °C to 10 °C.

In a 7th aspect, for the methods described above or in the 1st, 2nd, 3rd, 4th, 5th or 6th aspect, the reaction of step (a) is carried out for 1 minute to 48 hours, 5 minutes to 36 hours, 10 minutes to 24 hours, 30 minutes to 24 hours, 30 minutes to 20 hours, 1 hour to 20 hours, 1 hour to 15 hours, 1 hour to 10 hours, 2 hours to 10 hours, 3 hours to 9 hours, 3 hours to 8 hours, 4 hours to 6 hours, or 1 hour to 4 hours. In some embodiments, the reaction is allowed to proceed for 4, to 6 hours. In other embodiments, the reaction is allowed to proceed for 10 minutes, 15 minutes, 20 minutes, 30 minutes, 1 hours, 2 hours, 3 hours, 4 hours, 5 hours, 6 hours, 7 hours, 8 hours, 9 hours, 10 hours, 11 hours, 12 hours, 13 hours, 14 hours, 15 hours, etc. In other embodiments, the reaction is allowed to proceed for 4, hours. In yet other embodiments, the reaction is allowed to proceed for 2 hours.

In a 8th aspect, for the methods disclosed herein or in the 1st, 2nd, 3rd, 4th, 5th, 6th or 7th aspect, the reaction of step (b) is carried out at a pH of 4 to 9. In some embodiments, the reaction of step (b) is carried out at a pH of 4.5 to 8.5, 5 to 8.5, 5 to 8, 5 to 7.5, 5 to 7, 5 to 6.5, or 5.5 to 6.5. In other embodiments, the reaction of step (b) is carried out at pH 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0.

In some embodiments, for the methods described above or in the 1st, 2nd, 3rd, 4th, 5th, 6th, 7th or 8th aspect, the reaction of step (b) is carried out in an aqueous solution comprising a mixture of water and an organic solvent. Any suitable organic solvent described above can be used. More specifically, the organic solvent is DMA. In some embodiments, the aqueous solution comprises less than 50%, less than 40%, less than 30%, less than 25%, less than 20%, less than 15%, less than 10%, less than 5%, less than 3%, less than 2%, or less than 1% of the organic solvent (e.g., DMA) by volume.

In some embodiments, for the methods disclosed herein or in the 1st, 2nd, 3rd, 4th, 5th, 6th, 7th or 8th aspect, the bisulfite salt is sodium or potassium bisulfite and the metabisulfite salt is sodium or potassium metabisulfite. In a specific embodiment, the bisulfite salt is sodium bisulfite and the metabisulfite salt is sodium metabisulfite.

In some embodiments, for the methods disclosed herein or in the 1st, 2nd, 3rd, 4th, 5th, 6th, 7th or 8th aspect, the modified cytotoxic agent is not purified before reacting with the cell-binding agent in step (b). Alternatively, the modified cytotoxic agent is purified before reacting with the cell-binding agent in step (b). Any suitable methods disclosed herein can be used to purify the modified cytotoxic agent.

In some embodiments, for the methods described above, the reaction of step (a) results in no substantial sulfonation of the maleimide group. In some embodiments, less than 50%, 40%, 30%, 20%, 10%, 9%, 8%, 7%, 6%, 5%, 4%, 3%, 2%, or 1% of the maleimide group is sulfonated. The percentage of maleimide sulfonation is equal to the total amount of the maleimide-sulfonated cytotoxic agent (the cytotoxic agent having sulfonation on the maleimide only) and the di-sulfonated cytotoxic agent (the cytotoxic agent having sulfonation on both the maleimide and the imine moieties) divided by the starting amount of the imine-containing cytotoxic agent before its reaction with the bisulfite salt or the metabisulfite salt.

In some embodiments, the immunoconjugates prepared by any methods described above is subject to a purification step. In this regard, the immunoconjugate can be purified from the other components of the mixture using tangential flow filtration (TFF), non-adsorptive chromatography, adsorptive chromatography, adsorptive filtration, selective precipitation, or any other suitable purification process, as well as combinations thereof.

In some embodiments, the immunoconjugate is purified using a single purification step (e.g., TFF). In some embodiments, the conjugate is purified and exchanged into the appropriate formulation using a single purification step (e.g., TFF). In other embodiments, the immunoconjugate is purified using two sequential purification steps. For example, the immunoconjugate can be first purified by selective precipitation, adsorptive filtration, absorptive chromatography or non-absorptive chromatography, followed by purification with TFF. One of ordinary skill in the art will appreciate that purification of the immunoconjugate enables the isolation of a stable conjugate comprising the cell-binding agent chemically coupled to the cytotoxic agent.

Any suitable TFF systems may be utilized for purification, including a Pellicon type system (Millipore, Billerica, Mass.), a Sartocon Cassette system (Sartorius AG, Edgewood, N.Y.), and a Centrasette type system (Pall Corp., East Hills, N.Y.)

Any suitable adsorptive chromatography resin may be utilized for purification. Particular adsorptive chromatography resins include hydroxyapatite chromatography, hydrophobic charge induction chromatography (HCIC), hydrophobic interaction chromatography (HIC), ion exchange chromatography, mixed mode ion exchange chromatography, immobilized metal affinity chromatography (IMAC), dye ligand chromatography, affinity chromatography, reversed phase chromatography, and combinations thereof. Examples of suitable hydroxyapatite resins include ceramic hydroxyapatite (CHT Type I and Type II, Bio-Rad Laboratories, Hercules, Calif.), HA Ultrogel hydroxyapatite (Pall Corp., East Hills, N.Y.), and ceramic fluoroapatite (CFT Type I and Type II, Bio-Rad Laboratories, Hercules, Calif.). An example of a suitable HCIC resin is MEP Hypercel resin (Pall Corp., East Hills, N.Y.). Examples of suitable HIC resins include Butyl-Sepharose, Hexyl-Sepharose, Phenyl-Sepharose, and Octyl Sepharose resins (all from GE Healthcare, Piscataway, N.J.), as well as Macro-prep Methyl and Macro-Prep t Butyl resins (Biorad Laboratories, Hercules, Calif.). Examples of suitable ion exchange resins include SP-Sepharose, CM-Sepharose, and Q-Sepharose resins (all from GE Healthcare, Piscataway, N.J.), and Unosphere S resin (Bio-Rad Laboratories, Hercules, Calif.). Examples of suitable mixed mode ion exchangers include Bakerbond CBAx resin (JT Baker, Phillipsburg N.J.) Examples of suitable IMAC resins include Chelating Sepharose resin (GE Healthcare, Piscataway, N.J.) and Profinity IMAC resin (Bio-Rad Laboratories, Hercules, Calif.). Examples of suitable dye ligand resins include Blue Sepharose resin (GE Healthcare, Piscataway, N.J.) and Affi-gel Blue resin (Bio-Rad Laboratories, Hercules, Calif.). Examples of suitable affinity resins include Protein A Sepharose resin (e.g., MabSelect, GE Healthcare, Piscataway, N.J.), where the cell-binding agent is an antibody, and lectin affinity resins, e.g., Lentil Lectin Sepharose resin (GE Healthcare, Piscataway, N.J.), where the cell-binding agent bears appropriate lectin binding sites. Alternatively an antibody specific to the cell-binding agent may be used. Such an antibody can be immobilized to, for instance, Sepharose 4 Fast Flow resin (GE Healthcare, Piscataway, N.J.). Examples of suitable reversed phase resins include C4, C8, and C18 resins (Grace Vydac, Hesperia, Calif.).

Any suitable non-adsorptive chromatography resin may be utilized for purification. Examples of suitable non-adsorptive chromatography resins include, but are not limited to, SEPHADEX™ G-25, G-50, G-100, SEPHACRYL™ resins (e.g., S-200 and S-300), SUPERDEX™ resins (e.g., SUPERDEX™ 75 and SUPERDEX™ 200), BIO-GEL® resins (e.g., P-6, P-10, P-30, P-60, and P-100), and others known to those in the art.

The immunoconjugates comprising an EpBA covalently linked to a maytansinoid compound described in the 3^{rd} embodiment herein can be prepared according to any suitable methods known in the art.

In some embodiments, the immunoconjugates prepared by any methods described above is subject to a purification step. In this regard, the immunoconjugate can be purified from the other components of the mixture using tangential flow filtration (TFF), non-adsorptive chromatography, adsorptive chromatography, adsorptive filtration, selective precipitation, or any other suitable purification process, as well as combinations thereof.

In some embodiments, the immunoconjugate is purified using a single purification step (e.g., TFF). In some embodiments, the conjugate is purified and exchanged into the appropriate formulation using a single purification step (e.g., TFF). In other embodiments of the invention, the immunoconjugate is purified using two sequential purification steps. For example, the immunoconjugate can be first purified by selective precipitation, adsorptive filtration, absorptive chromatography or non-absorptive chromatography, followed by purification with TFF. One of ordinary skill in the art will appreciate that purification of the immunoconjugate enables the isolation of a stable conjugate comprising the cell-binding agent chemically coupled to the cytotoxic agent.

Any suitable TFF systems may be utilized for purification, including a Pellicon type system (Millipore, Billerica, Mass.), a Sartocon Cassette system (SartonusAG, Edgewood, N.Y.), and a Centrasette type system (Pall Corp., East Hills, N.Y.)

Any suitable adsorptive chromatography resin may be utilized for purification. Particular adsorptive chromatography resins include hydroxyapatite chromatography, hydrophobic charge induction chromatography (HCIC), hydrophobic interaction chromatography (HIC), ion exchange chromatography, mixed mode ion exchange chromatography, immobilized metal affinity chromatography (IMAC), dye ligand chromatography, affinity chromatography, reversed phase chromatography, and combinations thereof. Examples of suitable hydroxyapatite resins include ceramic hydroxyapatite (CHT Type I and Type II, Bio-Rad Laboratories, Hercules, Calif.), HA Ultrogel hydroxyapatite (Pall Corp., East Hills, N.Y.), and ceramic fluoroapatite (CFT Type I and Type II, Bio-Rad Laboratories, Hercules, Calif.). An example of a suitable HCIC resin is MEP Hypercel resin (Pall Corp., East Hills, N.Y.). Examples of suitable HIC resins include Butyl-Sepharose, Hexyl-Sepharose, Phenyl-Sepharose, and Octyl Sepharose resins (all from GE Healthcare, Piscataway, N.J.), as well as Macro-prep Methyl and Macro-Prep t-Butyl resins (Biorad Laboratories, Hercules, Calif.). Examples of suitable ion exchange resins include SP-Sepharose, CM-Sepharose, and Q-Sepharose resins (all from GE Healthcare, Piscataway, N.J.), and Unosphere S resin (Bio-Rad Laboratories, Hercules, Calif.). Examples of suitable mixed mode ion exchangers include Bakerbond ABx resin (JT Baker, Phillipsburg N.J.) Examples of suitable IMAC resins include Chelating Sepharose resin (GE Healthcare, Piscataway, N.J.) and Profinity IMAC resin (Bio-Rad Laboratories, Hercules, Calif.). Examples of suitable dye ligand resins include Blue Sepharose resin (GE Healthcare, Piscataway, N.J.) and Affi-gel Blue resin (Bio-Rad Laboratories, Hercules, Calif.). Examples of suitable affinity resins include Protein A Sepharose resin (e.g., MabSelect, GE Healthcare, Piscataway, N.J.), where the cell-binding agent is an antibody, and lectin affinity resins, e.g. Lentil Lectin Sepharose resin (GE Healthcare, Piscataway, N.J.), where the cell-binding agent bears appropriate lectin binding sites. Alternatively an antibody specific to the cell-binding agent may be used. Such an antibody can be immobilized to, for instance, Sepharose 4 Fast Flow resin (GE Healthcare, Piscataway, N.J.). Examples of suitable reversed phase resins include C4, C8, and C18 resins (Grace Vydac, Hesperia, Calif.).

Any suitable non-adsorptive chromatography resin may be utilized for purification. Examples of suitable non-adsorptive chromatography resins include, but are not limited to, SEPHADEX™ G-25, G-50, G-100, SEPHACRYL™ resins (e.g., S-200 and S-300), SUPERDEX™ resins (e.g., SUPERDEX™ 75 and SUPERDEX™ 200), BIO-GEL® resins (e.g., P-6, P-10, P-30, P-60, and P-100), and others known to those of ordinary skill in the art.

### 6.6.1 Immunoconjugates Produced by the Methods Provided

Also provided herein are immunoconjugates produced by a method described herein.

In some embodiments, provided herein are immunoconjugates obtainable by a method comprising: (a) mixing an activatable EpCAM antibody consisting of a heterotetramer of two heavy chains having the amino acid sequence of SEQ ID NO:103 and two light chains with the amino acid sequence of SEQ ID NO:179 with mixture of EPPS buffer and EDTA solution to form an activatable EpCAM reaction mixture; (b) adding a TCEP solution to the activatable EpCAM reaction mixture to form a reduced activatable EpCAM reaction mixture; (c) mixing the reduced activatable EpCAM reaction mixture with a camptothecin linker-toxin stock solution comprising a linker-toxin having the structure

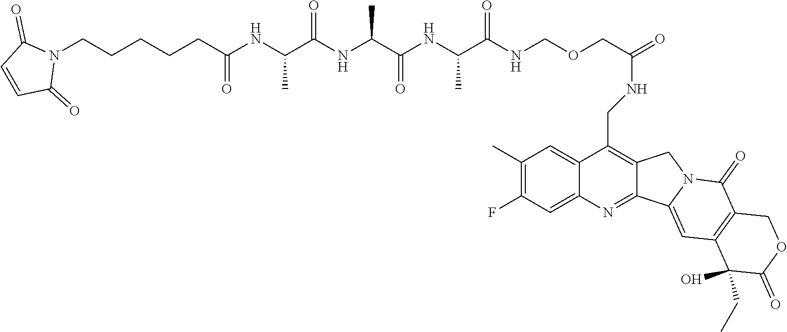 [SMILES: CC[C@@]1(O)C(=O)OCc2c1cc1n(c2=O)Cc2c-1nc1cc(F)c(C)cc1c2CNC(=O)COCNC(=O)[C@H](C)NC(=O)[C@H](C)NC(=O)[C@H](C)NC(=O)CCCCCN1C(=O)C=CC1=O]

and (d) terminating the conjugation process. In some embodiments, the method further comprises preparing the camptothecin linker-toxin stock solution by mixing the linker-toxin with anhydrous DMA. In particular embodiments, the linker-toxin and DMA are vortexed until all solids are dissolved.

In some embodiments, provided herein are immunoconjugates obtainable by the method described in Example 7.4.1.

### 6.7 Therapeutic Applications

Also included are methods for inhibiting the growth of cells expressing EpCAM (e.g., human EpCAM or cynomolgous EpCAM). As provided herein, the disclosed EpCAM immunoconjugates have the ability to bind EpCAM present on the surface of a cell (e.g., a human cell or a cynomolgous cell) and mediate cell killing. In particular embodiments, the immunoconjugates comprise a cytotoxic payload. e.g., a camptothecin derivative linker-payload, are internalized and mediate cell killing via the activity of the cytotoxic payload. Such cell killing activity may be augmented by the immunoconjugate inducing antibody-dependent cell-mediated cytotoxicity (ADCC) and/or complement dependent cytotoxicity (CDC).

As used herein the terms “inhibit” and “inhibiting” include any inhibitory effect on cell growth, including cell death. The inhibitory effects include temporary effects, sustained effects and permanent effects.

The therapeutic applications provided herein include methods of treating a subject having a disease. The diseases treated with the provided methods are those characterized by EpCAM expression (e.g., EpCAM overexpression). Such diseases include for example, breast cancer, lung cancer, non-small cell lung cancer, stomach cancer, prostate cancer, ovarian cancer, colorectal cancer, colon cancer, esophageal cancer, tracheal cancer, gastric cancer, bladder cancer, uterine cancer, rectal cancer or cancer of the small intestine, pancreatic cancer, head and neck cancer, endometrial cancer, epithelial cancer, or metastases associated therewith. In some embodiments, the cancer is colorectal cancer. In some embodiments, the cancer is gastric cancer. In some embodiments, the cancer is head and neck cancer. In some embodiments, the cancer is breast cancer. The skilled artisan will understand that the methods of the present disclosure may also be used to treat other diseases yet to be described but characterized by the expression (e.g., overexpression) of EpCAM.

The disclosed EpCAM immunoconjugates are useful in the treatment cancers expressing EpCAM. In some embodiments, the cancer is an epithelial or squamous cancer. In some embodiments, the cancer is breast cancer, lung cancer, non-small cell lung cancer, stomach cancer, prostate cancer, ovarian cancer, colorectal cancer, colon cancer, esophageal cancer, tracheal cancer, gastric cancer, bladder cancer, uterine cancer, rectal cancer or cancer of the small intestine, pancreatic cancer, head and neck cancer, endometrial cancer, or metastases associated therewith.

The therapeutic applications provided herein can also be practiced in vitro and ex vivo.

The disclosure also provides therapeutic applications of the disclosed EpCAM immunoconjugates wherein the antibodies, antibody fragments, activatable antibodies, or conjugates are administered to a subject, in a pharmaceutically acceptable dosage form. They can be administered intravenously as a bolus or by continuous infusion over a period of time, by intramuscular, subcutaneous, parenteral, intra-articular, intrasynovial, intrathecal, oral, topical, or inhalation routes. They may also be administered by intratumoral, peritumoral, intralesional, or perilesional routes, to exert local as well as systemic therapeutic effects.

Also provided are methods of treating, preventing and/or delaying the onset or progression of, or alleviating a symptom associated with aberrant expression and/or activity of EpCAM in a subject using EpCAM immunoconjugates that bind and neutralize or otherwise inhibit at least one biological activity of EpCAM and/or EpCAM-mediated signaling.

In some embodiments, the disclosure provides methods of treating, preventing and/or delaying the onset or progression of, or alleviating a symptom associated with the presence, growth, proliferation, metastasis, and/or activity of cells which are expressing EpCAM or aberrantly expressing EpCAM in a subject using EpCAM immunoconjugates that bind, target, neutralize, kill, or otherwise inhibit at least one biological activity of cells which are expressing or aberrantly expressing EpCAM. The disclosure also provides methods of treating, preventing and/or delaying the onset or progression of, or alleviating a symptom associated with the presence, growth, proliferation, metastasis, and/or activity of cells which are expressing EpCAM in a subject using EpCAM immunoconjugates that bind, target, neutralize, kill, or otherwise inhibit at least one biological activity of cells which are expressing EpCAM.

The disclosure also provides methods of treating, preventing and/or delaying the onset or progression of, or alleviating a symptom associated with the presence, growth, proliferation, metastasis, and/or activity of cells which are aberrantly expressing EpCAM in a subject using EpCAM immunoconjugates that bind, target, neutralize, kill, or otherwise inhibit at least one biological activity of cells which are aberrantly expressing EpCAM.

The disclosure provides methods of preventing, delaying the progression of, treating, alleviating a symptom of, or otherwise ameliorating an EpCAM mediated disease in a subject by administering a therapeutically effective amount of an EpCAM immunoconjugate disclosed herein to a subject in need thereof.

The disclosure also provides methods of preventing, delaying the progression of, treating, alleviating a symptom of, or otherwise ameliorating cancer (e.g., epithelial cancer and metastases thereof) in a subject by administering a therapeutically effective amount of an EpCAM immunoconjugate disclosed herein to a subject in need thereof. EpCAM is known to be expressed in a variety of cancers, including most cancers (and metastases) of epithelial origin.

In some embodiments, the cancer is an epithelial or squamous cancer.

In some embodiments, the cancer is head and neck cancer, breast cancer, lung cancer, non-small cell lung cancer, stomach cancer, prostate cancer, ovarian cancer, colorectal cancer, colon cancer, esophageal cancer, tracheal cancer, gastric cancer, bladder cancer, uterine cancer, endometrial cancer, rectal cancer, pancreatic cancer, or cancer of the small intestine.

In some embodiments, the cancer is breast cancer, lung cancer, stomach cancer, colorectal cancer, colon cancer, rectal cancer, cancer of the small intestine, ovarian cancer, gastric cancer, or esophageal cancer.

In some embodiments, the cancer is ovarian cancer, uterine cancer, gastric cancers, pancreatic cancer, or colorectal cancer.

In some embodiments, the cancer is ovarian cancer.

In some embodiments, the cancer is uterine cancer.

In some embodiments, the cancer is gastric cancer.

In some embodiments, the cancer is pancreatic cancer.

In some embodiments, the cancer is colorectal cancer.

In some embodiments, the cancer is head and neck cancer.

In some embodiments, the cancer is breast cancer. In certain embodiments, the cancer is triple negative breast cancer.

In some embodiments, the cancer is lung cancer. In some embodiments, the lung cancer is non-small cell lung cancer. In some embodiments, the non-small cell lung cancer is non-squamous non-small cell lung cancer.

An EpCAM immunoconjugate used in any of the embodiments of these methods and uses can be administered at any stage of the disease. For example, such an EpCAM immunoconjugate can be administered to a patient suffering cancer of any stage, from early to metastatic. The terms subject and patient are used interchangeably herein.

In some embodiments, the subject is a mammal, such as a human, non-human primate, companion animal (e.g., cat, dog, horse), farm animal, work animal, or zoo animal. In some embodiments, the subject is a human. In some embodiments, the subject is a companion animal. In some embodiments, the subject is an animal in the care of a veterinarian.

The EpCAM immunoconjugates and therapeutic formulations thereof are administered to a subject suffering from or susceptible to a disease or disorder associated with aberrant EpCAM expression and/or activity, such as cancer. A subject suffering from or susceptible to a disease or disorder associated with aberrant EpCAM expression and/or activity is identified using any of a variety of methods known in the art. For example, subjects suffering from cancer or other neoplastic condition are identified using any of a variety of clinical and/or laboratory tests such as, physical examination and blood, urine and/or stool analysis to evaluate health status. For example, subjects suffering from inflammation and/or an inflammatory disorder are identified using any of a variety of clinical and/or laboratory tests such as physical examination and/or bodily fluid analysis, e.g., blood, urine and/or stool analysis, to evaluate health status.

Administration of an EpCAM immunoconjugates to a patient suffering from a disease or disorder associated with aberrant EpCAM expression and/or activity (e.g., a cancer such as a carcinoma) is considered successful if any of a variety of laboratory or clinical objectives is achieved. For example, administration of an EpCAM immunoconjugate to a patient suffering from a disease or disorder associated with aberrant EpCAM expression and/or activity is considered successful if one or more of the symptoms associated with the disease or disorder is alleviated, reduced, inhibited or does not progress to a further, i.e., worse, state. Administration of an EpCAM immunoconjugate to a patient suffering from a disease or disorder associated with aberrant EpCAM expression and/or activity is considered successful if the disease or disorder enters remission or does not progress to a further, i.e., worse, state.

In some embodiments, the EpCAM immunoconjugate and therapeutic formulations thereof are administered to a subject suffering from or susceptible to a disease or disorder, such as subjects suffering from cancer or other neoplastic condition, wherein the subject's diseased cells are expressing EpCAM. In some embodiments, the diseased cells are associated with aberrant EpCAM expression and/or activity. In some embodiments, the diseased cells are associated with normal EpCAM expression and/or activity. A subject suffering from or susceptible to a disease or disorder wherein the subject's diseased cells express EpCAM is identified using any of a variety of methods known in the art. For example, subjects suffering from cancer or other neoplastic condition are identified using any of a variety of clinical and/or laboratory tests such as, physical examination and blood, urine and/or stool analysis to evaluate health status. For example, subjects suffering from inflammation and/or an inflammatory disorder are identified using any of a variety of clinical and/or laboratory tests such as physical examination and/or bodily fluid analysis, e.g., blood, urine and/or stool analysis, to evaluate health status.

In some embodiments, the EpCAM immunoconjugate and therapeutic formulations thereof are administered to a subject suffering from or susceptible to a disease or disorder associated with cells expressing EpCAM or the presence, growth, proliferation, metastasis, and/or activity of such cells, such as subjects suffering from cancer or other neoplastic conditions. In some embodiments, the cells are associated with aberrant EpCAM expression and/or activity. In some embodiments, the cells are associated with normal EpCAM expression and/or activity. A subject suffering from or susceptible to a disease or disorder associated with cells that express EpCAM is identified using any of a variety of methods known in the art. For example, subjects suffering from cancer or other neoplastic condition are identified using any of a variety of clinical and/or laboratory tests such as, physical examination and blood, urine and/or stool analysis to evaluate health status. For example, subjects suffering from inflammation and/or an inflammatory disorder are identified using any of a variety of clinical and/or laboratory tests such as physical examination and/or bodily fluid analysis, e.g., blood, urine and/or stool analysis, to evaluate health status.

Administration of an EpCAM immunoconjugate to a patient suffering from a disease or disorder associated with cells expressing EpCAM (e.g., a cancer such as a carcinoma) is considered successful if any of a variety of laboratory or clinical objectives is achieved. For example, administration of an EpCAM immunoconjugate to a patient suffering from a disease or disorder associated with cells expressing EpCAM is considered successful if one or more of the symptoms associated with the disease or disorder is alleviated, reduced, inhibited or does not progress to a further, i.e., worse, state. Administration of an EpCAM immunoconjugate to a patient suffering from a disease or disorder associated with cells expressing EpCAM is considered successful if the disease or disorder enters remission or does not progress to a further, i.e., worse, state.

The disclosure provides conjugated EpCAM antibodies that are useful in methods of treating, preventing, delaying the progression of, ameliorating and/or alleviating a symptom of a disease or disorder associated with aberrant EpCAM expression and/or activity. For example, the conjugated EpCAM activatable antibodies are used in methods of treating, preventing, delaying the progression of, ameliorating and/or alleviating a symptom of a cancer or other neoplastic condition.

The disclosure provides conjugated EpCAM antibodies that are useful in methods of treating, preventing, delaying the progression of, ameliorating and/or alleviating a symptom of a disease or disorder associated with cells expressing EpCAM. In some embodiments, the cells are associated with aberrant EpCAM expression and/or activity. In some embodiments, the cells are associated with normal EpCAM expression and/or activity. For example, the EpCAM activatable antibodies can be used in methods of treating, preventing, delaying the progression of, ameliorating and/or alleviating a symptom of a cancer or other neoplastic condition.

The disclosure provides EpCAM antibodies and antibody fragments, and/or conjugated EpCAM activatable antibodies that are useful in methods of treating, preventing, delaying the progression of, ameliorating and/or alleviating a symptom of a disease or disorder in which diseased cells express EpCAM. In some embodiments, the diseased cells are associated with aberrant EpCAM expression and/or activity. In some embodiments, the diseased cells are associated with normal EpCAM expression and/or activity. For example, the EpCAM conjugated activatable antibodies are used in methods of treating, preventing, delaying the progression of, ameliorating and/or alleviating a symptom of a cancer or other neoplastic condition.

In some embodiments, the conjugated EpCAM antibodies or activatable antibodies provided herein have an excellent therapeutic index. In certain embodiments, the conjugated EpCAM antibodies or activatable antibodies have a therapeutic index of about 3 to about 6. In some embodiments, the conjugated EpCAM antibodies or activatable antibodies have a therapeutic index of about 3. In some embodiments, the conjugated EpCAM antibodies or activatable antibodies have a therapeutic index of about 4. In some embodiments, the conjugated EpCAM antibodies or activatable antibodies have a therapeutic index of about 5. In some embodiments, the conjugated EpCAM antibodies or activatable antibodies have a therapeutic index of about 6. The therapeutic index of the conjugated EpCAM antibodies or activatable antibodies described herein is significantly improve relative to that of other conjugated EpCAM antibodies or activatable antibodies. For example, huEpCAM23Gv4.2-GMBS-DM21L (see, e.g., Example 7.4.5) has a therapeutic index of about 1.

The EpCAM antibodies or activatable antibodies provided herein have relatively low systemic toxicities. In cynomolgus monkeys, for example, EpCAM-CPT66 was tolerated up to 60 mg/kg (see Example 7.7). This was in stark contrast to EpCAM-DM21, an immunoconjugate comprising the same EpCAM activatable antibody as EpCAM-CPT66 but with the auristatin-based DM21 linker-payload, which was only tolerated up to 6 mg/kg. Tolerability of EpCAM-CPT66 is also favorable when compared to fam-trastuzumab deruxtecan-nxki, where the HNSTD was determined to be 30 mg/kg in cynomolgus monkeys.

### 6.7.1 Pharmaceutical Compositions

The provided compositions include bulk drug compositions useful in the manufacture of pharmaceutical compositions (e.g., impure or non-sterile compositions) and pharmaceutical compositions (i.e., compositions that are suitable for administration to a subject or patient) that can be used in the preparation of unit dosage forms. Such compositions comprise a prophylactically or therapeutically effective amount of the provided immunoconjugates and a pharmaceutically acceptable carrier.

In some embodiments, provided compositions comprise a prophylactically or therapeutically effective amount of a disclosed immunoconjugate and a pharmaceutically acceptable carrier.

In a specific embodiment, the term “pharmaceutically acceptable” means approved by a regulatory agency of the Federal or a state government or listed in the US Pharmacopeia or other generally recognized pharmacopeia for use in animals, and more particularly in humans. The term “carrier” refers to a diluent, adjuvant (e.g., Freund's adjuvant (complete and incomplete), excipient, or vehicle with which the therapeutic is administered. Generally, the ingredients of the compositions provide herein are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The disclosure also provides a pharmaceutical pack or kit comprising one or more containers filled with an immunoconjugate provided herein, alone or with such pharmaceutically acceptable carrier. The disclosure also provides a pharmaceutical pack or kit comprising one or more containers filled with one or more of the ingredients of the pharmaceutical compositions provided herein. Optionally associated with such container(s) can be a notice in the form prescribed by a governmental agency regulating the manufacture, use or sale of pharmaceuticals or biological products, which notice reflects approval by the agency of manufacture, use or sale for human administration.

The disclosure provides kits that can be used in the above methods. A kit can comprise any of the immunoconjugates disclosed herein.

### 6.7.2 Methods of Administration

The disclosed compositions may be provided for the treatment, prophylaxis, and amelioration of one or more symptoms associated with a disease, disorder by administering to a subject a therapeutically effective amount an immunoconjugate provided herein. In a particular aspect, such compositions are substantially purified (i.e., substantially free from substances that limit its effect or produce undesired side effects). In a specific embodiment, the subject is an animal, in some instances a mammal such as non-primate (e.g., bovine, equine, feline, canine, rodent, etc.) or a primate (e.g., monkey such as, a cynomolgus monkey, human, etc.). In a particular embodiment, the subject is a human.

Methods of administering an immunoconjugate provided herein include, but are not limited to, parenteral administration (e.g., intradermal, intramuscular, intraperitoneal, intravenous and subcutaneous), epidural, and mucosal (e.g., intranasal and oral routes). In a specific embodiment, the immunoconjugates provided herein are administered intramuscularly, intravenously, or subcutaneously. The compositions may be administered by any convenient route, for example, by infusion or bolus injection, and may be administered together with other biologically active agents. Administration can be systemic or local.

The disclosure also provides that preparations of the disclosed immunoconjugates are packaged in a hermetically sealed container such as an ampoule or sachette indicating the quantity of the molecule. In one embodiment, such molecules are supplied as a dry sterilized lyophilized powder or water free concentrate in a hermetically sealed container and can be reconstituted, e.g., with water or saline to the appropriate concentration for administration to a subject. In some embodiments, the immunoconjugates are supplied as a dry sterile lyophilized powder in a hermetically sealed container.

The lyophilized preparations of the immunoconjugates provided herein should be stored at between 2 °C and 8 °C in their original container and the molecules should be administered within 12 hours, in some instances within 6 hours, within 5 hours, within 3 hours, or within 1 hour after being reconstituted. In an alternative embodiment, such molecules are supplied in liquid form in a hermetically sealed container indicating the quantity and concentration of the molecule, fusion protein, or conjugated molecule. In some embodiments, such immunoconjugates when provided in liquid form are supplied in a hermetically sealed container.

As used herein, an “therapeutically effective amount” of a pharmaceutical composition is an amount sufficient to effect beneficial or desired results including, without limitation, clinical results such as decreasing a symptom of cancer (e.g., the proliferation, of cancer cells, tumor presence, tumor metastases, etc.), thereby increasing the quality of life of those suffering from the disease, decreasing the dose of other medications required to treat the disease, enhancing the effect of another medication such as via targeting and/or internalization, delaying the progression of the disease, and/or prolonging survival of individuals.

A therapeutically effective amount can be administered in one or more administrations. For purposes of this disclosure, a therapeutically effective amount of drug, compound, or pharmaceutical composition is an amount sufficient to reduce the proliferation of (or the effect of) viral presence and to reduce and/or delay the development of the viral disease, either directly or indirectly.

### 6.8 Diagnostic and Research Applications

In addition to the therapeutic uses of the immunoconjugates discussed herein, the immunoconjugates provided herein can be employed in many known diagnostic and research applications. The provided EpCAM immunoconjugates may be used, for example, in the purification, detection, and targeting of EpCAM, included in both in vitro and in vivo diagnostic methods. For example, the antibodies and/or fragments may be used in immunoassays for qualitatively and quantitatively measuring levels of EpCAM (e.g., human EpCAM or cynomolgous EpCAM) expressed by cells in biological samples. See, e.g., Harlow et al., Antibodies: A Laboratory Manual (Cold Spring Harbor Laboratory Press, 2nd ed. 1988), the entire contents of which is herein incorporated by reference.

The provided EpCAM immunoconjugates may be used in, for example, competitive binding assays, direct and indirect sandwich assays, and immunoprecipitation assays (Zola, Monoclonal Antibodies: A Manual of Techniques, pp. 147-158 (CRC Press, Inc., 1987)).

Detectably labeling an EpCAM immunoconjugates can be accomplished by linkage to an enzyme for use in an enzyme immunoassay (EIA), or enzyme-linked immunosorbent assay (ELISA). The linked enzyme reacts with the exposed substrate to generate a chemical moiety which can be detected, for example, by spectrophotometric, fluorometric or by visual means. Enzymes which can be used to detectably label for example the disclosed EpCAM immunoconjugates, include, but are not limited to, malate dehydrogenase, staphylococcal nuclease, delta-S-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase.

By radioactively labeling the EpCAM immunoconjugates, it is possible to detect EpCAM through the use of a radioimmunoassay (RIA) (see, e.g., Work, et al., Laboratory Techniques and Biochemistry in Molecular Biology, North Holland Publishing Company, N.Y. (1978)). The radioactive isotope can be detected by such means as the use of a gamma counter or a scintillation counter or by autoradiography. Isotopes which are particularly useful for the purpose of the present disclosure are: ³H, ¹²⁵I, ¹³¹I, ³⁵S, ¹⁴C, and, in some instances, ¹²⁵I.

It is also possible to label the EpCAM immunoconjugates with a fluorescent compound. When the fluorescent labeled antibody, antibody fragment, or activatable antibody, is exposed to light of the proper wave length, its presence can then be detected due to fluorescence. Among the most commonly used fluorescent labelling compounds are fluorescein isothiocyanate, rhodamine, phycoerythrin, phycocyanin, allophycocyanin, o-phthaldehyde and fluorescamine.

The EpCAM immunoconjugates can also be detectably labeled using fluorescence-emitting metals such as 125Eu, or others of the lanthanide series. These metals can be attached to the EpCAM antibodies, EpCAM-binding antibody fragments thereof, and EpCAM activatable antibodies using such metal chelating groups as diethylenetriaminepentaacetic acid (DTPA) or ethylenediamine-tetraacetic acid (EDTA).

In additional embodiments, the EpCAM immunoconjugates are detectably labeled by coupling to a chemiluminescent compound. The presence of the chemiluminescently labeled antibody, or antibody fragment is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester. Likewise, a bioluminescent compound can be used to label the EpCAM antibodies, EpCAM-binding antibody fragments thereof, EpCAM activatable antibodies, or derivatives thereof. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

EpCAM immunoconjugates are useful for in vivo imaging, wherein an EpCAM immunoconjugates, labeled with a detectable moiety such as a radio-opaque agent or radioisotope is administered to a subject, in some instances into the bloodstream, and the presence and location of the labeled antibody or antibody fragment in the host is assayed. This imaging technique is useful in the staging and treatment of malignancies. The EpCAM immunoconjugates may be labeled with any moiety that is detectable in a host, whether by nuclear magnetic resonance, radiology, or other detection means known in the art.

The label used according to the disclosed methods can be any detectable moiety that is capable of producing, either directly or indirectly, a detectable signal. For example, the label may be a biotin label, an enzyme label (e.g., luciferase, alkaline phosphatase, beta-galactosidase and horseradish peroxidase), a radio-label (e.g., ³H, ¹⁴C, ³²P, ³⁵S, and ¹²⁵I), a fluorophore such as fluorescent or chemiluminescent compound (e.g., fluorescein isothiocyanate, rhodamine), an imaging agent (e.g., Tc-m⁹⁹ and indium (¹¹¹In)) and a metal ion (e.g., gallium and europium).

Any method known in the art for conjugating the EpCAM immunoconjugates, to the label may be employed, including those exemplary methods described by Hunter et al., Nature 144:945 (1962); David et al., Biochemistry 13:1014 (1974): Pain et al., J. Immunol. Meth. 40:219 (1981); Nygren, Histochem. and Cytochem. 30:407 (1982).

### 7. EXAMPLES

### 7.1 Example 1. Epcam Antibodies and Fragments Thereof

Generation and selection of monoclonal antibodies against human and cynomolgus (cyno) EpCAM is described in detail in WO 2020/086665, the disclosure of which is hereby incorporated by reference in its entirety for all purposes. Generation, selection, and humanization of the EpCAM antibodies and fragments thereof of the disclosure is summarized below.

Three different immunization protocols were used to generate the monoclonal antibodies. In the first immunization protocol, wild type BALB/c female mice were injected subcutaneously three times with the cyno-EpCAM expressing 300-19 cell line, which is a BALB/c derived pre-B cell, and were then injected two times with the human-EpCAM expressing 300-19 cell line. In the second immunization protocol, wild type BALB/c female mice were injected subcutaneously four times with the NSCLC cell line H1568, and were then injected four times with cyno primary kidney epithelial cells. In the third immunization protocol, FcgammaR2b ko/ko BALB/c female mice (model #579, Taconic) were injected subcutaneously with human-EpCAM expressing 300-19 cells three times, and were then injected with cyno-EpCAM expressing 300-19 cells two times. In all three protocols, cells were prepared in PBS and injected into mice at a dose of 5×10⁶ cells/mouse/injection with two weeks interval between injections. To boost the immune response, anti GITR Ab (clone DTA-1) was injected one week after the first immunization. Three days before being sacrificed for Hybridoma generation, the immunized mice received intraperitoneal injection of another dose of the human-EpCAM expressing 300-19 cells. Hybridoma clones were prepared for antibody screening.

Other techniques of immunization and Hybridoma production can also be used, including those described in J. Langone and H. Vunakis (Eds., Methods in Enzymology, Vol. 121, Immunochemical Techniques, Part I, Academic Press, Florida); and E. Harlow and D. Lane (Antibodies: A Laboratory Manual, 1988, Cold Spring Harbor Laboratory Press, New York, NY).

Hybridoma screening was performed using a flow cytometry binding assay with human EpCAM expressing 300-19 cells and wild-type 300-19 cells. Hybridomas with positive binding to human and cyno EpCAM antigens but negative on wild type 300-19 cells were further subcloned by limiting dilution. One subclone from each Hybridoma, which showed specific binding to human and cyno EpCAM antigens, was selected for subsequent analysis.

A total of 20 fusions were conducted over the course of this investigation. 63 Hybridomas specific for human and cynomolgus EpCAM antigens were generated and 29 Hybridomas were subcloned. Stable subclones were cultured and the isotype of the monoclonal antibody was identified using commercial mouse IgG isotyping reagents.

Murine antibodies were purified and assayed for binding affinity by a flow cytometry binding assay using purified antibody mEpCAM23 and performed with HSC2 cells. The apparent K_{d} of the mEpCAM23 antibody ranged from 3.8×10⁻¹⁰ to 7.9×10⁻¹⁰. The sequence of mEpCAM23 was identified, and an mEpCAM23 clone was selected for chimerization, humanization and further evaluation.

The VL and VH chains of the EpCAM hybridomas were cloned and sequenced prior to the variable region amino acid sequences for the murine EpCAM antibodies were codon-optimized, synthesized and cloned in-frame with human IgG1 constant regions by GenScript (New Jersey) to build chimeric versions of the EpCAM antibodies.

Antibody humanization was performed using complementarity determining region (CDR) grafting procedures essentially as described in Jones et al., Nature 321: 604-608 (1986), Verhoeyen et al., Science 239:1534-1536 (1988), U.S. Pat. Nos. 5,225,539 and 5,585,089. CDR grafting generally consists of replacing the Fv framework regions (FRs) of a mouse antibody with human antibody Fv framework regions while preserving the mouse CDR residues critical for the specific antigen-binding properties of the parent antibody. Exemplary CDRs of the murine EpCAM-23 antibody following the Kabat CDR definitions are indicated in Table 13 below.

TABLE 13

Murine EpCAM-23 CDRs

Heavy Chain

| | |
|---|---|
| CDR-H1: | CDR-H1: NYYIH (SEQ ID NO: 13) |
| CDR-H2: | CDR-H2: WIYPGNVYIQYNEKFKG (SEQ ID NO: 14) |
| CDR-H3: | CDR-H3: DGPWFAY(SEQ ID NO: 15) |

Light Chain

| | |
|---|---|
| CDR-L1: | CDR-L1: RSSKSLLHSDGFTYLY (SEQ ID NO: 39) |
| CDR-L2: | CDR-L2: QTSNLAS (SEQ ID NO: 40) |
| CDR-L3: | CDR-L3: AQNLELPNT (SEQ ID NO: 41) |

An initial CDR graft of muEpCAM-23 was created by grafting Kabat positions 24-34 (CDR-L1), 50-56 (CDR-L2), and 89-97 (CDR-L2) of the VL, and Kabat positions 31-35 (CDR-H1), 50-65 (CDR-H2), and 95-102 (CDR-H3) of the VH, into the corresponding human germline IGKV2D-29*01 and IGHV1-3*01 frameworks. The initial CDR grafted version contained 12 framework residue substitutions in VL and 27 framework residue changes in VH. Additionally, variants containing one or more back-mutations of the vernier zone residues were also made, and were subsequently evaluated for EpCAM-binding.

Several humanized versions of EpCAM23 retained binding to human HSC2 cells. However, only version Gv2.2 (VL Gv2 and VH Gv2) and version Gv4.2 (VL Gv4 and VH Gv2) also retained binding to cyno primary kidney epithelial cells. Affinity variants of the humanized antibody Gv4.2 were created. The variants were created either by mutagenizing solvent exposed CDR residues or VH/VL framework residues that rarely occur in the mouse/human natural repertoire (<5% frequency).

An initial set of humanized anti-EpCAM23Gv4.2 affinity variants was tested in an enzyme-linked immunosorbent assay (ELISA) using mFc-tagged huEpCAM or cynoEpCAM protein. Two affinity variants, huEpCAM23Gv4.2-1361-H and huEpCAM23Gv4.2-1565-Y, were selected for further evaluation. In particular, these variants were selected due to their somewhat lower binding affinities on HSC2 cells relative to the parent antibody (i.e., about a 3-fold reduced Kd compared to the parent antibody on HSC2 cells) and their different mutation sites.

### 7.2 Example 2. Mask Discovery

The identification and characterization of masking moieties (MM or “masks”) for use in activatable anti-EpCAM antibodies and fragments thereof (“activatable antibodies”) of the disclosure is described in detail in WO 2020/086665, and is described below.

A humanized anti-human EpCAM monoclonal antibody (EpCAM23Gv4.2) that is cross-reactive with human and cynomolgus EpCAM was used to screen a random X₁₅ peptide library where X is any amino acid, using a method similar to that described in PCT International Publication Number WO 2010/081173, published 15 Jul. 2010, the disclosure of which is incorporated herein by reference in its entirety. The screening consisted of one round of MACS and three rounds of FACS sorting. The initial MACS sorting was done with protein-A Dynabeads (Invitrogen) with the anti-EpCAM antibody. For MACS, anti-EpCAM antibody was conjugated with DyLight-488 (ThermoFisher), EpCAM binding activity was confirmed, and anti-EpCAM-488 was used as a fluorescent probe for all FACS rounds. Individual peptide clones were identified by sequence analysis from each FACS round.

Two heavy chain variants of the humanized anti-human EpCAM monoclonal antibody (EpCAM23(1361-H) and EpCAM23(1565-Y)) were generated and used to screen a random X₁₅ peptide library where X is any amino acid, using a method similar to that described in PCT International Publication Number WO 2010/081173, published 15 Jul. 2010. Masks EP101 to EP104 were identified using the EpCAM23(1565-Y) heavy chain variant. Masks EP105 to EP110 were identified using the EpCAM23(1361-H) heavy chain variant. Mutations of the lysine residue in the Ep107 masking moiety were also generated

The sequences of selected anti-EpCAM masking moieties are listed in Table 14.

TABLE 14
Masking Moleties for EpCAM
Activatable Antibodies.

| | | |
|---|---|---|
| Anti-EpCAM | | |
| Masking Moiety | Amino Acid Sequence | SEQ ID NO: |

Masks for Gv4.2 Humanized Antibody

| | | |
|---|---|---|
| EP01 | PLMTCSDYYTCKNNL | 331 |
| EP01-1 | PLMTCSDYYTCHNNL | 332 |
| EP01-2 | PLMTCSDYYTCLNNL | 151 |
| EP01-3 | PLMTCSDYYTQRNNL | 333 |
| EP02 | LSCTHSRYDMHCPHM | 152 |
| EP03 | HYCHSRTDTITHQNA | 153 |
| EP04 | WCPRLFDRPSMGCPT | 154 |
| EP05 | WWPPCQGGAWCEQRI | 155 |
| EP06 | KFSCTTMPHKNCMKP | 334 |
| EP07 | HSGCPRLFDRQSAPA | 156 |
| EP08 | PLMTCTKYNNCINNL | 335 |
| EP09 | PNYMCKLNTCTHHIA | 336 |
| EP10 | AACAGGVLFDRQSVD | 337 |
| EP11 | FICPTLYDRPHCMHT | 157 |
| EP12 | NMPACQGGSWCRPGY | 338 |
| EP13 | TLCARPPSYQLCSHV | 339 |
| EP14 | NLCPPGETRTQCQIT | 340 |
| EP15 | LYCALYSVQLFCVDL | 341 |
| EP16 | KMAPCDAWYCHQQTA | 342 |
| EP17 | QDECSNKEAENCSDV | 343 |
| EP18 | QTCYGWSWSGSCLWY | 344 |
| EP19 | SNPCISTGHTHCQKI | 345 |
| EP20 | QICFGGSKNRYDCFM | 346 |
| EP21 | PTAPCHFNPPCRNTG | 347 |
| EP22 | VCSGSSPGHPRVCGD | 348 |
| EP23 | LLPCPTSMPSYCFNY | 349 |
| EP24 | RGTPCTINTCTHHFS | 350 |
| EP25 | KNGGCKVPIPCKSWS | 351 |

Masks for Affinity Variants 1361-H and 1565-Y

| | | |
|---|---|---|
| EP101 | SWCHSATDTILPCSN | 158 |
| EP102 | SPACSDRYYQTCVLN | 159 |
| EP103 | MSCVVDRFDRQCPHL | 160 |
| EP104 | TTRCEHYWFTCPLSP | 161 |
| EP105 | DCTGYSPSVLPACRV | 162 |
| EP106 | FCSGYSPSVLPSCLM | 163 |
| EP107 | SKPCSYMHPYCFYNS | 164 |
| EP107-1 | SRPCSYMHPYCFYNS | 352 |
| EP107-2 | SHPCSYMHPYCFYNS | 353 |
| EP107-3 | SLPCSYMHPYCFYNS | 354 |
| EP108 | LTRCTIAHPYCYYNY | 165 |
| EP109 | PNTCMSERRICSLTY | 166 |
| EP110 | PRPHCAILRQCLAAT | 167 |

These masking peptides were used to generate anti-EpCAM activatable antibodies of the disclosure. The amino acid sequences for certain of these anti-EpCAM activatable antibodies are shown below in Table 15. In some embodiments, these anti-EpCAM activatable antibodies of the present disclosure include ISSGLLSGRSDNH (SEQ ID NO: 312), AVGLLAPPGGLSGRSDNH (SEQ ID NO: 313), ISSGLLSGRSDIH (SEQ ID NO: 314), ISSGLLSGRSDQH (SEQ ID NO: 315), ISSGLLSGRSDNP (SEQ ID NO: 316), ISSGLLSGRSANP (SEQ ID NO: 317). ISSGLLSGRSANI (SEQ ID NO: 318), ISSGLLSGRSDNI (SEQ ID NO: 169), AVGLLAPPGGLSGRSDIH (SEQ ID NO: 319), AVGLLAPPGGLSGRSDQH (SEQ ID NO: 320), AVGLLAPPGGLSGRSDNP (SEQ ID NO: 321), AVGLLAPPGGLSGRSANP (SEQ ID NO: 322), AVGLLAPPGGLSGRSANI (SEQ ID NO: 323), orAVGLLAPPGGLSGRSDNI (SEQ ID NO: 168). In some embodiments, certain anti-EpCAM activatable antibodies of the present disclosure include a non-cleavable moiety “NSUB” which is not expected to be cleaved.

While certain light chain sequences of activatable antibodies of the present disclosure shown below include the N-terminal spacer sequence of QGQSGQG (SEQ ID NO: 324), those of ordinary skill in the art appreciate that the activatable anti-EpCAM antibodies of the disclosure can include any suitable spacer sequence, such as, for example, a spacer sequence selected from the group consisting of QGQSGQ (SEQ ID NO: 325), QGQSG (SEQ ID NO: 326), QGQS (SEQ ID NO: 327), QGQ, QG, GQSGQG (SEQ ID NO: 328), QSGQG (SEQ ID NO: 329), SGQG (SEQ ID NO: 330), GQG, G, or Q. In some embodiments, the light chains of activatable anti-EpCAM antibodies of the disclosure can have no spacer sequence joined to the N-terminus.

TABLE 15
Variable light chain amino acid sequences of activatable
antibodies of the disclosure.

| Activatable antibody | | |
|---|---|---|
| Light Chain Variable | | |
| Domain | Amino Acid Sequence | SEQ ID NO: |
| EP01-2014-Gv4 | QGQSGQGPLMTCSDYYTCKNNLGGGSSGGSISSGLLSGRSDNI | 355 |
| | GGSDIVLTQTPLSLSVTPGQPASISCRSSRSLLHSDGFTYLYWFL | |
| | QKPGQSPQLLIYQTSNLASGVPDRESSSGSGTDFTLKISRVEAE | |
| | DVGVYYCAQNLELPNTFGQGTKLEIK | |
| EP01-1-2014-Gv4 | QGQSGQGPLMTCSDYYTCHNNLGGGSSGGSISSGLLSGRSDN | 356 |
| | IGGSDIVLTQTPLSLSVTPGQPASISCRSSRSLLHSDGFTYLYWF | |
| | LQKPGQSPQLLIYQTSNLASGVPDRESSSGSGTDFTLKISRVEA | |
| | EDVGVYYCAQNLELPNTFGQGTKLEIK | |
| EP01-2-2014-Gv4 | QGQSGQGPLMTCSDYYTCLNNLGGGSSGGSISSGLLSGRSDNI | 357 |
| | GGSDIVLTQTPLSLSVTPGQPASISCRSSRSLLHSDGFTYLYWFL | |
| | QKPGQSPQLLIYQTSNLASGVPDRESSSGSGTDFTLKISRVEAE | |
| | DVGVYYCAQNLELPNTFGQGTKLEIK | |
| EP01-3-2014-Gv4 | QGQSGQGPLMTCSDYYTCRNNLGGGSSGGSISSGLLSGRSDN | 358 |
| | IGGSDIVLTQTPLSLSVTPGQPASISCRSSRSLLHSDGFTYLYWF | |
| | LQKPGQSPQLLIYQTSNLASGVPDRESSSGSGTDFTLKISRVEA | |
| | EDVGVYYCAQNLELPNTFGQGTKLEIK | |
| EP02-2014-Gv4 | QGQSGQGLSCTHSRYDMHCPHMGGGSSGGSISSGLLSGRSD | 359 |
| | NIGGSDIVLTQTPLSLSVTPGQPASISCRSSRSLLHSDGFTYLYW | |
| | FLQKPGQSPQLLIYQTSNLASGVPDRESSSGSGTDFTLKISRVE | |
| | AEDVGVYYCAQNLELPNTFGQGTKLEIK | |
| EP03-2014-Gv4 | QGQSGQGHYCHSRTDTITHCNAGGGSSGGSISSGLLSGRSDNI | 360 |
| | GGSDIVLTQTPLSLSVTPGQPASISCRSSRSLLHSDGFTYLYWFL | |
| | QKPGQSPQLLIYQTSNLASGVPDRFSSSGSGTDFTLKISRVEAE | |
| | DVGVYYCAQNLELPNTFGQGTKLEIK | |
| EP04-2014-Gv4 | QGQSGQGWCPRLFDRPSMGCPTGGGSSGGSISSGLLSGRSD | 361 |
| | NIGGSDIVLTQTPLSLSVTPGQPASISCRSSRSLLHSDGFTYLYW | |
| | FLQKPGQSPQLLIYQTSNLASGVPDRFSSSGSGTDFTLKISRVE | |
| | AEDVGVYYCAQNLELPNTFGQGTKLEIK | |
| EP05-2014-Gv4 | QGQSGQGWWPPCQGGAWCEQRIGGGSSGGSISSGLLSGRSD | 362 |
| | NIGGSDIVLTQTPLSLSVTPGQPASISCRSSRSLLHSDGFTYLYW | |
| | FLQKPGQSPQLLIYQTSNLASGVPDRFSSSGSGTDFTLKISRVE | |
| | AEDVGVYYCAQNLELPNTFGQGTKLEIK | |
| EP06-2014-Gv4 | QGQSGQGKFSCTTMPHKNCMKPGGGSSGGSISSGLLSGRSDN | 363 |
| | IGGSDIVLTQTPLSLSVTPGQPASISCRSSRSLLHSDGFTYLYWF | |
| | LQKPGQSPQLLIYQTSNLASGVPDRFSSSGSGTDFTLKISRVEA | |
| | EDVGVYYCAQNLELPNTFGQGTKLEIK | |
| EP07-2014-Gv4 | QGQSGQGHSGCPRLFDRCSAPAGGGSSGGSISSGLLSGRSDN | 36 |
| | IGGSDIVLTQTPLSLSVTPGQPASISCRSSRSLLHSDGFTYLYWF | |
| | LQKPGQSPQLLIYQTSNLASGVPDRESSSGSGTDFTLKISRVEA | |
| | EDVGVYYCAQNLELPNTFGQGTKLEIK | |
| EP08-2014-Gv4 | QGQSGQGPLMTCTKYNNCINNLGGGSSGGSISSGLLSGRSDNI | 365 |
| | GGSDIVLTQTPLSLSVTPGQPASISCRSSRSLLHSDGFTYLYWFL | |
| | QKPGQSPQLLIYQTSNLASGVPDRFSSSGSGTDFTLKISRVEAE | |
| | DVGVYYCAQNLELPNTFGQGTKLEIK | |
| EP09-2014-Gv4 | QGQSGQGPNYMCKLNTCTHHIAGGGSSGGSISSGLLSGRSDNI | 366 |
| | GGSDIVLTQTPLSLSVTPGQPASISCRSSRSLLHSDGFTYLYWFL | |
| | QKPGQSPQLLIYQTSNLASGVPDRESSSGSGTDFTLKISRVEAE | |
| | DVGVYYCAQNLELPNTFGQGTKLEIK | |
| EP10-2014-Gv4 | QGQSGQGAACAGGVLFDRCSVDGGGSSGGSISSGLLSGRSDN | 367 |
| | IGGSDIVLTQTPLSLSVTPGQPASISCRSSRSLLHSDGFTYLYWF | |
| | LQKPGQSPQLLIYQTSNLASGVPDRESSSGSGTDFTLKISRVEA | |
| | EDVGVYYCAQNLELPNTFGQGTKLEIK | |
| EP11-2014-Gv4 | QGQSGQGFICPTLYDRPHCMHTGGGSSGGSISSGLLSGRSDNI | 368 |
| | GGSDIVLTQTPLSLSVTPGQPASISCRSSRSLLHSDGFTYLYWFL | |
| | QKPGQSPQLLIYQTSNLASGVPDRESSSGSGTDFTLKISRVEAE | |
| | DVGVYYCAQNLELPNTFGQGTKLEIK | |
| EP12-2014-Gv4 | QGQSGQGNMPACQGGSWCRPGYGGGSSGGSISSGLLSGRSD | 369 |
| | NIGGSDIVLTQTPLSLSVTPGQPASISCRSSRSLLHSDGFTYLYW | |
| | FLQKPGQSPQLLIYQTSNLASGVPDRFSSSGSGTDFTLKISRVE | |
| | AEDVGVYYCAQNLELPNTFGQGTKLEIK | |
| EP01-3014-Gv4 | QGQSGQGPLMTCSDYYTCKNNLGGGSSGGAVGLLAPPGGLS | 370 |
| | GRSDNIGGSDIVLTQTPLSLSVTPGQPASISCRSSRSLLHSDGFT | |
| | YLYWFLQKPGQSPQLLIYQTSNLASGVPDRFSSSGSGTDFTLKI | |
| | SRVEAEDVGVYYCAQNLELPNTFGQGTKLEIK | |
| EP01-1-3014-Gv4 | QGQSGQGPLMTCSDYYTCHNNLGGGSSGGAVGLLAPPGGLS | 371 |
| | GRSDNIGGSDIVLTQTPLSLSVTPGQPASISCRSSRSLLHSDGFT | |
| | YLYWFLQKPGQSPQLLIYQTSNLASGVPDRFSSSGSGTDFTLKI | |
| | SRVEAEDVGVYYCAQNLELPNTFGQGTKLEIK | |
| EP01-2-3014-Gv4 | QGQSGQGPLMTCSDYYTCLNNLGGGSSGGAVGLLAPPGGLSG | 372 |
| | RSDNIGGSDIVLTQTPLSLSVTPGQPASISCRSSRSLLHSDGFTY | |
| | LYWFLQKPGQSPQLLIYQTSNLASGVPDRFSSSGSGTDFTLKIS | |
| | RVEAEDVGVYYCAQNLELPNTFGQGTKLEIK | |
| EP01-3-3014-Gv4 | QGQSGQGPLMTCSDYYTQRNNLGGGSSGGAVGLLAPPGGLS | 373 |
| | GRSDNIGGSDIVLTQTPLSLSVTPGQPASISCRSSRSLLHSDGFT | |
| | YLYWFLQKPGQSPQLLIYQTSNLASGVPDRFSSSGSGTDFTLKI | |
| | SRVEAEDVGVYYCAQNLELPNTFGQGTKLEIK | |
| EP02-3014-Gv4 | QGQSGQGLSCTHSRYDMHCPHMGGGSSGGAVGLLAPPGGLS | 374 |
| | GRSDNIGGSDIVLTQTPLSLSVTPGQPASISCRSSRSLLHSDGFT | |
| | YLYWFLQKPGQSPQLLIYQTSNLASGVPDRFSSSGSGTDFTLKI | |
| | SRVEAEDVGVYYCAQNLELPNTFGQGTKLEIK | |
| EP03-3014-Gv4 | QGQSGQGHYCHSRTDTITHCNAGGGSSGGAVGLLAPPGGLSG | 375 |
| | RSDNIGGSDIVLTQTPLSLSVTPGQPASISCRSSRSLLHSDGFTY | |
| | LYWFLQKPGQSPQLLIYQTSNLASGVPDRFSSSGSGTDFTLKIS | |
| | RVEAEDVGVYYCAQNLELPNTFGQGTKLEIK | |
| EP04-3014-Gv4 | QGQSGQGWCPRLFDRPSMGCPTGGGSSGGAVGLLAPPGGLS | 376 |
| | GRSDNIGGSDIVLTQTPLSLSVTPGQPASISCRSSRSLLHSDGFT | |
| | YLYWFLQKPGQSPQLLIYQTSNLASGVPDRFSSSGSGTDFTLKI | |
| | SRVEAEDVGVYYCAQNLELPNTFGQGTKLEIK | |
| EP05-3014-Gv4 | QGQSGQGWWPPCQGGAWCEQRIGGGSSGGAVGLLAPPGGL | 377 |
| | SGRSDNIGGSDIVLTQTPLSLSVTPGQPASISCRSSRSLLHSDGF | |
| | TYLYWFLQKPGQSPQLLIYQTSNLASGVPDRFSSSGSGTDFTLK | |
| | ISRVEAEDVGVYYCAQNLELPNTFGQGTKLEIK | |
| EP06-3014-Gv4 | QGQSGQGKFSCTTMPHKNCMKPGGGSSGGAVGLLAPPGGLS | 378 |
| | GRSDNIGGSDIVLTQTPLSLSVTPGQPASISCRSSRSLLHSDGFT | |
| | YLYWFLQKPGQSPQLLIYQTSNLASGVPDRFSSSGSGTDFTLKI | |
| | SRVEAEDVGVYYCAQNLELPNTFGQGTKLEIK | |
| EP07-3014-Gv4 | QGQSGQGHSGCPRLFDRCSAPAGGGSSGGAVGLLAPPGGLS | 379 |
| | GRSDNIGGSDIVLTQTPLSLSVTPGQPASISCRSSRSLLHSDGFT | |
| | YLYWFLQKPGQSPQLLIYQTSNLASGVPDRFSSSGSGTDFTLKI | |
| | SRVEAEDVGVYYCAQNLELPNTFGQGTKLEIK | |
| EP08-3014-Gv4 | QGQSGQGPLMTCTKYNNCINNLGGGSSGGAVGLLAPPGGLSG | 380 |
| | RSDNIGGSDIVLTQTPLSLSVTPGQPASISCRSSRSLLHSDGFTY | |
| | LYWFLQKPGQSPQLLIYQTSNLASGVPDRFSSSGSGTDFTLKIS | |
| | RVEAEDVGVYYCAQNLELPNTFGQGTKLEIK | |
| EP09-3014-Gv4 | QGQSGQGPNYMCKLNTCTHHIAGGGSSGGAVGLLAPPGGLSG | 381 |
| | RSDNIGGSDIVLTQTPLSLSVTPGQPASISCRSSRSLLHSDGFTY | |
| | LYWFLQKPGQSPQLLIYQTSNLASGVPDRFSSSGSGTDFTLKIS | |
| | RVEAEDVGVYYCAQNLELPNTFGQGTKLEIK | |
| EP10-3014-Gv4 | QGQSGQGAACAGGVLFDRCSVDGGGSSGGAVGLLAPPGGLS | 382 |
| | GRSDNIGGSDIVLTQTPLSLSVTPGQPASISCRSSRSLLHSDGFT | |
| | YLYWFLQKPGQSPQLLIYQTSNLASGVPDRFSSSGSGTDFTLKI | |
| | SRVEAEDVGVYYCAQNLELPNTFGQGTKLEIK | |
| EP11-3014-Gv4 | QGQSGQGFICPTLYDRPHCMHTGGGSSGGAVGLLAPPGGLSG | 383 |
| | RSDNIGGSDIVLTQTPLSLSVTPGQPASISCRSSRSLLHSDGFTY | |
| | LYWFLQKPGQSPQLLIYQTSNLASGVPDRFSSSGSGTDFTLKIS | |
| | RVEAEDVGVYYCAQNLELPNTFGQGTKLEIK | |
| EP12-3014-Gv4 | QGQSGQGNMPACQGGSWCRPGYGGGSSGGAVGLLAPPGGL | 384 |
| | SGRSDNIGGSDIVLTQTPLSLSVTPGQPASISCRSSRSLLHSDGF | |
| | TYLYWFLQKPGQSPQLLIYQTSNLÅSGVPDRFSSSGSGTDFTLK | |
| | ISRVEAEDVGVYYCAQNLELPNTFGQGTKLEIK | |
| Variable Light Chains for Affinity Variants 1361-H and 1565-Y | | |
| EP101-3014-Gv4 | QGQSGQGSWCHSATDTILPCSNGGGSSGGAVGLLAPPGGLSG | 385 |
| | RSDNIGGSDIVLTQTPLSLSVTPGQPASISCRSSRSLLHSDGFTY | |
| | LYWFLQKPGQSPQLLIYQTSNLASGVPDRFSSSGSGTDFTLKIS | |
| | RVEAEDVGVYYCAQNLELPNTFGQGTKLEIK | |
| EP102-3014-Gv4 | QGQSGQGSPACSDRYYQTCVLNGGGSSGGAVGLLAPPGGLS | 386 |
| | GRSDNIGGSDIVLTQTPLSLSVTPGQPASISCRSSRSLLHSDGFT | |
| | YLYWFLQKPGQSPQLLIYQTSNLASGVPDRFSSSGSGTDFTLKI | |
| | SRVEAEDVGVYYCAQNLELPNTFGQGTKLEIK | |
| EP103-3014-Gv4 | QGQSGQGMSCVVDRFDRQCPHLGGGSSGGAVGLLAPPGGLS | 387 |
| | GRSDNIGGSDIVLTQTPLSLSVTPGQPASISCRSSRSLLHSDGFT | |
| | YLYWFLQKPGQSPQLLIYQTSNLASGVPDRFSSSGSGTDFTLKI | |
| | SRVEAEDVGVYYCAQNLELPNTFGQGTKLEIK | |
| EP104-3014-Gv4 | QGQSGQGTTRCEHYWFTCPLSPGGGSSGGAVGLLAPPGGLS | 388 |
| | GRSDNIGGSDIVLTQTPLSLSVTPGQPASISCRSSRSLLHSDGFT | |
| | YLYWFLQKPGQSPQLLIYQTSNLASGVPDRFSSSGSGTDFTLKI | |
| | SRVEAEDVGVYYCAQNLELPNTFGQGTKLEIK | |
| EP105-3014-Gv4 | QGQSGQGDCTGYSPSVLPACRVGGGSSGGAVGLLAPPGGLS | 389 |
| | GRSDNIGGSDIVLTQTPLSLSVTPGQPASISCRSSRSLLHSDGFT | |
| | YLYWFLQKPGQSPQLLIYQTSNLASGVPDRFSSSGSGTDFTLKI | |
| | SRVEAEDVGVYYCAQNLELPNTFGQGTKLEIK | |
| EP106-3014-Gv4 | QGQSGQGFCSGYSPSVLPSCLMGGGSSGGAVGLLAPPGGLS | 390 |
| | GRSDNIGGSDIVLTQTPLSLSVTPGQPASISCRSSRSLLHSDGFT | |
| | YLYWFLQKPGQSPQLLIYQTSNLASGVPDRFSSSGSGTDFTLKI | |
| | SRVEAEDVGVYYCAQNLELPNTFGQGTKLEIK | |
| EP107-3014-Gv4 | QGQSGQGSKPCSYMHPYCFYNSGGGSSGGAVGLLAPPGGLS | 391 |
| | GRSDNIGGSDIVLTQTPLSLSVTPGQPASISCRSSRSLLHSDGFT | |
| | YLYWFLQKPGQSPQLLIYQTSNLASGVPDRFSSSGSGTDFTLKI | |
| | SRVEAEDVGVYYCAQNLELPNTFGQGTKLEIK | |
| EP108-3014-Gv4 | QGQSGQGLTRCTIAHPYCYYNYGGGSSGGAVGLLAPPGGLSG | 392 |
| | RSDNIGGSDIVLTQTPLSLSVTPGQPASISCRSSRSLLHSDGFTY | |
| | LYWFLQKPGQSPQLLIYQTSNLASGVPDRFSSSGSGTDFTLKIS | |
| | RVEAEDVGVYYCAQNLELPNTFGQGTKLEIK | |
| EP109-3014-Gv4 | QGQSGQGPNTCMSERRICSLTYGGGSSGGAVGLLAPPGGLSG | 393 |
| | RSDNIGGSDIVLTQTPLSLSVTPGQPASISCRSSRSLLHSDGFTY | |
| | LYWFLQKPGQSPQLLIYQTSNLASGVPDRFSSSGSGTDFTLKIS | |
| | RVEAEDVGVYYCAQNLELPNTFGQGTKLEIK | |
| EP110-3014-Gv4 | QGQSGQGPRPHCAILRQCLAATGGGSSGGAVGLLAPPGGLSG | 394 |
| | RSDNIGGSDIVLTQTPLSLSVTPGQPASISCRSSRSLLHSDGFTY | |
| | LYWFLQKPGQSPQLLIYQTSNLASGVPDRFSSSGSGTDFTLKIS | |
| | RVEAEDVGVYYCAQNLELPNTFGQGTKLEIK | |
| EP107-1-3014-Gv4 | QGQSGQGSRPCSYMHPYCFYNSGGGSSGGAVGLLAPPGGLS | 395 |
| | GRSDNIGGSDIVLTQTPLSLSVTPGQPASISCRSSRSLLHSDGFT | |
| | YLYWFLQKPGQSPQLLIYQTSNLASGVPDRFSSSGSGTDFTLKI | |
| | SRVEAEDVGVYYCAQNLELPNTFGQGTKLEIK | |
| EP107-2-3014-Gv4 | QGQSGQGSHPCSYMHPYCFYNSGGGSSGGAVGLLAPPGGLS | 396 |
| | GRSDNIGGSDIVLTQTPLSLSVTPGQPASISCRSSRSLLHSDGFT | |
| | YLYWFLQKPGQSPQLLIYQTSNLASGVPDRFSSSGSGTDFTLKI | |
| | SRVEAEDVGVYYCAQNLELPNTFGQGTKLEIK | |
| EP107-3-3014-Gv4 | QGQSGQGSLPCSYMHPYCFYNSGGGSSGGAVGLLAPPGGLS | 397 |
| | GRSDNIGGSDIVLTQTPLSLSVTPGQPASISCRSSRSLLHSDGFT | |
| | YLYWFLQKPGQSPQLLIYQTSNLASGVPDRFSSSGSGTDFTLKI | |
| | SRVEAEDVGVYYCAQNLELPNTFGQGTKLEIK | |

Exemplary anti-EpCAM activatable antibodies having the following heavy and light chains are shown below in Table 16.

TABLE 16
Anti-EpCAM Activatable Antibodies.

| Anti-EpCAM | | |
|---|---|---|
| Activatable Antibody | Heavy Chain | Light Chain |
| EP01-2014 | EpCAM23Gv4.2 HC | EP01-2014 LC |
| EP02-2014 | EpCAM23Gv4.2 HC | EP02-2014 LC |
| EP03-2014 | EpCAM23Gv4.2 HC | EP03-2014 LC |
| EP04-2014 | EpCAM23Gv4.2 HC | EP04-2014 LC |
| EP05-2014 | EpCAM23Gv4.2 HC | EP05-2014 LC |
| EP06-2014 | EpCAM23Gv4.2 HC | EP06-2014 LC |
| EP07-2014 | EpCAM23Gv4.2 HC | EP07-2014 LC |
| EP08-2014 | EpCAM23Gv4.2 HC | EP08-2014 LC |
| EP09-2014 | EpCAM23Gv4.2 HC | EP09-2014 LC |
| EP10-2014 | EpCAM23Gv4.2 HC | EP10-2014 LC |
| EP11-2014 | EpCAM23Gv4.2 HC | EP11-2014 LC |
| EP12-2014 | EpCAM23Gv4.2 HC | EP12-2014 LC |
| EP01-1-2014 | EpCAM23Gv4.2 HC | EP01-1-2014 LC |
| EP01-2-2014 | EpCAM23Gv4.2 HC | EP01-2-2014 LC |
| EP01-3-2014 | EpCAM23Gv4.2 HC | EP01-3-2014 LC |
| EP01-3014 | EpCAM23Gv4.2 HC | EP01-3014 LC |
| EP02-3014 | EpCAM23Gv4.2 HC | EP02-3014 LC |
| EP03-3014 | EpCAM23Gv4.2 HC | EP03-3014 LC |
| EP04-3014 | EpCAM23Gv4.2 HC | EP04-3014 LC |
| EP05-3014 | EpCAM23Gv4.2 HC | EP05-3014 LC |
| EP06-3014 | EpCAM23Gv4.2 HC | EP06-3014 LC |
| EP07-3014 | EpCAM23Gv4.2 HC | EP07-3014 LC |
| EP08-3014 | EpCAM23Gv4.2 HC | EP08-3014 LC |
| EP09-3014 | EpCAM23Gv4.2 HC | EP09-3014 LC |
| EP10-3014 | EpCAM23Gv4.2 HC | EP10-3014 LC |
| EP11-3014 | EpCAM23Gv4.2 HC | EP11-3014 LC |
| EP12-3014 | EpCAM23Gv4.2 HC | EP12-3014 LC |
| EP01-1-3014 | EpCAM23Gv4.2 HC | EP01-1-3014 LC |
| EP01-2-3014 | EpCAM23Gv4.2 HC | EP01-2-3014 LC |
| EP01-3-3014 | EpCAM23Gv4.2 HC | EP01-3-3014 LC |
| EP01-NSUB | EpCAM23Gv4.2 HC | EP01-NSUB LC |
| EP02-NSUB | EpCAM23Gv4.2 HC | EP02-NSUB LC |
| EP03-NSUB | EpCAM23Gv4.2 HC | EP03-NSUB LC |
| EP04-NSUB | EpCAM23Gv4.2 HC | EP04-NSUB LC |
| EP05-NSUB | EpCAM23Gv4.2 HC | EP05-NSUB LC |
| EP06-NSUB | EpCAM23Gv4.2 HC | EP06-NSUB LC |
| EP07-NSUB | EpCAM23Gv4.2 HC | EP07-NSUB LC |
| EP08-NSUB | EpCAM23Gv4.2 HC | EP08-NSUB LC |
| EP09-NSUB | EpCAM23Gv4.2 HC | EP09-NSUB LC |
| EP10-NSUB | EpCAM23Gv4.2 HC | EP10-NSUB LC |
| EP11-NSUB | EpCAM23Gv4.2 HC | EP11-NSUB LC |
| EP12-NSUB | EpCAM23Gv4.2 HC | EP12-NSUB LC |
| EP01-1-NSUB | EpCAM23Gv4.2 HC | EP01-1-NSUB LC |
| EP01-2-NSUB | EpCAM23Gv4.2 HC | EP01-2-NSUB LC |
| EP01-3-NSUB | EpCAM23Gv4.2 HC | EP01-3-NSUB LC |
| EP101-3014 (1565-Y) | EpCAM23(1565-Y) HC | EP101-3014 LC |
| EP102-3014 (1565-Y) | EpCAM23(1565-Y) HC | EP102-3014 LC |
| EP103-3014 (1565-Y) | EpCAM23(1565-Y) HC | EP103-3014 LC |
| EP104-3014 (1565-Y) | EpCAM23(1565-Y) HC | EP104-3014 LC |
| EP105-3014 (1361-H) | EpCAM23(1361-H) HC | EP105-3014 LC |
| EP106-3014 (1361-H) | EpCAM23(1361-H) HC | EP106-3014 LC |
| EP107-3014 (1361-H) | EpCAM23(1361-H) HC | EP107-3014 LC |
| EP108-3014 (1361-H) | EpCAM23(1361-H) HC | EP108-3014 LC |
| EP109-3014 (1361-H) | EpCAM23(1361-H) HC | EP109-3014 LC |
| EP110-3014 (1361-H) | EpCAM23(1361-H) HC | EP110-3014 LC |
| EP107-1-3014 (1361-H) | EpCAM23(1361-H) HC | EP107-1-3014 LC |
| EP107-1-3014 (1361-H) | EpCAM23(1361-H) HC | EP107-2-3014 LC |
| EP107-1-3014 (1361-H) | EpCAM23(1361-H) HC | EP107-3-3014 LC |
| EP01-3014 (1565-Y) | EpCAM23(1565-Y) HC | EP01-3014 LC |
| EP02-3014 (1565-Y) | EpCAM23(1565-Y) HC | EP02-3014 LC |
| EP03-3014 (1565-Y) | EpCAM23(1565-Y) HC | EP03-3014 LC |
| EP04-3014 (1565-Y) | EpCAM23(1565-Y) HC | EP04-3014 LC |
| EP05-3014 (1565-Y) | EpCAM23(1565-Y) HC | EP05-3014 LC |
| EP06-3014 (1565-Y) | EpCAM23(1565-Y) HC | EP06-3014 LC |
| EP07-3014 (1565-Y) | EpCAM23(1565-Y) HC | EP07-3014 LC |
| EP08-3014 (1565-Y) | EpCAM23(1565-Y) HC | EP08-3014 LC |
| EP09-3014 (1565-Y) | EpCAM23(1565-Y) HC | EP09-3014 LC |
| EP10-3014 (1565-Y) | EpCAM23(1565-Y) HC | EP10-3014 LC |
| EP11-3014 (1565-Y) | EpCAM23(1565-Y) HC | EP11-3014 LC |
| EP12-3014 (1565-Y) | EpCAM23(1565-Y) HC | EP12-3014 LC |
| EP01-3014 (1361-H) | EpCAM23(1361-H) HC | EP01-3014 LC |
| EP02-3014 (1361-H) | EpCAM23(1361-H) HC | EP02-3014 LC |
| EP03-3014 (1361-H) | EpCAM23(1361-H) HC | EP03-3014 LC |
| EP04-3014 (1361-H) | EpCAM23(1361-H) HC | EP04-3014 LC |
| EP05-3014 (1361-H) | EpCAM23(1361-H) HC | EP05-3014 LC |
| EP06-3014 (1361-H) | EpCAM23(1361-H) HC | EP06-3014 LC |
| EP07-3014 (1361-H) | EpCAM23(1361-H) HC | EP07-3014 LC |
| EP08-3014 (1361-H) | EpCAM23(1361-H) HC | EP08-3014 LC |
| EP09-3014 (1361-H) | EpCAM23(1361-H) HC | EP09-3014 LC |
| EP10-3014 (1361-H) | EpCAM23(1361-H) HC | EP10-3014 LC |
| EP11-3014 (1361-H) | EpCAM23(1361-H) HC | EP11-3014 LC |
| EP12-3014 (1361-H) | EpCAM23(1361-H) HC | EP12-3014 LC |

A solid-phase binding assay was used to demonstrate the binding of anti-human EpCAM antibodies of the present disclosure. Briefly, recombinant human EpCAM-mFC protein (Immunogen) was coated on ELISA plates (50 μL of 1 μg/mL), and then incubated with serially-diluted anti-EpCAM antibody (starting at 62.5 nM) or activatable anti-EpCAM antibodies (starting at 1 μM), where in the activatable antibodies were assayed in their uncleaved form. The amount of bound antibody was detected using anti-human IgG (anti-Fab) conjugated to horseradish peroxidase (Sigma) with Ultra TMB-ELISA reagent (Thermo Fisher Scientific) and the OD was measured at 450 nM. The K_{D} were measured for each antibody and activatable antibody, and the ELISA masking efficiency (ME) for each activatable antibody relative to the unmasked antibody was calculated, with exemplary results shown in Table 17 and Table 18. Collectively, these data show that anti-human EpCAM activatable antibodies of the present disclosure demonstrate a shifted binding affinity to recombinant EpCAM protein compared to the parental anti-EpCAM antibody of the present disclosure.

TABLE 17
Anti-EpCAM Activatable Antibodies Masking Efficiencies.

| Anti-EpCAM Activatable | ELISA K_{D} | ELISA masking |
|---|---|---|
| Antibody | (nM) | efficiency |
| EP01-2014 | 36 | 99 |
| EP02-2014 | 7 | 21 |
| EP03-2014 | 11 | 31 |
| EP04-2014 | 12 | 34 |
| EP05-2014 | 17 | 50 |
| EP06-2014 | 1 | 3 |
| EP07-2014 | 12 | 38 |
| EP08-2014 | 15 | 48 |
| EP09-2014 | 22 | 67 |
| EP10-2014 | 15 | 42 |
| EP11-2014 | 21 | 59 |
| EP12-2014 | 16 | 46 |
| EP01-1-2014 | 53 | 81 |
| EP01-2-2014 | 46 | 100 |
| EP01-3-2014 | 52 | 86 |
| EP01-3014 | 35 | 100 |
| EP02-3014 | 7 | 19 |
| EP03-3014 | 9 | 25 |
| EP04-3014 | 11 | 31 |
| EP05-3014 | 16 | 44 |
| EP06-3014 | 1 | 4 |
| EP07-3014 | 10 | 27 |
| EP08-3014 | 13 | 37 |
| EP09-3014 | 18 | 52 |
| EP10-3014 | 13 | 35 |
| EP11-3014 | 20 | 53 |
| EP12-3014 | 14 | 38 |
| EP01-1-3014 | 59 | 76 |
| EP01-2-3014 | 46 | 102 |
| EP01-3-3014 | 64 | 80 |
| EP101-3014 (1565-Y) | 389 | 930 |
| EP102-3014 (1565-Y) | 72 | 173 |
| EP103-3014 (1565-Y) | 140 | 335 |
| EP104-3014 (1565-Y) | 126 | 302 |
| EP105-3014 (1361-H) | 201 | 343 |
| EP106-3014 (1361-H) | 348 | 592 |
| EP107-3014 (1361-H) | 147 | 250 |
| EP108-3014 (1361-H) | 58 | 99 |
| EP109-3014 (1361-H) | 512 | 872 |
| EP110-3014 (1361-H) | 336 | 571 |

TABLE 18
Anti-EpCAM Activatable Antibodies Masking Efficiencies.

| Anti-EpCAM Activatable | ELISA K_{D} | ELISA masking |
|---|---|---|
| Antibody | (nM) | efficiency |
| EP01-3014 (1565-Y) | 0.66 | 1 |
| EP02-3014 (1565-Y) | 34.5 | 67 |
| EP03-3014 (1565-Y) | 64.4 | 125 |
| EP04-3014 (1565-Y) | 26.9 | 52 |
| EP05-3014 (1565-Y) | 8.6 | 17 |
| EP07-3014 (1565-Y) | 37.6 | 73 |
| EP11-3014 (1565-Y) | 38.0 | 74 |
| EP101-3014 (1565-Y) | 388.8 | 756 |
| EP102-3014 (1565-Y) | 72.5 | 141 |
| EP103-3014 (1565-Y) | 140.2 | 272 |
| EP104-3014 (1565-Y) | 126.4 | 246 |
| EP01-3014 (1361-H) | 18.2 | 32 |
| EP02-3014 (1361-H) | 0.44 | 1 |
| EP03-3014 (1361-H) | 4.0 | 7 |
| EP04-3014 (1361-H) | 1.9 | 3 |
| EP05-3014 (1361-H) | 0.46 | 1 |
| EP07-3014 (1361-H) | 1.5 | 3 |
| EP11-3014 (1361-H) | 30.5 | 54 |
| EP105-3014 (1361-H) | 201 | 358 |
| EP106-3014 (1361-H) | 348 | 619 |
| EP107-3014 (1361-H) | 147 | 261 |
| EP108-3014 (1361-H) | 58 | 103 |
| EP109-3014 (1361-H) | 512 | 911 |
| EP110-3014 (1361-H) | 336 | 597 |
| EP107-1-3014 (1361-H) | 31 | 87 |
| EP107-2-3014 (1361-H) | 44 | 122 |
| EP107-3-3014 (1361-H) | 44 | 123 |

As depicted in Table 17 and Table 18, these exemplary results demonstrated that the anti-human EpCAM activatable antibodies showed a range of masking efficiencies relative to an unmasked anti-EpCAM antibody.

### 7.3 Example 3. Epitope Mapping

The human CD326 antigen, EpCAM (epithelial cell adhesion molecule), is composed of 314 amino acids, containing a 265-amino acid extra-cellular domain, a 23-amino acid transmembrane domain, and a cytoplasmic tail of 26 amino acids. The extracellular domain can be further divided into three domains (D1, D2 and D3). The extracellular domain contains two cysteine rich epidermal growth factor-like (EGF-like) repeats, which include a first domain comprising a region from the glutamine at position 24 of the mature protein (i.e., prior to signal peptide cleavage) to the cysteine at position 59, and a second domain comprising a region from cysteine at position 66 to the cysteine at position 135. Then, in tandem with the first two domains, there is also a cysteine-free third domain (D3) which includes amino acid residues 136-243.

TABLE 19
EpCAM Domains.

| | |
|---|---|
| >NP_002345.2 | QEECVCENYKLAVNCFVNNNRQCQCTSVGAQN |
| EpCAM D1 domain | TVIC |
| | (SEQ ID NO: 2) |
| EpCAM | CLVMKAEMNGSKLGRRAKPEGALQNNDGLYDA |
| D2 domain | ADCDESGLFKAKQCNGTSMCWCVNTAGVRRTD |
| | KDTEITC |
| | (SEQ ID NO: 3) |
| EpCAM | SERVRTYWIIIELKHKAREKPYDSKSLRTALQ |
| D3 domain | KEITTRYQLDPKFITSILYENNVITIDLVQNS |
| | SQKTQNDVDIADVAYYFEKDVKGESLFHSKKM |
| | DLTVNGEQLDLDPGQTLIYYVDEKAPEFSMQG |
| | LK |
| | (SEQ ID NO: 4) |

Epitopes for the humanized EpCAM antibody Gv4.2 were mapped by engineering chimeric proteins utilizing combinations of the extracellular domains of human and mouse EpCAM. Because human and mouse EpCAM share over 82% amino acid sequence identity and 85% similarity, the topology of the chimeric proteins should remain intact.

The extracellular region of human EpCAM (residues 1-265) was codon optimized, synthesized and cloned in-frame into a vector (pGSmuFc2ANL) containing the mouse IgG2a Fc region utilizing Hindill and BamHI restriction sites at Genscript. Similarly, other expression vectors containing various chimeric variants of the human/mouse EpCAM extracellular domain were synthesized by replacing residues corresponding to human EpCAM domain D1 (24-59), domain D2 (66-135), domain D3 (136-265) or a combination thereof with corresponding mouse residues. These expression constructs were transiently produced in suspension adapted HEK-293T cells using PEI as a transfection reagent in shake flasks. The transfections were incubated for one week, harvested, and the filtered supernatants purified using a combination of protein A and CHT chromatography.

The humanized EpCAM antibody huEpCAM23Gv4.2 was tested in an enzyme-linked immunosorbent assay (ELISA) format for binding to the EpCAM proteins described above. Briefly, each mFc-tagged EpCAM protein was purified using a combination of protein A and CHT chromatography. Each mFc-tagged EpCAM protein was diluted to 0.5 ug/mL in 50 mM sodium bicarbonate buffer pH 9.6, and 100 μL was added to each well. After a 16 hr incubation at 4 °C, the plates were washed with Tris-buffered saline with 0.1% Tween-20 (TBST), then blocked with 200 μL blocking buffer (TBS with 1% BSA). Next, 100 μL of primary antibody, huEpCAM23Gv4.2 serially diluted in blocking buffer, was added in duplicate to the ELISA wells and incubated at room temperature for 1 hr. The plates were then washed 3 times with TBST before adding 100 μL of anti-human IgG (H+L)-HRP to each well. The plates were again incubated for 1 hr at room temperature followed by three washes with TBST. Finally, 100 μL of TMB one component HRP microwell substrate was added to each well and incubated for 5 min. The reaction was stopped by adding 100 μL stopping solution and absorbances were read at 450 nm in a multiwell plate reader. OD450 was plotted against the antibody concentration in a semi-log plot. A dose-response curve was generated by non-linear regression and the EC₅₀ value of each curve was calculated using GraphPad Prism v6.

Binding of the huEpCAM23Gv4.2 antibody to the chimeric EpCAM proteins was evaluated in comparison to the wild type EpCAM. The huEpCAM23Gv4.2 antibody binds to both EpCAM-D2 (66-135) and EpCAM-D3 (136-265) with similar affinities as to that of the wild type EpCAM. Conversely, the huEpCAM23Gv4.2 antibody does not bind to the EpCAM-D1 and D1/D2 constructs and binding is all but eliminated for the chimeric protein EpCAM-D1(24-59) construct. These results indicate that the epitope of the huEpCAM23Gv4.2 antibody is located primarily within the D1 (24-59) domain of EpCAM.

### 7.4 Example 4. Preparation of Epcam Antibody Drug Conjugates and Activatable Antibody Drug Conjugates

### 7.4.1 Preparation of Huepcam23Gv4.2-LALALALALALA-CPT66

The following example provides a method for producing an activatable EpCAM antibody drug conjugate according to embodiments of the present disclosure. The activatable EpCAM drug conjugate obtainable by the following method includes heavy chains having the amino acid sequence of SEQ ID NO:103 and light chains having the amino acid sequence of SEQ ID NO:179, and a drug-to-antibody ratio of 8, wherein the linker payload is

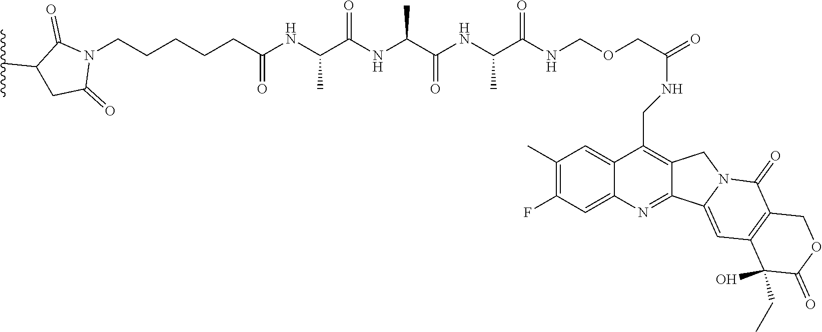 [SMILES: *C1CC(=O)N(CCCCCC(=O)N[C@@H](C)C(=O)N[C@@H](C)C(=O)N[C@@H](C)C(=O)NCOCC(=O)NCc2c3c(nc4cc(F)c(C)cc24)-c2cc4c(c(=O)n2C3)COC(=O)[C@]4(O)CC)C1=O]

wherein the denotes the covalent attachment position to the activatable EpCAM antibody via one of eight cysteine residues of the activatable EpCAM antibody normally involved in the formation of four disulfide bonds.

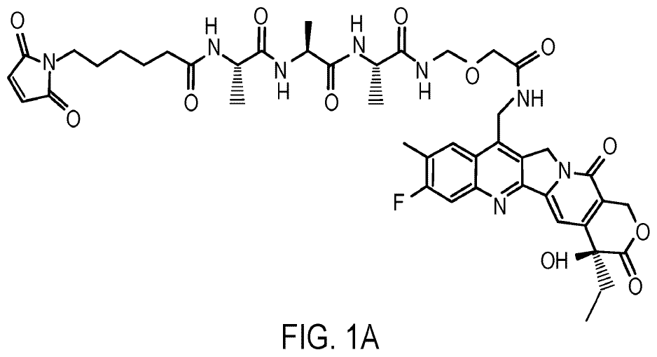

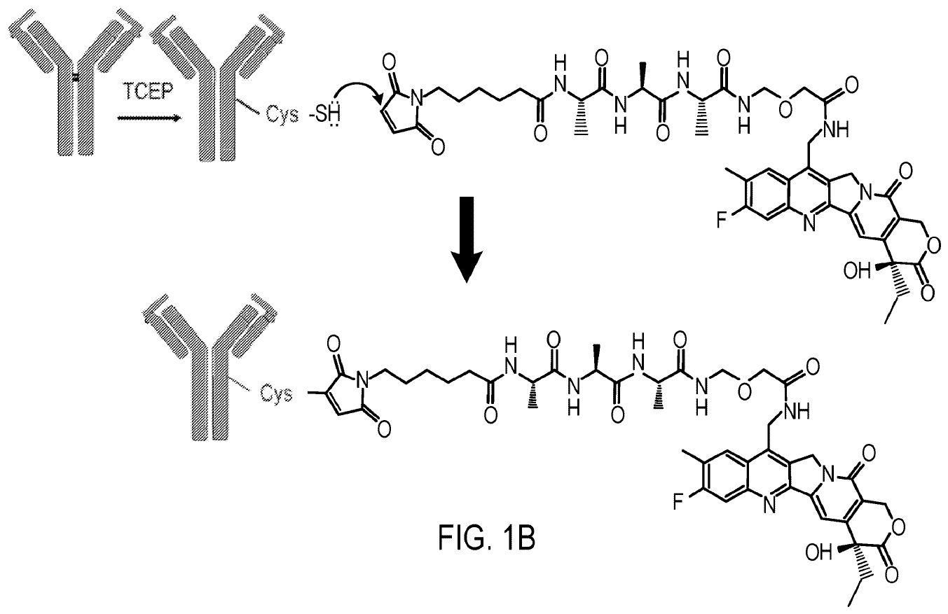

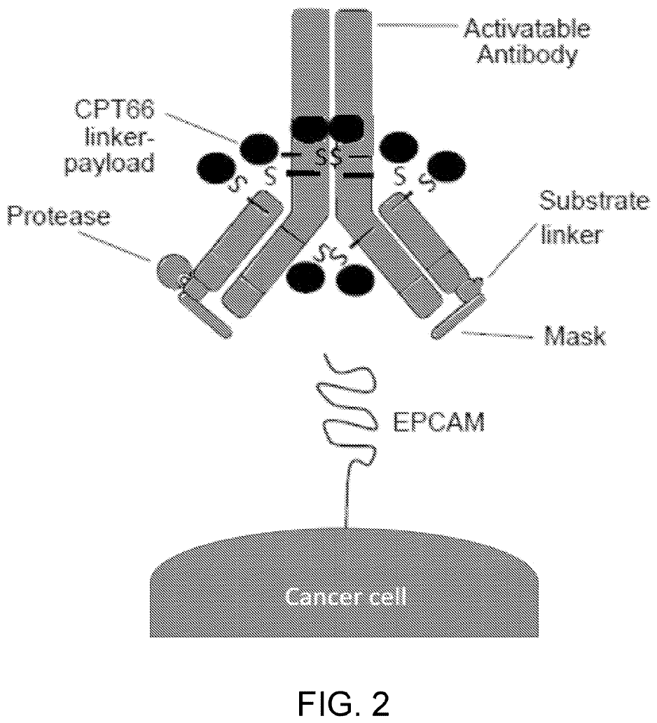

FIG. 1A shows the camptothecin derivative linker-payload used to make the EpCAM antibody drug conjugates. As shown in FIG. 2, a generated EpCAM antibody drug conjugate comprises an EpCAM antibody, a cleavable substrate linker, and a mask. In an uncleaved (inactive) state, the mask inhibits the binding of the EpCAM antibody to EpCAM. The cleavable substrate linker is cleavable by a protease. Upon cleavage, the mask is released and the antibody is free to bind to EpCAM. The depicted embodiment includes 8 conjugated camptothecin-derived (CPT66) linker-payloads. The conjugation of the linker-payloads to the activatable EpCAM antibody is stochastic, with conjugation occurring at the antibody's inter-chain cysteines.

Briefly, to make the CPT linker-toxin (CPT-LT), stock solution (approx. 40 mM), 9.1 mL of anhydrous DMA was added to a vial of 336.8 mg of CPT-LT solid and the vial was vortexed until all solids dissolved. The concentration of this stock solution was then determined using the UV spectrometry method to be 45.57 mM.

2 mL (0.05 volume) of EPPS buffer (1M, pH 7.80) was transferred into a 100 mL 3-neck round bottom flask, followed by adding 0.4 mL (0.01 volume) of 0.5 M EDTA solution and mixing with stirring. 37.6 mL of CX-231 stock solution (24.45 g/L; 0.94 volume; previously thawed at 4 °C) was then added and the reaction mixture was stirred at RT for 5 min to fully mix all starting materials.

To the reaction vessel, 4.5 Molar Eq of TCEP (2.673 mL of 10 mM stock solution) was added. The reaction time was set to T=0 immediately after the addition of TCEP solution. The reaction mixture was stirred at RT for 3 h at 500 rpm, so that all four inter-chain disulfide bonds were fully reduced.

To the reaction vessel, 2.316 mL of DMA and 4.0 Molar Eq of CPT-LT (521 uL of 45.57 mM stock solution) were added at T=3 h using pipet. Another 2 batches of CPT-LT, 4.0 Molar Eq each (521 uL of 45.57 mM stock solution), were subsequently added at T=4 h and 5 h. Total amount of CPT-LT was 12.0 Molar Eq (1.563 mL of 45.57 mM stock solution). Total volume of DMA, including DMA in CPT-LT stock solution as well as additional DMA, is 3.879 mL, comprising 8.3% of the reaction volume. The total reaction time was 7 h, including 3 h for TCEP reduction and 4 h for CPT-LT conjugation.

At T=7 h, 24 Molar Eq of NAC (2.326 mL of 10 g/L solution) was added to the reaction and stirred for 15 min at RT and 500 rpm to terminate the conjugation process. The crude reaction mixture was purified by tangential flow filtration.

The purified EpCAM-Camp66 immunoconjugate is tested with the following QC assays:
concentration and drug-to-antibody (or activatable antibody) ratio (DAR) are measured with UV-vis spectrophotometry method using a SoloVPE instrument;
% monomer is measured with the SEC-HPLC method;
content of free drug-related impurities (FDRI) is evaluated with the HiSep-HPLC method.

Purity and integrity of the immunoconjugate was confirmed using LC-MS and CE-SDS assays.

The above-described conjugation reaction is depicted schematically in FIG. 1B. As shown, the activatable EpCAM antibody is reduced using TCEP, which exposes reactive cysteines that typically form disulfide bridges between the heavy chains of the activatable antibody, as well as between light and heavy chains.

Importantly, the one or more disulfide bonds occurring within the mask moiety of the activatable EpCAM antibody are left undisturbed by the reducing conditions. The maleimide functional group of the linker-payload of FIG. 1A reacts with a thiol group of the exposed reactive cysteines to create a covalent thioester linkage.

### 7.4.2 Preparation of Huepcam23Gv4.2-MCGGFG-CPT66

Maleimide GGFG-CPT66 linker toxin was synthesized at Abzena and dissolved in DMSO. CX-231 activatable antibody was buffer exchanged to 50 mM HEPES, 2 mM EDTA, pH 7.0 and reduced with 10 molar equivalents of TCEP at room temperature for 2 hr. Reduced activatable antibody was buffer exchanged to 50 mM HEPES, 2 mM EDTA by a desalting column. 10% DMSO was added to the reduced activatable antibody along with 12 molar equivalents of maleimide GGFG-CPT66 and incubated at room temperature for 45 minutes. Reaction was quenched with N-acetyl cysteine followed by buffer exchange to 20 mM Succinate, 6% sucrose, pH 6.5. The purified EpCAM-GGFG-DXd immunoconjugate is tested with the following QC assays:
concentration and drug-to-antibody (or activatable antibody) ratio (DAR) are measured with UV-vis spectrophotometry method using a SoloVPE instrument;
% monomer is measured with the SEC-HPLC method;
content of free drug-related impurities (FDRI) is evaluated with the HiSep-HPLC method.

Purity and integrity of the immunoconjugate was confirmed using LC-MS and CE-SDS assays.

### 7.4.3 Preparation of Huepcam23Gv4.2-McGGFG-DXd

10 mg of Maleimide GGFG-DXd linker toxin was purchased from MedChem Express and dissolved in 1 mL of DMA to get 9.67 mM solution. To 4 ml of CX-231 at 25.2 mg/mL concentration in 20 mM succinate, 4% sucrose, pH 6.5, added 5 mM EDTA and 12 molar equivalents of TCEP (in 10 mM HEPES buffer, pH 7.53). The reduction reaction was carried out at room temperature for 3 hr. Excess TCEP was removed by a desalting column. To reduced CX-231 solution, 12 molar equivalents of maleimide GGFG-DXd was added along with DMA to a final concentration of 10%. The reaction mixture was incubated at room temperature overnight followed by buffer exchange to 20 mM succinate, 8% sucrose, pH 6.5 using a desalting column. The purified EpCAM-GGFG-DXd immunoconjugate is tested with the following QC assays:
concentration and drug-to-antibody (or activatable antibody) ratio (DAR) are measured with UV-vis spectrophotometry method using a SoloVPE instrument;
% monomer is measured with the SEC-HPLC method;
content of free drug-related impurities (FDRI) is evaluated with the HiSep-HPLC method.

Purity and integrity of the immunoconjugate was confirmed using LC-MS and CE-SDS assays.

### 7.4.4 Preparation of Huepcam23Gv4.2-Mmat

The activatable CX-231 antibody conjugation to P2-MMAT linker toxin is usually a 3-day process. The tris(2-carboxyethyl)phosphine (TCEP) reduction is performed on day 1 to remove the capping on L328C cysteines, which also breaks up hinge disulfide bonds in probodies. On day 2 the excessive TCEP is removed via buffer exchange; the hinge disulfide bonds are reformed during the dehydroabietic acid (DHAA) oxidation; then the de-capped L328C cysteine conjugate to the AGL-01332-93I. The conjugates are purified on day 3 via buffer exchange which removes free drug-linkers.

### Day 1: TCEP Reduction

Activatable antibody in PBS, 1-10 mg/mL
Add 1/100 V of EDTA (500 mM stock solution pH 8.2) to 5 mM.
Add 12 eq TCEP (10 mM stock solution in 50 mM HEPES buffer pH 7.5)
Stir RT 2 h. Store at 4 C ON.

### Day 2: DHAA Oxidation and AGL01332-93I Conjugation

Buffer exchange with PBS using protein concentrator.
Re-dissolve reduced probodies in PBS to 2-10 mg/mL
Add 1/100 V of EDTA (500 mM stock solution pH 8.2) to 5 mM.
Add 10 eq of DHAA (10 mM in DMSO).
Stir RT 1 h. Proceed to next step.
AGL-01332-93I prepared before (6.26 mM in DMSO, ~100% pure by RP-HPLC).
Add 1/10 V of borate buffer (500 mM pH 8.2) to adjust pH.
Add 6-9 eq of AGL01332-93I, depending on the reactivity of the activatable antibody. Stir RT ON.

### Day 3: Clean Up and Analysis

Buffer exchange with PBS using protein concentrators. Concentrate conjugates to 5-10 mg/mL in PBS.

The purified EpCAM-P2-MMAT immunoconjugate is tested with the following QC assays:
concentration and drug-to-antibody (or activatable antibody) ratio (DAR) are measured with UV-vis spectrophotometry method using a SoloVPE instrument;
% monomer is measured with the SEC-HPLC method;
content of free drug-related impurities (FDRI) is evaluated with the HiSep-HPLC method.

Purity and integrity of the immunoconjugate was confirmed using LC-MS and CE-SDS assays.

### 7.4.5 Preparation of Huepcam23Gv4.2-Gmbs-Dm21L

To make the DM21-L-G stock solution (approx. 50 mM), 1.517 mL of anhydrous DMA was added to a vial of 106.6 mg of DM21-L-G solid and the vial was vortexed until all solids dissolved. The concentration of this stock solution was then determined using the UV spectrometry method (see section 5.1) to be 53.95 mM.

34.87 mL (approx. 7 volume; 34.78 g) of EPPS buffer 2 was transferred into a 3-neck round bottom flask, followed by adding 5 mL (1 volume; 4.7 g) of DMA and mixing with stirring. 10.13 mL of CX-231 stock solution (24.69 g/L; approx. 2 volume; 10.23 g; previously thawed at 4 °C) was then added and the reaction vessel was equilibrated to 16 °C by stirring in a dry bath. At this point the reaction pH was measured to be 8.088. The reaction time was set to T=0 immediately after the first addition of DM21-L-G.

To the reaction vessel, 2.0 Molar Eq of DM21-L-G (59.89 uL of 53.95 mM stock solution) was added at T=0; 2 h; 4 h. The total amount of DM21-L-G was 6.0 Molar Eq (179.67 uL of 53.95 mM stock solution; 0.16 g); total reaction time was 6 h. At T=6 h, 1/9 volume of the quenching buffer (5.55 mL: 5.66 g) was added to each reaction to terminate the conjugation process and the crude reaction mixture was stored at −80 °C until purification.

EpCAM-DM21 immunoconjugate was purified by buffer exchanging against 10 V of the EpCAM-DM21 formulation buffer via TFF. The clearance of FDRI was monitored by sampling the permeate and analyzed by RP-HPLC. The product was filtered with a 0.2 um syringe filter after TFF.

The purified EpCAM-P2-MMAT immunoconjugate is tested with the following QC assays:
concentration and drug-to-antibody (or activatable antibody) ratio (DAR) are measured with UV-vis spectrophotometry method using a SoloVPE instrument;
% monomer is measured with the SEC-HPLC method;
content of free drug-related impurities (FDRI) is evaluated with the HiSep-HPLC method.

Purity and integrity of the immunoconjugate was confirmed using LC-MS and CE-SDS assays

### 7.5 Example 5. In Vitro Cytotoxicity of Epcam Activatable Antibody Immunoconjugates

The ability of EpCAM-targeting antibody-drug conjugates and activatable antibody drug-conjugates comprising a camptothecin-derived linker-payload to kill tumor cells was assayed using in vitro cytotoxicity assays.

In vitro cytotoxicity of ADC/immunoconjugate was done by incubating the molecules with EpCAM target expressing cancer cell lines, and cell viability was determined by Celltiter-glo following 5 days of incubation. Cells were seeded 2,000 cells per well in a 96-well plate and were allowed to adhere to the plates overnight. Test articles were serially titrated 5-fold for 9 points starting at 100 nM and added to the cells. Cells were then incubated for 5 days at 37 °C. Cell-Titer Glo reagent was added to each well, and luminescence was measured on a SpectroMax M5 plate reader.

The percent viability was calculated by dividing each treated sample value by the average value of wells with untreated cells. Data was analyzed in excel and plotted in GraphPad Prism. EC₅₀ values were determined using non-linear regression 4-parameter-logistic curve fit in Prism. The EC₅₀ and Area Under the Curve (AUC) was determined from cytotoxicity levels vs. concentration of drug plots.

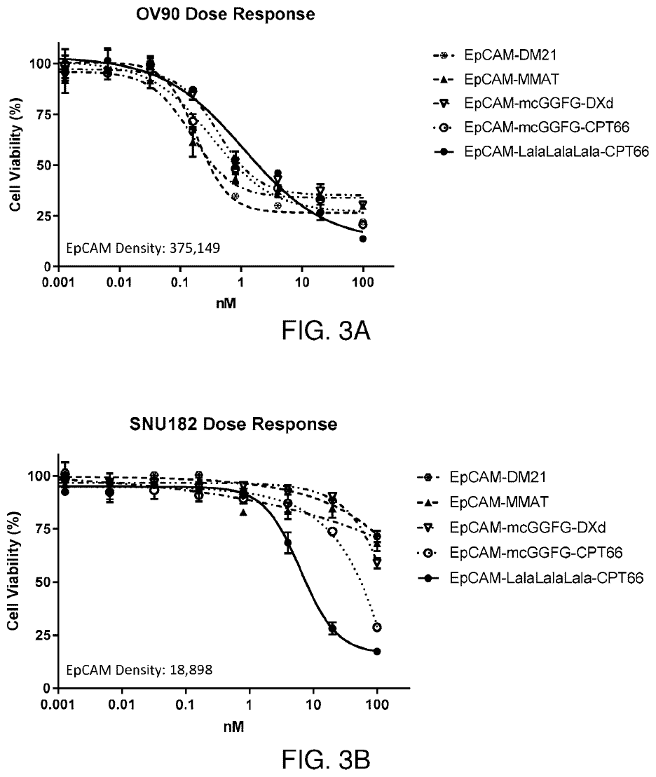

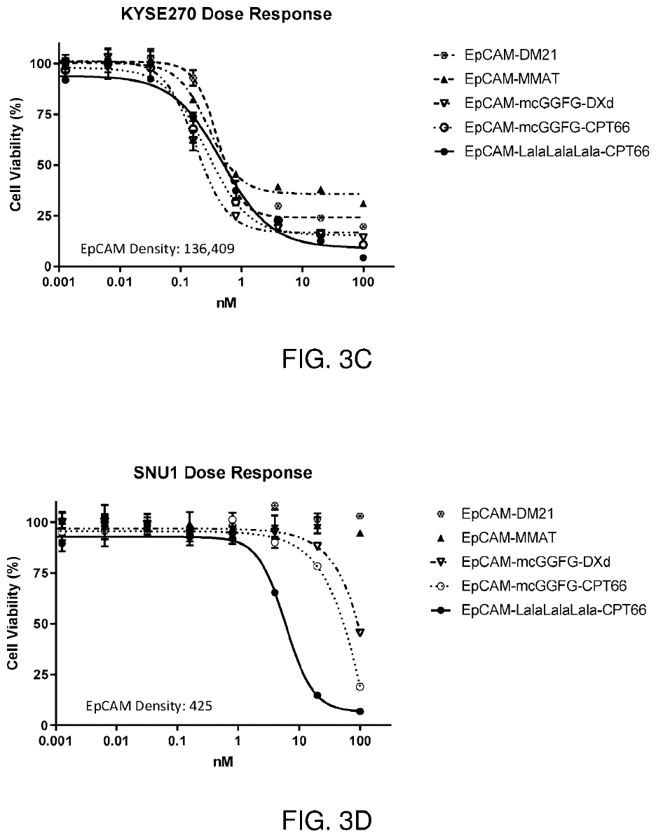

FIGS. 3A-3D show cytotoxicity assessment for OV90 (FIG. 3A), SNU182 (FIG. 3B), KYSE270 (FIG. 3C), and SNU1 (FIG. 3D) cancer cell lines with various EpCAM linker-payloads. EpCAM-DM21 was conjugated to DM21 maytansinoid linker payload with a DAR of 4, EpCAM-MMAT was a site-specific conjugation of auristatin derivative via a valine-citrulline linker with a DAR of 2, EpCAM-mGGFG-DXd was conjugated to linker-payload used for fam-trastuzumab deruxtecan-nxki with a DAR of 8, EpCAM-mGGFG-CPT66 is CPT66 payload conjugated with GGFG linker (SEQ ID NO:310) from fam-trastuzumab deruxtecan-nxki with DAR of 8, and EpCAM-Lala-ala-ala-CPT66 was conjugated to CPT66 payload using a tripeptide linker (ala-ala-ala) with DAR of 8. OV90 is an ovarian cancer cell line with -375,149 EpCAM receptors expressed on the cell surface, SNU182 is a liver cancer cell line with 18,898 EpCAM receptors, KYSE270 is an esophageal cancer cell line with 136,409 EpCAM receptors, and SNU1 is a gastric cancer cell line with 425 EpCAM receptors. The potency of EpCAM-CPT was comparable to that of EpCAM-GGFG-DXd ADC across cell lines with varying EpCAM receptor expression.

Generally, the cytotoxicity potency of EpCAM-CPT66 was comparable to that of EpCAM-GGFG-DXd. Median EC₅₀ across all tested cancer cell lines from various indication was 1.5 nM, with the lowest EC₅₀ being from ovarian (0.16), head and neck (0.86), colorectal (0.9), and breast (1.5).

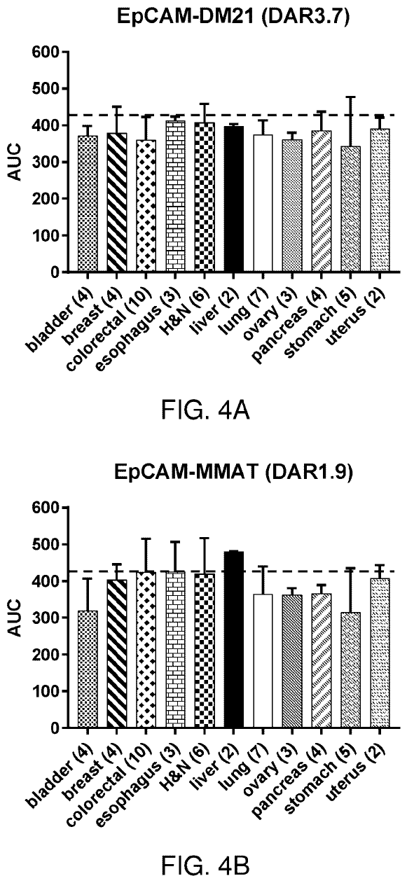

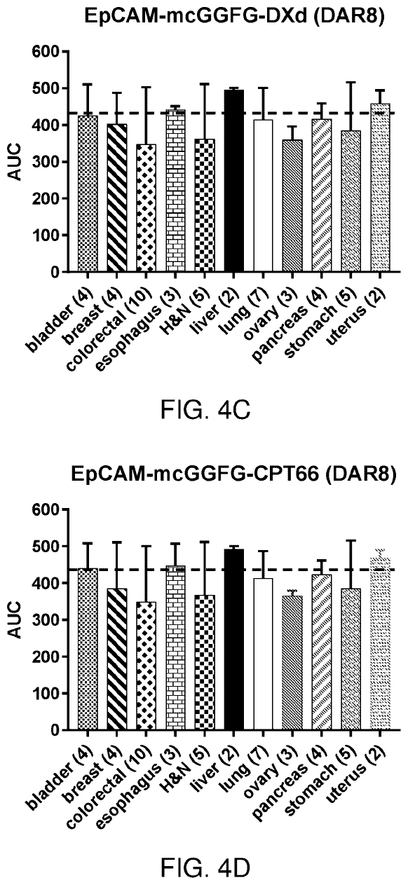

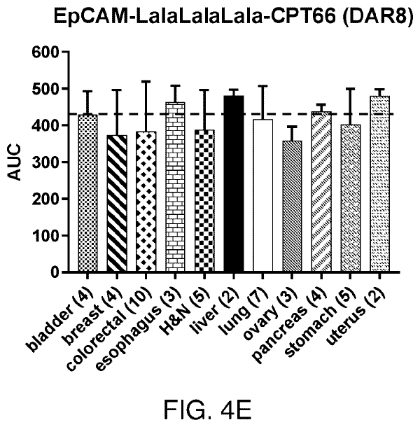

In vitro cytotoxicity suggested that ovarian, head and neck, colorectal, and breast cancers were relatively sensitive to EpCAM-CPT66, whereas bladder, uterine, liver, and esophagus cancer cell lines were relatively insensitive to the ADC. FIGS. 4A-4E show cytotoxicity assessment of 43 cancer cell lines treated with various EpCAM linker payloads listed in FIGS. 3A-3D. The cytotoxicity activity was shown as area under the curve (AUC) that integrated the AUC of the cytotoxicity curves shown in FIGS. 3A-D, and the lower the AUC, the higher the potency of the ADC. Average AUC for each cancer indications was plotted, and the number of cell lines tested were indicated in parenthesis. Error bars represented standard error measurements. Data suggest that breast, CRC, ovarian, and head and neck cancer cell lines are relatively sensitive to EpCAM-CPT66 ADC, whereas bladder, uterine, liver, and esophagus cancer cell lines are relatively insensitive to the ADC.

Anti-tumor efficacy of EpCAM-CPT66 was evaluated by administering the immunoconjugate for cell line derived tumor (CDX) models and patient-derived xenograft (PDX) models. For CDX models, tumor-bearing mice were given a single injection of 2.5-10 mg/kg of EpCAM-CPT66 and tumor volume were monitored twice per week.

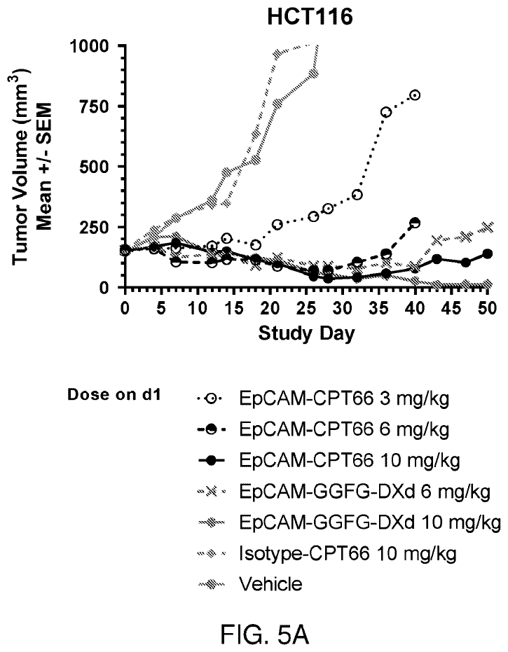

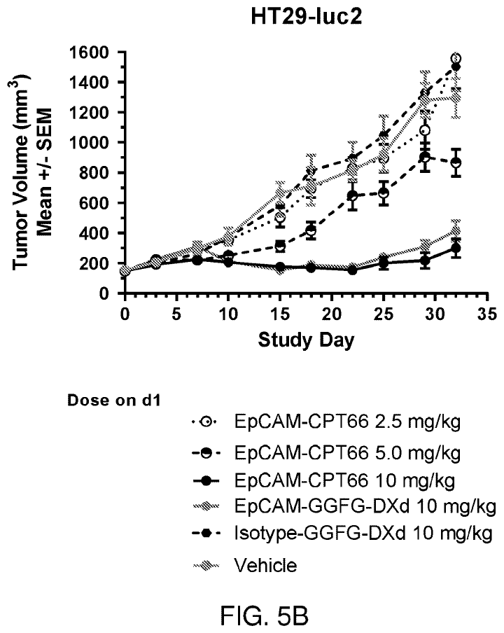

FIGS. 5A-5B shows an in vivo anti-tumor assessment of the EpCAM-CPT66 immunoconjugate in two colorectal cancer cell line-derived xenograft models, HCT116 (FIG. 5A) and HT29 (FIG. 5B). EpCAM-CPT66 was conjugated to CPT66 with a tripeptide (ala-ala-ala) linker and DAR of 8, and it was compared to EpCAM-GGFG-DXd conjugated to the linker-payload similar to that of fam-trastuzumab deruxtecan-nxki with DAR of 8. All immunoconjugate and Isotype control-ADC were given as a single dose with the indicated mg/kg (mpk) via intravenous injection at day 0. Anti-tumor activity of EpCAM-CPT66 was comparable to that of EpCAM-GGFG-DXd.

EpCAM-CPT appears to induce tumor-regression at 10 mg/kg dosing, and the anti-tumor activity was dose-dependent from 2.5 to 10 mg/kg. Anti-tumor activity of EpCAM-CPT66 was comparable to that of EpCAM-GGFG-DXd.

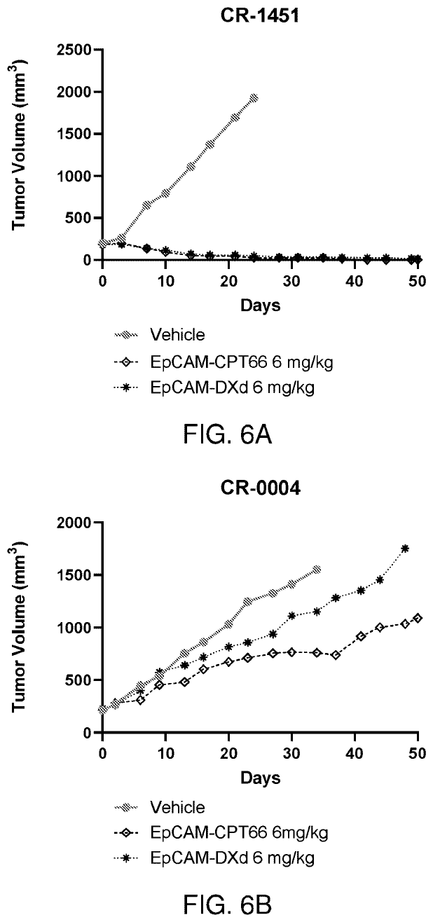

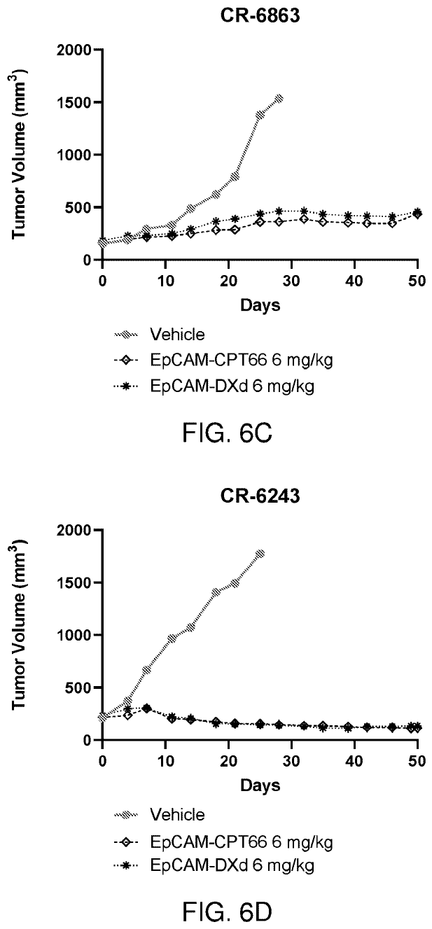

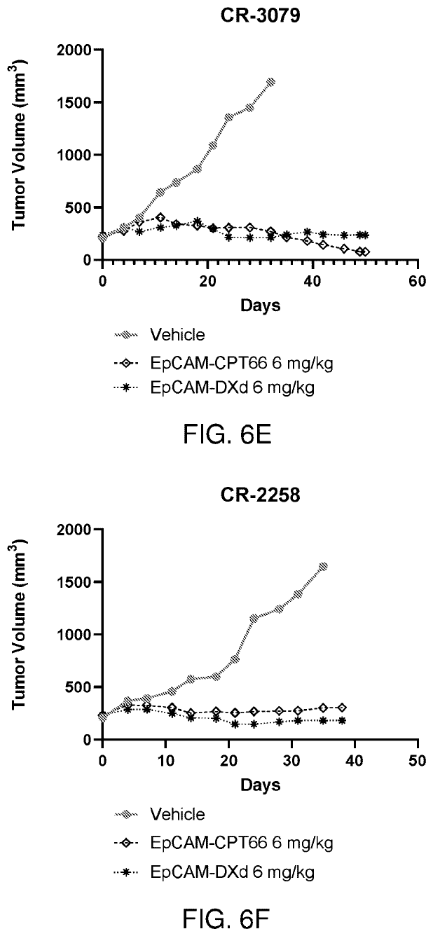

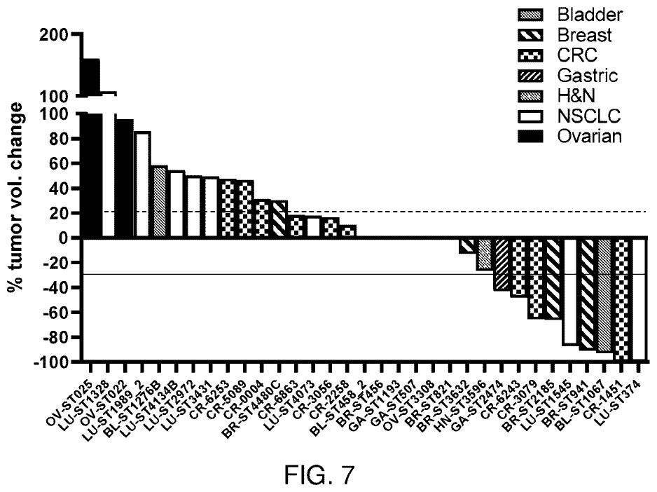

For PDX models, tumor-bearing mice were dosed with 6-12 mg/kg every two weeks for total of 3 drug administration (Q2W×3). FIGS. 6A-6F shows an in vivo anti-tumor assessment of EpCAM-CPT66 in colorectal patient-derived xenograft (PDX) models. Each model was dosed with 6 mg/kg (mpk) of EpCAM-CPT66 and was compared with the same dose of EpCAM-GGFG-DXd. The immunoconjugate dosing schedule was once every two weeks for a total of 3 doses (Q2W×3). Greater than 30% tumor volume reduction were observed for -32% of the models tested, and breast and CRC were the most sensitive tumor indications for EpCAM-CPT66. See FIG. 7. In a head-to-head comparison of antitumor activity between EpCAM-CPT66 vs. EpCAM-GGFG-DXd in CRC PDX models, the anti-tumor activity appeared to be similar between the EpCAM conjugates. Overall anti-tumor activity of EpCAM-CPT66 was comparable to that of EpCAM-GGFG-DXd.

These data suggested that EpCAM-CPT66 compared favorably to the benchmark linker-payload of fam-trastuzumab deruxtecan-nxki.

### 7.6 Example 6. Expanded In Vitro Cytotoxicity Study of Epcam Activatable Antibody Immunoconjugates

An objective of this expanded study was to evaluate the in vitro cytotoxic effects of EpCAM-CPT66 on the human colorectal cancer cell line HCT116 using a 5-day cell viability assay. The cell viability assay measured the total cellular adenosine triphosphate (ATP) to evaluate cell viability at the end of a 5-day incubation with test articles (e.g., EpCAM(−)-CPT66, which comprises an EpCAM antibody (not activatable) conjugated to CPT66; and EpCAM-CPT66, which comprises an activatable EpCAM antibody conjugated to CPT-66). By comparing the cytotoxic activity of intact and protease-activated EpCAM-CPT66 with EpCAM(−)-CPT66, the effect of the masking moiety of the activatable antibody on cytotoxic characteristics can be evaluated.

A second objective of the expanded study was to evaluate the cytotoxic activity of the unmasked immunoconjugate EpCAM(−)-CPT66 to understand its potency on a panel of human cancer cell lines from various indications.

### 7.6.1 Materials and Methods

### 7.6.1.1 Test Articles

TABLE 20
Test Articles

| Name | Description |
|---|---|
| EpCAM-CPT66 | Activatable EpCAM antibody conjugated to CPT66 |
| EpCAM-CPT66 + | EpCAM-CPT66 digested with matriptase |
| MT-SP1 | |
| EpCAM(−)-CPT66 | Unmasked EpCAM antibody conjugated to CPT66 |

MT-SP1 = matriptase or membrane-type serine protease 1

Test articles were prepared in formulation buffer of 20 millimolar (mM) succinate, 8% sucrose, 0.01% polysorbate 20, pH 6.0, and kept frozen in −80 degrees Celsius (°C). Test articles were thawed and diluted in formulation buffer or in phosphate buffered saline (PBS) immediately prior to use.

### 7.6.1.2 Cell Lines

HCT116 was purchased from American Type Culture Collection (Manassas, Virginia, catalog no. CCL-247). Cells were cultured in McCoy's 5a Medium Modified (Thermo Fisher, catalog no. 16600-082) supplemented with 10% fetal bovine serum (FBS) (Thermo Fisher, catalog no. 10082-147) and maintained in a humidified atmosphere of 5% CO₂ at 37 °C.

A total of 57 cell lines were used in this study with 53 cell lines purchased from American Type Culture Collection (ATCC) and four purchased from the Leibniz Institute DSMZ (Brunswick, Germany). Cell lines were cultured in recommended media according to vendor specifications and are summarized in Table 23 below.

### 7.6.1.3 Protease Activation of Epcam-CPT66

EpCAM-CPT66 was incubated overnight at 37 °C with matriptase (R&D systems, catalog no. 3946-SEB) at 7:1 molar ratio of immunoconjugate (1 milligram/milliliter [mg/mL]) to matriptase. Reactions were carried out in a final volume of 100 or 200 microliters (μL) in 0.5 mL Eppendorf tubes.

### 7.6.1.4 Cytotoxicity Assay

Cells were harvested using Accutase (Thermo Fisher, catalog no. 00-4555-56) and seeded at 2,000 cells per well in a 96-well flat-bottom plate (Corning, catalog no. 07-200-587). Cells were allowed to adhere to the plates overnight. Next, test articles were serially titrated 5-fold for a 9-point dilution series starting at 100 nanomolar (nM). Cells were then incubated for 5 days at 37 °C with test articles in duplicates. Then, 100 μL of Cell-Titer Glo reagent (Promega, catalog no. G7573) was added to each well, and luminescence was measured on a SpectroMax M5 plate reader (Molecular Devices, Sunnyvale, CA). Data was analyzed using Microsoft Excel and plotted using GraphPad Prism (GraphPad Software, La Jolla, California). Half maximal effective concentration (EC₅₀) values were determined using a non-linear regression 4-parameter-logistic (4-PL) curve fit in Prism.

### 7.6.2 Results

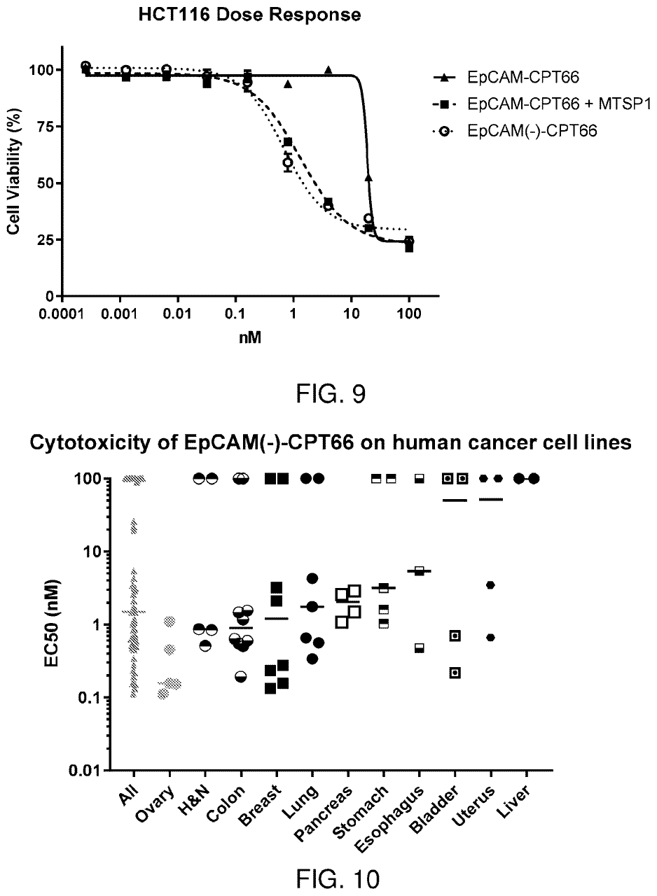

Cytotoxicity of EpCAM-CPT66 (intact and matriptase-activated) and the corresponding EpCAM(−)-CPT66 (i.e., with no masking moiety) were assessed using a 5-day cell viability assay on HCT116, a human colorectal cancer cell line. As depicted in FIG. 9, in HCT116 colorectal carcinoma cell line, intact EpCAM-CPT66 showed attenuated cell killing potential with an EC₅₀ value of 19.1 nM when compared to EpCAM(−)-CPT66. EpCAM(−)-CPT66 showed potent cytotoxicity with an EC₅₀ value of 0.69 nM. Activation of EpCAM-CPT66 by matriptase restored the cell killing potential of EpCAM-CPT66 to within 2-fold of EpCAM(−)-CPT66, with an EC₅₀ value of 1.34 nM. This data showed that cytotoxicity was significantly reduced by the masking moiety of EpCAM-CPT66 but can be restored after activation with matriptase to comparable activity as seen with the unmasked EpCAM(−)-CPT66. Cytotoxicity dose-response curves with HCT116 are shown in FIG. 9 and EC₅₀ values are summarized in Table 21.

TABLE 21
Cytotoxicity EC₅₀ Values of Test Articles on HCT116 Cells

| Test Article | EC₅₀ (nM) |
|---|---|
| EpCAM-CPT66 | 19.1 |
| EpCAM-CPT66 + MT-SP1 | 1.34 |
| EpCAM(−)-CPT66 | 0.69 |

Cytotoxicity of the unmasked ADC, EpCAM(−)-CPT66, was assessed on 57 cell lines from 11 cancer indications (Table 22 and FIG. 10). Overall, EpCAM(−)-CPT66 is potent to varying degrees on all 57 cell lines tested with a median EC₅₀ value of 1.49 nM. Ovarian, head & neck, and colorectal cancer cells were most sensitive to EpCAM(−)-CPT66, with median EC₅₀ values <1 nM. Cancer cells of breast, lung, pancreas, stomach, and esophagus origin responded moderately to EpCAM(−)-CPT66, with median EC₅₀ values ranging from 1.2 to 5.4 nM. Lastly, bladder, uterine, and liver cancer cells were most resistant with EC₅₀ values >50 nM. Table 22 lists the median EC₅₀ values for each cancer indication. Table 23 shows the EC₅₀ values for all human cell lines tested.

TABLE 22
Cytotoxicity EC50 Values of the Unmasked ADC, EpCAM(−)-CPT66,
on Human Cancer Cell Lines by Indication

| Tissue | Median EC₅₀ (nM) | # of Cell Lines |
|---|---|---|
| Ovary | 0.16 | 5 |
| H & N | 0.86 | 5 |
| Colon | 0.90 | 10 |
| Breast | 1.20 | 8 |
| Lung | 1.76 | 7 |
| Pancreas | 2.04 | 4 |
| Stomach | 3.17 | 5 |
| Esophagus | 5.40 | 3 |
| Bladder | 50.35 | 4 |
| Uterus | 51.74 | 4 |
| Liver | 59.80 | 2 |

TABLE 23
Cytotoxicity EC₅₀ Values of EpCAM(−)-CPT66 on Human Cancer Cell Lines

| | | EpCAM(−)-CPT66 | |
|---|---|---|---|
| Cell Line | Tissue | EC50 (nM) | Culture Media |
| 5637 | Bladder | 0.220 | RPMI-1640 + 10% FBS |
| HT-1197 | Bladder | 0.701 | EMEM + 10% FBS |
| RT4 | Bladder | 100* | McCoy's 5a Medium Modified + 10% FBS |
| SW780 | Bladder | 100* | RPMI-1640 + 10% FBS |
| HCC1806 | Breast | 0.133 | RPMI-1640 + 10% FBS |
| HCC1569 | Breast | 0.156 | RPMI-1640 + 10% FBS |
| MCF7 | Breast | 0.235 | EMEM + 10% FBS |
| T47D | Breast | 0.278 | RPMI-1640 + 10% FBS |
| HCC38 | Breast | 2.114 | RPMI-1640 + 10% FBS |
| ZR751 | Breast | 3.235 | RPMI-1640 + 10% FBS |
| HCC1143 | Breast | 100* | RPMI-1640 + 10% FBS |
| HCC1937 | Breast | 100* | RPMI-1640 + 10% FBS |
| COLO205 | Colon | 0.193 | RPMI-1640 + 10% FBS |
| LS174T | Colon | 0.504 | EMEM + 10% FBS |
| SW480 | Colon | 0.549 | RPMI-1640 + 10% FBS |
| LS513 | Colon | 0.599 | RPMI-1640 + 10% FBS |
| LS180 | Colon | ~0.638 | EMEM + 10% FBS |
| DLD1 | Colon | 1.164 | RPMI-1640 + 10% FBS |
| SNU-C1 | Colon | 1.466 | RPMI-1640 + 10% FBS |
| HCT116 | Colon | 1.557 | McCoy's 5a Medium Modified + 10% FBS |
| HT29 | Colon | 100* | McCoy's 5a Medium Modified + 10% FBS |
| LoVo | Colon | 100* | F12K + 10% FBS |
| KYSE270 | Esophagus | 0.478 | 49% RPMI-1640 + 49% Ham's F12 + |
| | | | 2% FBS |
| KYSE30 | Esophagus | 5.403 | 45% RPMI-1640 + 45% Ham's F12 + |
| | | | 10% FBS |
| KYSE150 | Esophagus | 100* | 49% RPMI-1640 + 49% Ham's F12 + |
| | | | 2% FBS |
| FaDu | H & N | 0.512 | EMEM + 10% FBS |
| CAL27 | H & N | 0.839 | DMEM + 10% FBS |
| Detroit562 | H & N | 0.861 | EMEM + 10% FBS |
| A253 | H & N | 100* | McCoy's 5a Medium Modified + 10% FBS |
| BHY | H & N | 100* | DMEM + 10% FBS |
| HepG2 | Liver | 100* | EMEM + 10% FBS |
| Hep3B217 | Liver | 100* | EMEM + 10% FBS |
| Calu-3 | Lung | 0.342 | EMEM + 10% FBS |
| NCI-H358 | Lung | 0.563 | RPMI-1640 + 10% FBS |
| NCI-H520 | Lung | 0.656 | RPMI-1640 + 10% FBS |
| NCI-H441 | Lung | 1.760 | RPMI-1640 + 10% FBS |
| NCI-H292 | Lung | 4.285 | RPMI-1640 + 10% FBS |
| NCI-H1975 | Lung | 100* | RPMI-1640 + 10% FBS |
| NCI-H2110 | Lung | 100* | RPMI-1640 + 10% FBS |
| OVCAR3 | Ovary | 0.112 | RPMI-1640 + 20% FBS + 0.01 mg/mL |
| | | | Bovine Insulin |
| Caov3 | Ovary | 0.152 | DMEM + 10% FBS |
| Caov4 | Ovary | 0.157 | RPMI-1640 + 10% FBS |
| SW626 | Ovary | 0.453 | RPMI-1640 + 10% FBS |
| OV90 | Ovary | 1.102 | Medium 199 + 15% FBS + 2 mg/mL Sodium |
| | | | Bicarbonate |
| Panc02.13 | Pancreas | 1.079 | RPMI-1640 + 15% FBS + 20 units/mL |
| | | | Human Recombinant Insulin |
| HPAFII | Pancreas | 1.493 | EMEM + 10% FBS |
| BxPC3 | Pancreas | 2.582 | RPMI-1640 + 10% FBS |
| Panc04.03 | Pancreas | 2.869 | RPMI-1640 + 15% FBS + 20 units/mL |
| | | | Human Recombinant Insulin |
| AGS | Stomach | 1.019 | F12K + 10% FBS |
| SNU16 | Stomach | 1.621 | RPMI-1640 + 10% FBS |
| NCI-N87 | Stomach | 3.173 | RPMI-1640 + 10% FBS |
| KATOIII | Stomach | 100* | IMDM + 20% |
| SNU-5 | Stomach | 100* | IMDM + 20% |
| RL95-2 | Uterus | 0.661 | DMEM:F12 + 10% FBS + 5 ug/mL Insulin |
| HEC1A | Uterus | 3.478 | McCoy's 5a Medium Modified + 10% FBS |
| HEC1B | Uterus | 100* | EMEM + 10% FBS |
| KLE | Uterus | 100* | DMEM:F12 + 10% FBS |

Representative EC50 value is tabulated for experiments that were repeated more than once.
*EC50 was assigned as 100 nM for non-saturating curves, in cell lines where maximal reduction in cell viability >90% was not observed at the highest drug concentration tested (100 nM).
DMEM = Dulbecco's Modified Eagle's Medium; EC₅₀ = half maximal effective concentration; EMEM = Eagle's Minimum Essential Medium; FBS = fetal bovine serum; H & N = head and neck; IMDM = Iscove's Modified Dulbecco's Medium; mg = milligram; mL = milliliter; nM = nanomolar; RPMI = Roswell Park Memorial Institute Medium; μg = microgram

Proteolytic cleavage of a cleavable moiety within the activatable EpCAM antibodies of the disclosure by tumor-associated proteases is necessary to release the peptide mask and allow the unmasked/activated therapeutic to bind to the target antigen, deliver the cytotoxic payload to the target cell, and elicit cell killing. As reported here, the cytotoxic activity of intact/masked EpCAM-CPT66 was attenuated ~28-fold when compared to the unmasked antibody drug conjugate EpCAM(−)-CPT66. After matriptase digestion of the protease-cleavable substrate in EpCAM-CPT66, cytotoxicity was restored to within 2-fold of EpCAM(−)-CPT66. These results demonstrate that the mask and cleavable moieties of EpCAM-CPT66 are functioning as designed.

The potency of the unmasked ADC, EpCAM(−)-CPT66, was evaluated on 57 cell lines to assess drug sensitivity across 11 cancer indications. EpCAM(−)-CPT66 was found most effective at killing cancer cells of the ovary, head and neck, and colon (median EC₅₀ values <1 nM). Breast, lung, pancreatic, stomach, and esophageal cancer cells were more moderately sensitive to EpCAM(−)-CPT66 (median EC₅₀ values from 1.2 to 5.4 nM). By contrast, bladder, uterine, and liver cancer cells were the most resistant to EpCAM(−)-CPT66 (median EC₅₀ values >50 nM).

### 7.7 Example 7. Evaluation of Tolerability of Epcam Activatable Antibody Immunoconjugates

To assess potential toxicities and maximum tolerated dose, EpCAM-CPT66 was administered by intravenous dosing in cynomolgus monkeys with 10, 30, 60 mg/kg on Days 1, 15 and 29 (Q2W x 3) and with 90 mg/kg on Days 1 and 15 (Q2W×2) with or without 5 weeks of recovery. Cage side observation and mortality checks were performed daily. Individual body weights, qualitative food consumptions measured weekly and ophthalmic examinations were performed pretreatment and at week 5. EpCAM-CPT66 was clinically tolerated up to 60 mg/kg (3/3 animal), while at 90 mg/kg it was not tolerated as 1 out of 2 animal was found deceased at Day 9 after drug administration. This was in contrast to EpCAM-DM21, which was clinically tolerated up to 6 mg/kg. The 60 mg/kg tolerated dose of EpCAM-CPT66 also compared favorably to fam-trastuzumab deruxtecan-nxki, where the HNSTD was determined to be 30 mg/kg in Cyno.

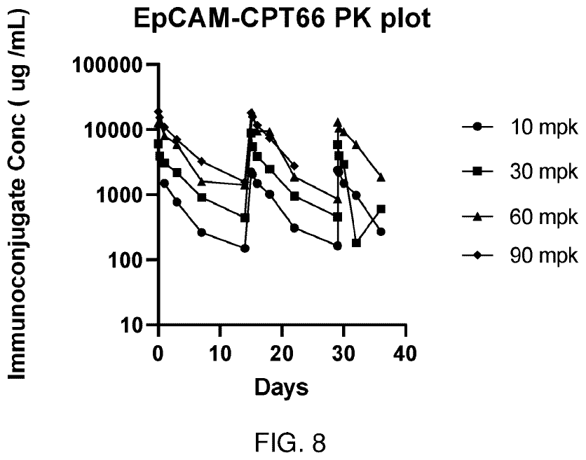

Clinical signs included, BW loss, liquid feces, poor appetite and hunched posture (≥60 mg/kg). Changes in red blood cell parameters were observed at ≥60 mg/kg, which appeared to resolve on recovery. Mild decrease in A/G ratio ≥60 mg/kg, showed a lack of recovery. There was no test article (TA) related findings at 10 and 30 mg/kg both during treatment and recovery phase. At the end of the treatment period, no TA-related gross findings were noted, and dose-dependent microscopic findings were noted in the small and large intestines including duodenum, jejunum, ileum, cecum, and/or colon) and lymphoid tissues including spleen, thymus, gut-associated lymphoid tissue, and mesenteric lymph node at ≥60 mg/kg and liver at 90 mg/kg/dose. At recovery euthanasia, no TA-related macroscopic or microscopic findings were noted suggesting complete recovery. At ≥60 mg/kg, TA-related organ weight changes were noted in the thymus (decrease) and the end of dosing. The thymic weight decreases partially recovered with a 5-week dose-free interval. The cause of early death was attributed to TA-related epithelial degeneration and inflammation in the GI tract; minimal bone marrow decreased cellularity and systemic decreased lymphoid cellularity. There was no lung toxicity observed at any dose level tested. Lack of lung toxicity findings in the tolerability study also suggests that EpCAM-CPT66 may be better tolerate than fam-trastuzumab deruxtecan-nxki, as lung toxicity was seen at 30 mg/kg in Cyno for fam-trastuzumab deruxtecan-nxki which translated to interstitial lung disease (ILD) observed in the clinic. Tolerability is summarized in Table 24, while toxicokinetics of EpCAM-CPT66 are summarized in Table 25. FIG. 8 depicts a PK plot demonstrating good EpCAM-CPT66 exposure after each of two or three doses, with dose-proportional increases in exposure. The drug exposure was consistent following 2 or 3 doses, suggesting no anti-drug antibody that affects clearance of the immunoconjugate was seen.

TABLE 24
Clinical tolerability of EpCAM-CPT66

| Dosing | EpCAM-CPT66 | Isotype |
|---|---|---|
| 10 mg/kg (Q2W × 3) | Tolerated (3/3) | Not tested |
| 30 mg/kg (Q2W × 3) | Tolerated (2/2) | Not tested |
| 60 mg/kg (Q2W × 3) | Tolerated (3/3) | Tolerated (2/2) |
| 90 mg/kg (Q2W × 2) | Non-tolerated dose (1/2) | Not tested |

TABLE 25
Toxicokinetics profile for EpCAM-CPT66

TK Parameters

| Dose | Cmax (μg/mL) | AUC 0-t (Day* μg/mL) |
|---|---|---|
| 10 mg/kg | 238 | 722 |
| 30 mg/kg | 665 | 1890 |
| 60 mg/kg | 1420 | 4397 |
| 90 mg/kg | 1940 | 6730 |

### 7.7.1 1-Month Repeat Dose GLP Toxicity Study of Epcam-CPT66 in Cynomolgus Monkeys

Tolerability of EpCAM-CPT66 was also assessed in a 1-month repeat dose GLP toxicity study where EpCAM-CPT66 was administered by intravenous dosing in cynomolgus monkeys at 15, 45 and 75 mg/kg on Days 1, 15 and 29 (Q2W×3) with 6 weeks of recovery. EpCAM-CPT66 was well tolerated at 15 and 45 mg/kg. 1 out of 10 animals did not tolerate a dose of 75 mg/kg and was euthanized early on Day 15.

### 7.7.2 Comparison Between Immunoconjugates Comprising Epcam Antibody and Epcam Activatable Antibody

Unmasked immunoconjugate (EpCAM antibody with conjugated CPT66 linker-payload; EpCAM(−)-CPT66) was also evaluated in a tolerability assay, where it was dosed intravenously in cynomolgus monkey at 10 mg/kg at Q2W×2. This animal made it to scheduled necropsy with mild intervention, mild clinical signs including vomitus and low food consumption. Hematology revealed mild signs of neutropenia and reticulocytosis. At the end of treatment period gross observation was noted in the cecum. Microscopic findings were noted in the lung. GI tract, lymphoid tissues. In contrast, there was no test article-related findings at 10 mg/kg dosing of EpCAM-CPT66 (i.e., with EpCAM activatable antibody) in cynomolgus monkeys.

### 8. NUMBERED EMBODIMENTS

While various specific embodiments have been illustrated and described, it will be appreciated that various changes can be made without departing from the spirit and scope of the disclosure(s). The present disclosure is exemplified by the numbered embodiments set forth below:

1. An immunoconjugate comprising:
(a) an activatable antibody comprising a full length human IgG1 EpCAM antibody having eight cysteines which, when in a native conformation, form four interchain disulfide bonds, the full length human IgG1 EpCAM antibody comprising a heavy chain comprising or consisting of the amino acid sequence of SEQ ID NO:103 and a light chain comprising or consisting of the amino acid sequence of SEQ ID NO:179; and
(b) eight linker-payloads depicted by the structure:

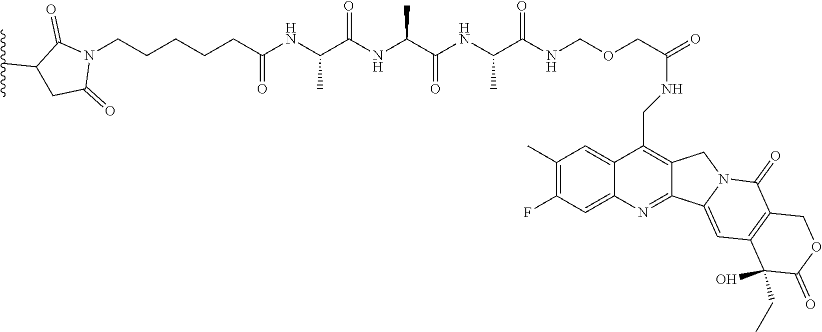 [SMILES: *C1CC(=O)N(CCCCCC(=O)N[C@@H](C)C(=O)N[C@@H](C)C(=O)N[C@@H](C)C(=O)NCOCC(=O)NCc2c3c(nc4cc(F)c(C)cc24)-c2cc4c(c(=O)n2C3)COC(=O)[C@]4(O)CC)C1=O]

or a pharmaceutically acceptable salt thereof, wherein each of the eight linker-payloads is individually covalently bound to the activatable antibody via one of the eight cysteines.

2. An immunoconjugate comprising:
(a) an activatable antibody comprising a full length human IgG1 EpCAM antibody having eight cysteines which, when in a native conformation, form four interchain disulfide bonds, the full length human IgG1 EpCAM antibody comprising a heavy chain consisting of the amino acid sequence of SEQ ID NO:103 and a light chain comprising or consisting of the amino acid sequence of SEQ ID NO:179; and
(b) eight linker-payloads depicted by the structure:

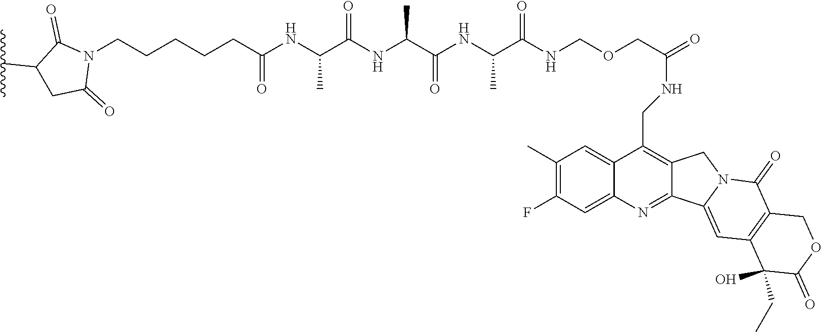 [SMILES: *C1CC(=O)N(CCCCCC(=O)N[C@@H](C)C(=O)N[C@@H](C)C(=O)N[C@@H](C)C(=O)NCOCC(=O)NCc2c3c(nc4cc(F)c(C)cc24)-c2cc4c(c(=O)n2C3)COC(=O)[C@]4(O)CC)C1=O]

or a pharmaceutically acceptable salt thereof, wherein each of the eight linker-payloads is individually covalently bound to the activatable antibody via one of the eight cysteines.

3. An immunoconjugate comprising:
(a) an activatable antibody comprising:
   (i) an EpCAM antibody or EpCAM-binding fragment thereof, comprising:
      (1) a heavy chain complementarity determining region 1 (VH-CDR1) comprising of the amino acid sequence NYYIH (SEQ ID NO:13);
      (2) a heavy chain complementarity determining region 2 (VH-CDR2) comprising the amino acid sequence WIYPGNVYIQYNEKFKG (SEQ ID NO:14);
      (3) a heavy chain complementarity determining region 3 (CDR3) (VH-CDR3) comprising of the amino acid sequence DGPWFAY (SEQ ID NO:15);
      (4) a light chain complementarity determining region 1 (VL-CDR1) comprising the amino acid sequence RSSRSLLHSDGFTYLY (SEQ ID NO:42);
      (5) a light chain complementarity determining region 2 (VL-CDR2) comprising the amino acid sequence QTSNLAS (SEQ ID NO:40); and
      (6) a light chain complementarity determining region 3 (VL-CDR3) comprising the amino acid sequence AQNLELPNT (SEQ ID NO:41);
   (ii) a cleavable moiety coupled to the EpCAM antibody or EpCAM-binding fragment thereof that comprises the amino acid sequence AVGLLAPPGGLSGRSDNI (SEQ ID NO:168) or the amino acid sequence ISSGLLSGRSDNI (SEQ ID NO:169); and
   (iii) a masking moiety coupled to the EpCAM antibody or EpCAM-binding fragment thereof that comprises the amino acid sequence WWPPCQGGAWCEQRI (SEQ ID NO:155), wherein the masking moiety inhibits the binding of the antibody or antibody fragment to EpCAM when the activatable antibody is in an uncleaved state,
      wherein the activatable antibody in an uncleaved state has a structural arrangement from N-terminus to C-terminus of: (masking moiety)-(cleavable moiety)-(antibody or antibody fragment) or (antibody or antibody fragment)-(cleavable moiety)-(masking moiety); and
(b) at least one linker-payload depicted by the structure:

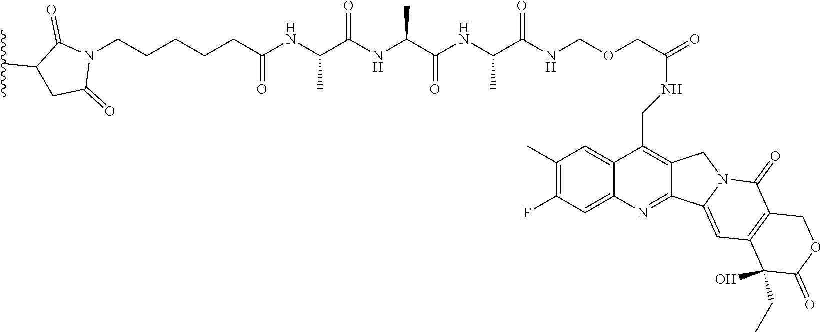 [SMILES: *C1CC(=O)N(CCCCCC(=O)N[C@@H](C)C(=O)N[C@@H](C)C(=O)N[C@@H](C)C(=O)NCOCC(=O)NCc2c3c(nc4cc(F)c(C)cc24)-c2cc4c(c(=O)n2C3)COC(=O)[C@]4(O)CC)C1=O]

or a pharmaceutically acceptable salt thereof.

4. The immunoconjugate of embodiment 3, wherein the cleavable moiety comprises the amino acid sequence ISSGLLSGRSDNI (SEQ ID NO:169).

5. The immunoconjugate of embodiment 3, wherein the cleavable moiety comprises the amino acid sequence AVGLLAPPGGLSGRSDNI (SEQ ID NO:168).

6. The immunoconjugate of any one of embodiments 3 to 5, wherein the immunoconjugate has a drug-to-antibody ratio (DAR) of 8.

7. The immunoconjugate of any one of embodiments 3 to 6, wherein the activatable antibody is a full length human IgG1 EpCAM antibody having eight cysteines which, when in a native conformation, form four interchain disulfide bonds, and wherein each linker-payload is individually covalently bound to the activatable antibody via one of the eight cysteines.

8. An immunoconjugate comprising:
(c) an activatable antibody comprising:
   (i) an EpCAM antibody or EpCAM-binding fragment thereof, comprising:
      (1) a heavy chain complementarity determining region 1 (VH-CDR1) consisting of the amino acid sequence NYYIH (SEQ ID NO:13):
      (2) a heavy chain complementarity determining region 2 (VH-CDR2) consisting of the amino acid sequence WIYPGNVYIQYNEKFKG (SEQ ID NO:14);
      (3) a heavy chain complementarity determining region 3 (CDR3) (VH-CDR3) consisting of the amino acid sequence DGPWFAY (SEQ ID NO:15);
      (4) a light chain complementarity determining region 1 (VL-CDR1) consisting of the amino acid sequence RSSRSLLHSDGFTYLY (SEQ ID NO:42);
      (5) a light chain complementarity determining region 2 (VL-CDR2) consisting of the amino acid sequence QTSNLAS (SEQ ID NO:40); and
      (6) a light chain complementarity determining region 3 (VL-CDR3) consisting of the amino acid sequence AQNLELPNT (SEQ ID NO:41);
   (ii) a cleavable moiety coupled to the EpCAM antibody or EpCAM-binding fragment thereof that consists of the amino acid sequence AVGLLAPPGGLSGRSDNI (SEQ ID NO:168) or the amino acid sequence ISSGLLSGRSDNI (SEQ ID NO:169); and
   (iii) a masking moiety coupled to the EpCAM antibody or EpCAM-binding fragment thereof that consists of the amino acid sequence WWPPCQGGAWCEQRI (SEQ ID NO:155), wherein the masking moiety inhibits the binding of the antibody or antibody fragment to EpCAM when the activatable antibody is in an uncleaved state,
      wherein the activatable antibody in an uncleaved state has a structural arrangement from N-terminus to C-terminus of: (masking moiety)-(cleavable moiety)-(antibody or antibody fragment) or (antibody or antibody fragment)-(cleavable moiety)-(masking moiety); and
(d) at least one linker-payload depicted by the structure:

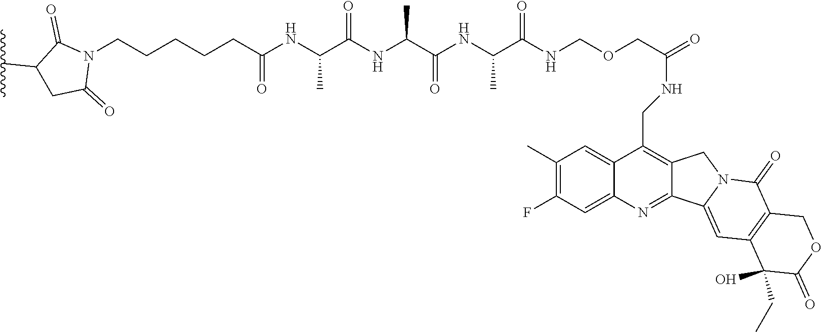 [SMILES: *C1CC(=O)N(CCCCCC(=O)N[C@@H](C)C(=O)N[C@@H](C)C(=O)N[C@@H](C)C(=O)NCOCC(=O)NCc2c3c(nc4cc(F)c(C)cc24)-c2cc4c(c(=O)n2C3)COC(=O)[C@]4(O)CC)C1=O]

or a pharmaceutically acceptable saft thereof.

9. The immunoconjugate of embodiment 8, wherein the cleavable moiety consists of the amino acid sequence ISSGLLSGRSDNI (SEQ ID NO:169).

10. The immunoconjugate of embodiment 8, wherein the cleavable moiety consists of the amino acid sequence AVGLLAPPGGLSGRSDNI (SEQ ID NO:168).

11. The immunoconjugate of any one of embodiments 8 to 10, wherein the immunoconjugate has a drug-to-antibody ratio (DAR) of 8.

12. The immunoconjugate of any one of embodiments 8 to 11, wherein the activatable antibody is a full length human IgG1 EpCAM antibody having eight cysteines which, when in a native conformation form four interchain disulfide bonds, and wherein each linker-payload is individually covalently bound to the activatable antibody via one of the eight cysteines.

13. An immunoconjugate comprising:
(a) an activatable antibody comprising:
   (i) a full length human IgG1 EpCAM antibody having eight cysteines which, when in a native conformation, form four interchain disulfide bonds, the full length human IgG1 EpCAM antibody comprising:
      (1) a heavy chain complementarity determining region 1 (VH-CDR1) comprising the amino acid sequence NYYIH (SEQ ID NO:13);
      (2) a heavy chain complementarity determining region 2 (VH-CDR2) comprising the amino acid sequence WIYPGNVYIQYNEKFKG (SEQ ID NO:14);
      (3) a heavy chain complementarity determining region 3 (CDR3) (VH-CDR3) comprising the amino acid sequence DGPWFAY (SEQ ID NO:15);
      (4) a light chain complementarity determining region 1 (VL-CDR1) comprising the amino acid sequence RSSRSLLHSDGFTYLY (SEQ ID NO:42);
      (5) a light chain complementarity determining region 2 (VL-CDR2) comprising the amino acid sequence QTSNLAS (SEQ ID NO:40); and
      (6) a light chain complementarity determining region 3 (VL-CDR3) comprising the amino acid sequence AQNLELPNT (SEQ ID NO:41);
   (ii) a cleavable moiety coupled to the EpCAM antibody or EpCAM-binding fragment thereof that comprises the amino acid sequence ISSGLLSGRSDNI (SEQ ID NO:169); and
   (iii) a masking moiety coupled to the EpCAM antibody or EpCAM-binding fragment thereof that comprises of the amino acid sequence WWPPCQGGAWCEQRI (SEQ ID NO:155), wherein the masking moiety inhibits the binding of the antibody or antibody fragment to EpCAM when the activatable antibody is in an uncleaved state,
      wherein the activatable antibody in an uncleaved state has a structural arrangement from N-terminus to C-terminus of: (masking moiety)-(cleavable moiety)-(antibody) or (antibody)-(cleavable moiety)-(masking moiety); and
(b) eight linker-payloads each depicted by the structure:

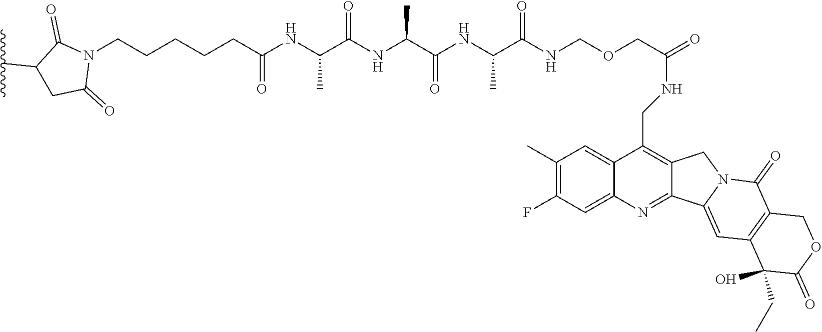 [SMILES: *C1CC(=O)N(CCCCCC(=O)N[C@@H](C)C(=O)N[C@@H](C)C(=O)N[C@@H](C)C(=O)NCOCC(=O)NCc2c3c(nc4cc(F)c(C)cc24)-c2cc4c(c(=O)n2C3)COC(=O)[C@]4(O)CC)C1=O]

or a pharmaceutically acceptable salt thereof, wherein each of the eight linker-payloads is individually covalently bound to the activatable antibody via one of the eight cysteines.

14. The immunoconjugate of embodiment 13, wherein the full length human IgG1 EpCAM antibody comprises a heavy chain variable domain (VH) comprising the amino acid sequence of SEQ ID NO:54 and a light chain variable domain (VL) comprising the amino acid sequence of SEQ ID NO: 89.

15. The immunoconjugate of embodiment 13, wherein the full length human IgG1 EpCAM antibody comprises a heavy chain comprising the amino acid sequence of SEQ ID NO:103 and a light chain comprising the amino acid sequence of SEQ ID NO:179.

16. An immunoconjugate comprising:
(c) an activatable antibody comprising:
   (iv) a full length human IgG1 EpCAM antibody having eight cysteines which, when in a native conformation, form four interchain disulfide bonds, the full length human IgG1 EpCAM antibody comprising:
      (7) a heavy chain complementarity determining region 1 (VH-CDR1) consisting of the amino acid sequence NYYIH (SEQ ID NO:13):
      (8) a heavy chain complementarity determining region 2 (VH-CDR2) consisting of the amino acid sequence WIYPGNVYIQYNEKFKG (SEQ ID NO:14);
      (9) a heavy chain complementarity determining region 3 (CDR3) (VH-CDR3) consisting of the amino acid sequence DGPWFAY (SEQ ID NO:15);
      (10) a light chain complementarity determining region 1 (VL-CDR1) consisting of the amino acid sequence RSSRSLLHSDGFTYLY (SEQ ID NO:42);
      (11) a light chain complementarity determining region 2 (VL-CDR2) consisting of the amino acid sequence QTSNLAS (SEQ ID NO:40); and
      (12) a light chain complementarity determining region 3 (VL-CDR3) consisting of the amino acid sequence AQNLELPNT (SEQ ID NO:41);
   (v) a cleavable moiety coupled to the EpCAM antibody or EpCAM-binding fragment thereof that consists of the amino acid sequence ISSGLLSGRSDNI (SEQ ID NO:169); and
   (vi) a masking moiety coupled to the EpCAM antibody or EpCAM-binding fragment thereof that consists of the amino acid sequence WWPPCQGGAWCEQRI (SEQ ID NO:155), wherein the masking moiety inhibits the binding of the antibody or antibody fragment to EpCAM when the activatable antibody is in an uncleaved state,
      wherein the activatable antibody in an uncleaved state has a structural arrangement from N-terminus to C-terminus of: (masking moiety)-(cleavable moiety)-(antibody) or (antibody)-(cleavable moiety)-(masking moiety); and
(d) eight linker-payloads each depicted by the structure:

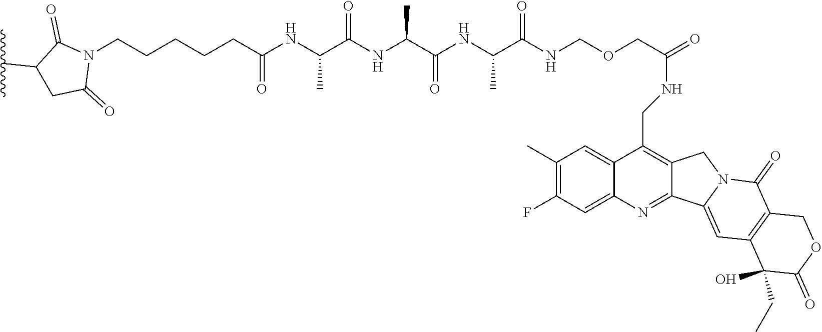 [SMILES: *C1CC(=O)N(CCCCCC(=O)N[C@@H](C)C(=O)N[C@@H](C)C(=O)N[C@@H](C)C(=O)NCOCC(=O)NCc2c3c(nc4cc(F)c(C)cc24)-c2cc4c(c(=O)n2C3)COC(=O)[C@]4(O)CC)C1=O]

or a pharmaceutically acceptable salt thereof, wherein each of the eight linker-payloads is individually covalently bound to the activatable antibody via one of the eight cysteines. 17. The immunoconjugate of embodiment 16, wherein the full length human IgG1 EpCAM antibody comprises a heavy chain variable domain (VH) consisting of the amino acid sequence of SEQ ID NO:54 and a light chain variable domain (VL) consisting of the amino acid sequence of SEQ ID NO: 89.

18. The immunoconjugate of embodiment 16, wherein the full length human IgG1 EpCAM antibody comprises a heavy chain consisting of the amino acid sequence of SEQ ID NO:103 and a light chain consisting of the amino acid sequence of SEQ ID NO:179.

19. An immunoconjugate obtainable by the method of Example 7.4.1.

20. An immunoconjugate obtainable by a method comprising:
(a) mixing an activatable EpCAM antibody consisting of a heterotetramer of two heavy chains each consisting of the amino acid sequence of SEQ ID NO:103 and two light chains each consisting of the amino acid sequence of SEQ ID NO:179 with a mixture of EPPS buffer and an aqueous EDTA solution to form an activatable EpCAM reaction mixture;
(b) adding a TCEP solution to the activatable EpCAM reaction mixture, thereby forming a reduced activatable EpCAM reaction mixture:
(c) mixing the reduced activatable EpCAM reaction mixture with a camptothecin linker-toxin stock solution comprising a linker-toxin represented by the structure

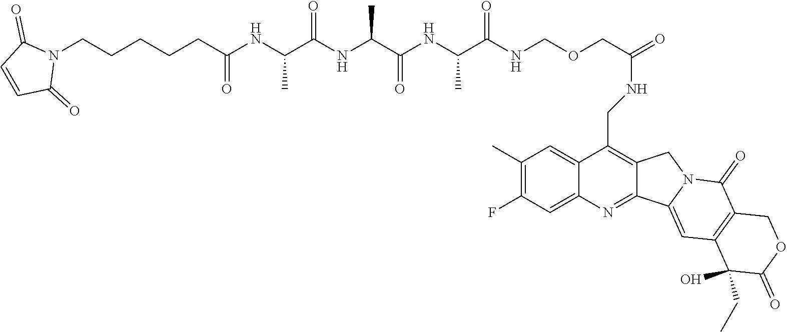 [SMILES: CC[C@@]1(O)C(=O)OCc2c1cc1n(c2=O)Cc2c-1nc1cc(F)c(C)cc1c2CNC(=O)COCNC(=O)[C@H](C)NC(=O)[C@H](C)NC(=O)[C@H](C)NC(=O)CCCCCN1C(=O)C=CC1=O]

thereby forming the immunoconjugate.

21. A nudeic acid comprising one or more coding regions for the activatable antibody of the immunoconjugate of any one of embodiments 1 to 20.

22. A vector comprising the nudeic acid of embodiment 21.

23. The vector of embodiment 22, which is a viral vector.

24. A method of producing an immunoconjugate, comprising:
(d) culturing a host cell comprising the vector of embodiment 23:
(e) isolating an activatable antibody from the host cell; and
(f) conjugating to the activatable antibody at least one linker-payload reactant comprising camptothecin or a derivative thereof.

25. The method of embodiment 24, wherein the linker-payload reactant comprises a compound of Formula I:

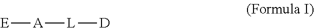

wherein:

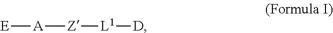

wherein:
D is represented by the following structural formula:

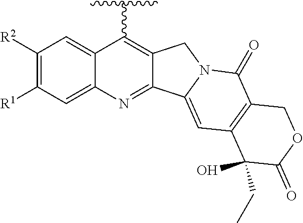

wherein R¹ is F and R² is methyl;
L¹-Z′—* is —(C₁-C₄ alkylene)-O—CH₂—NR⁸—*, —(C₁-C₄ alkylene)-NR⁸—*, or —(C₁-C₅ alkylene)-NR⁵C(═O )—(C₁-C₆ alkylene)-O—CH₂—NR⁶—*, where * is a site covalently attached to A;
each R⁵ is independently H, methyl, or benzyl;
each R⁸ is independently H, methyl, or benzyl;
A is a peptide comprising 2 to 4 amino acids; and
E is —C(═O)—(C₁-C₁₀ alkylene)-X³ wherein X³ is

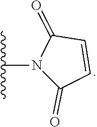 [SMILES: *N1C(=O)C=CC1=O]

26. The method of embodiment 24, wherein the linker-payload reactant is:

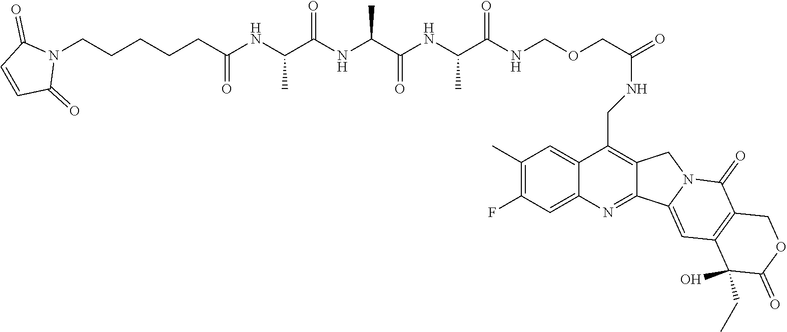 [SMILES: CC[C@@]1(O)C(=O)OCc2c1cc1n(c2=O)Cc2c-1nc1cc(F)c(C)cc1c2CNC(=O)COCNC(=O)[C@H](C)NC(=O)[C@H](C)NC(=O)[C@H](C)NC(=O)CCCCCN1C(=O)C=CC1=O]

or a pharmaceutically acceptable salt thereof.

27. The method of embodiment 24, wherein the linker-payload reactant is:

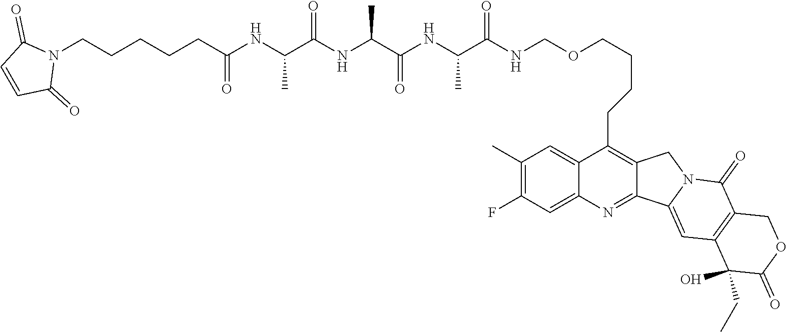 [SMILES: CC[C@@]1(O)C(=O)OCc2c1cc1n(c2=O)Cc2c-1nc1cc(F)c(C)cc1c2CCCCOCNC(=O)[C@H](C)NC(=O)[C@H](C)NC(=O)[C@H](C)NC(=O)CCCCCN1C(=O)C=CC1=O]

or a pharmaceutically acceptable salt thereof.

28. The method of embodiment 24, wherein the linker-payload reactant comprises exatecan.

29. The method of embodiment 24, wherein the linker-payload reactant comprises deruxtecan.

30. A method of producing an immunoconjugate, comprising conjugating at least one linker-payload reactant comprising camptothecin or a derivative thereof to the activatable antibody of any one of embodiments 1 to 20.

31. The method of embodiment 30, wherein the linker-payload reactant comprises a compound of Formula I:

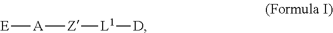

wherein:
D is represented by the following structural formula:

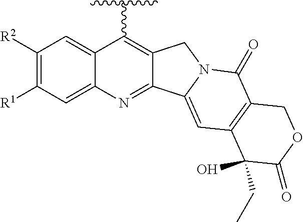

wherein R¹ is F and R² is methyl;
L¹-Z′—* is —(C₁-C₄ alkylene)-O—CH₂—NR⁸—*, —(C₁-C₄ alkylene)-NR⁸—*, or —(C₁-C₅ alkylene)-NR⁵C(═O)—(C₁-C₅ alkylene)-O—CH₂—NR⁶—*, where * is a site covalently attached to A;
each R⁵ is independently H, methyl, or benzyl;
each R⁸ is independently H, methyl, or benzyl;
A is a peptide comprising 2 to 4 amino acids; and
E is —C(═O)—(C₁-C₁₀ alkylene)-X³ wherein X is

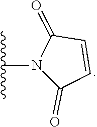 [SMILES: *N1C(=O)C=CC1=O]

32. The method of embodiment 30, wherein the linker-payload reactant is:

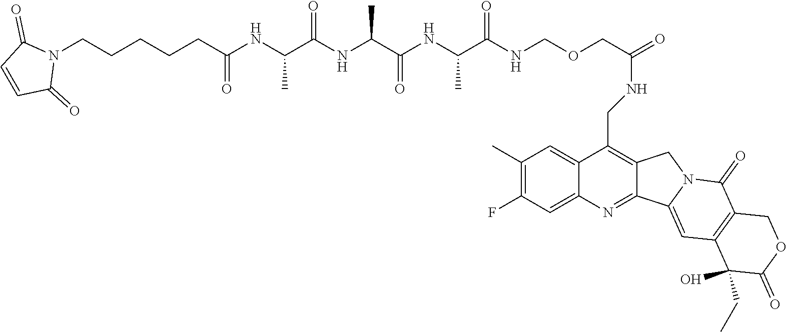 [SMILES: CC[C@@]1(O)C(=O)OCc2c1cc1n(c2=O)Cc2c-1nc1cc(F)c(C)cc1c2CNC(=O)COCNC(=O)[C@H](C)NC(=O)[C@H](C)NC(=O)[C@H](C)NC(=O)CCCCCN1C(=O)C=CC1=O]

or a pharmaceutically acceptable salt thereof.

33. The method of embodiment 30, wherein the linker-payload reactant is:

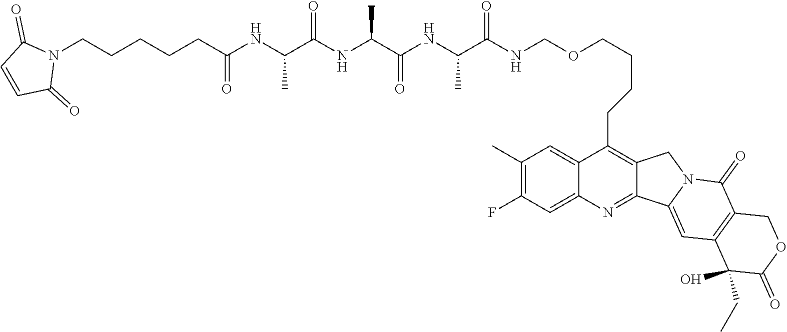 [SMILES: CC[C@@]1(O)C(=O)OCc2c1cc1n(c2=O)Cc2c-1nc1cc(F)c(C)cc1c2CCCCOCNC(=O)[C@H](C)NC(=O)[C@H](C)NC(=O)[C@H](C)NC(=O)CCCCCN1C(=O)C=CC1=O]

or a pharmaceutically acceptable salt thereof.

34. The method of embodiment 30, wherein the linker-payload reactant comprises exatecan.

35. The method of embodiment 30, wherein the linker-payload reactant comprises deruxtecan.

36. A composition comprising:
(g) the immunoconjugate of any one of embodiments 1 to 20; and
(h) a pharmaceutically acceptable carrier.

37. The immunoconjugate of any one of embodiments 1 to 20 or the composition of embodiment 36 for use in the treatment of an EpCAM-expressing cancer.

38. An immunoconjugate, comprising:
(a) an activatable antibody comprising:
   (i) an EpCAM antibody or EpCAM binding fragment thereof, comprising a heavy chain CDR1 (VH-CDR1), a heavy chain CDR2 (VH-CDR2), a heavy chain CDR3 (VH-CDR3), a light chain CDR1 (VL-CDR1), a light chain CDR2 (VL-CDR2), and a light chain CDR3 (VL-CDR3) comprising or consisting of amino acid sequences selected from the group consisting of:

(1)
(SEQ ID NO: 13)
NYYIH,
(SEQ ID NO: 14)
WIYPGNVYIQYNEKFKG,
(SEQ ID NO: 15)
DGPWFAY,
(SEQ ID NO: 42)
RSSRSLLHSDGFTYLY,
(SEQ ID NO: 40)
QTSNLAS,
and
(SEQ ID NO: 41)
AQNLELPNT,
respectively;
(2)
(SEQ ID NO: 13)
NYYIH,
(SEQ ID NO: 14)
WIYPGNVYIQYNEKFKG,
(SEQ ID NO: 15)
DGPWFAY,
(SEQ ID NO: 39)
RSSKSLLHSDGFTYLY,
(SEQ ID NO: 40)
QTSNLAS,
and
(SEQ ID NO: 41)
AQNLELPNT,
respectively;
(3)
(SEQ ID NO: 13)
NYYIH,
(SEQ ID NO: 26)
WIYPGNVYIQYSQKFQG,
(SEQ ID NO: 15)
DGPWFAY,
(SEQ ID NO: 39)
RSSKSLLHSDGFTYLY,
(SEQ ID NO: 40)
QTSNLAS,
and
(SEQ ID NO: 41)
AQNLELPNT,
respectively; and
(4)
(SEQ ID NO: 13)
NYYIH,
(SEQ ID NO: 26)
WIYPGNVYIQYSQKFQG,
(SEQ ID NO: 15)
DGPWFAY,
(SEQ ID NO: 42)
RSSRSLLHSDGFTYLY,
(SEQ ID NO: 40)
QTSNLAS,
and
(SEQ ID NO: 41)
AQNLELPNT,
respectively

(ii) a cleavable moiety coupled to the EpCAM antibody or EpCAM-binding fragment thereof, wherein the cleavable moiety is a polypeptide that functions as a substrate for a protease; and
(iii) a masking moiety coupled to the EpCAM antibody or EpCAM-binding fragment thereof, wherein the masking moiety inhibits the binding of the antibody or antibody fragment to EpCAM when the activatable antibody is in an uncleaved state,
wherein the activatable antibody in the uncleaved state has a structural arrangement from N-terminus to C-terminus of: (masking moiety)-(cleavable moiety)-(antibody or antibody fragment) or (antibody or antibody fragment)-(cleavable moiety)-(masking moiety); and
(b) at least one linker-payload comprising camptothecin or a derivative thereof.

39. The immunoconjugate of embodiment 38, wherein the EpCAM antibody or EpCAM binding fragment thereof comprises a VH-CDR1 comprising the amino acid sequence of SEQ ID NO:13, a VH-CDR2 comprising or consisting of the amino acid sequence of SEQ ID NO:14, a VH-CDR3 comprising or consisting of the amino acid sequence of SEQ ID NO:15, a VL-CDR1 comprising or consisting of the amino acid sequence of SEQ ID NO:42, a VL-CDR2 comprising or consisting of the amino acid sequence of SEQ ID NO:40, and a VL-CDR3 comprising or consisting of the amino acid sequence of SEQ ID NO:41.

40. The immunoconjugate of embodiment 38, wherein the EpCAM antibody or EpCAM binding fragment thereof comprises a VH-CDR1 comprising the amino acid sequence of SEQ ID NO:13, a VH-CDR2 comprising or consisting of the amino acid sequence of SEQ ID NO:14, a VH-CDR3 comprising or consisting of the amino acid sequence of SEQ ID NO:15, a VL-CDR1 comprising or consisting of the amino acid sequence of SEQ ID NO:39, a VL-CDR2 comprising or consisting of the amino acid sequence of SEQ ID NO:40, and a VL-CDR3 comprising or consisting of the amino acid sequence of SEQ ID NO:41.

41. The immunoconjugate of any one of embodiments 38 to 40, wherein the masking moiety comprises or consists of an amino acid sequence selected from the group consisting of: PLMTCSDYYTCLNNL (SEQ ID NO:151). LSCTHSRYDMHCPHM (SEQ ID NO:152), HYCHSRTDTITHCNA (SEQ ID NO:153), WCPRLFDRPSMGCPT (SEQ ID NO:154), WWPPCQGGAWCEQRI (SEQ ID NO:155), HSGCPRLFDRCSAPA (SEQ ID NO:156), FICPTLYDRPHCMHT (SEQ ID NO:157), DCTGYSPSVLPACRV (SEQ ID NO:162), FCSGYSPSVLPSCLM (SEQ ID NO:163), SKPCSYMHPYCFYNS (SEQ ID NO:164), LTRCTIAHPYCYYNY (SEQ ID NO:165), PNTCMSERRICSLTY (SEQ ID NO:166), PRPHCAILRQCLAAT (SEQ ID NO:167).

42. The immunoconjugate of any one of embodiments 38 to 41, wherein the masking moiety comprises or consists of an amino acid sequence of WWPPCQGGAWCEQRI (SEQ ID NO:155).

43. The immunoconjugate of any one of embodiments 38 to 42, wherein the cleavable moiety comprises or consists of an amino acid sequence of AVGLLAPPGGLSGRSDNI (SEQ ID NO:168) or of ISSGLLSGRSDNI (SEQ ID NO:169).

44. The immunoconjugate of any one of embodiments 38 to 43, wherein the cleavable moiety comprises or consists of an amino acid sequence of AVGLLAPPGGLSGRSDNI (SEQ ID NO:168).

45. The immunoconjugate of any one of embodiments 38 to 43, wherein the cleavable moiety comprises or consists of an amino acid sequence of ISSGLLSGRSDNI (SEQ ID NO:169).

46. The immunoconjugate of embodiment 38 or embodiment 39, wherein:
(a) the EpCAM antibody or EpCAM binding fragment thereof comprises a VH-CDR1 comprising or consisting of the amino acid sequence of SEQ ID NO:13, a VH-CDR2 comprising or consisting of the amino acid sequence of SEQ ID NO:14, a VH-CDR3 comprising or consisting of the amino acid sequence of SEQ ID NO:15, a VL-CDR1 comprising or consisting of the amino acid sequence of SEQ ID NO:42, a VL-CDR2 comprising or consisting of the amino acid sequence of SEQ ID NO:40, and a VL-CDR3 comprising or consisting of the amino acid sequence of SEQ ID NO:41;
(b) the masking moiety comprises or consists of an amino acid sequence of WWPPCQGGAWCEQRI (SEQ ID NO:155); and
(c) the cleavable moiety comprises or consists of an amino acid sequence of ISSGLLSGRSDNI (SEQ ID NO:169).

47. The immunoconjugate of embodiment 38 or embodiment 39, wherein:
(a) the EpCAM antibody or EpCAM binding fragment thereof comprises a VH-CDR1 comprising or consisting of the amino acid sequence of SEQ ID NO:13, a VH-CDR2 comprising or consisting of the amino acid sequence of SEQ ID NO:14, a VH-CDR3 comprising or consisting of the amino acid sequence of SEQ ID NO:15, a VL-CDR1 comprising or consisting of the amino acid sequence of SEQ ID NO:42, a VL-CDR2 comprising or consisting of the amino acid sequence of SEQ ID NO:40, and a VL-CDR3 comprising or consisting of the amino acid sequence of SEQ ID NO:41;
(b) the masking moiety comprises or consists of an amino acid sequence of WWPPCQGGAWCEQRI (SEQ ID NO:155); and
(c) the cleavable moiety comprises or consists of an amino acid sequence of AVGLLAPPGGLSGRSDNI (SEQ ID NO:168).

48. The immunoconjugate of any one of embodiments 38 to 47, wherein the EpCAM antibody or EpCAM-binding fragment thereof comprises a heavy chain variable domain (VH) and a light chain variable domain (VL) selected from the group consisting of:
(a) a VH comprising or consisting of an amino acid sequence having at least 90%, at least 91%, at least 92%, at least 93%, at least 94%, at least 95%, at least 96%, at least 97%, at least 98%, at least 99%, or 100% sequence identity to the amino acid sequence of SEQ ID NO:54, and a VL comprising or consisting of an amino acid sequence having at least 90%, at least 91%, at least 92%, at least 93%, at least 94%, at least 95%, at least 96%, at least 97%, at least 98%, at least 99%, or 100% sequence identity to the amino acid sequence of SEQ ID NO:89;
(b) a VH comprising or consisting of an amino acid sequence having at least 90%, at least 91%, at least 92%, at least 93%, at least 94%, at least 95%, at least 96%, at least 97%, at least 98%, at least 99%, or 100% sequence identity to the amino acid sequence of SEQ ID NO:54, and a VL comprising or consisting of an amino acid sequence having at least 90%, at least 91%, at least 92%, at least 93%, at least 94%, at least 95%, at least 96%, at least 97%, at least 98%, at least 99%, or 100% sequence identity to the amino acid sequence of SEQ ID NO:87;
(c) a VH comprising or consisting of an amino acid sequence having at least 90%, at least 91%, at least 92%, at least 93%, at least 94%, at least 95%, at least 96%, at least 97%, at least 98%, at least 99%, or 100% sequence identity to the amino acid sequence of SEQ ID NO:55, and a VL comprising or consisting of an amino acid sequence having at least 90%, at least 91%, at least 92%, at least 93%, at least 94%, at least 95%, at least 96%, at least 97%, at least 98%, at least 99%, or 100% sequence identity to the amino acid sequence of SEQ ID NO:87;
(d) a VH comprising or consisting of an amino acid sequence having at least 90%, at least 91%, at least 92%, at least 93%, at least 94%, at least 95%, at least 96%, at least 97%, at least 98%, at least 99%, or 100% sequence identity to the amino acid sequence of SEQ ID NO:56, and a VL comprising or consisting of an amino acid sequence having at least 90%, at least 91%, at least 92%, at least 93%, at least 94%, at least 95%, at least 96%, at least 97%, at least 98%, at least 99%, or 100% sequence identity to the amino acid sequence of SEQ ID NO:88;
(e) a VH comprising or consisting of an amino acid sequence having at least 90%, at least 91%, at least 92%, at least 93%, at least 94%, at least 95%, at least 96%, at least 97%, at least 98%, at least 99%, or 100% sequence identity to the amino acid sequence of SEQ ID NO:55, and a VL comprising or consisting of an amino acid sequence having at least 90%, at least 91%, at least 92%, at least 93%, at least 94%, at least 95%, at least 96%, at least 97%, at least 98%, at least 99%, or 100% sequence identity to the amino acid sequence of SEQ ID NO:89; and
(f) a VH comprising or consisting of an amino acid sequence having at least 90%, at least 91%, at least 92%, at least 93%, at least 94%, at least 95%, at least 96%, at least 97%, at least 98%, at least 99%, or 100% sequence identity to the amino acid sequence of SEQ ID NO:56, and a VL comprising or consisting of an amino acid sequence having at least 90%, at least 91%, at least 92%, at least 93%, at least 94%, at least 95%, at least 96%, at least 97%, at least 98%, at least 99%, or 100% sequence identity to the amino acid sequence of SEQ ID NO:89.

49. The immunoconjugate of embodiment 48, wherein the sequence identity in each instance is at least 95%.

50. The immunoconjugate of embodiment 48, wherein the sequence identity in each instance is at least 98%.

51. The immunoconjugate of any one of embodiments 38 to 50, wherein the EpCAM antibody or EpCAM-binding fragment thereof comprises a heavy chain variable domain (VH) and a light chain variable domain (VL) selected from the group consisting of:
(a) a VH comprising or consisting of the amino acid sequence of SEQ ID NO:54, and a VL comprising or consisting of the amino acid sequence of SEQ ID NO:89;
(b) a VH comprising or consisting of the amino acid sequence of SEQ ID NO:54, and a VL comprising or consisting of the amino acid sequence of SEQ ID NO:87;
(c) a VH comprising or consisting of the amino acid sequence of SEQ ID NO:55, and a VL comprising or consisting of the amino acid sequence of SEQ ID NO:87;
(d) a VH comprising or consisting of the amino acid sequence of SEQ ID NO:56, and a VL comprising or consisting of the amino acid sequence of SEQ ID NO:88;
(e) a VH comprising or consisting of the amino acid sequence of SEQ ID NO:55, and a VL comprising or consisting of the amino acid sequence of SEQ ID NO:89; and
(f) a VH comprising or consisting of the amino acid sequence of SEQ ID NO:56, and a VL comprising or consisting of the amino acid sequence of SEQ ID NO:89.

52. The immunoconjugate of any one of embodiments 38 to 51, wherein the EpCAM antibody or EpCAM-binding fragment thereof comprises a heavy chain variable domain (VH) comprising or consisting of the amino acid sequence of SEQ ID NO:54 and a light chain variable domain (VL) comprising or consisting of the amino acid sequence of SEQ ID NO: 89.

53. The immunoconjugate of any one of embodiments 38 to 45, wherein the EpCAM antibody or EpCAM-binding fragment thereof comprises a heavy chain and a light chain selected from the group consisting of:
(a) a heavy chain comprising or consisting of an amino acid sequence having at least 90%, at least 91%, at least 92%, at least 93%, at least 94%, at least 95%, at least 96%, at least 97%, at least 98%, at least 99%, or 100% sequence identity to the amino acid sequence of SEQ ID NO:103; and a light chain comprising or consisting of an amino acid sequence having at least 90%, at least 91%, at least 92%, at least 93%, at least 94%, at least 95%, at least 96%, at least 97%, at least 98%, at least 99%, or 100% sequence identity to the amino acid sequence of SEQ ID NO:179;
(b) a heavy chain comprising or consisting of an amino acid sequence having at least 90%, at least 91%, at least 92%, at least 93%, at least 94%, at least 95%, at least 96%, at least 97%, at least 98%, at least 99%, or 100% sequence identity to the amino acid sequence of SEQ ID NO:103; and a light chain comprising or consisting of an amino acid sequence having at least 90%, at least 91%, at least 92%, at least 93%, at least 94%, at least 95%, at least 96%, at least 97%, at least 98%, at least 99%, or 100% sequence identity to the amino acid sequence of SEQ ID NO:174;
(c) a heavy chain comprising or consisting of an amino acid sequence having at least 90%, at least 91%, at least 92%, at least 93%, at least 94%, at least 95%, at least 96%, at least 97%, at least 98%, at least 99%, or 100% sequence identity to the amino acid sequence of SEQ ID NO:103; and a light chain comprising (i) the cleavable moiety, (ii) the masking moiety, and (iii) an amino acid sequence having at least 90%, at least 91%, at least 92%, at least 93%, at least 94%, at least 95%, at least 96%, at least 97%, at least 98%, at least 99%, or 100% sequence identity to the amino acid sequence of SEQ ID NO:140;
(d) a heavy chain comprising or consisting of an amino acid sequence having at least 90%, at least 91%, at least 92%, at least 93%, at least 94%, at least 95%, at least 96%, at least 97%, at least 98%, at least 99%, or 100% sequence identity to the amino acid sequence of SEQ ID NO:103; and a light chain comprising (i) the cleavable moiety, (ii) the masking moiety, and (iii) an amino acid sequence having at least 90%, at least 91%, at least 92%, at least 93%, at least 94%, at least 95%, at least 96%, at least 97%, at least 98%, at least 99%, or 100% sequence identity to the amino acid sequence of SEQ ID NO:138;
(e) a heavy chain comprising or consisting of an amino acid sequence having at least 90%, at least 91%, at least 92%, at least 93%, at least 94%, at least 95%, at least 96%, at least 97%, at least 98%, at least 99%, or 100% sequence identity to the amino acid sequence of SEQ ID NO:105; and a light chain comprising (i) the cleavable moiety, (ii) the masking moiety, and (iii) an amino acid sequence having at least 90%, at least 91%, at least 92%, at least 93%, at least 94%, at least 95%, at least 96%, at least 97%, at least 98%, at least 99%, or 100% sequence identity to the amino acid sequence of SEQ ID NO:139;
(f) a heavy chain comprising or consisting of an amino acid sequence having at least 90%, at least 91%, at least 92%, at least 93%, at least 94%, at least 95%, at least 96%, at least 97%, at least 98%, at least 99%, or 100% sequence identity to the amino acid sequence of SEQ ID NO:106; and a light chain comprising (i) the cleavable moiety, (ii) the masking moiety, and (iii) an amino acid sequence having at least 90%, at least 91%, at least 92%, at least 93%, at least 94%, at least 95%, at least 96%, at least 97%, at least 98%, at least 99%, or 100% sequence identity to the amino acid sequence of SEQ ID NO:139;
(g) a heavy chain comprising or consisting of an amino acid sequence having at least 90%, at least 91%, at least 92%, at least 93%, at least 94%, at least 95%, at least 96%, at least 97%, at least 98%, at least 99%, or 100% sequence identity to the amino acid sequence of SEQ ID NO:105; and a light chain comprising (i) the cleavable moiety, (ii) the masking moiety, and (iii) an amino acid sequence having at least 90%, at least 91%, at least 92%, at least 93%, at least 94%, at least 95%, at least 96%, at least 97%, at least 98%, at least 99%, or 100% sequence identity to the amino acid sequence of SEQ ID NO:140; and
(h) a heavy chain comprising or consisting of an amino acid sequence having at least 90%, at least 91%, at least 92%, at least 93%, at least 94%, at least 95%, at least 96%, at least 97%, at least 98%, at least 99%, or 100% sequence identity to the amino acid sequence of SEQ ID NO:106; and a light chain comprising (i) the cleavable moiety, (ii) the masking moiety, and (iii) an amino acid sequence having at least 90%, at least 91%, at least 92%, at least 93%, at least 94%, at least 95%, at least 96%, at least 97%, at least 98%, at least 99%, or 100% sequence identity to the amino acid sequence of SEQ ID NO:140.

54. The immunoconjugate of embodiment 53, wherein the sequence identity in each instance is at least 95%.

55. The immunoconjugate of embodiment 53, wherein the sequence identity in each instance is at least 98%.

56. The immunoconjugate of any one of embodiments 38 to 45, wherein the EpCAM antibody or EpCAM-binding fragment thereof comprises a heavy chain and a light chain selected from the group consisting of:
(a) a heavy chain comprising or consisting of an amino acid sequence having the amino acid sequence of SEQ ID NO:103 and a light chain comprising or consisting of the amino acid sequence of SEQ ID NO:179;
(b) a heavy chain comprising or consisting of an amino acid sequence having the amino acid sequence of SEQ ID NO:103 and a light chain comprising or consisting of the amino acid sequence of SEQ ID NO:174;
(c) a heavy chain comprising or consisting of the amino acid sequence of SEQ ID NO:103, and a light chain comprising (i) the cleavable moiety, (ii) the masking moiety, and (iii) the amino acid sequence of SEQ ID NO:140;
(d) a heavy chain comprising or consisting of the amino acid sequence of SEQ ID NO:103, and a light chain comprising (i) the cleavable moiety, (ii) the masking moiety, and (iii) the amino acid sequence of SEQ ID NO:138;
(e) a heavy chain comprising or consisting of the amino acid sequence of SEQ ID NO:105, and a light chain comprising (i) the cleavable moiety, (ii) the masking moiety, and (iii) the amino acid sequence of SEQ ID NO:139;
(f) a heavy chain comprising or consisting of the amino acid sequence of SEQ ID NO:106, and a light chain comprising (i) the cleavable moiety, (ii) the masking moiety, and (iii) the amino acid sequence of SEQ ID NO:139;
(g) a heavy chain comprising or consisting of the amino acid sequence of SEQ ID NO:105, and a light chain comprising (i) the cleavable moiety, (ii) the masking moiety, and (iii) the amino acid sequence of SEQ ID NO:140; and
(h) a heavy chain comprising or consisting of the amino acid sequence of SEQ ID NO:106, and a light chain comprising (i) the cleavable moiety, (ii) the masking moiety, and (iii) the amino acid sequence of SEQ ID NO:140.

57. The immunoconjugate of embodiment 38, wherein the EpCAM antibody or EpCAM-binding fragment thereof comprises a heavy chain comprising or consisting of the amino acid sequence of SEQ ID NO:103 and a light chain comprising or consisting of the amino acid sequence of SEQ ID NO:179.

58. The immunoconjugate of any one of embodiments 38 to 57, wherein the EpCAM antibody is a full-length antibody.

59. The immunoconjugate of embodiment 58, wherein the EpCAM antibody is a human IgG1 antibody.

60. The immunoconjugate of embodiment 58, wherein the EpCAM antibody is a humanized IgG1 antibody.

61. The immunoconjugate of any one of embodiments 38 to 57, wherein the EpCAM antibody or EpCAM-binding fragment thereof is selected from the group consisting of: a Fab, a Fab′, a F(ab′)₂, an scFv, and a disulfide-linked Fv (dsFv).

62. The immunoconjugate of any one of embodiments 38 to 61, wherein the at least one linker-payload comprises a structure of Formula Ia or a pharmaceutically acceptable salt thereof:

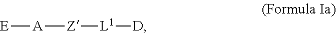

wherein:
D is represented by the following structural formula:

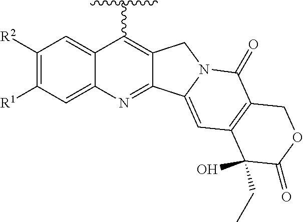

wherein R¹ is F and R² is methyl;
L¹-Z′—* is —(C₁-C₄ alkylene)-O—CH₂—NR⁸—*, —(C₁-C₄ alkylene)-NR⁸, or —(C₁-C₅ alkylene)-NR⁵C(═O)—(C₁-C₅ alkylene)-O—CH₂—NR⁸*, where * is a site covalently attached to A;
each R⁵ is independently H, methyl, or benzyl;
each R⁸ is independently H, methyl, or benzyl;
A is a peptide comprising 2 to 4 amino acids; and
E is —C(═O)—(C₁-C₁ alkylene)-X³ wherein X³ is

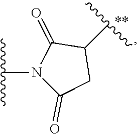 [SMILES: *C1CC(=O)N(*)C1=O]

wherein ** is a site covalently attached to the EpCAM antibody or EpCAM binding fragment thereof.

63. The immunoconjugate of embodiment 62, wherein A is substituted with one or more polyols.

64. The immunoconjugate of embodiment 63, wherein the one or more polyols is —(C₁-C₆ alkylene)-X⁵—Y³, wherein
(a) X⁵ is —NR¹²C(═O)— or —C(═O)NR¹²—;
(a) Y³ is a C₆ alkyl substituted with 5 OH groups; and
(b) R¹² is —H, a C₁-C₆ alkyl, a C₁-C₆ fluoroalkyl, a C₃-C₆ cycloalkyl, an aryl, a heteroaryl, or benzyl.

65. The immunoconjugate of embodiment 63, wherein the one or more polyols is

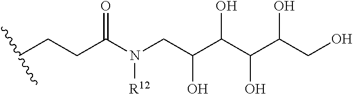

wherein R¹² is H or methyl.

66. The immunoconjugate of any one of embodiments 38 to 62, wherein the linker-payload is

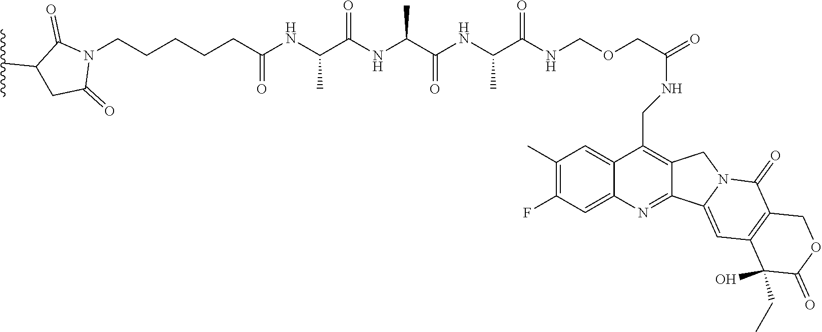 [SMILES: *C1CC(=O)N(CCCCCC(=O)N[C@@H](C)C(=O)N[C@@H](C)C(=O)N[C@@H](C)C(=O)NCOCC(=O)NCc2c3c(nc4cc(F)c(C)cc24)-c2cc4c(c(=O)n2C3)COC(=O)[C@]4(O)CC)C1=O]

or a pharmaceutically acceptable salt thereof.

67. The immunoconjugate of any one of embodiments 38 to 62, wherein the linker-payload is

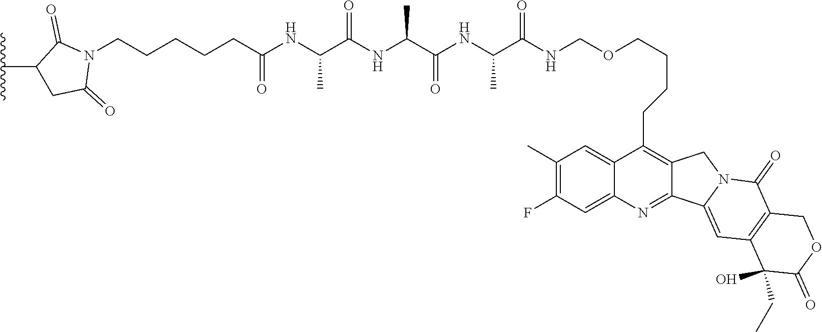 [SMILES: *C1CC(=O)N(CCCCCC(=O)N[C@@H](C)C(=O)N[C@@H](C)C(=O)N[C@@H](C)C(=O)NCOCCCCc2c3c(nc4cc(F)c(C)cc24)-c2cc4c(c(=O)n2C3)COC(=O)[C@]4(O)CC)C1=O]

or a pharmaceutically acceptable salt thereof.

68. The immunoconjugate of any one of embodiments 38 to 61, wherein the linker-payload comprises exatecan.

69. The immunoconjugate of any one of embodiments 38 to 61, wherein the linker-payload comprises reduced deruxtecan.

70. The immunoconjugate of any one of embodiments 38 to 69, having a drug-to-antibody ratio (DAR) selected from the group consisting of: 2, 3, 4, 5, 6, 7, and 8.

71. The immunoconjugate of any one of embodiments 38 to 70, having a DAR of 8.

72. The immunoconjugate of any one of embodiments 1 to 71, wherein the at least one linker-payload covalently attaches to the activatable antibody via a cysteine residue of the activatable antibody.

73. The immunoconjugate of any one of embodiments 38 to 72, wherein the EpCAM antibody is a humanized IgG1 antibody having eight cysteines which, when in a native conformation form, four interchain disulfide bonds, and wherein each of the linker-payloads is individually covalently bound to the activatable antibody via one of the eight cysteines.

74. The immunoconjugate of any one of embodiments 38 to 60 and 70 to 73, wherein:
(a) the EpCAM antibody or EpCAM binding fragment thereof comprises a VH-CDR1 comprising or consisting of the amino acid sequence of SEQ ID NO:13, a VH-CDR2 comprising or consisting of the amino acid sequence of SEQ ID NO:14, a VH-CDR3 comprising or consisting of the amino acid sequence of SEQ ID NO:15, a VL-CDR1 comprising or consisting of the amino acid sequence of SEQ ID NO:42, a VL-CDR2 comprising or consisting of the amino acid sequence of SEQ ID NO:40, and a VL-CDR3 comprising or consisting of the amino acid sequence of SEQ ID NO:41;
(b) the masking moiety comprises or consists of an amino acid sequence of WWPPCQGGAWCEQRI (SEQ ID NO:155);
(c) the cleavable moiety comprises or consists of an amino acid sequence of ISSGLLSGRSDNI (SEQ ID NO:169);
(d) the EpCAM antibody is a humanized IgG1 antibody having eight cysteines which, when in a native conformation, form four interchain disulfide bonds;
(e) the linker-payload is

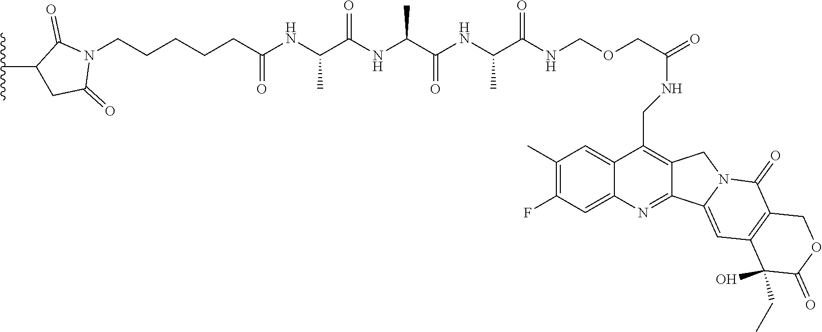 [SMILES: *C1CC(=O)N(CCCCCC(=O)N[C@@H](C)C(=O)N[C@@H](C)C(=O)N[C@@H](C)C(=O)NCOCC(=O)NCc2c3c(nc4cc(F)c(C)cc24)-c2cc4c(c(=O)n2C3)COC(=O)[C@]4(O)CC)C1=O]

or a pharmaceutically acceptable salt thereof; and
(f) the immunoconjugate has a DAR of 8, wherein each of the linker-payloads is individually covalently bound to the activatable antibody via one of the eight cysteines.

75. The immunoconjugate of any one of embodiments 38 to 60 and 70 to 72, wherein:
(a) the EpCAM antibody or EpCAM binding fragment thereof comprises a VH-CDR1 comprising or consisting of the amino acid sequence of SEQ ID NO:13, a VH-CDR2 comprising or consisting of the amino acid sequence of SEQ ID NO:14, a VH-CDR3 comprising or consisting of the amino acid sequence of SEQ ID NO:15, a VL-CDR1 comprising or consisting of the amino acid sequence of SEQ ID NO:42, a VL-CDR2 comprising or consisting of the amino acid sequence of SEQ ID NO:40, and a VL-CDR3 comprising or consisting of the amino acid sequence of SEQ ID NO:41;
(b) the masking moiety comprises or consists of an amino acid sequence of WWPPCQGGAWCEQRI (SEQ ID NO:155);
(c) the cleavable moiety comprises or consists of an amino acid sequence of AVGLLAPPGGLSGRSDNI (SEQ ID NO:168);
(d) the EpCAM antibody is a humanized IgG1 antibody having eight cysteines which, when in a native conformation, form four interchain disulfide bonds;
(e) the linker-payload is

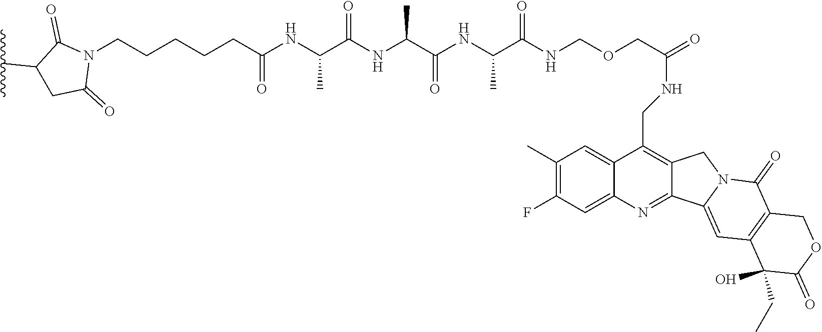 [SMILES: *C1CC(=O)N(CCCCCC(=O)N[C@@H](C)C(=O)N[C@@H](C)C(=O)N[C@@H](C)C(=O)NCOCC(=O)NCc2c3c(nc4cc(F)c(C)cc24)-c2cc4c(c(=O)n2C3)COC(=O)[C@]4(O)CC)C1=O]

or a pharmaceutically acceptable salt thereof; and
(f) the immunoconjugate has a DAR of 8, wherein each of the eight linker-payloads is individually covalently bound to the activatable antibody via one of the eight cysteines.

76. A nucleic acid comprising one or more coding regions for the activatable antibody of the immunoconjugate of any one of embodiments 38 to 75.

77. The nucleic acid of embodiment 76 wherein the one or more coding regions are codon-optimized for expression in a host cell.

78. A vector comprising the nucleic acid of embodiment 76 or embodiment 77.

79. The vector of embodiment 78, which is a viral vector.

80. A host cell engineered to express the nucleic acid embodiment 76 or embodiment 77 or the vector of embodiment 78 or embodiment 79.

81. A method of producing an immunoconjugate, comprising:
(a) culturing the host cell of embodiment 80;
(b) isolating an activatable antibody from the host cell; and
(c) conjugating to the activatable antibody at least one linker-payload reactant comprising camptothecin or a derivative thereof.

82. The method of embodiment 81, wherein the linker-payload reactant comprises a compound of Formula I:

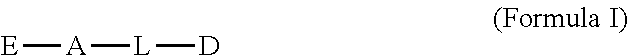

wherein:

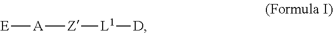

wherein:
D is represented by the following structural formula:

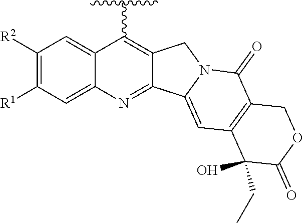

wherein R¹ is F and R² is methyl;
L¹-Z′—* is —(C₁-C₄ alkylene)-O—CH₂—NR⁸—*, —(C₁-C₄ alkylene)-NR⁸, or —(C₁-C₅ alkylene)-NR⁵C(═O)—(C₁-C₅ alkylene)-O—CH₂—NR⁸*, where * is a site covalently attached to A;
each R⁵ is independently H, methyl, or benzyl;
each R⁸ is independently H, methyl, or benzyl;
A is a peptide comprising 2 to 4 amino acids; and
E is —C(═O)—(C₁-C₁₀ alkylene)-X³ wherein X³ is

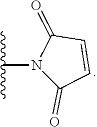 [SMILES: *N1C(=O)C=CC1=O]

83. The method of embodiment 81, wherein the linker-payload reactant is:

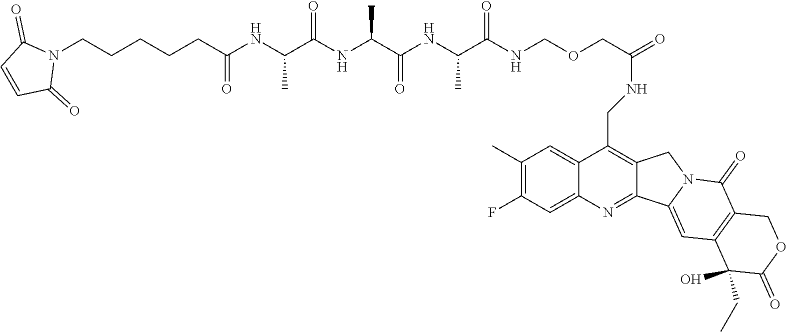 [SMILES: CC[C@@]1(O)C(=O)OCc2c1cc1n(c2=O)Cc2c-1nc1cc(F)c(C)cc1c2CNC(=O)COCNC(=O)[C@H](C)NC(=O)[C@H](C)NC(=O)[C@H](C)NC(=O)CCCCCN1C(=O)C=CC1=O]

or a pharmaceutically acceptable salt thereof.

84. The method of embodiment 81, wherein the linker-payload reactant is:

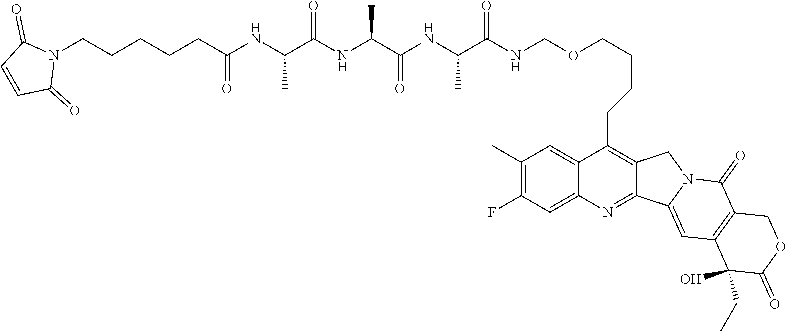 [SMILES: CC[C@@]1(O)C(=O)OCc2c1cc1n(c2=O)Cc2c-1nc1cc(F)c(C)cc1c2CCCCOCNC(=O)[C@H](C)NC(=O)[C@H](C)NC(=O)[C@H](C)NC(=O)CCCCCN1C(=O)C=CC1=O]

or a pharmaceutically acceptable salt thereof.

85. The method of embodiment 81, wherein the linker-payload reactant comprises exatecan.

86. The method of embodiment 81, wherein the linker-payload reactant comprises deruxtecan.

87. A method of producing an immunoconjugate, comprising conjugating at least one linker-payload reactant comprising camptothecin or a derivative thereof to the activatable antibody of any one of embodiments 38 to 75.

88. The method of embodiment 87, wherein the linker-payload reactant comprises a compound of Formula I:

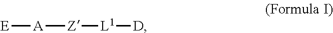

wherein:
D is represented by the following structural formula:

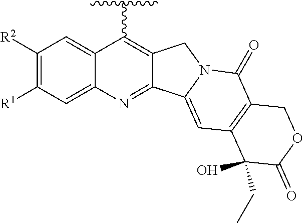

wherein R¹ is F and R² is methyl;
L¹-Z′—* is —(C₁-C₄ alkylene)-O—CH₂—NR⁸—*, —(C₁-C₄ alkylene)-NR⁸, or —(C₁-C₅ alkylene)-NR⁵C(═O)—(C₁-C₅, alkylene)-O—CH₂—NR⁸*, where * is a site covalently attached to A;
each R⁵ is independently H, methyl, or benzyl;
each R⁸ is independently H, methyl, or benzyl;
A is a peptide comprising 2 to 4 amino acids; and
E is —C(═O)—(C₁-C₁₀ alkylene)-X³ wherein X³ is

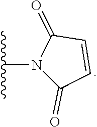 [SMILES: *N1C(=O)C=CC1=O]

89. The method of embodiment 87, wherein the linker-payload reactant is:

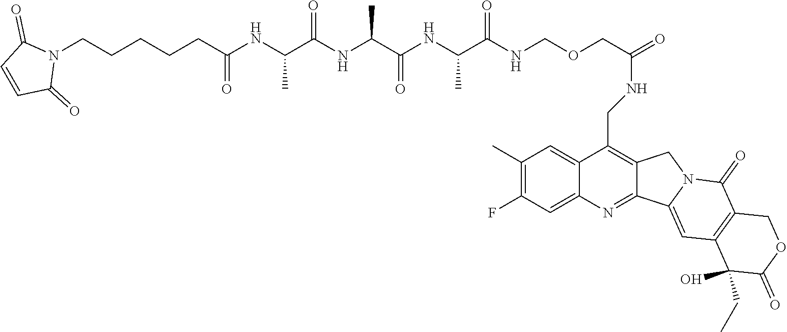 [SMILES: CC[C@@]1(O)C(=O)OCc2c1cc1n(c2=O)Cc2c-1nc1cc(F)c(C)cc1c2CNC(=O)COCNC(=O)[C@H](C)NC(=O)[C@H](C)NC(=O)[C@H](C)NC(=O)CCCCCN1C(=O)C=CC1=O]

or a pharmaceutically acceptable salt thereof.

90. The method of embodiment 87, wherein the linker-payload reactant is:

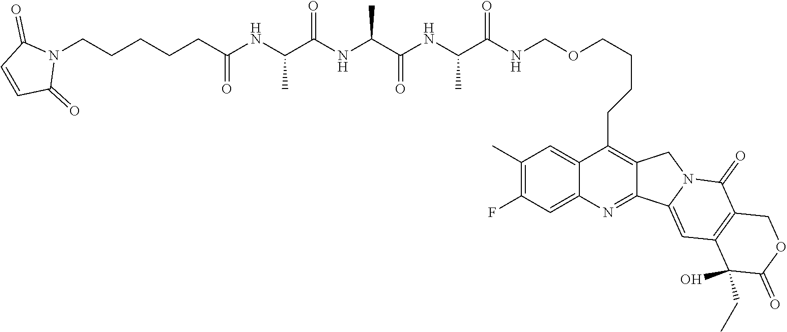 [SMILES: CC[C@@]1(O)C(=O)OCc2c1cc1n(c2=O)Cc2c-1nc1cc(F)c(C)cc1c2CCCCOCNC(=O)[C@H](C)NC(=O)[C@H](C)NC(=O)[C@H](C)NC(=O)CCCCCN1C(=O)C=CC1=O]

or a pharmaceutically acceptable salt thereof.

91. The method of embodiment 87, wherein the linker-payload reactant comprises exatecan.

92. The method of embodiment 87, wherein the linker-payload reactant comprises deruxtecan.

93. A composition comprising:
(a) the immunoconjugate of any one of embodiments 38 to 75; and
(b) a pharmaceutically acceptable carrier.

94. The immunoconjugate of any one of embodiments 38 to 75 or the composition of embodiment 93 for use in the treatment of an EpCAM-expressing cancer.

95. The immunoconjugate of any one of embodiments 38 to 75 or the composition of embodiment 93, wherein the cancer is selected from the group consisting of: epithelial cancer, breast cancer, lung cancer, non-small cell lung cancer, stomach cancer, colorectal cancer, prostate cancer, bladder cancer, ovarian cancer, colon cancer, rectal cancer, uterine cancer, gastric cancer, head and neck cancer, endometrial cancer, and pancreatic cancer.

96. The immunoconjugate of any one of embodiments 38 to 75 or the composition of embodiment 93, wherein the cancer is selected from the group consisting of colorectal cancer, gastric cancer, head and neck cancer, and breast cancer.

97. An immunoconjugate, comprising:
(a) an EpCAM antibody or EpCAM-binding fragments thereof, comprising:
   (iv) a heavy chain complementarity determining region 1 (VH-CDR1) comprising the amino acid sequence X₁YX₃X₄H, wherein X₁ is selected from N and S, X₃ is selected from Y, N, F, S, H, D, L, I, and W, and X₄ is selected from I and M;
   (v) a heavy chain complementarity determining region 2 (VH-CDR2) comprising the amino acid sequence WX₂X₃PGX₆VYIQYX₁₂X₁₃KFX₁₇G, wherein X₂ is selected from I and F, X₃ is selected from Y and N, X₆ is selected from N and D, X₁₂ is selected from N and S, X₁₃ is selected from E and Q, and X₁₇ is selected from K and Q (SEQ ID NO:7);
   (vi) heavy chain complementarity determining region 3 (VH-CDR3) comprising the amino acid sequence X₁GX₃X₄FAY, wherein X₁ is selected from D and E, X₃ is selected from P, A, S, Y, F, G. T, and V, and X₄ is selected from Y and W (SEQ ID NO:8);
   (vii) a light chain complementarity determining region 1 (VL-CDR1) comprising the amino acid sequence RSSX₄SLLHSX₁₀GX₁₂TY LX₁₆, wherein X₄ is selected from R and K, X₁₀ is selected from N and D, X₁₂ is selected from F and I, and X₁₆ is selected from Y and S (SEQ ID NO:10);
   (viii) a light chain complementarity determining region 2 (VL-CDR2) comprising the amino acid sequence QTSNLAS (SEQ ID NO:14); and
   (ix) a light chain complementarity determining region 3 (VL-CDR3) comprising the amino acid sequence X₁QX₃LELPX₈T, wherein X₁ is selected from A, L, and Q, X₃ is selected from S, G, Y, and N, and Xe is selected from N and W (SEQ ID NO:11);
(b) at least one linker-payload comprising a compound of Formula I or a pharmaceutically acceptable salt thereof (wherein the compound of Formula I or a pharmaceutically acceptable salt thereof represents the linker-payload reactant, i.e. the linker-payload prior to conjugation to the EpCAM antibody or EpCAM-binding fragments thereof):

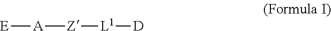

wherein:
D is represented by the following structural formula:

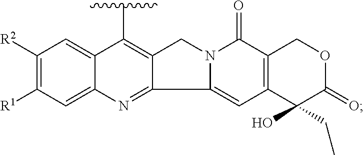

R¹ is F;
R² is methyl;
L¹-Z′—* is —(C₁-C₄ alkylene)-O—CH₂—NR⁸—*, —(C₁-C₄ alkylene)-NR⁸, or —(C₁-C₅ alkylene)-NR⁵C(═O)—(C₁-C₅ alkylene)-O—CH₂—NR⁸*, where* is a site covalently attached to A;
each R⁵ is independently H, methyl, or benzyl;
each R⁸ is independently H, methyl, or benzyl;
A is a peptide comprising 2 to 4 amino acids; and
E is C(═O)—(C₁-C₁₀ alkylene)-X³ wherein X³ is

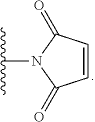 [SMILES: *N1C(=O)C=CC1=O]

98. The immunoconjugate of embodiment 97, wherein the EpCAM antibody or EpCAM-binding fragments thereof comprises:
(a) a VH-CDR1 comprising the amino acid sequence NYX₃IH, wherein X₃s is selected from Y, N, F, S, H, D, L, I, and W (SEQ ID NO:6);
(b) a VH-CDR2 comprising the amino acid sequence WXₑX₃PGX₆VYIQYX₁₂X₁₃KFX₁₇G, wherein X₂ is selected from I and F, Xs is selected from Y and N, X₆ is selected from N and D, X₁₂ is selected from N and S, X₁₃ is selected from E and Q, and X₁₇ is selected from K and Q (SEQ ID NO:7);
(c) a VH-CDR3 comprising the amino acid sequence DGPX₄FAY, wherein Xa is selected from Y and W (SEQ ID NO:9);
(d) a VL-CDR1 comprising the amino acid sequence RSSX₄SLLHSX₁₀GX₁₂TYLX₁₆, wherein X₄ is selected from R and K, X₁₀ is selected from N and D, X₁₂ is selected from F and I, and Xie is selected from Y and S (SEQ ID NO:10);
(e) a VL-CDR2 comprising the amino acid sequence QTSNLAS (SEQ ID NO:40); and
(f) a VL-CDR3 comprising the amino acid sequence AQX₃LELPNT, wherein X₃ is selected from S, G, Y, and N (SEQ ID NO:12).

99. The immunoconjugate of embodiment 97 or embodiment 98, wherein the EpCAM antibody or EpCAM-binding fragments thereof binds with a Ko of 3.0 nM or less to both human EpCAM and cynomolgus EpCAM.

100. The immunoconjugate of any one of embodiments 97 to 99, wherein the EpCAM antibody or EpCAM-binding fragments thereof binds to an epitope within the extracellular domain of human EpCAM (QEECVCENYKLAVNCFVNNNRQCQCTSVGAQNTVIC) (SEQ ID NO:2).

101. The immunoconjugate of any one of embodiments 97 to 100, wherein the EpCAM antibody or EpCAM-binding fragments thereof comprises:
(a) a VH-CDR1 comprising the amino acid sequence NYYIH (SEQ ID NO:13):
(b) a VH-CDR2 comprising the amino acid sequence WIYPGNVYIQYNEKFKG (SEQ ID NO:14);
(c) a VH-CDR3 comprising the amino acid sequence DGPWFAY (SEQ ID NO:15);
(d) a VL-CDR1 comprising the amino acid sequence RSSRSLLHSDGFTYLY (SEQ ID NO:42);
(e) a VL-CDR2 comprising the amino acid sequence QTSNLAS (SEQ ID NO:40); and
(f) a VL-CDR3 comprising the amino acid sequence AQNLELPNT (SEQ ID NO:41).

102. The immunoconjugate of any one of embodiments 97 to 100, wherein the EpCAM antibody or EpCAM-binding fragments thereof comprises:
(a) a VH-CDR1 comprising the amino acid sequence NYYIH (SEQ ID NO:13);
(b) a VH-CDR2 comprising the amino acid sequence WIYPGNVYIQYSQKFQG (SEQ ID NO:26);
(c) a VH-CDR3 comprising the amino acid sequence DGPWFAY (SEQ ID NO:15);
(d) a VL-CDR1 comprising the amino acid sequence RSSRSLLHSDGFTYLY (SEQ ID NO:42);
(e) a VL-CDR2 comprising the amino acid sequence QTSNLAS (SEQ ID NO:40); and
(f) a VL-CDR3 comprising the amino acid sequence AQNLELPNT (SEQ ID NO:41).

103. The immunoconjugate of any one of embodiments 97 to 100, wherein the EpCAM antibody or EpCAM-binding fragments thereof comprises:
(a) a VH-CDR1 comprising the amino acid amino acid sequence NYYIH (SEQ ID NO:13);
(b) a VH-CDR2 comprising the amino acid sequence WIYPGNVYIQYNEKFKG (SEQ ID NO:14);
(c) a VH-CDR3 comprising the amino acid sequence DGPWFAY (SEQ ID NO:15);
(d) a VL-CDR1 comprising the amino acid sequence RSSKSLLHSDGFTYLY (SEQ ID NO:39);
(e) a VL-CDR2 comprising the amino acid sequence QTSNLAS (SEQ ID NO:40); and
(f) a VL-CDR3 comprising the amino acid sequence AQNLELPNT (SEQ ID NO:41).

104. The immunoconjugate of any one of embodiments 97 to 100, wherein the EpCAM antibody or EpCAM-binding fragments thereof comprises:
(a) a VH-CDR1 comprising the amino acid sequence NYYIH (SEQ ID NO:13);
(b) a VH-CDR2 comprising the amino acid sequence WIYPGNVYIQYSQKFQG (SEQ ID NO:26);
(c) a VH-CDR3 comprising the amino acid sequence DGPWFAY (SEQ ID NO:15);
(d) a VL-CDR1 comprising the amino acid sequence RSSKSLLHSDGFTYLY (SEQ ID NO:39);
(e) a VL-CDR2 comprising the amino acid sequence QTSNLAS (SEQ ID NO:40); and
(f) a VL-CDR3 comprising the amino acid sequence AQNLELPNT (SEQ ID NO:41).

105. The immunoconjugate of any one of embodiments 97 to 104, wherein the EpCAM antibody or EpCAM-binding fragments thereof comprises:
(a) a heavy chain variable domain (VH) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYYIHWVRQAPGQRLEYIG WIYPGNVYIQYNEKFKGRATLTADKSASTAYMELSSLRSEDTAVYYCARD GPWFAYWGQGTLVTVSS (SEQ ID NO:54); and
(b) a light chain variable domain (VL) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVLTQTPLSLSVTPGQPASISCRSSRSLLHSDGFTYLYWFLQKPGQSPQ LLIYQTSNLASGVPDRFSSSGSGTDFTLKISRVEAEDVGVYYCAQNLELP NTFGQGTKLEIK (SEQ ID NO:89).

106. The immunoconjugate of any one of embodiments 97 to 104, wherein the EpCAM antibody or EpCAM-binding fragments thereof comprises:
(a) a heavy chain variable domain (VH) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYYIHWVRQAPGQRLEYIG WIYPGNVYIQYNEKFKGRATLTADKSASTAYMELSSLRSEDTAVYYCARD GPWFAYWGQGTLVTVSS (SEQ ID NO:54); and
(b) a light chain variable domain (VL) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVLTQTPLSLSVTPGQPASISCRSSKSLLHSDGFTYLYWFLQKPGQSPQ LLIYQTSNLASGVPDRFSSSGSGTDFTLKISRVEAEDVGVYYCAQNLELP NTFGQGTKLEIK (SEQ ID NO:87).

107. The immunoconjugate of any one of embodiments 97 to 104, wherein the EpCAM antibody or EpCAM-binding fragments thereof comprises:
(a) a heavy chain variable domain (VH) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYYIHWVRQAPGQRLEW MGWIYPGNVYIQYNEKFKGRVTITRDTSASTAYMELSSLRSEDTAVYYCA RDGPWFAYWGQGTLVTVSS (SEQ ID NO:55); and
(b) a light chain variable domain (VL) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVLTQTPLSLSVTPGQPASISCRSSKSLLHSDGFTYLYWFLQKPGQSPQ LLIYQTSNLASGVPDRFSSSGSGTDFTLKISRVEAEDVGVYYCAQNLELP NTFGQGTKLEIK (SEQ ID NO:87).

108. The immunoconjugate of any one of embodiments 97 to 104, wherein the EpCAM antibody or EpCAM-binding fragments thereof comprises:
(a) a heavy chain variable domain (VH) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYYIHWVRQAPGQRLEYIG WIYPGNVYIQYSQKFQGRVTITADKSASTAYMELSSLRSEDTAVYYCARD GPWFAYWGQGTLVTVSS (SEQ ID NO:56); and
(b) a light chain variable domain (VL) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVMTQTPLSLSVTPGQPASISCRSSKSLLHSDGFTYLYWFLQKPGQSPQ LLIYQTSNLASGVPDRFSGSGSGTDFTLKISRVEAEDVGVYYCAQNLELP NTFGQGTKLEIK (SEQ ID NO:88).

109. The immunoconjugate of any one of embodiments 97 to 100, wherein the EpCAM antibody or EpCAM-binding fragments thereof comprises:
(a) a heavy chain variable domain (VH) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYYIHWVRQAPGQRLEW MGWIYPGNVYIQYNEKFKGRVTITRDTSASTAYMELSSLRSEDTAVYYCA RDGPWFAYWGQGTLVTVSS (SEQ ID NO:55); and
(b) a light chain variable domain (VL) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVLTQTPLSLSVTPGQPASISCRSSRSLLHSDGFTYLYWFLQKPGQSPQ LLIYQTSNLASGVPDRFSSSGSGTDFTLKISRVEAEDVGVYYCAQNLELP NTFGQGTKLEIK (SEQ ID NO:89).

110. The immunoconjugate of any one of embodiments 97 to 100, wherein the EpCAM antibody or EpCAM-binding fragments thereof comprises:
(a) a heavy chain variable domain (VH) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYYIHWVRQAPGQRLEYIG WIYPGNVYIQYSQKFQGRVTITADKSASTAYMELSSLRSEDTAVYYCARD GPWFAYWGQGTLVTVSS (SEQ ID NO:56); and
(b) a light chain variable domain (VL) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVLTQTPLSLSVTPGQPASISCRSSRSLLHSDGFTYLYWFLQKPGQSPQ LLIYQTSNLASGVPDRFSSSGSGTDFTLKISRVEAEDVGVYYCAQNLELP NTFGQGTKLEIK (SEQ ID NO:89).

111. The immunoconjugate of any one of embodiments 97 to 100, wherein the EpCAM antibody or EpCAM-binding fragments thereof comprises:
(a) a heavy chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYYIHWVRQAPGQRLEYIG WIYPGNVYIQYNEKFKGRATLTADKSASTAYMELSSLRSEDTAVYYCARD GPWFAYWGQGTLVTVSSASTKGPSVFPLAPSSKSTSGGTAALGCLVKDY FPEPVTVSWNSGALTSGVHTFPAVLQSSGLYSLSSVVTVPSSSLGTQTYI CNVNHKPSNTKVDKKVEPKSCDKTHTCPPCPAPELLGGPSVFLFPPKPK DTLMISRTPEVTCVVDVSHEDPEVKFNWYVDGVEVHNAKTKPREEQY NSTYRWSVLTVLHQDWLNGKEYKCKVSNKALPAPIEKTISKAKGQPREP QVYTLPPSRDELTKNQVSLTCLVKGFYPSDIAVEWESNGQPENNYKTTP PVLDSDGSFFLYSKLTVDKSRWQQGNVFSCSVMHEALHNHYTQKSLSL SPG (SEQ ID NO:103); and
(b) a light chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVLTQTPLSLSVTPGQPASISCRSSRSLLHSDGFTYLYWFLQKPGQSPQ LLIYQTSNLASGVPDRFSSSGSGTDFTLKISRVEAEDVGVYYCAQNLELP NTFGQGTKLEIKRTVAAPSVFIFPPSDEQLKSGTASWCLLNNFYPREAK VQWKVDNALQSGNSQESVTEQDSKDSTYSLSSTLTLSKADYEKHKVYA CEVTHQGLSSPVTKSFNRGEC (SEQ ID NO:140).

112. The immunoconjugate of any one of embodiments 97 to 100, wherein the EpCAM antibody or EpCAM-binding fragments thereof comprises:
(a) a heavy chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYYIHWVRQAPGQRLEYIG WIYPGNVYIQYNEKFKGRATLTADKSASTAYMELSSLRSEDTAVYYCARD GPWFAYWGQGTLVTVSSASTKGPSVFPLAPSSKSTSGGTAALGCLVKDY FPEPVTVSWNSGALTSGVHTFPAVLQSSGLYSLSSVVTVPSSSLGTQTYI CNVNHKPSNTKVDKKVEPKSCDKTHTCPPCPAPELLGGPSVFLFPPKPK DTLMISRTPEVTCVVVDVSHEDPEVKFNWYVDGVEVHNAKTKPREEQY NSTYRWSVLTVLHQDWLNGKEYKCKVSNKALPAPIEKTISKAKGQPREP QVYTLPPSRDELTKNQVSLTCLVKGFYPSDIAVEWESNGQPENNYKTTP PVLDSDGSFFLYSKLTVDKSRWQQGNVFSCSVMHEALHNHYTQKSLSL SPG (SEQ ID NO:103); and
(b) a light chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVLTQTPLSLSVTPGQPASISCRSSKSLLHSDGFTYLYWFLQKPGQSPQ LLIYQTSNLASGVPDRFSSSGSGTDFTLKISRVEAEDVGVYYCAQNLELP NTFGQGTKLEIKRTVAAPSVFIFPPSDEQLKSGTASWCLLNNFYPREAK VQWKVDNALQSGNSQESVTEQDSKDSTYSLSSTLTLSKADYEKHKVYA CEVTHQGLSSPVTKSFNRGEC (SEQ ID NO:138).

113. The immunoconjugate of any one of embodiments 97 to 100, wherein the EpCAM antibody or EpCAM-binding fragments thereof comprises:
(a) a heavy chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYYIHWVRQAPGQRLEW MGWIYPGNVYIQYNEKFKGRVTITRDTSASTAYMELSSLRSEDTAVYYCA RDGPWFAYWGQGTLVTVSSASTKGPSVFPLAPSSKSTSGGTAALGCLVK DYFPEPVTVSWNSGALTSGVHTFPAVLQSSGLYSLSSVVTVPSSSLGTQ TYICNVNHKPSNTKVDKKVEPKSCDKTHTCPPCPAPELLGGPSVFLFPPK PKDTLMISRTPEVTCVWDVSHEDPEVKFNWYVDGVEVHNAKTKPREE QYNSTYRWSVLTVLHQDWLNGKEYKCKVSNKALPAPIEKTISKAKGQPR EPQVYTLPPSRDELTKNQVSLTCLVKGFYPSDIAVEWESNGQPENNYKT TPPVLDSDGSFFLYSKLTVDKSRWQQGNVFSCSVMHEALHNHYTQKSL SLSPG (SEQ ID NO:105); and
(b) a light chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVMTQTPLSLSVTPGQPASISCRSSKSLLHSDGFTYLYWFLQKPGQSPQ LLIYQTSNLASGVPDRFSGSGSGTDFTLKISRVEAEDVGVYYCAQNLELP NTFGQGTKLEIKRTVAAPSVFIFPPSDEQLKSGTASWCLLNNFYPREAK VQWKVDNALQSGNSQESVTEQDSKDSTYSLSSTLTLSKADYEKHKVYA CEVTHQGLSSPVTKSFNRGEC (SEQ ID NO:139).

114. The immunoconjugate of any one of embodiments 97 to 100, wherein the EpCAM antibody or EpCAM-binding fragments thereof comprises:
(a) a heavy chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYYIHWVRQAPGQRLEYIG WIYPGNVYIQYSQKFQGRVTITADKSASTAYMELSSLRSEDTAVYYCARD GPWFAYWGQGTLVTVSSASTKGPSVFPLAPSSKSTSGGTAALGCLVKDY FPEPVTVSWNSGALTSGVHTFPAVLQSSGLYSLSSVVTVPSSSLGTQTYI CNVNHKPSNTKVDKKVEPKSCDKTHTCPPCPAPELLGGPSVFLFPPKPK DTLMISRTPEVTCVVDVSHEDPEVKFNWYVDGVEVHNAKTKPREEQY NSTYRWSVLTVLHQDWLNGKEYKCKVSNKALPAPIEKTISKAKGQPREP QVYTLPPSRDELTKNQVSLTCLVKGFYPSDIAVEWESNGQPENNYKTTP PVLDSDGSFFLYSKLTVDKSRWQQGNVFSCSVMHEALHNHYTQKSLSL SPG (SEQ ID NO:106); and
(b) a light chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVMTQTPLSLSVTPGQPASISCRSSKSLLHSDGFTYLYWFLQKPGQSPQ LLIYQTSNLASGVPDRFSGSGSGTDFTLKISRVEAEDVGVYYCAQNLELP NTFGQGTKLEIKRTVAAPSVFIFPPSDEQLKSGTASWCLLNNFYPREAK VQWKVDNALQSGNSQESVTEQDSKDSTYSLSSTLTLSKADYEKHKVYA CEVTHQGLSSPVTKSFNRGEC (SEQ ID NO:139).

115. The immunoconjugate of any one of embodiments 97 to 100, wherein the EpCAM antibody or EpCAM-binding fragments thereof comprises:
(a) a heavy chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYYIHWVRQAPGQRLE W MGWIYPGNVYIQYNEKFKGRVTITRDTSASTAYMELSSLRSEDTAVYYCA RDGPWFAYWGQGTLVTVSSASTKGPSVFPLAPSSKSTSGGTAALGCLVK DYFPEPVTVSWNSGALTSGVHTFPAVLQSSGLYSLSSWTVPSSSLGTQ TYICNVNHKPSNTKVDKKVEPKSCDKTHTCPPCPAPELLGGPSVFLFPPK PKDTLMISRTPEVTCVWDVSHEDPEVKFNWYVDGVEVHNAKTKPREE QYNSTYRWSVLTVLHQDWLNGKEYKCKVSNKALPAPIEKTISKAKGQPR EPQVYTLPPSRDELTKNQVSLTCLVKGFYPSDIAVEWESNGQPENNYKT TPPVLDSDGSFFLYSKLTVDKSRWQQGNVFSCSVMHEALHNHYTQKSL SLSPG (SEQ ID NO:105); and
(b) a light chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVLTQTPLSLSVTPGQPASISCRSSRSLLHSDGFTYLYWFLQKPGQSPQ LLIYQTSNLASGVPDRFSSSGSGTDFTLKISRVEAEDVGVYYCAQNLELP NTFGQGTKLEIKRTVAAPSVFIFPPSDEQLKSGTASWCLLNNFYPREAK VQWKVDNALQSGNSQESVTEQDSKDSTYSLSSTLTLSKADYEKHKVYA CEVTHQGLSSPVTKSFNRGEC (SEQ ID NO:140).

116. The immunoconjugate of any one of embodiments 97 to 100, wherein the EpCAM antibody or EpCAM-binding fragments thereof comprises:

(a) a heavy chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYYIHWVRQAPGQRLEYIG WIYPGNVYIQYSQKFQGRVTITADKSASTAYMELSSLRSEDTAVYYCARD GPWFAYWGQGTLVTVSSASTKGPSVFPLAPSSKSTSGGTAALGCLVKDY FPEPVTVSWNSGALTSGVHTFPAVLQSSGLYSLSSVVTVPSSSLGTQTYI CNVNHKPSNTKVDKKVEPKSCDKTHTCPPCPAPELLGGPSVFLFPPKPK DTLMISRTPEVTCVVVDVSHEDPEVKFNWYVDGVEVHNAKTKPREEQY NSTYRWSVLTVLHQDWLNGKEYKCKVSNKALPAPIEKTISKAKGQPREP QVYTLPPSRDELTKNQVSLTCLVKGFYPSDIAVEWESNGQPENNYKTTP PVLDSDGSFFLYSKLTVDKSRWQQGNVFSCSVMHEALHNHYTQKSLSL SPG (SEQ ID NO:106); and
(b) a light chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVLTQTPLSLSVTPGQPASISCRSSRSLLHSDGFTYLYWFLQKPGQSPQ LLIYQTSNLASGVPDRFSSSGSGTDFTLKISRVEAEDVGVYYCAQNLELP NTFGQGTKLEIKRTVAAPSVFIFPPSDEQLKSGTASWCLLNNFYPREAK VQWKVDNALQSGNSQESVTEQDSKDSTYSLSSTLTLSKADYEKHKVYA CEVTHQGLSSPVTKSFNRGEC (SEQ ID NO:140).

117. The immunoconjugate of any one of embodiments 97 to 100, wherein the EpCAM antibody or EpCAM-binding fragments thereof comprises:
(a) a VH-CDR1 comprising the amino acid sequence NYHIH (SEQ ID NO:22);
(b) a VH-CDR2 comprising the amino acid sequence WIYPGNVYIQYNEKFKG (SEQ ID NO:14);
(c) a VH-CDR3 comprising the amino acid sequence DGPWFAY (SEQ ID NO:15);
(d) a VL-CDR1 comprising the amino acid sequence RSSRSLLHSDGFTYLY (SEQ ID NO:42);
(e) a VL-CDR2 comprising the amino acid sequence QTSNLAS (SEQ ID NO:40; and
(f) a VL-CDR3 comprising the amino acid sequence AQNLELPNT (SEQ ID NO:41).

118. The immunoconjugate of any one of embodiments 97 to 100, wherein the EpCAM antibody or EpCAM-binding fragments thereof comprises:
(a) a VH-CDR1 comprising the amino acid sequence NYYIH (SEQ ID NO:13);
(b) a VH-CDR2 comprising the amino acid sequence WIYPGNVYIQYNEKFKG (SEQ ID NO:14);
(c) a VH-CDR3 comprising the amino acid sequence DGYWFAY (SEQ ID NO:33);
(d) a VL-CDR1 comprising the amino acid sequence RSSRSLLHSDGFTYLY (SEQ ID NO:42);
(e) a VL-CDR2 comprising the amino acid sequence QTSNLAS (SEQ ID NO:14); and
(f) a VL-CDR3 comprising the amino acid sequence AQNLELPNT (SEQ ID NO:41).

119. The immunoconjugate of any one of embodiments 97 to 100, wherein the EpCAM antibody or EpCAM-binding fragments thereof comprises:
(a) a VH-CDR1 comprising the amino acid sequence NYDIH (SEQ ID NO:23);
(b) a VH-CDR2 comprising the amino acid sequence WIYPGNVYIQYNEKFKG (SEQ ID NO:14);
(c) a VH-CDR3 comprising the amino acid sequence DGPWFAY (SEQ ID NO:15);
(d) a VL-CDR1 comprising the amino acid sequence RSSRSLLHSDGFTYLY (SEQ ID NO:42);
(e) a VL-CDR2 comprising the amino acid sequence QTSNLAS (SEQ ID NO:40); and
(f) a VL-CDR3 comprising the amino acid sequence AQNLELPNT (SEQ ID NO:41).

120. The immunoconjugate of any one of embodiments 97 to 100, wherein the EpCAM antibody or EpCAM-binding fragments thereof comprises:
(a) a VH-CDR1 comprising the amino acid sequence NYLIH (SEQ ID NO:25);
(b) a VH-CDR2 comprising the amino acid sequence WIYPGNVYIQYNEKFKG (SEQ ID NO:14;
(c) a VH-CDR3 comprising the amino acid sequence DGPWFAY (SEQ ID NO:15);
(d) a VL-CDR1 comprising the amino acid sequence RSSRSLLHSDGFTYLY (SEQ ID NO:42);
(e) a VL-CDR2 comprising the amino acid sequence QTSNLAS (SEQ ID NO:40); and
(f) a VL-CDR3 comprising the amino acid sequence AQNLELPNT (SEQ ID NO:41).

121. The immunoconjugate of any one of embodiments 97 to 100 and 117 to 120, wherein the EpCAM antibody or EpCAM-binding fragments thereof comprises:
(a) a heavy chain variable domain (VH) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYHIHWVRQAPGQRLEYI GWIYPGNVYIQYNEKFKGRATLTADKSASTAYMELSSLRSEDTAVYYCAR DGPWFAYWGQGTLVTVSS (SEQ ID NO:75); and
(b) a light chain variable domain (VL) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVLTQTPLSLSVTPGQPASISCRSSRSLLHSDGFTYLYWFLQKPGQSPQ LLIYQTSNLASGVPDRFSSSGSGTDFTLKISRVEAEDVGVYYCAQNLELP NTFGQGTKLEIK (SEQ ID NO:89).

122. The immunoconjugate of any one of embodiments 97 to 100 and 117 to 120, wherein the EpCAM antibody or EpCAM-binding fragments thereof comprises:
(a) a heavy chain variable domain (VH) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYYIHWVRQAPGQRLEYIG WIYPGNVYIQYNEKFKGRATLTADKSASTAYMELSSLRSEDTAVYYCARD GYWFAYWGQGTLVTVSS (SEQ ID NO:77); and
(b) a light chain variable domain (VL) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVLTQTPLSLSVTPGQPASISCRSSRSLLHSDGFTYLYWFLQKPGQSPQ LLIYQTSNLASGVPDRFSSSGSGTDFTLKISRVEAEDVGVYYCAQNLELP NTFGQGTKLEIK (SEQ ID NO:89).

123. The immunoconjugate of any one of embodiments 97 to 100 and 117 to 120, wherein the EpCAM antibody or EpCAM-binding fragments thereof comprises:
(a) a heavy chain variable domain (VH) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYDIHWVRQAPGQRLEYI GWIYPGNVYIQYNEKFKGRATLTADKSASTAYMELSSLRSEDTAVYYCAR DGPWFAYWGQGTLVTVSS (SEQ ID NO:76); and
(b) a light chain variable domain (VL) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVLTQTPLSLSVTPGQPASISCRSSRSLLHSDGFTYLYWFLQKPGQSPQ LLIYQTSNLASGVPDRFSSSGSGTDFTLKISRVEAEDVGVYYCAQNLELP NTFGQGTKLEIK (SEQ ID NO:89).

124. The immunoconjugate of any one of embodiments 97 to 100 and 117 to 120, wherein the EpCAM antibody or EpCAM-binding fragments thereof comprises:
(a) a heavy chain variable domain (VH) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYLIHWVRQAPGQRLEYIG WIYPGNVYIQYNEKFKGRATLTADKSASTAYMELSSLRSEDTAVYYCARD GPWFAYWGQGTLVTVSS (SEQ ID NO:84); and
(b) a light chain variable domain (VL) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVLTQTPLSLSVTPGQPASISCRSSRSLLHSDGFTYLYWFLQKPGQSPQ LLIYQTSNLASGVPDRFSSSGSGTDFTLKISRVEAEDVGVYYCAQNLELP NTFGQGTKLEIK (SEQ ID NO:89).

125. The immunoconjugate of any one of embodiments 97 to 100 and 117 to 120, wherein the EpCAM antibody or EpCAM-binding fragments thereof comprises:
(a) a heavy chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYHIHWVRQAPGQRLEYI GWIYPGNVYIQYNEKFKGRATLTADKSASTAYMELSSLRSEDTAVYYCAR DGPWFAYWGQGTLVTVSSASTKGPSVFPLAPSSKSTSGGTAALGCLVKD YFPEPVTVSWNSGALTSGVHTFPAVLQSSGLYSLSSVVTVPSSSLGTQTY ICNVNHKPSNTKVDKKVEPKSCDKTHTCPPCPAPELLGGPSVFLFPPKPK DTLMISRTPEVTCWVDVSHEDPEVKFNWYVDGVEVHNAKTKPREEQY NSTYRWSVLTVLHQDWLNGKEYKCKVSNKALPAPIEKTISKAKGQPREP QVYTLPPSRDELTKNQVSLTCLVKGFYPSDIAVEWESNGQPENNYKTTP PVLDSDGSFFLYSKLTVDKSRWQQGNVFSCSVMHEALHNHYTQKSLSL SPG (SEQ ID NO:125); and
(b) a light chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVLTQTPLSLSVTPGQPASISCRSSRSLLHSDGFTYLYWFLQKPGQSPQ LLIYQTSNLASGVPDRFSSSGSGTDFTLKISRVEAEDVGVYYCAQNLELP NTFGQGTKLEIKRTVAAPSVFIFPPSDEQLKSGTASWCLLNNFYPREAK VQWKVDNALQSGNSQESVTEQDSKDSTYSLSSTLTLSKADYEKHKVYA CEVTHQGLSSPVTKSFNRGEC (SEQ ID NO:140).

126. The immunoconjugate of any one of embodiments 97 to 100 and 117 to 120, wherein the EpCAM antibody or EpCAM-binding fragments thereof comprises:
(a) a heavy chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYYIHWVRQAPGQRLEYIG WIYPGNVYIQYNEKFKGRATLTADKSASTAYMELSSLRSEDTAVYYCARD GYWFAYWGQGTLVTVSSASTKGPSVFPLAPSSKSTSGGTAALGCLVKDY FPEPVTVSWNSGALTSGVHTFPAVLQSSGLYSLSSVVTVPSSSLGTQTYI CNVNHKPSNTKVDKKVEPKSCDKTHTCPPCPAPELLGGPSVFLFPPKPK DTLMISRTPEVTCWVDVSHEDPEVKFNWYVDGVEVHNAKTKPREEQY NSTYRWSVLTVLHQDWLNGKEYKCKVSNKALPAPIEKTISKAKGQPREP QVYTLPPSRDELTKNQVSLTCLVKGFYPSDIAVEWESNGQPENNYKTTP PVLDSDGSFFLYSKLTVDKSRWQQGNVFSCSVMHEALHNHYTQKSLSL SPG (SEQ ID NO:127); and
(b) a light chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVLTQTPLSLSVTPGQPASISCRSSRSLLHSDGFTYLYWFLQKPGQSPQ LLIYQTSNLASGVPDRFSSSGSGTDFTLKISRVEAEDVGVYYCAQNLELP NTFGQGTKLEIKRTVAAPSVFIFPPSDEQLKSGTASWCLLNNFYPREAK VQWKVDNALQSGNSQESVTEQDSKDSTYSLSSTLTLSKADYEKHKVYA CEVTHQGLSSPVTKSFNRGEC (SEQ ID NO:140).

127. The immunoconjugate of any one of embodiments 97 to 100 and 117 to 120, wherein the EpCAM antibody or EpCAM-binding fragments thereof comprises:
(a) a heavy chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYDIHWVRQAPGQRLEYI GWIYPGNVYIQYNEKFKGRATLTADKSASTAYMELSSLRSEDTAVYYCAR DGPWFAYWGQGTLVTVSSASTKGPSVFPLAPSSKSTSGGTAALGCLVKD YFPEPVTVSWNSGALTSGVHTFPAVLQSSGLYSLSSWTVPSSSLGTQTY ICNVNHKPSNTKVDKKVEPKSCDKTHTCPPCPAPELLGGPSVFLFPPKPK DTLMISRTPEVTCVWDVSHEDPEVKFNWYVDGVEVHNAKTKPREEQY NSTYRWSVLTVLHQDWLNGKEYKCKVSNKALPAPIEKTISKAKGQPREP QVYTLPPSRDELTKNQVSLTCLVKGFYPSDIAVEWESNGQPENNYKTTP PVLDSDGSFFLYSKLTVDKSRWQQGNVFSCSVMHEALHNHYTQKSLSL SPG (SEQ ID NO:126); and
(b) a light chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVLTQTPLSLSVTPGQPASISCRSSRSLLHSDGFTYLYWFLQKPGQSPQ LLIYQTSNLASGVPDRFSSSGSGTDFTLKISRVEAEDVGVYYCAQNLELP NTFGQGTKLEIKRTVAAPSVFIFPPSDEQLKSGTASWCLLNNFYPREAK VQWKVDNALQSGNSQESVTEQDSKDSTYSLSSTLTLSKADYEKHKVYA CEVTHQGLSSPVTKSFNRGEC (SEQ ID NO:140).

128. The immunoconjugate of any one of embodiments 97 to 100 and 117 to 120, wherein the EpCAM antibody or EpCAM-binding fragments thereof comprises:
(a) a heavy chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYLIHWVRQAPGQRLEYIG WIYPGNVYIQYNEKFKGRATLTADKSASTAYMELSSLRSEDTAVYYCARD GPWFAYWGQGTLVTVSSASTKGPSVFPLAPSSKSTSGGTAALGCLVKDY FPEPVTVSWNSGALTSGVHTFPAVLQSSGLYSLSSVVTVPSSSLGTQTYI CNVNHKPSNTKVDKKVEPKSCDKTHTCPPCPAPELLGGPSVFLFPPKPK DTLMISRTPEVTCVVVDVSHEDPEVKFNWYVDGVEVHNAKTKPREEQY NSTYRWSVLTVLHQDWLNGKEYKCKVSNKALPAPIEKTISKAKGQPREP QVYTLPPSRDELTKNQVSLTCLVKGFYPSDIAVEWESNGQPENNYKTTP PVLDSDGSFFLYSKLTVDKSRWQQGNVFSCSVMHEALHNHYTQKSLSL SPG (SEQ ID NO:134); and
(b) a light chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVLTQTPLSLSVTPGQPASISCRSSRSLLHSDGFTYLYWFLQKPGQSPQ LLIYQTSNLASGVPDRFSSSGSGTDFTLKISRVEAEDVGVYYCAQNLELP NTFGQGTKLEIKRTVAAPSVFIFPPSDEQLKSGTASWCLLNNFYPREAK VQWKVDNALQSGNSQESVTEQDSKDSTYSLSSTLTLSKADYEKHKVYA CEVTHQGLSSPVTKSFNRGEC (SEQ ID NO:140).

129. The immunoconjugate of any one of embodiments 97 to 128, wherein the EpCAM antibody or EpCAM-binding fragments thereof is a non-human mammal antibody.

130. The immunoconjugate of any one of embodiments 97 to 129, wherein the EpCAM antibody or EpCAM-binding fragments thereof is a murine antibody.

131. The immunoconjugate of any one of embodiments 97 to 128, wherein the EpCAM antibody or EpCAM-binding fragments thereof is a chimeric antibody.

132. The immunoconjugate of any one of embodiments 97 to 128, wherein the EpCAM antibody or EpCAM-binding fragments thereof is a humanized antibody.

133. The immunoconjugate of any one of embodiments 97 to 128, wherein the EpCAM antibody or EpCAM-binding fragments thereof is a human antibody.

134. The immunoconjugate of any one of embodiments 97 to 133, wherein the EpCAM antibody is a full-length antibody.

135. The immunoconjugate of embodiment 134, wherein the EpCAM antibody is a human IgG1 antibody.

136. The immunoconjugate of any one of embodiments 97 to 133, wherein the EpCAM antibody or EpCAM-binding fragments thereof is a Fab.

137. The immunoconjugate of any one of embodiments 97 to 133, wherein the EpCAM antibody or EpCAM-binding fragments thereof is a Fab′.

138. The immunoconjugate of any one of embodiments 97 to 133, wherein the EpCAM antibody or EpCAM-binding fragments thereof is a F(ab′)₂.

139. The immunoconjugate of any one of embodiments 97 to 133, wherein the EpCAM antibody or EpCAM-binding fragments thereof is an scFv.

140. The immunoconjugate of any one of embodiments 97 to 133, wherein the EpCAM antibody or EpCAM-binding fragments thereof is a disulfide-linked Fv (dsFv).

141. The immunoconjugate of any one of embodiments 97 to 140, wherein A is substituted with one or more polyols.

142. The immunoconjugate of embodiment 141, wherein the one or more polyols is —(C₁-C₆ alkylene)-X⁵—Y³, wherein X⁵ is —NR¹²C(═O)— or —C(═O)NR¹²;
wherein:
   Y³ is C₆ alkyl substituted with 5 OH groups; and
   R¹² is —H, C₁-C₆ alkyl, C₁-C₆ fluoroalkyl, C₃-C₆ cycloalkyl, aryl, heteroaryl, or benzyl.

143. The immunoconjugate of embodiment 141, wherein the one or more polyols is

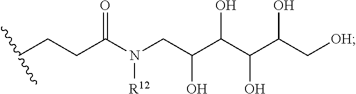

wherein R¹² is H or methyl.

144. The immunoconjugate of any one of embodiments 97 to 140, wherein the compound is

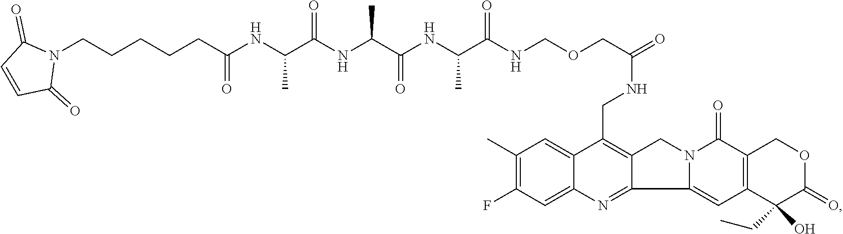 [SMILES: CC[C@@]1(O)C(=O)OCc2c1cc1n(c2=O)Cc2c-1nc1cc(F)c(C)cc1c2CNC(=O)COCNC(=O)[C@H](C)NC(=O)[C@H](C)NC(=O)[C@H](C)NC(=O)CCCCCN1C(=O)C=CC1=O]

or a pharmaceutically acceptable salt thereof.

145. The immunoconjugate of any one of embodiments 97 to 140, wherein the compound is

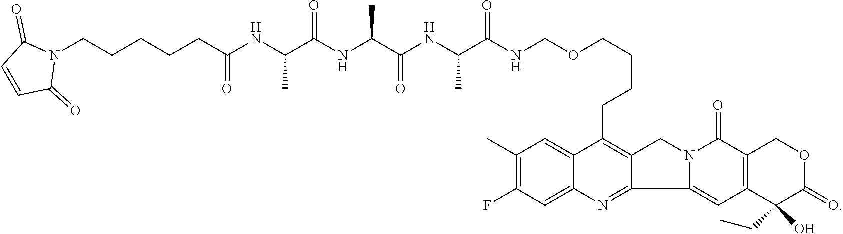 [SMILES: CC[C@@]1(O)C(=O)OCc2c1cc1n(c2=O)Cc2c-1nc1cc(F)c(C)cc1c2CCCCOCNC(=O)[C@H](C)NC(=O)[C@H](C)NC(=O)[C@H](C)NC(=O)CCCCCN1C(=O)C=CC1=O]

or a pharmaceutically acceptable salt thereof.

146. The immunoconjugate of any one of embodiments 97 to 145, having a drug-to-antibody ratio (DAR) of at least 2.

147. The immunoconjugate of any one of embodiments 97 to 146, having a DAR of at least 3.

148. The immunoconjugate of any one of embodiments 97 to 147, having a DAR of at least 4.

149. The immunoconjugate of any one of embodiments 97 to 148, having a DAR of 4.

150. The immunoconjugate of any one of embodiments 97 to 149, having a DAR of at least 5.

151. The immunoconjugate of any one of embodiments 97 to 150, having a DAR of 5.

152. The immunoconjugate of any one of embodiments 97 to 151, having a DAR of at least 6.

153. The immunoconjugate of any one of embodiments 97 to 152, having a DAR of 6.

154. The immunoconjugate of any one of embodiments 97 to 153, having a DAR of at least 7.

155. The immunoconjugate of any one of embodiments 97 to 154, having a DAR of 7.

156. The immunoconjugate of any one of embodiments 97 to 155, having a DAR of at least 8

157. The immunoconjugate of any one of embodiments 97 to 156, having a DAR of 8.

158. The immunoconjugate of any one of embodiments 97 to 157, having a DAR of at least 9.

159. The immunoconjugate of any one of embodiments 97 to 158, having a DAR of 9.

160. The immunoconjugate of any one of embodiments 97 to 159, having a DAR of at least 10.

161. The immunoconjugate of any one of embodiments 97 to 160, having a DAR of 10.

162. The immunoconjugate of any one of embodiments 97 to 161, having a DAR of at least 11.

163. The immunoconjugate of any one of embodiments 97 to 162, having a DAR of 11.

164. The immunoconjugate of any one of embodiments 97 to 163, having a DAR of at least 12.

165. The immunoconjugate of any one of embodiments 97 to 164, having a DAR of 12.

166. The immunoconjugate of any one of embodiments 97 to 165, comprising:
(a) a cleavable moiety coupled to the EpCAM antibody or EpCAM-binding fragment thereof, wherein the cleavable moiety is a polypeptide that functions as a substrate for a protease; and
(b) a masking moiety coupled to the EpCAM antibody or EpCAM-binding fragment thereof, wherein the masking moiety inhibits the binding of the antibody or antibody fragment to EpCAM when the activatable antibody is in an uncleaved state, wherein the activatable antibody in the uncleaved state has a structural arrangement from N-terminus to C-terminus of: (masking moiety)-(cleavable moiety)-(antibody or antibody fragment) or (antibody or antibody fragment)-(cleavable moiety)-(masking moiety).

167. The immunoconjugate of embodiment 166, wherein the masking moiety comprises the amino acid sequence PLMTCSDYYTCLNNL (SEQ ID NO:151).

168. The immunoconjugate of embodiment 166, wherein the masking moiety comprises the amino acid sequence LSCTHSRYDMHCPHM (SEQ ID NO:152).

169. The immunoconjugate of embodiment 166, wherein the masking moiety comprises the amino acid sequence HYCHSRTDTITHCNA (SEQ ID NO:153).

170. The immunoconjugate of embodiment 166, wherein the masking moiety comprises the amino acid sequence WCPRLFDRPSMGCPT (SEQ ID NO:154).

171. The immunoconjugate of embodiment 166, wherein the masking moiety comprises the amino acid sequence WWPPCQGGAWCEQRI (SEQ ID NO:155).

172. The immunoconjugate of embodiment 166, wherein the masking moiety comprises the amino acid sequence HSGCPRLFDRCSAPA (SEQ ID NO:156).

173. The immunoconjugate of embodiment 166, wherein the masking moiety comprises the amino acid sequence FICPTLYDRPHCMHT (SEQ ID NO:157).

174. The immunoconjugate of embodiment 166, wherein the masking moiety comprises the amino acid sequence DCTGYSPSVLPACRV (SEQ ID NO:162).

175. The immunoconjugate of embodiment 166, wherein the masking moiety comprises the amino acid sequence FCSGYSPSVLPSCLM (SEQ ID NO:163).

176. The immunoconjugate of embodiment 166, wherein the masking moiety comprises the amino acid sequence SKPCSYMHPYCFYNS (SEQ ID NO:164).

177. The immunoconjugate of embodiment 166, wherein the masking moiety comprises the amino acid sequence LTRCTIAHPYCYYNY (SEQ ID NO:165).

178. The immunoconjugate of embodiment 166, wherein the masking moiety comprises the amino acid sequence PNTCMSERRICSLTY (SEQ ID NO:166).

179. The immunoconjugate of embodiment 166, wherein the masking moiety comprises the amino acid sequence PRPHCAILRQCLAAT (SEQ ID NO:167).

180. The immunoconjugate of any one of embodiments 166 to 179, wherein the cleavable moiety comprises the amino acid sequence AVGLLAPPGGLSGRSDNI (SEQ ID NO:168).

181. The immunoconjugate of any one of embodiments 166 to 179, wherein the cleavable moiety comprises the amino acid sequence ISSGLLSGRSDNI (SEQ ID NO:169).

182. An immunoconjugate, comprising:
(a) an activatable antibody comprising:
   (x) an EpCAM antibody or EpCAM-binding fragment thereof, comprising:
      (5) a heavy chain complementarity determining region 1 (VH-CDR1) comprising the amino acid sequence X₁YX₃X₄H, wherein X₁ is selected from N and S, X₃ is selected from Y, N, F, S, H, D, L. I, and W, and X₄ is selected from I and M;
      (6) a heavy chain complementarity determining region 2 (VH-CDR2) comprising the amino acid sequence WX₂X₃PGX₆VYIQYX₁₂X₁₃KFX₁₇G, wherein X₂ is selected from I and F, X₃ is selected from Y and N, X₆ is selected from N and D, X₁₂ is selected from N and S, X₁₃ is selected from E and Q, and X₁₇ is selected from K and Q (SEQ ID NO:7);
      (7) heavy chain complementarity determining region 3 (CDR3) (VH-CDR3) comprising the amino acid sequence X₁GX₃X₄FAY, wherein X₁ is selected from D and E, X₃ is selected from P, A, S, Y, F, G, T, and V, and X₄ is selected from Y and W (SEQ ID NO:8);
      (8) a light chain complementarity determining region 1 (VL-CDR1) comprising the amino acid sequence RSSX₄SLLHSX₁₀GX₁₂TY LX₁₆, wherein X₄ is selected from R and K, X₁₀ is selected from N and D, X₁₂ is selected from F and I, and X₁₆ is selected from Y and S SEQ ID NO:10);
      (9) a light chain complementarity determining region 2 (VL-CDR2) comprising the amino acid sequence QTSNLAS (SEQ ID NO:40); and
      (10) a light chain complementarity determining region 3 (VL-CDR3) comprising the amino acid sequence X₁QX₃LELPX₈T, wherein X₁ is selected from A, L, and Q, X₃ is selected from S, G, Y, and N, and X₈ is selected from N and W (SEQ ID NO:11);
   (xi) a cleavable moiety coupled to the EpCAM antibody or EpCAM-binding fragment thereof, wherein the cleavable moiety is a polypeptide that functions as a substrate for a protease; and
   (xii) a masking moiety coupled to the EpCAM antibody or EpCAM-binding fragment thereof, wherein the masking moiety inhibits the binding of the antibody or antibody fragment to EpCAM when the activatable antibody is in an uncleaved state,
wherein the activatable antibody in the uncleaved state has a structural arrangement from N-terminus to C-terminus of: (masking moiety)-(cleavable moiety)-(antibody or antibody fragment) or (antibody or antibody fragment)-(cleavable moiety)-(masking moiety); and
(b) at least one linker-payload comprising a compound of Formula I or a pharmaceutically acceptable salt thereof (wherein the compound of Formula I or a pharmaceutically acceptable salt thereof represents the linker-payload reactant, i.e. the linker-payload prior to conjugation to the EpCAM antibody or EpCAM-binding fragment thereof):

E-A-Z′-L¹-D (Formula I)
wherein:
D is represented by the following structural formula:

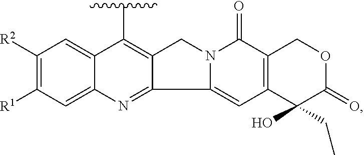

R¹ is F;
R² is methyl;
L¹-Z′—* is —(C₁-C₄ alkylene)-O—CH₂—NR⁸—*, —(C₁-C₄ alkylene)-NR⁸, or —(C₁-C₅ alkylene)-NR⁵C(═O)—(C₁-C₅ alkylene)-O—CH₂—NR⁸*, where* is a site covalently attached to A;
each R⁵ is independently H, methyl, or benzyl;
each R⁸ is independently H, methyl, or benzyl;
A is a peptide comprising 2 to 4 amino acids; and
E is C(═O)—(C₁-C₁₀ alkylene)-X³ wherein X³ is

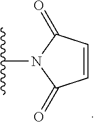 [SMILES: *N1C(=O)C=CC1=O]

183. The immunoconjugate of embodiment 182, wherein the masking moiety comprises the amino acid sequence PLMTCSDYYTCLNNL (SEQ ID NO:151).

184. The immunoconjugate of embodiment 182, wherein the masking moiety comprises the amino acid sequence LSCTHSRYDMHCPHM (SEQ ID NO:152).

185. The immunoconjugate of embodiment 182, wherein the masking moiety comprises the amino acid sequence HYCHSRTDTITHCNA (SEQ ID NO:153).

186. The immunoconjugate of embodiment 182, wherein the masking moiety comprises the amino acid sequence WCPRLFDRPSMGCPT (SEQ ID NO:154).

187. The immunoconjugate of embodiment 182, wherein the masking moiety comprises the amino acid sequence WWPPCQGGAWCEQRI (SEQ ID NO:155).

188. The immunoconjugate of embodiment 182, wherein the masking moiety comprises the amino acid sequence HSGCPRLFDRCSAPA (SEQ ID NO:156).

189. The immunoconjugate of embodiment 182, wherein the masking moiety comprises the amino acid sequence FICPTLYDRPHCMHT (SEQ ID NO:157).

190. The immunoconjugate of embodiment 182, wherein the masking moiety comprises the amino acid sequence DCTGYSPSVLPACRV (SEQ ID NO:162).

191. The immunoconjugate of embodiment 182, wherein the masking moiety comprises the amino acid sequence FCSGYSPSVLPSCLM (SEQ ID NO:163).

192. The immunoconjugate of embodiment 182, wherein the masking moiety comprises the amino acid sequence SKPCSYMHPYCFYNS (SEQ ID NO:164).

193. The immunoconjugate of embodiment 182, wherein the masking moiety comprises the amino acid sequence LTRCTIAHPYCYYNY (SEQ ID NO:165).

194. The immunoconjugate of embodiment 182, wherein the masking moiety comprises the amino acid sequence PNTCMSERRICSLTY (SEQ ID NO:166).

195. The immunoconjugate of embodiment 182, wherein the masking moiety comprises the amino acid sequence PRPHCAILRQCLAAT (SEQ ID NO:167).

196. The immunoconjugate of any one of embodiments 182 to 195, wherein the cleavable moiety comprises the amino acid sequence AVGLLAPPGGLSGRSDNI (SEQ ID NO:168).

197. The immunoconjugate of any one of embodiments 182 to 195, wherein the cleavable moiety comprises the amino acid sequence ISSGLLSGRSDNI (SEQ ID NO:169).

198. The immunoconjugate of any one of embodiments 182 to 197, wherein the EpCAM antibody or EpCAM-binding fragment thereof comprises:
(a) a VH-CDR1 comprising the amino acid sequence NYX₃IH, wherein X₃ is selected from Y, N, F, S, H, D, L, I, and W (SEQ ID NO:6);
(b) a VH-CDR2 comprising the amino acid sequence WX₂X₃PGX₆VYIQYX₁₂X₁₃KFX₁₇G, wherein X₂ is selected from I and F, X₃ is selected from Y and N, X₆ is selected from N and D, X₁₂ is selected from N and S, X₁₃ is selected from E and Q, and X₁₇ is selected from K and Q (SEQ ID NO:7);
(c) a VH-CDR3 comprising the amino acid sequence DGPX₄FAY, wherein X₄ is selected from Y and W (SEQ ID NO:9);
(d) a VL-CDR1 comprising the amino acid sequence RSSX₄SLLHSX₁₀GX₁₂TYLX₁₆, wherein X₄ is selected from R and K, X₁₀ is selected from N and D, X₁₂ is selected from F and I, and Xie is selected from Y and S (SEQ ID NO:10);
(e) a VL-CDR2 comprising the amino acid sequence QTSNLAS (SEQ ID NO:40); and
(f) a VL-CDR3 comprising the amino acid sequence AQX₃LELPNT, wherein X₃ is selected from S, G, Y, and N (SEQ ID NO:12).

199. The immunoconjugate of any one of embodiments 182 to 198, wherein the EpCAM antibody or EpCAM-binding fragment thereof binds with a Ko of 3.0 nM or less to both human EpCAM and cynomolgus EpCAM.

200. The immunoconjugate of any one of embodiments 182 to 199, wherein the EpCAM antibody or EpCAM-binding fragment thereof binds to an epitope within the extracellular domain of human EpCAM (QEECVCENYKLAVNCFVNNNRQCQCTSVGAQNTVIC) (SEQ ID NO:2).

201. The immunoconjugate of any one of embodiments 182 to 200, wherein the EpCAM antibody or EpCAM-binding fragment thereof comprises:
(a) a VH-CDR1 comprising the amino acid sequence NYYIH (SEQ ID NO:13);
(b) a VH-CDR2 comprising the amino acid sequence WIYPGNVYIQYNEKFKG (SEQ ID NO:14);
(c) a VH-CDR3 comprising the amino acid sequence DGPWFAY (SEQ ID NO:15);
(d) a VL-CDR1 comprising the amino acid sequence RSSRSLLHSDGFTYLY (SEQ ID NO:42);
(e) a VL-CDR2 comprising the amino acid sequence QTSNLAS (SEQ ID NO:40); and
(f) a VL-CDR3 comprising the amino acid sequence AQNLELPNT (SEQ ID NO:41).

202. The immunoconjugate of any one of embodiments 182 to 200, wherein the EpCAM antibody or EpCAM-binding fragment thereof comprises:
(a) a VH-CDR1 comprising the amino acid sequence NYYIH (SEQ ID NO:13);
(b) a VH-CDR2 comprising the amino acid sequence WIYPGNVYIQYSQKFQG (SEQ ID NO:26);
(c) a VH-CDR3 comprising the amino acid sequence DGPWFAY (SEQ ID NO:15);
(d) a VL-CDR1 comprising the amino acid sequence RSSRSLLHSDGFTYLY (SEQ ID NO:42);
(e) a VL-CDR2 comprising the amino acid sequence QTSNLAS (SEQ ID NO:40); and
(f) a VL-CDR3 comprising the amino acid sequence AQNLELPNT (SEQ ID NO:41).

203. The immunoconjugate of any one of embodiments 182 to 200, wherein the EpCAM antibody or EpCAM-binding fragment thereof comprises:
(a) a VH-CDR1 comprising the amino acid amino acid sequence NYYIH (SEQ ID NO:13);
(b) a VH-CDR2 comprising the amino acid sequence WIYPGNVYIQYNEKFKG (SEQ ID NO:14);
(c) a VH-CDR3 comprising the amino acid sequence DGPWFAY (SEQ ID NO:15);
(d) a VL-CDR1 comprising the amino acid sequence RSSKSLLHSDGFTYLY (SEQ ID NO:39);
(e) a VL-CDR2 comprising the amino acid sequence QTSNLAS (SEQ ID NO:40); and
(f) a VL-CDR3 comprising the amino acid sequence AQNLELPNT (SEQ ID NO:41).

204. The immunoconjugate of any one of embodiments 182 to 200, wherein the EpCAM antibody or EpCAM-binding fragment thereof comprises:
(a) a VH-CDR1 comprising the amino acid sequence NYYIH (SEQ ID NO:13);
(b) a VH-CDR2 comprising the amino acid sequence WIYPGNVYIQYSQKFQG (SEQ ID NO:26);
(c) a VH-CDR3 comprising the amino acid sequence DGPWFAY (SEQ ID NO:15);
(d) a VL-CDR1 comprising the amino acid sequence RSSKSLLHSDGFTYLY (SEQ ID NO:39);
(e) a VL-CDR2 comprising the amino acid sequence QTSNLAS (SEQ ID NO:40); and
(f) a VL-CDR3 comprising the amino acid sequence AQNLELPNT (SEQ ID NO:41).

205. The immunoconjugate of any one of embodiments 182 to 204, wherein the EpCAM antibody or EpCAM-binding fragment thereof comprises:
(a) a heavy chain variable domain (VH) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYYIHWVRQAPGQRLEYIG WIYPGNVYIQYNEKFKGRATLTADKSASTAYMELSSLRSEDTAVYYCARD GPWFAYWGQGTLVTVSS (SEQ ID NO:54); and
(b) a light chain variable domain (VL) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVLTQTPLSLSVTPGQPASISCRSSRSLLHSDGFTYLYWFLQKPGQSPQ LLIYQTSNLASGVPDRFSSSGSGTDFTLKISRVEAEDVGVYYCAQNLELP NTFGQGTKLEIK (SEQ ID NO:89).

206. The immunoconjugate of any one of embodiments 182 to 204, wherein the EpCAM antibody or EpCAM-binding fragment thereof comprises:
(a) a heavy chain variable domain (VH) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYYIHWVRQAPGQRLEYIG WIYPGNVYIQYNEKFKGRATLTADKSASTAYMELSSLRSEDTAVYYCARD GPWFAYWGQGTLVTVSS (SEQ ID NO:54); and
(b) a light chain variable domain (VL) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVLTQTPLSLSVTPGQPASISCRSSKSLLHSDGFTYLYWFLQKPGQSPQ LLIYQTSNLASGVPDRFSSSGSGTDFTLKISRVEAEDVGVYYCAQNLELP NTFGQGTKLEIK (SEQ ID NO:87).

207. The immunoconjugate of any one of embodiments 182 to 204, wherein the EpCAM antibody or EpCAM-binding fragment thereof comprises:
(a) a heavy chain variable domain (VH) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYYIHWVRQAPGQRLEW MGWIYPGNVYIQYNEKFKGRVTITRDTSASTAYMELSSLRSEDTAVYYCA RDGPWFAYWGQGTLVTVSS (SEQ ID NO:55); and
(b) a light chain variable domain (VL) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVLTQTPLSLSVTPGQPASISCRSSKSLLHSDGFTYLYWFLQKPGQSPQ LLIYQTSNLASGVPDRFSSSGSGTDFTLKISRVEAEDVGVYYCAQNLELP NTFGQGTKLEIK (SEQ ID NO:87).

208. The immunoconjugate of any one of embodiments 182 to 204, wherein the EpCAM antibody or EpCAM-binding fragment thereof comprises:
(a) a heavy chain variable domain (VH) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYYIHWVRQAPGQRLEYIG WIYPGNVYIQYSQKFQGRVTITADKSASTAYMELSSLRSEDTAVYYCARD GPWFAYWGQGTLVTVSS (SEQ ID NO:56); and
(b) a light chain variable domain (VL) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVMTQTPLSLSVTPGQPASISCRSSKSLLHSDGFTYLYWFLQKPGQSPQ LLIYQTSNLASGVPDRFSGSGSGTDFTLKISRVEAEDVGVYYCAQNLELP NTFGQGTKLEIK (SEQ ID NO:88).

209. The immunoconjugate of any one of embodiments 182 to 204, wherein the EpCAM antibody or EpCAM-binding fragment thereof comprises:
(a) a heavy chain variable domain (VH) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYYIHWVRQAPGQRLEW MGWIYPGNVYIQYNEKFKGRVTITRDTSASTAYMELSSLRSEDTAVYYCA RDGPWFAYWGQGTLVTVSS (SEQ ID NO:55); and
(b) a light chain variable domain (VL) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVLTQTPLSLSVTPGQPASISCRSSRSLLHSDGFTYLYWFLQKPGQSPQ LLIYQTSNLASGVPDRFSSSGSGTDFTLKISRVEAEDVGVYYCAQNLELP NTFGQGTKLEIK (SEQ ID NO:89).

210. The immunoconjugate of any one of embodiments 182 to 204, wherein the EpCAM antibody or EpCAM-binding fragment thereof comprises:
(a) a heavy chain variable domain (VH) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYYIHWVRQAPGQRLEYIG WIYPGNVYIQYSQKFQGRVTITADKSASTAYMELSSLRSEDTAVYYCARD GPWFAYWGQGTLVTVSS (SEQ ID NO:56); and
(b) a light chain variable domain (VL) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVLTQTPLSLSVTPGQPASISCRSSRSLLHSDGFTYLYWFLQKPGQSPQ LLIYQTSNLASGVPDRFSSSGSGTDFTLKISRVEAEDVGVYYCAQNLELP NTFGQGTKLEIK (SEQ ID NO:89).

211. The immunoconjugate of any one of embodiments 182 to 200, wherein the EpCAM antibody or EpCAM-binding fragment thereof comprises:
(a) a heavy chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYYIHWVRQAPGQRLEYIG WIYPGNVYIQYNEKFKGRATLTADKSASTAYMELSSLRSEDTAVYYCARD GPWFAYWGQGTLVTVSSASTKGPSVFPLAPSSKSTSGGTAALGCLVKDY FPEPVTVSWNSGALTSGVHTFPAVLQSSGLYSLSSVVTVPSSSLGTQTYI CNVNHKPSNTKVDKKVEPKSCDKTHTCPPCPAPELLGGPSVFLFPPKPK DTLMISRTPEVTCVVVDVSHEDPEVKFNWYVDGVEVHNAKTKPREEQY NSTYRWSVLTVLHQDWLNGKEYKCKVSNKALPAPIEKTISKAKGQPREP QVYTLPPSRDELTKNQVSLTCLVKGFYPSDIAVEWESNGQPENNYKTTP PVLDSDGSFFLYSKLTVDKSRWQQGNVFSCSVMHEALHNHYTQKSLSL SPG (SEQ ID NO:103); and
(b) a light chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVLTQTPLSLSVTPGQPASISCRSSRSLLHSDGFTYLYWFLQKPGQSPQ LLIYQTSNLASGVPDRFSSSGSGTDFTLKISRVEAEDVGVYYCAQNLELP NTFGQGTKLEIKRTVAAPSVFIFPPSDEQLKSGTASWCLLNNFYPREAK VQWKVDNALQSGNSQESVTEQDSKDSTYSLSSTLTLSKADYEKHKVYA CEVTHQGLSSPVTKSFNRGEC (SEQ ID NO:140).

212. The immunoconjugate of any one of embodiments 182 to 200, wherein the EpCAM antibody or EpCAM-binding fragment thereof comprises:
(a) a heavy chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYYIHWVRQAPGQRLEYIG WIYPGNVYIQYNEKFKGRATLTADKSASTAYMELSSLRSEDTAVYYCARD GPWFAYWGQGTLVTVSSASTKGPSVFPLAPSSKSTSGGTAALGCLVKDY FPEPVTVSWNSGALTSGVHTFPAVLQSSGLYSLSSVVTVPSSSLGTQTYI CNVNHKPSNTKVDKKVEPKSCDKTHTCPPCPAPELLGGPSVFLFPPKPK DTLMISRTPEVTCVVDVSHEDPEVKFNWYVDGVEVHNAKTKPREEQY NSTYRWSVLTVLHQDWLNGKEYKCKVSNKALPAPIEKTISKAKGQPREP QVYTLPPSRDELTKNQVSLTCLVKGFYPSDIAVEWESNGQPENNYKTTP PVLDSDGSFFLYSKLTVDKSRWQQGNVFSCSVMHEALHNHYTQKSLSL SPG (SEQ ID NO:103); and
(b) a light chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVLTQTPLSLSVTPGQPASISCRSSKSLLHSDGFTYLYWFLQKPGQSPQ LLIYQTSNLASGVPDRFSSSGSGTDFTLKISRVEAEDVGVYYCAQNLELP NTFGQGTKLEIKRTVAAPSVFIFPPSDEQLKSGTASWCLLNNFYPREAK VQWKVDNALQSGNSQESVTEQDSKDSTYSLSSTLTLSKADYEKHKVYA CEVTHQGLSSPVTKSFNRGEC (SEQ ID NO:138).

213. The immunoconjugate of any one of embodiments 182 to 200, wherein the EpCAM antibody or EpCAM-binding fragment thereof comprises:
(a) a heavy chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYYIHWVRQAPGQRLEW MGWIYPGNVYIQYNEKFKGRVTITRDTSASTAYMELSSLRSEDTAVYYCA RDGPWFAYWGQGTLVTVSSASTKGPSVFPLAPSSKSTSGGTAALGCLVK DYFPEPVTVSWNSGALTSGVHTFPAVLQSSGLYSLSSWTVPSSSLGTQ TYICNVNHKPSNTKVDKKVEPKSCDKTHTCPPCPAPELLGGPSVFLFPPK PKDTLMISRTPEVTCVWDVSHEDPEVKFNWYVDGVEVHNAKTKPREE QYNSTYRWSVLTVLHQDWLNGKEYKCKVSNKALPAPIEKTISKAKGQPR EPQVYTLPPSRDELTKNQVSLTCLVKGFYPSDIAVEWESNGQPENNYKT TPPVLDSDGSFFLYSKLTVDKSRWQQGNVFSCSVMHEALHNHYTQKSL SLSPG (SEQ ID NO:105); and
(b) a light chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVMTQTPLSLSVTPGQPASISCRSSKSLLHSDGFTYLYWFLQKPGQSPQ LLIYQTSNLASGVPDRFSGSGSGTDFTLKISRVEAEDVGVYYCAQNLELP NTFGQGTKLEIKRTVAAPSVFIFPPSDEQLKSGTASWCLLNNFYPREAK VQWKVDNALQSGNSQESVTEQDSKDSTYSLSSTLTLSKADYEKHKVYA CEVTHQGLSSPVTKSFNRGEC (SEQ ID NO:139).

214. The immunoconjugate of any one of embodiments 182 to 200, wherein the EpCAM antibody or EpCAM-binding fragment thereof comprises:
(a) a heavy chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYYIHWVRQAPGQRLEYIG WIYPGNVYIQYSQKFQGRVTITADKSASTAYMELSSLRSEDTAVYYCARD GPWFAYWGQGTLVTVSSASTKGPSVFPLAPSSKSTSGGTAALGCLVKDY FPEPVTVSWNSGALTSGVHTFPAVLQSSGLYSLSSVVTVPSSSLGTQTYI CNVNHKPSNTKVDKKVEPKSCDKTHTCPPCPAPELLGGPSVFLFPPKPK DTLMISRTPEVTCVVDVSHEDPEVKFNWYVDGVEVHNAKTKPREEQY NSTYRWSVLTVLHQDWLNGKEYKCKVSNKALPAPIEKTISKAKGQPREP QVYTLPPSRDELTKNQVSLTCLVKGFYPSDIAVEWESNGQPENNYKTTP PVLDSDGSFFLYSKLTVDKSRWQQGNVFSCSVMHEALHNHYTQKSLSL SPG (SEQ ID NO:106); and
(b) a light chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVMTQTPLSLSVTPGQPASISCRSSKSLLHSDGFTYLYWFLQKPGQSPQ LLIYQTSNLASGVPDRFSGSGSGTDFTLKISRVEAEDVGVYYCAQNLELP NTFGQGTKLEIKRTVAAPSVFIFPPSDEQLKSGTASWCLLNNFYPREAK VQWKVDNALQSGNSQESVTEQDSKDSTYSLSSTLTLSKADYEKHKVYA CEVTHQGLSSPVTKSFNRGEC (SEQ ID NO:139).

215. The immunoconjugate of any one of embodiments 182 to 200, wherein the EpCAM antibody or EpCAM-binding fragment thereof comprises:
(a) a heavy chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYYIHWVRQAPGQRLEW MGWIYPGNVYIQYNEKFKGRVTITRDTSASTAYMELSSLRSEDTAVYYCA RDGPWFAYWGQGTLVTVSSASTKGPSVFPLAPSSKSTSGGTAALGCLVK DYFPEPVTVSWNSGALTSGVHTFPAVLQSSGLYSLSSWTVPSSSLGTQ TYICNVNHKPSNTKVDKKVEPKSCDKTHTCPPCPAPELLGGPSVFLFPPK PKDTLMISRTPEVTCVWDVSHEDPEVKFNWYVDGVEVHNAKTKPREE QYNSTYRWSVLTVLHQDWLNGKEYKCKVSNKALPAPIEKTISKAKGQPR EPQVYTLPPSRDELTKNQVSLTCLVKGFYPSDIAVEWESNGQPENNYKT TPPVLDSDGSFFLYSKLTVDKSRWQQGNVFSCSVMHEALHNHYTQKSL SLSPG (SEQ ID NO:105); and
(b) a light chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVLTQTPLSLSVTPGQPASISCRSSRSLLHSDGFTYLYWFLQKPGQSPQ LLIYQTSNLASGVPDRFSSSGSGTDFTLKISRVEAEDVGVYYCAQNLELP NTFGQGTKLEIKRTVAAPSVFIFPPSDEQLKSGTASWCLLNNFYPREAK VQWKVDNALQSGNSQESVTEQDSKDSTYSLSSTLTLSKADYEKHKVYA CEVTHQGLSSPVTKSFNRGEC (SEQ ID NO:140).

216. The immunoconjugate of any one of embodiments 182 to 200, wherein the EpCAM antibody or EpCAM-binding fragment thereof comprises:
(a) a heavy chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYYIHWVRQAPGQRLEYIG WIYPGNVYIQYSQKFQGRVTITADKSASTAYMELSSLRSEDTAVYYCARD GPWFAYWGQGTLVTVSSASTKGPSVFPLAPSSKSTSGGTAALGCLVKDY FPEPVTVSWNSGALTSGVHTFPAVLQSSGLYSLSSVVTVPSSSLGTQTYI CNVNHKPSNTKVDKKVEPKSCDKTHTCPPCPAPELLGGPSVFLFPPKPK DTLMISRTPEVTCVVVDVSHEDPEVKFNWYVDGVEVHNAKTKPREEQY NSTYRWSVLTVLHQDWLNGKEYKCKVSNKALPAPIEKTISKAKGQPREP QVYTLPPSRDELTKNQVSLTCLVKGFYPSDIAVEWESNGQPENNYKTTP PVLDSDGSFFLYSKLTVDKSRWQQGNVFSCSVMHEALHNHYTQKSLSL SPG (SEQ ID NO:106); and
(b) a light chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVLTQTPLSLSVTPGQPASISCRSSRSLLHSDGFTYLYWFLQKPGQSPQ LLIYQTSNLASGVPDRFSSSGSGTDFTLKISRVEAEDVGVYYCAQNLELP NTFGQGTKLEIKRTVAAPSVFIFPPSDEQLKSGTASWCLLNNFYPREAK VQWKVDNALQSGNSQESVTEQDSKDSTYSLSSTLTLSKADYEKHKVYA CEVTHQGLSSPVTKSFNRGEC (SEQ ID NO:140).

217. The immunoconjugate of any one of embodiments 182 to 200, wherein the EpCAM antibody or EpCAM-binding fragment thereof comprises:
(a) a VH-CDR1 comprising the amino acid sequence NYHIH (SEQ ID NO:22);
(b) a VH-CDR2 comprising the amino acid sequence WIYPGNVYIQYNEKFKG (SEQ ID NO:14);
(c) a VH-CDR3 comprising the amino acid sequence DGPWFAY (SEQ ID NO:15);
(d) a VL-CDR1 comprising the amino acid sequence RSSRSLLHSDGFTYLY (SEQ ID NO:42);
(e) a VL-CDR2 comprising the amino acid sequence QTSNLAS (SEQ ID NO:40); and
(f) a VL-CDR3 comprising the amino acid sequence AQNLELPNT (SEQ ID NO:41).

218. The immunoconjugate of any one of embodiments 182 to 200, wherein the EpCAM antibody or EpCAM-binding fragment thereof comprises:
(a) a VH-CDR1 comprising the amino acid sequence NYYIH (SEQ ID NO:13);
(b) a VH-CDR2 comprising the amino acid sequence WIYPGNVYIQYNEKFKG (SEQ ID NO:14);
(c) a VH-CDR3 comprising the amino acid sequence DGYWFAY (SEQ ID NO:33);
(d) a VL-CDR1 comprising the amino acid sequence RSSRSLLHSDGFTYLY (SEQ ID NO:42);
(e) a VL-CDR2 comprising the amino acid sequence QTSNLAS (SEQ ID NO:40); and
(f) a VL-CDR3 comprising the amino acid sequence AQNLELPNT (SEQ ID NO:41).

219. The immunoconjugate of any one of embodiments 182 to 200, wherein the EpCAM antibody or EpCAM-binding fragment thereof comprises:
(a) a VH-CDR1 comprising the amino acid sequence NYDIH (SEQ ID NO:23);
(b) a VH-CDR2 comprising the amino acid sequence WIYPGNVYIQYNEKFKG (SEQ ID NO:14);
(c) a VH-CDR3 comprising the amino acid sequence DGPWFAY (SEQ ID NO:15);
(d) a VL-CDR1 comprising the amino acid sequence RSSRSLLHSDGFTYLY (SEQ ID NO:42);
(e) a VL-CDR2 comprising the amino acid sequence QTSNLAS (SEQ ID NO:40); and
(f) a VL-CDR3 comprising the amino acid sequence AQNLELPNT (SEQ ID NO:41).

220. The immunoconjugate of any one of embodiments 182 to 200, wherein the EpCAM antibody or EpCAM-binding fragment thereof comprises:
(a) a VH-CDR1 comprising the amino acid sequence NYLIH (SEQ ID NO:25);
(b) a VH-CDR2 comprising the amino acid sequence WIYPGNVYIQYNEKFKG (SEQ ID NO:14);
(c) a VH-CDR3 comprising the amino acid sequence DGPWFAY (SEQ ID NO:15);
(d) a VL-CDR1 comprising the amino acid sequence RSSRSLLHSDGFTYLY (SEQ ID NO:42);
(e) a VL-CDR2 comprising the amino acid sequence QTSNLAS (SEQ ID NO:40); and
(f) a VL-CDR3 comprising the amino acid sequence AQNLELPNT (SEQ ID NO:41).

221. The immunoconjugate of any one of embodiments 182 to 200 and 217 to 220, wherein the EpCAM antibody or EpCAM-binding fragment thereof comprises:
(a) a heavy chain variable domain (VH) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYHIHWVRQAPGQRLEYI GWIYPGNVYIQYNEKFKGRATLTADKSASTAYMELSSLRSEDTAVYYCAR DGPWFAYWGQGTLVTVSS (SEQ ID NO:75); and
(b) a light chain variable domain (VL) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVLTQTPLSLSVTPGQPASISCRSSRSLLHSDGFTYLYWFLQKPGQSPQ LLIYQTSNLASGVPDRFSSSGSGTDFTLKISRVEAEDVGVYYCAQNLELP NTFGQGTKLEIK (SEQ ID NO:89).

222. The immunoconjugate of any one of embodiments 182 to 200 and 217 to 220, wherein the EpCAM antibody or EpCAM-binding fragment thereof comprises:
(a) a heavy chain variable domain (VH) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYYIHWVRQAPGQRLEYIG WIYPGNVYIQYNEKFKGRATLTADKSASTAYMELSSLRSEDTAVYYCARD GYWFAYWGQGTLVTVSS (SEQ ID NO:77); and
(b) a light chain variable domain (VL) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVLTQTPLSLSVTPGQPASISCRSSRSLLHSDGFTYLYWFLQKPGQSPQ LLIYQTSNLASGVPDRFSSSGSGTDFTLKISRVEAEDVGVYYCAQNLELP NTFGQGTKLEIK (SEQ ID NO:89).

223. The immunoconjugate of any one of embodiments 182 to 200 and 217 to 220, wherein the EpCAM antibody or EpCAM-binding fragment thereof comprises:
(a) a heavy chain variable domain (VH) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYDIHWVRQAPGQRLEYI GWIYPGNVYIQYNEKFKGRATLTADKSASTAYMELSSLRSEDTAVYYCAR DGPWFAYWGQGTLVTVSS (SEQ ID NO:76); and
(b) a light chain variable domain (VL) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVLTQTPLSLSVTPGQPASISCRSSRSLLHSDGFTYLYWFLQKPGQSPQ LLIYQTSNLASGVPDRFSSSGSGTDFTLKISRVEAEDVGVYYCAQNLELP NTFGQGTKLEIK (SEQ ID NO:89).

224. The immunoconjugate of any one of embodiments 182 to 200 and 217 to 220, wherein the EpCAM antibody or EpCAM-binding fragment thereof comprises:
(a) a heavy chain variable domain (VH) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYLIHWVRQAPGQRLEYIG WIYPGNVYIQYNEKFKGRATLTADKSASTAYMELSSLRSEDTAVYYCARD GPWFAYWGQGTLVTVSS (SEQ ID NO:84); and
(b) a light chain variable domain (VL) comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVLTQTPLSLSVTPGQPASISCRSSRSLLHSDGFTYLYWFLQKPGQSPQ LLIYQTSNLASGVPDRFSSSGSGTDFTLKISRVEAEDVGVYYCAQNLELP NTFGQGTKLEIK (SEQ ID NO:89).

225. The immunoconjugate of any one of embodiments 182 to 200 and 217 to 220, wherein the EpCAM antibody or EpCAM-binding fragment thereof comprises:
(a) a heavy chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYHIHWVRQAPGQRLEYI GWIYPGNVYIQYNEKFKGRATLTADKSASTAYMELSSLRSEDTAVYYCAR DGPWFAYWGQGTLVTVSSASTKGPSVFPLAPSSKSTSGGTAALGCLVKD YFPEPVTVSWNSGALTSGVHTFPAVLQSSGLYSLSSVVTVPSSSLGTQTY ICNVNHKPSNTKVDKKVEPKSCDKTHTCPPCPAPELLGGPSVFLFPPKPK DTLMISRTPEVTCWVDVSHEDPEVKFNWYVDGVEVHNAKTKPREEQY NSTYRWSVLTVLHQDWLNGKEYKCKVSNKALPAPIEKTISKAKGQPREP QVYTLPPSRDELTKNQVSLTCLVKGFYPSDIAVEWESNGQPENNYKTTP PVLDSDGSFFLYSKLTVDKSRWQQGNVFSCSVMHEALHNHYTQKSLSL SPG (SEQ ID NO:125); and
(b) a light chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVLTQTPLSLSVTPGQPASISCRSSRSLLHSDGFTYLYWFLQKPGQSPQ LLIYQTSNLASGVPDRFSSSGSGTDFTLKISRVEAEDVGVYYCAQNLELP NTFGQGTKLEIKRTVAAPSVFIFPPSDEQLKSGTASWCLLNNFYPREAK VQWKVDNALQSGNSQESVTEQDSKDSTYSLSSTLTLSKADYEKHKVYA CEVTHQGLSSPVTKSFNRGEC (SEQ ID NO:140).

226. The immunoconjugate of any one of embodiments 182 to 200 and 217 to 220, wherein the EpCAM antibody or EpCAM-binding fragment thereof comprises:
(a) a heavy chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYYIHWVRQAPGQRLEYIG WIYPGNVYIQYNEKFKGRATLTADKSASTAYMELSSLRSEDTAVYYCARD GYWFAYWGQGTLVTVSSASTKGPSVFPLAPSSKSTSGGTAALGCLVKDY FPEPVTVSWNSGALTSGVHTFPAVLQSSGLYSLSSVVTVPSSSLGTQTYI CNVNHKPSNTKVDKKVEPKSCDKTHTCPPCPAPELLGGPSVFLFPPKPK DTLMISRTPEVTCWVDVSHEDPEVKFNWYVDGVEVHNAKTKPREEQY NSTYRWSVLTVLHQDWLNGKEYKCKVSNKALPAPIEKTISKAKGQPREP QVYTLPPSRDELTKNQVSLTCLVKGFYPSDIAVEWESNGQPENNYKTTP PVLDSDGSFFLYSKLTVDKSRWQQGNVFSCSVMHEALHNHYTQKSLSL SPG (SEQ ID NO:127); and
(b) a light chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVLTQTPLSLSVTPGQPASISCRSSRSLLHSDGFTYLYWFLQKPGQSPQ LLIYQTSNLASGVPDRFSSSGSGTDFTLKISRVEAEDVGVYYCAQNLELP NTFGQGTKLEIKRTVAAPSVFIFPPSDEQLKSGTASWCLLNNFYPREAK VQWKVDNALQSGNSQESVTEQDSKDSTYSLSSTLTLSKADYEKHKVYA CEVTHQGLSSPVTKSFNRGEC (SEQ ID NO:140).

227. The immunoconjugate of any one of embodiments 182 to 200 and 217 to 220, wherein the EpCAM antibody or EpCAM-binding fragment thereof comprises:
(a) a heavy chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYDIHWVRQAPGQRLEYI GWIYPGNVYIQYNEKFKGRATLTADKSASTAYMELSSLRSEDTAVYYCAR DGPWFAYWGQGTLVTVSSASTKGPSVFPLAPSSKSTSGGTAALGCLVKD YFPEPVTVSWNSGALTSGVHTFPAVLQSSGLYSLSSWTVPSSSLGTQTY ICNVNHKPSNTKVDKKVEPKSCDKTHTCPPCPAPELLGGPSVFLFPPKPK DTLMISRTPEVTCVWDVSHEDPEVKFNWYVDGVEVHNAKTKPREEQY NSTYRWSVLTVLHQDWLNGKEYKCKVSNKALPAPIEKTISKAKGQPREP QVYTLPPSRDELTKNQVSLTCLVKGFYPSDIAVEWESNGQPENNYKTTP PVLDSDGSFFLYSKLTVDKSRWQQGNVFSCSVMHEALHNHYTQKSLSL SPG (SEQ ID NO:126); and
(b) a light chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVLTQTPLSLSVTPGQPASISCRSSRSLLHSDGFTYLYWFLQKPGQSPQ LLIYQTSNLASGVPDRFSSSGSGTDFTLKISRVEAEDVGVYYCAQNLELP NTFGQGTKLEIKRTVAAPSVFIFPPSDEQLKSGTASWCLLNNFYPREAK VQWKVDNALQSGNSQESVTEQDSKDSTYSLSSTLTLSKADYEKHKVYA CEVTHQGLSSPVTKSFNRGEC (SEQ ID NO:140).

228. The immunoconjugate of any one of embodiments 182 to 200 and 217 to 220, wherein the EpCAM antibody or EpCAM-binding fragment thereof comprises:
(a) a heavy chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence QVQLVQSGAEVKKPGASVKVSCKASGYTFTNYLIHWVRQAPGQRLEYIG WIYPGNVYIQYNEKFKGRATLTADKSASTAYMELSSLRSEDTAVYYCARD GPWFAYWGQGTLVTVSSASTKGPSVFPLAPSSKSTSGGTAALGCLVKDY FPEPVTVSWNSGALTSGVHTFPAVLQSSGLYSLSSVVTVPSSSLGTQTYI CNVNHKPSNTKVDKKVEPKSCDKTHTCPPCPAPELLGGPSVFLFPPKPK DTLMISRTPEVTCVVVDVSHEDPEVKFNWYVDGVEVHNAKTKPREEQY NSTYRWSVLTVLHQDWLNGKEYKCKVSNKALPAPIEKTISKAKGQPREP QVYTLPPSRDELTKNQVSLTCLVKGFYPSDIAVEWESNGQPENNYKTTP PVLDSDGSFFLYSKLTVDKSRWQQGNVFSCSVMHEALHNHYTQKSLSL SPG (SEQ ID NO:134); and
(b) a light chain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence DIVLTQTPLSLSVTPGQPASISCRSSRSLLHSDGFTYLYWFLQKPGQSPQ LLIYQTSNLASGVPDRFSSSGSGTDFTLKISRVEAEDVGVYYCAQNLELP NTFGQGTKLEIKRTVAAPSVFIFPPSDEQLKSGTASWCLLNNFYPREAK VQWKVDNALQSGNSQESVTEQDSKDSTYSLSSTLTLSKADYEKHKVYA CEVTHQGLSSPVTKSFNRGEC (SEQ ID NO:140).

229. The immunoconjugate of any one of embodiments 182 to 228, wherein the EpCAM antibody or EpCAM-binding fragment thereof is a non-human mammal antibody.

230. The immunoconjugate of any one of embodiments 182 to 229, wherein the EpCAM antibody or EpCAM-binding fragment thereof is a murine antibody.

231. The immunoconjugate of any one of embodiments 182 to 228, wherein the EpCAM antibody or EpCAM-binding fragment thereof is a chimeric antibody.

232. The immunoconjugate of any one of embodiments 182 to 228, wherein the EpCAM antibody or EpCAM-binding fragment thereof is a humanized antibody.

233. The immunoconjugate of any one of embodiments 182 to 228, wherein the EpCAM antibody or EpCAM-binding fragment thereof is a human antibody.

234. The immunoconjugate of any one of embodiments 182 to 233, wherein the EpCAM antibody is a full-length antibody.

235. The immunoconjugate of embodiment 234, wherein the EpCAM antibody is a human IgG1 antibody.

236. The immunoconjugate of any one of embodiments 182 to 233, wherein the EpCAM antibody or EpCAM-binding fragment thereof is a Fab.

237. The immunoconjugate of any one of embodiments 182 to 233, wherein the EpCAM antibody or EpCAM-binding fragment thereof is a Fab′.

238. The immunoconjugate of any one of embodiments 182 to 233, wherein the EpCAM antibody or EpCAM-binding fragment thereof is a F(ab′)₂.

239. The immunoconjugate of any one of embodiments 182 to 233, wherein the EpCAM antibody or EpCAM-binding fragment thereof is an scFv.

240. The immunoconjugate of any one of embodiments 182 to 233, wherein the EpCAM antibody or EpCAM-binding fragment thereof is a disulfide-linked Fv (dsFv).

241. The immunoconjugate of any one of embodiments 182 to 240, wherein A is substituted with one or more polyols.

242. The immunoconjugate of embodiment 241, wherein the one or more polyols is —(C₁-C₆ alkylene)-X⁵-Y³, wherein X⁵ is —NR¹²C(═O)— or —C(═O)NR¹²;
wherein:
   Y³ is C₆ alkyl substituted with 5 OH groups; and
   R¹² is —H, C₁-C₆ alkyl, C₁-C₆ fluoroalkyl, C₃-C₆ cycloalkyl, aryl, heteroaryl, or benzyl.

243. The immunoconjugate of embodiment 241, wherein the one or more polyols is

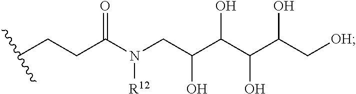

wherein R¹² is H or methyl.

244. The immunoconjugate of any one of embodiments 182 to 240, wherein the compound is

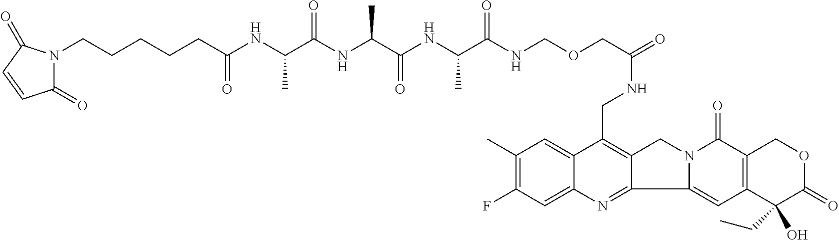 [SMILES: CC[C@@]1(O)C(=O)OCc2c1cc1n(c2=O)Cc2c-1nc1cc(F)c(C)cc1c2CNC(=O)COCNC(=O)[C@H](C)NC(=O)[C@H](C)NC(=O)[C@H](C)NC(=O)CCCCCN1C(=O)C=CC1=O]

or a pharmaceutically acceptable salt thereof.

245. The immunoconjugate of any one of embodiments 182 to 240, wherein the compound is

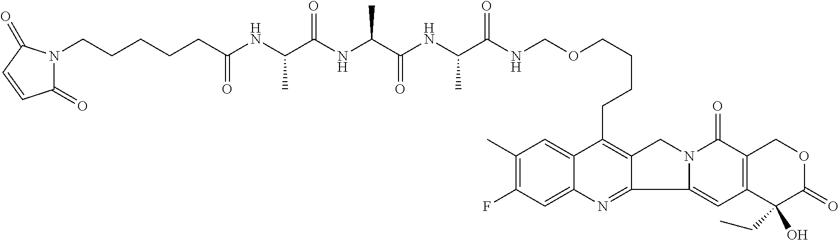 [SMILES: CC[C@@]1(O)C(=O)OCc2c1cc1n(c2=O)Cc2c-1nc1cc(F)c(C)cc1c2CCCCOCNC(=O)[C@H](C)NC(=O)[C@H](C)NC(=O)[C@H](C)NC(=O)CCCCCN1C(=O)C=CC1=O]

pharmaceutically acceptable salt thereof.

246. The immunoconjugate of any one of embodiments 86 to 245, having a DAR of at least 2.

247. The immunoconjugate of any one of embodiments 86 to 246, having a DAR of at least 3.

248. The immunoconjugate of any one of embodiments 86 to 247, having a DAR of at least 4.

249. The immunoconjugate of any one of embodiments 86 to 248, having a DAR of 4.

250. The immunoconjugate of any one of embodiments 86 to 249, having a DAR of at least 5.

251. The immunoconjugate of any one of embodiments 86 to 250, having a DAR of 5.

252. The immunoconjugate of any one of embodiments 86 to 251, having a DAR of at least 6.

253. The immunoconjugate of any one of embodiments 86 to 252, having a DAR of 6.

254. The immunoconjugate of any one of embodiments 86 to 253, having a DAR of at least 7.

255. The immunoconjugate of any one of embodiments 182 to 254, having a DAR of 7.

256. The immunoconjugate of any one of embodiments 182 to 255, having a DAR of at least 8

257. The immunoconjugate of any one of embodiments 182 to 256, having a DAR of 8.

258. The immunoconjugate of any one of embodiments 182 to 257, having a DAR of at least 9.

259. The immunoconjugate of any one of embodiments 182 to 258, having a DAR of 9.

260. The immunoconjugate of any one of embodiments 182 to 259, having a DAR of at least 10.

261. The immunoconjugate of any one of embodiments 182 to 260, having a DAR of 10.

262. The immunoconjugate of any one of embodiments 182 to 261, having a DAR of at least 11.

263. The immunoconjugate of any one of embodiments 182 to 262, having a DAR of 11.

264. The immunoconjugate of any one of embodiments 182 to 263, having a DAR of at least 12.

265. The immunoconjugate of any one of embodiments 182 to 264, having a DAR of 12.

266. An immunoconjugate comprising:
(a) an activatable antibody comprising:
   (i) an EpCAM antibody or EpCAM-binding fragment thereof, comprising:
      (11) a heavy chain complementarity determining region 1 (VH-CDR1) comprising the amino acid sequence NYYIH (SEQ ID NO:13);
      (12) a heavy chain complementarity determining region 2 (VH-CDR2) comprising the amino acid sequence WIYPGNVYIQYNEKFKG (SEQ ID NO:14);
      (13) heavy chain complementarity determining region 3 (CDR3) (VH-CDR3) comprising the amino acid sequence DGPWFAY (SEQ ID NO:15);
      (14) a light chain complementarity determining region 1 (VL-CDR1) comprising the amino acid sequence RSSRSLLHSDGFTYLY (SEQ ID NO:42);
      (15) a light chain complementarity determining region 2 (VL-CDR2) comprising the amino acid sequence QTSNLAS (SEQ ID NO:40); and
      (16) a light chain complementarity determining region 3 (VL-CDR3) comprising the amino acid sequence AQNLELPNT (SEQ ID NO:41);
   (ii) a cleavable moiety coupled to the activatable antibody wherein the EpCAM antibody or EpCAM-binding fragment thereof comprises the amino acid sequence AVGLLAPPGGLSGRSDNI (SEQ ID NO:168) or the amino acid sequence ISSGLLSGRSDNI (SEQ ID NO:169); and
   (iii) a masking moiety coupled to the activatable antibody wherein the EpCAM antibody or EpCAM-binding fragment thereof comprises the amino acid sequence WWPPCQGGAWCEQRI (SEQ ID NO:155), wherein the masking moiety inhibits the binding of the antibody or antibody fragment to EpCAM when the activatable antibody is in an uncleaved state,
wherein the activatable antibody in the uncleaved state has a structural arrangement from N-terminus to C-terminus of: (masking moiety)-(cleavable moiety)-(antibody or antibody fragment) or (antibody or antibody fragment)-(cleavable moiety)-(masking moiety); and
(b) at least one linker-payload comprising the compound (wherein the compound represents the linker-payload reactant, i.e. the linker-payload prior to conjugation to the EpCAM antibody or EpCAM-binding fragment thereof):

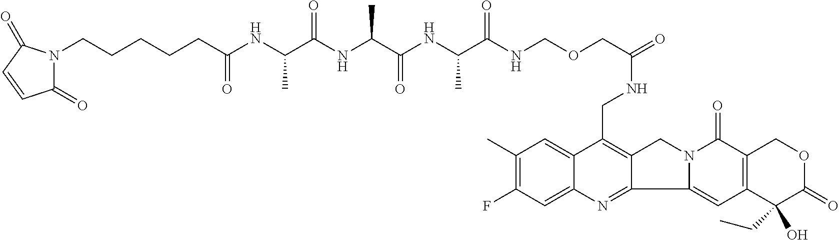 [SMILES: CC[C@@]1(O)C(=O)OCc2c1cc1n(c2=O)Cc2c-1nc1cc(F)c(C)cc1c2CNC(=O)COCNC(=O)[C@H](C)NC(=O)[C@H](C)NC(=O)[C@H](C)NC(=O)CCCCCN1C(=O)C=CC1=O]

or a pharmaceutically acceptable salt thereof.

267. A nucleic acid comprising a coding region for an EpCAM antibody or EpCAM-binding fragment thereof in the conjugate of any one of embodiments 97 to 266.

268. The nucleic acid of embodiment 267, comprising a coding region for a cleavable moiety and a masking moiety of any one of embodiments 166 to 266.

269. The nucleic acid of embodiment 267 or 268 in which the coding region is codon-optimized for expression in a host cell.

270. A vector comprising the nucleic acid of any one of embodiments 267 to 269.

271. The vector of embodiment 270, which is a viral vector.

272. A host cell engineered to express the nucleic acid of any one of embodiments 267 to 269 or the vector of embodiment 270 or embodiment 271.

273. A method of producing an immunoconjugate, the method comprising:
(a) culturing the host cell of embodiment 176;
(b) isolating the EpCAM antibody or EpCAM-binding fragment thereof from the host cell; and
(c) conjugating at least one linker-payload comprising a compound of Formula I or a pharmaceutically acceptable salt thereof to the EpCAM antibody or EpCAM-binding fragment thereof.

274. A diagnostic reagent comprising the immunoconjugate of any one of embodiments 1-170.

275. The diagnostic reagent of embodiment 178, wherein the immunoconjugate comprises a label.

276. The diagnostic reagent of embodiment 179, wherein the label is selected from: a radiolabel, a fluorophore, a chromophore, an imaging agent, or a metal ion.

277. A pharmaceutical composition comprising:
(a) the immunoconjugate of any one of embodiments 97 to 266; and
(b) a physiologically suitable buffer, adjuvant, diluent, or combination thereof.

278. A method of killing a cancer cell, comprising contacting the cancer cell with an effective amount of an immunoconjugate of any one of embodiments 97 to 266 or the pharmaceutical composition of embodiment 181.

279. The method of embodiment 278, wherein the cancer cell is an epithelial cancer cell.

280. The method of embodiment 278, wherein the cancer cell is a breast cancer cell.

281. The method of embodiment 278, wherein the cancer cell is a lung cancer cell.

282. The method of embodiment 278, wherein the cancer cell is a non-small cell lung cancer cell.

283. The method of embodiment 278, wherein the cancer cell is a stomach cancer cell.

284. The method of embodiment 278, wherein the cancer cell is a colorectal cancer cell.

285. The method of embodiment 278, wherein the cancer cell is a prostate cancer cell.

286. The method of embodiment 278, wherein the cancer cell is a bladder cancer cell.

287. The method of embodiment 278, wherein the cancer cell is an ovarian cancer cell.

288. The method of embodiment 278, wherein the cancer cell is a colon cancer cell.

289. The method of embodiment 278, wherein the cancer cell is a rectal cancer cell.

290. The method of embodiment 278, wherein the cancer cell is a cancer stem cell.

291. The method of embodiment 278, wherein the cancer cell is a uterine cancer cell.

292. The method of embodiment 278, wherein the cancer cell is a gastric cancer cell.

293. The method of embodiment 278, wherein the cancer cell is a pancreatic cancer cell.

294. A method of treating cancer comprising administering to a subject in need thereof an effective amount of the immunoconjugate of any one of embodiments 97 to 266 or the pharmaceutical composition of embodiment 277.

295. The method of embodiment 294, wherein the cancer expresses EpCAM.

296. The method of embodiment 294 or embodiment 295, wherein the cancer is epithelial cancer.

297. The method of embodiment 294 or embodiment 295, wherein the cancer is breast cancer.

298. The method of embodiment 294 or embodiment 295, wherein the cancer is lung cancer.

299. The method of embodiment 294 or embodiment 295, wherein the cancer is non-small cell lung cancer.

300. The method of embodiment 294 or embodiment 295, wherein the cancer is stomach cancer.

301. The method of embodiment 294 or embodiment 295, wherein the cancer is colorectal cancer.

302. The method of embodiment 294 or embodiment 295, wherein the cancer is prostate cancer.

303. The method of embodiment 294 or embodiment 295, wherein the cancer is bladder cancer.

304. The method of embodiment 294 or embodiment 295, wherein the cancer is ovarian cancer.

305. The method of embodiment 294 or embodiment 295, wherein the cancer is colon cancer.

306. The method of embodiment 294 or embodiment 295, wherein the cancer is rectal cancer.

307. The method of embodiment 294 or embodiment 295, wherein the cancer is uterine cancer.

308. The method of embodiment 294 or embodiment 295, wherein the cancer is gastric cancer.

309. The method of embodiment 294 or embodiment 295, wherein the cancer is pancreatic cancer.

310. The immunoconjugate of any one of embodiments 97 to 266 or the pharmaceutical composition of embodiment 277 for use in the treatment of a cancer.

311. The method of embodiment 310, wherein the cancer expresses EpCAM.

312. The method of embodiment 310 or embodiment 311, wherein the cancer is epithelial cancer.

313. The method of embodiment 310 or embodiment 311, wherein the cancer is breast cancer.

314. The method of embodiment 310 or embodiment 311, wherein the cancer is lung cancer.

315. The method of embodiment 310 or embodiment 311, wherein the cancer is non-small cell lung cancer.

316. The method of embodiment 310 or embodiment 311, wherein the cancer is stomach cancer.

317. The method of embodiment 310 or embodiment 311, wherein the cancer is colorectal cancer.

318. The method of embodiment 310 or embodiment 311, wherein the cancer is prostate cancer.

319. The method of embodiment 310 or embodiment 311, wherein the cancer is bladder cancer.

320. The method of embodiment 310 or embodiment 311, wherein the cancer is ovarian cancer.

321. The method of embodiment 310 or embodiment 311, wherein the cancer is colon cancer.

322. The method of embodiment 310 or embodiment 311, wherein the cancer is rectal cancer.

323. The method of embodiment 310 or embodiment 311, wherein the cancer is uterine cancer.

324. The method of embodiment 310 or embodiment 311, wherein the cancer is gastric cancer.

325. The method of embodiment 310 or embodiment 311, wherein the cancer is pancreatic cancer.

326. Use of the immunoconjugate of any one of embodiments 97 to 266 in the manufacture of a medicament for the treatment of a cancer.

327. The use of embodiment 326, wherein the cancer expresses EpCAM.

328. The method of embodiment 326 or embodiment 327, wherein the cancer is epithelial cancer.

329. The method of embodiment 326 or embodiment 327, wherein the cancer is breast cancer.

330. The method of embodiment 326 or embodiment 327, wherein the cancer is lung cancer.

331. The method of embodiment 326 or embodiment 327, wherein the cancer is non-small cell lung cancer.

332. The method of embodiment 326 or embodiment 327, wherein the cancer is stomach cancer.

333. The method of embodiment 326 or embodiment 327, wherein the cancer is colorectal cancer.

334. The method of embodiment 326 or embodiment 327, wherein the cancer is prostate cancer.

335. The method of embodiment 326 or embodiment 327, wherein the cancer is bladder cancer.

336. The method of embodiment 326 or embodiment 327, wherein the cancer is ovarian cancer.

337. The method of embodiment 326 or embodiment 327, wherein the cancer is colon cancer.

338. The method of embodiment 326 or embodiment 327, wherein the cancer is rectal cancer.

339. The method of embodiment 326 or embodiment 327, wherein the cancer is uterine cancer.

340. The method of embodiment 326 or embodiment 327, wherein the cancer is gastric cancer.

341. The method of embodiment 326 or embodiment 327, wherein the cancer is pancreatic cancer.

The present disclosure has been described above with the aid of exemplary headings and other functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments, will so fully reveal the general nature of the encompassed compositions and methods that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. The phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the following claims and their equivalents.

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

1. An immunoconjugate comprising:
(a) an activatable antibody comprising a full length human IgG1 EpCAM antibody having eight cysteines which, when in a native conformation, form four interchain disulfide bonds, the full length human IgG1 EpCAM antibody comprising a heavy chain comprising the amino acid sequence of SEQ ID NO:103 and a light chain comprising the amino acid sequence of SEQ ID NO:179; and
(b) eight linker-payloads depicted by the structure: 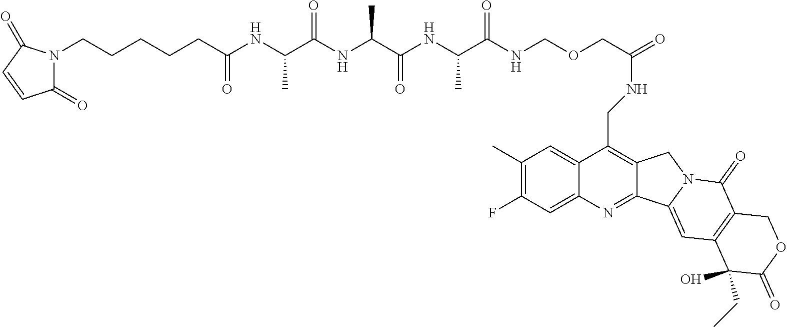 [SMILES: CC[C@@]1(O)C(=O)OCc2c1cc1n(c2=O)Cc2c-1nc1cc(F)c(C)cc1c2CNC(=O)COCNC(=O)[C@H](C)NC(=O)[C@H](C)NC(=O)[C@H](C)NC(=O)CCCCCN1C(=O)C=CC1=O]
or a pharmaceutically acceptable salt thereof, wherein each of the eight linker-payloads is individually covalently bound to the activatable antibody via one of the eight cysteines.

2. An immunoconjugate comprising:
(a) an activatable antibody comprising:
(i) an EpCAM antibody or EpCAM-binding fragment thereof, comprising:
(1) a heavy chain complementarity determining region 1 (VH-CDR1) comprising the amino acid sequence NYYIH (SEQ ID NO:13);
(2) a heavy chain complementarity determining region 2 (VH-CDR2) comprising the amino acid sequence WIYPGNVYIQYNEKFKG (SEQ ID NO:14);
(3) a heavy chain complementarity determining region 3 (CDR3) (VH-CDR3) comprising the amino acid sequence DGPWFAY (SEQ ID NO:15);
(4) a light chain complementarity determining region 1 (VL-CDR1) comprising the amino acid sequence RSSRSLLHSDGFTYLY (SEQ ID NO:42);
(5) a light chain complementarity determining region 2 (VL-CDR2) comprising the amino acid sequence QTSNLAS (SEQ ID NO:40); and
(6) a light chain complementarity determining region 3 (VL-CDR3) comprising the amino acid sequence AQNLELPNT (SEQ ID NO:41);
(ii) a cleavable moiety coupled to the EpCAM antibody or EpCAM-binding fragment thereof that comprises the amino acid sequence AVGLLAPPGGLSGRSDNI (SEQ ID NO:168) or the amino acid sequence ISSGLLSGRSDNI (SEQ ID NO:169); and
(iii) a masking moiety coupled to the EpCAM antibody or EpCAM-binding fragment thereof that comprises the amino acid sequence WWPPCQGGAWCEQRI (SEQ ID NO:155), wherein the masking moiety inhibits the binding of the antibody or antibody fragment to EpCAM when the activatable antibody is in an uncleaved state,
wherein the activatable antibody in an uncleaved state has a structural arrangement from N-terminus to C-terminus of: (masking moiety)-(cleavable moiety)-(antibody or antibody fragment) or (antibody or antibody fragment)-(cleavable moiety)-(masking moiety); and
(b) at least one linker-payload depicted by the structure: 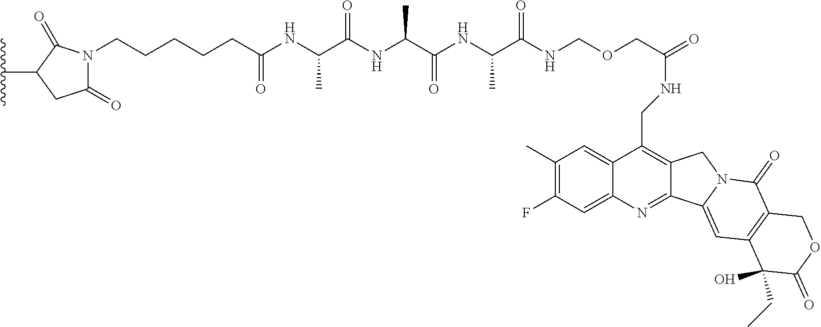 [SMILES: *C1CC(=O)N(CCCCCC(=O)N[C@@H](C)C(=O)N[C@@H](C)C(=O)N[C@@H](C)C(=O)NCOCC(=O)NCc2c3c(nc4cc(F)c(C)cc24)-c2cc4c(c(=O)n2C3)COC(=O)[C@]4(O)CC)C1=O]
or a pharmaceutically acceptable salt thereof.

3. The immunoconjugate of claim 2, wherein the cleavable moiety comprises the amino acid sequence ISSGLLSGRSDNI (SEQ ID NO:169).

4. The immunoconjugate of claim 2, wherein the cleavable moiety comprises the amino acid sequence AVGLLAPPGGLSGRSDNI (SEQ ID NO:168).

5. The immunoconjugate of any one of claims 2 to 4, wherein the immunoconjugate has a drug-to-antibody ratio (DAR) of 8.

6. The immunoconjugate of any one of claims 2 to 5, wherein the activatable antibody is a full length human IgG1 EpCAM antibody having eight cysteines which, when in a native conformation form four interchain disulfide bonds, and wherein each linker-payload is individually covalently bound to the activatable antibody via one of the eight cysteines.

7. An immunoconjugate comprising:
(a) an activatable antibody comprising:
(i) a full length human IgG1 EpCAM antibody having eight cysteines which, when in a native conformation, form four interchain disulfide bonds, the full length human IgG1 EpCAM antibody comprising:
(1) a heavy chain complementarity determining region 1 (VH-CDR1) comprising the amino acid sequence NYYIH (SEQ ID NO:13);
(2) a heavy chain complementarity determining region 2 (VH-CDR2) comprising the amino acid sequence WIYPGNVYIQYNEKFKG (SEQ ID NO:14);
(3) a heavy chain complementarity determining region 3 (CDR3) (VH-CDR3) comprising the amino acid sequence DGPWFAY (SEQ ID NO:15);
(4) a light chain complementarity determining region 1 (VL-CDR1) comprising the amino acid sequence RSSRSLLHSDGFTYLY (SEQ ID NO:42);
(5) a light chain complementarity determining region 2 (VL-CDR2) comprising the amino acid sequence QTSNLAS (SEQ ID NO:40); and
(6) a light chain complementarity determining region 3 (VL-CDR3) comprising the amino acid sequence AQNLELPNT (SEQ ID NO:41);
(ii) a cleavable moiety coupled to the EpCAM antibody or EpCAM-binding fragment thereof that comprises the amino acid sequence ISSGLLSGRSDNI (SEQ ID NO:169); and
(iii) a masking moiety coupled to the EpCAM antibody or EpCAM-binding fragment thereof that comprises the amino acid sequence WWPPCQGGAWCEQRI (SEQ ID NO:155), wherein the masking moiety inhibits the binding of the antibody or antibody fragment to EpCAM when the activatable antibody is in an uncleaved state,
wherein the activatable antibody in an uncleaved state has a structural arrangement from N-terminus to C-terminus of: (masking moiety)-(cleavable moiety)-(antibody) or (antibody)-(cleavable moiety)-(masking moiety); and
(b) eight linker-payloads each depicted by the structure: 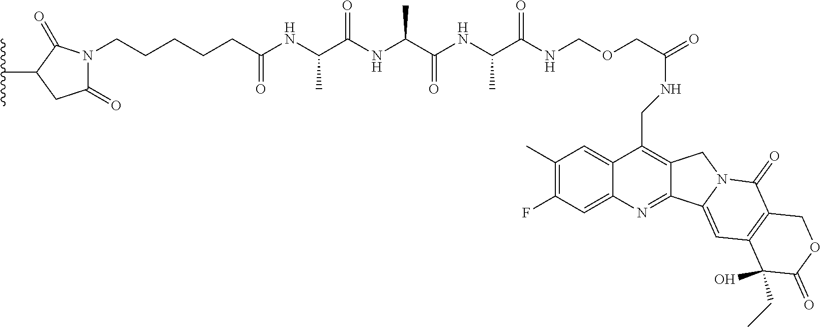 [SMILES: *C1CC(=O)N(CCCCCC(=O)N[C@@H](C)C(=O)N[C@@H](C)C(=O)N[C@@H](C)C(=O)NCOCC(=O)NCc2c3c(nc4cc(F)c(C)cc24)-c2cc4c(c(=O)n2C3)COC(=O)[C@]4(O)CC)C1=O]
or a pharmaceutically acceptable salt thereof, wherein each of the eight linker-payloads is individually covalently bound to the activatable antibody via one of the eight cysteines.

8. The immunoconjugate of claim 7, wherein the full length human IgG1 EpCAM antibody comprises a heavy chain variable domain (VH) comprising the amino acid sequence of SEQ ID NO:54 and a light chain variable domain (VL) comprising the amino acid sequence of SEQ ID NO: 89.

9. The immunoconjugate of claim 7, wherein the full length human IgG1 EpCAM antibody comprises a heavy chain comprising the amino acid sequence of SEQ ID NO:103 and a light chain comprising the amino acid sequence of SEQ ID NO:179.

10. An immunoconjugate obtainable by the method of Example 7.4.1.

11. An immunoconjugate obtainable by a method comprising:
(a) mixing an activatable EpCAM antibody consisting of a heterotetramer of two heavy chains each consisting of the amino acid sequence of SEQ ID NO:103 and two light chains each consisting of the amino acid sequence of SEQ ID NO:179 with a mixture of EPPS buffer and an aqueous EDTA solution to form an activatable EpCAM reaction mixture;
(b) mixing an activatable EpCAM antibody consisting of a heterotetramer of two heavy chains each consisting of the amino acid sequence of SEQ ID NO:103 and two light chains each consisting of the amino acid sequence of SEQ ID NO:179 with a mixture of EPPS buffer and an aqueous EDTA solution to form an activatable EpCAM reaction mixture; and
(c) mixing the reduced activatable EpCAM reaction mixture with a camptothecin linker-toxin stock solution comprising a linker-toxin represented by the structure 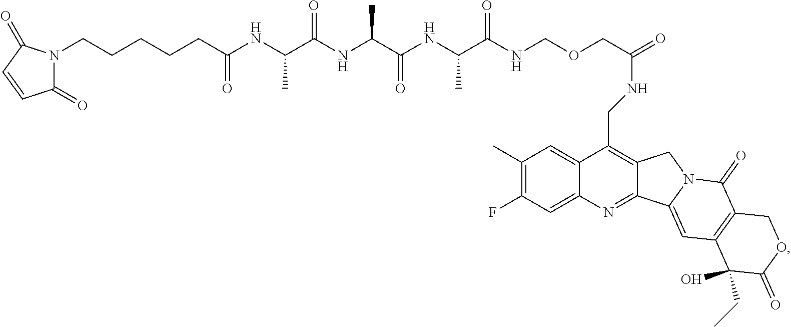 [SMILES: CC[C@@]1(O)C(=O)OCc2c1cc1n(c2=O)Cc2c-1nc1cc(F)c(C)cc1c2CNC(=O)COCNC(=O)[C@H](C)NC(=O)[C@H](C)NC(=O)[C@H](C)NC(=O)CCCCCN1C(=O)C=CC1=O]
thereby forming the immunoconjugate.

12. A nucleic acid comprising one or more coding regions for the activatable antibody of the immunoconjugate of any one of claims 1 to 11.

13. A vector comprising the nucleic acid of claim 12.

14. The vector of claim 13, which is a viral vector.

15. A method of producing an immunoconjugate, comprising:
(a) culturing a host cell comprising the vector of claim 14;
(b) isolating an activatable antibody from the host cell; and
(c) conjugating to the activatable antibody at least one linker-payload reactant comprising camptothecin or a derivative thereof.

16. The method of claim 15, wherein the linker-payload reactant comprises a compound of Formula I:
E-A-L-D (Formula I)
wherein:
E-A-Z′-L¹-D (Formula I),
wherein:
D is represented by the following structural formula: 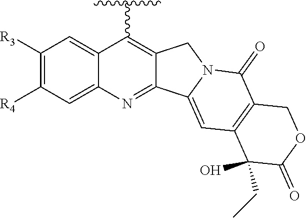
wherein R¹ is F and R² is methyl;
-L¹-Z′—* is —(C₁-C₄ alkylene)—O—CH₂—NR⁸—*, —(C₁-C₄ alkylene)-NR⁸—*, or —(C₁-C₅ alkylene)-NR⁵C(═O)—(C₁-C₅ alkylene)-O—CH₂—NR⁸—*, where * is a site covalently attached to A;
each R⁵ is independently H, methyl, or benzyl;
each R⁸ is independently H, methyl, or benzyl;
A is a peptide comprising 2 to 4 amino acids; and
E is —C(═O)—(C₁-C₁₀ alkylene)-X³ wherein X³ is

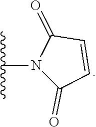 [SMILES: *N1C(=O)C=CC1=O]

17. The method of claim 15, wherein the linker-payload reactant is: 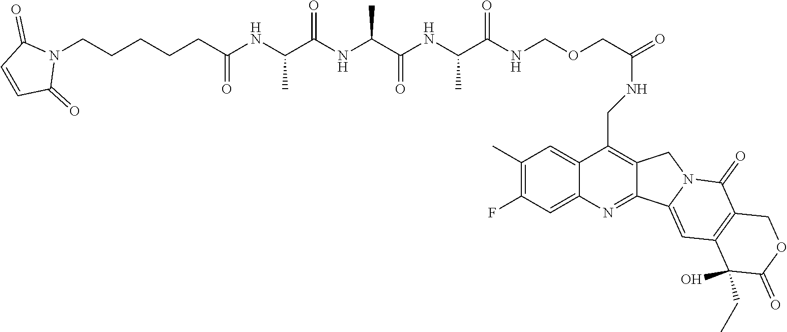 [SMILES: CC[C@@]1(O)C(=O)OCc2c1cc1n(c2=O)Cc2c-1nc1cc(F)c(C)cc1c2CNC(=O)COCNC(=O)[C@H](C)NC(=O)[C@H](C)NC(=O)[C@H](C)NC(=O)CCCCCN1C(=O)C=CC1=O]
or a pharmaceutically acceptable salt thereof.

18. The method of claim 15, wherein the linker-payload reactant is 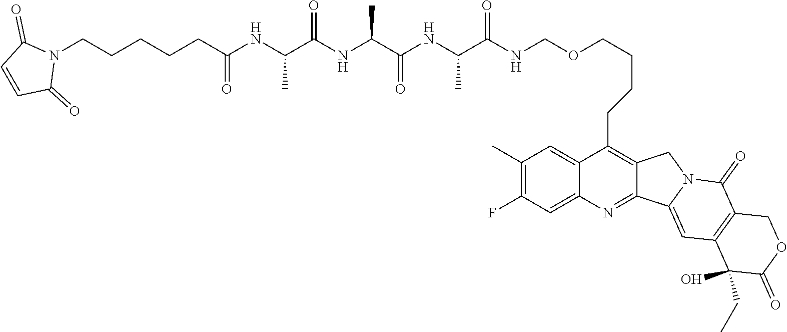 [SMILES: CC[C@@]1(O)C(=O)OCc2c1cc1n(c2=O)Cc2c-1nc1cc(F)c(C)cc1c2CCCCOCNC(=O)[C@H](C)NC(=O)[C@H](C)NC(=O)[C@H](C)NC(=O)CCCCCN1C(=O)C=CC1=O]
or a pharmaceutically acceptable salt thereof.

19. The method of claim 15, wherein the linker-payload reactant comprises exatecan.

20. The method of claim 15, wherein the linker-payload reactant comprises deruxtecan.

21. A method of producing an immunoconjugate, comprising conjugating at least one linker-payload reactant comprising camptothecin or a derivative thereof to the activatable antibody of any one of claims 1 to 11.

22. The method of claim 21, wherein the linker-payload reactant comprises a compound of Formula I:
E-A-Z′-L¹-D (Formula I),
wherein:
D is represented by the following structural formula: 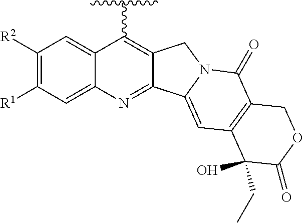
wherein R¹ is F and R² is methyl;
-L¹-Z′—* is —(C₁-C₄ alkylene)—O—CH₂—NR⁸—*, —(C₁-C₄ alkylene)-NR⁸—*, or —(C₁-C₅ alkylene)-NR⁵C(═O)—(C₁-C₅ alkylene)-O—CH₂—NR⁸—*, where * is a site covalently attached to A;
each R⁵ is independently H, methyl, or benzyl;
each R⁸ is independently H, methyl, or benzyl;
A is a peptide comprising 2 to 4 amino acids; and
E is —C(═O)—(C₁-C₁₀ alkylene)-X³ wherein X is

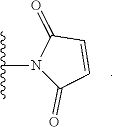 [SMILES: *N1C(=O)C=CC1=O]

23. The method of claim 21, wherein the linker-payload reactant is: 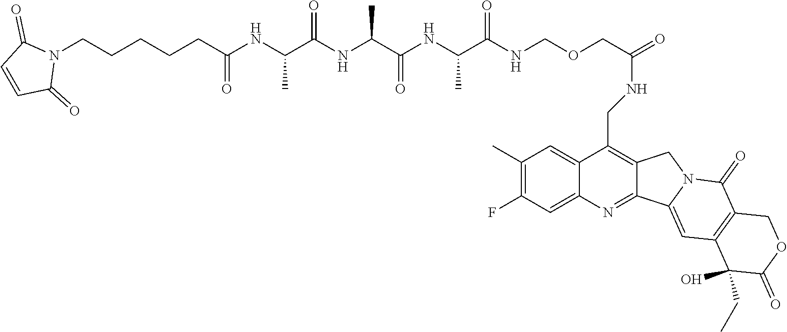 [SMILES: CC[C@@]1(O)C(=O)OCc2c1cc1n(c2=O)Cc2c-1nc1cc(F)c(C)cc1c2CNC(=O)COCNC(=O)[C@H](C)NC(=O)[C@H](C)NC(=O)[C@H](C)NC(=O)CCCCCN1C(=O)C=CC1=O]
or a pharmaceutically acceptable salt thereof.

24. The method of claim 21, wherein the linker-payload reactant is: 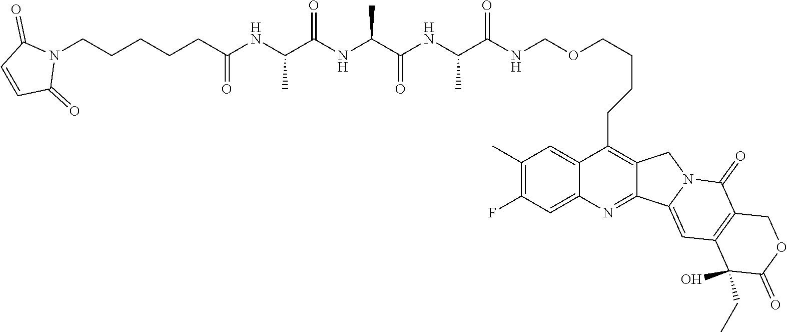 [SMILES: CC[C@@]1(O)C(=O)OCc2c1cc1n(c2=O)Cc2c-1nc1cc(F)c(C)cc1c2CCCCOCNC(=O)[C@H](C)NC(=O)[C@H](C)NC(=O)[C@H](C)NC(=O)CCCCCN1C(=O)C=CC1=O]
or a pharmaceutically acceptable salt thereof.

25. The method of claim 21, wherein the linker-payload reactant comprises exatecan.

26. The method of claim 21, wherein the linker-payload reactant comprises deruxtecan.

27. A composition comprising:
(a) the immunoconjugate of any one of claims 1 to 11; and
(b) a pharmaceutically acceptable carrier.

28. The immunoconjugate of any one of claims 1 to 11 or the composition of claim 27 for use in the treatment of an EpCAM-expressing cancer.
